(19) United States
(12) Patent Application Publication
(10) Pub. No.: US 2023/0242526 A1
(43) Pub. Date: Aug. 3, 2023

(54) Deuterated Pyridopyrimidinones And Their Use As Highly Selective Cyclin-Dependent Kinase 2 Inhibitors

(71) Applicant: Zentaur Therapeutics International Limited, KLN, Hong Kong (CN)

(72) Inventors: Jiaquan Wu, Jiangyin, Jiangsu (CN); Feng Fan, Jiangyin, Jiangsu (CN); Wenjun Gui, Jiangyin, Jiangsu (CN); Shuai Zhang, Jiangyin, Jiangsu (CN); Zhenghua Lu, Jiangyin, Jiangsu (CN); Wangdong Bian, Jiangyin, Jiangsu (CN); Minqi Gao, Jiangyin, Jiangsu (CN)

(21) Appl. No.: 18/009,504

(22) PCT Filed: Jun. 2, 2021
(86) PCT No.: PCT/CN2021/097920

§ 371 Date: Dec. 9, 2022

(30) Foreign Application Priority Data
Jun. 10, 2020 (CN) 202010570887.3

(51) Int. Cl. C07D 471/04; A61P 35/00; A61P 15/00

(52) U.S. Cl. CPC C07D 471/04; A61P 35/00; A61P 15/00

(57) ABSTRACT

The present invention relates to deuterated pyridopyrimidinone compounds and pharmaceutically acceptable salts thereof with inhibitory activity at cyclin dependent kinase 2 (CDK2) kinase, pharmaceutical composition comprising such compounds and methods of using such compounds for treating diseases associated with cell-cycle dysregulation.

### BACKGROUND OF THE INVENTION

Cell-cycle dysregulation is a hallmark of tumor cells. The ability of normal cells to undergo cell-cycle arrest after damage to DNA is crucial for the maintenance of genomic integrity. The biochemical pathways that stop the cell cycle in response to cellular stressors are called checkpoints. Cyclin-dependent kinases (CDKs) are serine/threonine kinases that drive cell cycle transitions. Their activities are regulated by their counterpart cyclins. Defective checkpoint function results in genetic modifications that contribute to tumorigenesis. Currently, there is an unmet need for such methods and agents that can be used for the treatment of such diseases and disorders.

### SUMMARY OF THE INVENTION

Accordingly, in various aspects, the present disclosure provides for deuterated pyridopyrimidinone compounds and pharmaceutically acceptable salts thereof with inhibitory activity upon cyclin-dependent kinase 2 (CDK2), pharmaceutical compositions comprising such compounds and methods of using such compounds for treating diseases associated with cell-cycle dysregulation.

In an embodiment, this disclosure provides a compound of Formula (1):

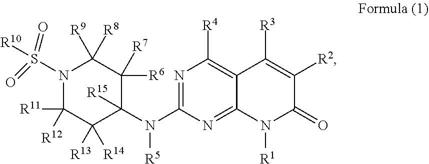

wherein,

R¹ is a cycloalkyl group having 5 to 6 carbon atoms optionally substituted with one or more substituents selected from the group of D (deuterium), F, —OH, and C1-C4 alkyl group optionally substituted with one or more D, —OH, —CN, —C(═O)—NH₂, CH₃—(CH₂)ₙ—O—, and C1-C4 fluoroalkoxyl group,

R² is a substituent selected from the group of H; D; F; Cl; Br; C1-C4 alkyl group optionally substituted with one or more D, —OH, —CN, Cl, —C(═O)—NH₂, CH₃—(CH₂)ₙ—O—, and C1-C4 fluoroalkoxyl group; and C1-C4 fluoroalkyl group optionally substituted with one or more D, —OH, —CN, —C(═O)—NH₂, CH₃—(CH₂)ₙ—O—, and C1-C4 fluoroalkoxyl group,

R³, R⁴, R⁵, R⁶, R⁷, R⁸, R⁹, R¹¹, R¹², R¹³, R¹⁴ and R¹⁵ are each independently hydrogen or D,

R¹⁰ is a substituent selected from the group of —NHR¹⁶; C1-C2 alkyl group optionally substituted with one or more of D, F, CH₃—(CH₂)ₙ—O—, C3-C5 cycloalkyl group, and C1-C2 fluoroalkoxyl group; C1-C2 fluoroalkyl group; cyclopropyl group;

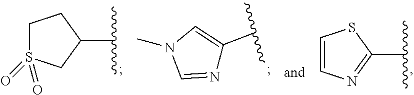

R¹⁶ is a substituent selected from the group of H, -Me (methyl), C1-C3 fluoroalkoxyl group,

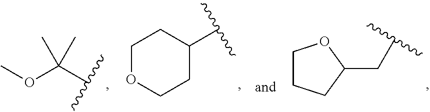

n is 0, 1, 2, or 3,

wherein the compound of Formula (1) is substituted with at least one deuterium atom, and

pharmaceutically acceptable salt, stereoisomer, cocrystal, prodrug, solvate, hydrate, or polymorph thereof.

In some embodiments, for the compounds of the Formula (1), R¹ is selected from the group of

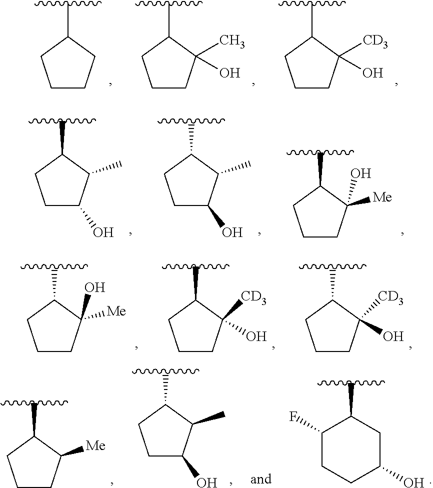

In some embodiments, for the compounds of the Formula (1), R¹ is

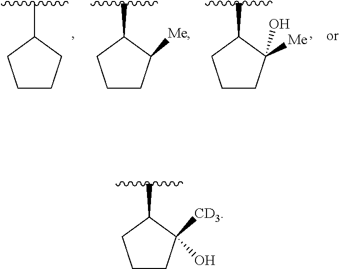

In some embodiments, for the compounds of the Formula (1), R¹ is

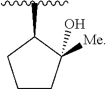 [SMILES: *[C@@H]1CCC[C@@]1(C)O]

In some embodiments, for the compounds of the Formula (1), R² is selected from the group of H, D, F, Cl, CH₃—, CH₃—CH₂—, —CH₂—OH, —CH₂—CN, —CH₂—C(═O)NH₂, —CH₂—CH₂—OH, —CH₂—CH₂—OMe, —CF₂H, —CFH₂, —CF₃, —CH₂—CF₂H, —CFD₂, —CF₂D, —CH₂—CF₂D, -CD₂-CF₂H, -CD₂-CF₂D, and —CD₃.

In some embodiments, for the compounds of the Formula (1), R² is selected from the group of CH₃—, CF₂H—, CF₂D-, and CD₃-.

In some embodiments, for the compounds of the Formula (1), R¹⁰ is a substituent selected from the group of —NH₂, —NHMe, —CH₃, —CH₂F, -CD₃, ethyl, cyclopropyl, and —CH₂—CH₂—OMe.

In some embodiments, for the compounds of the Formula (1), R¹⁰ is a substituent selected from the group of —CH₃, and —CD₃.

In some embodiments, for the compounds of the Formula (1), R⁶=R⁷=R¹³=R¹⁴=D.

In some embodiments, for the compounds of the Formula (1), R⁶=R⁷=R¹³=R¹⁴=R¹⁵=D.

In some embodiments, for the compounds of the Formula (1), R¹⁵=D.

In some embodiments, for the compounds of the Formula (1), R¹ is a substituent selected from the group of

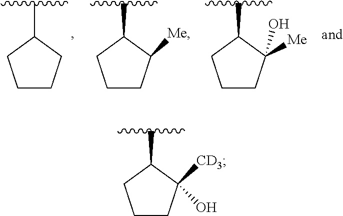

R² is a substituent selected from the group of CH₃—, CF₂H—, CF₂D-, and CD₃-; and R¹⁰ is a substituent selected from the group of —NH₂, —NHMe, —CH₃, —CH₂F, -CD₃, ethyl, cyclopropyl, and —CH₂—CH₂—OMe.

In some embodiments, for the compounds of the Formula (1), R¹ is

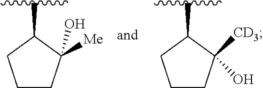 [SMILES: *[C@@H]1CCC[C@@]1(C)O ; *[C@@H]1CCC[C@]1(O)C([2H])([2H])[2H]]

R² is a substituent selected from the group of CH₃—, CF₂H—, CF₂D-, and CD₃-; and R¹⁰ is a substituent selected from the group of CH₃—, and CD₃-.

In some embodiments, for the compounds of the Formula (1), R¹ is

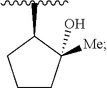 [SMILES: *[C@@H]1CCC[C@@]1(C)O]

R² is a substituent selected from the group of CH₃—, or CD₃-; and R¹⁰ is CH₃—.

In some embodiments, the compound of Formula (1) is selected from any one of the deuterated pyridopyrimidinone Comps. 7-213 disclosed in Table 1 below.

In an embodiment, this disclosure provides a pharmaceutical composition comprising one or more of the herein described deuterated pyridopyrimidinone compounds and at least one pharmaceutically acceptable carrier or diluent.

In some embodiments, the pharmaceutical composition further comprises an additional antineoplastic agent. In some embodiments, the additional antineoplastic agent is selected from the group of aromatase inhibitor, endocrine therapy, hormonal therapy, selective estrogen receptor degrader, cytotoxic agents, PD-1 antagonist, PD-L1 antagonist, AR inhibitor, inhibitor of glutaminase, CDK4/6 inhibitor, CDK9 inhibitor, and Akt inhibitor. In some embodiments, the additional antineoplastic agent is tamoxifen, docetaxel, paclitaxel, cisplatin, capecitabine, gemcitabine, vinorelbine, exemestane, letrozole, fulvestrant, anastrozole, or trastuzumab.

In some embodiments, any one of the herein described pharmaceutical composition comprises an oral dosage selected from the group of tablets, capsules, troches, pellets, granules, powders, solutions, syrups, elixirs, suspensions, and dispersions.

In some embodiments, any one of the herein described pharmaceutical composition comprises a unitary dosage form.

In some embodiments, any one of the herein described pharmaceutical composition comprises a mixture of about 90% or more by weight of the (−)-enantiomer and about 10% or less by weight of the (+)-enantiomer by the total weight of the herein described pharmaceutical composition.

In an embodiment, this disclosure provides a method for treating a cancer in a subject in need thereof (or use of any of the herein described deuterated pyridopyrimidinone compound for the manufacture of a medicament in the treatment of cancer) comprising administering to the subject a therapeutically effective amount of the herein described deuterated pyridopyrimidinone compound.

In some embodiments, the cancer treatable by the herein described deuterated pyridopyrimidinone compounds is selected from the group of breast cancer, triple negative breast cancer, ovarian cancer, neuroblastoma, glioblastoma, B-cell lymphoma, prostate cancer, liver cancer, acute myeloid leukemia, and melanoma.

In some embodiments, the cancer treatable by the herein described deuterated pyridopyrimidinone compounds is ovarian cancer. In some embodiments, the ovarian cancer treatable by the herein described deuterated pyridopyrimidinone compounds is epithelial ovarian cancer.

In some embodiments, the cancer treatable by the herein described deuterated pyridopyrimidinone compounds is breast cancer. In some embodiments, the breast cancer treatable by the herein described deuterated pyridopyrimidinone compounds is triple negative breast cancer. In some embodiments, the breast cancer treatable by the herein described deuterated pyridopyrimidinone compounds is HR-positive and HER2⁻ breast cancer. In some embodiments, the breast cancer treatable by the herein described deuterated pyridopyrimidinone compounds is ER⁺ HER2⁻ breast cancer. In some embodiments, the breast cancer treatable by the herein described deuterated pyridopyrimidinone compounds is tamoxifen resistant breast cancer.

In some embodiments, for any one of the herein described method or use, the subject is a mammal. In some embodiments, for any one of the herein described method or use, the subject is a human. In some embodiments, for any one of the herein described method or use, the human is an adult human. In some embodiments, for any one of the herein described method or use, the subject is a postmenopausal women or a premenopausal women with ERα⁺ HER2⁻ advanced or metastatic breast cancer.

In some embodiments, any one of the herein described method further comprises administering radiation, surgery, an additional antineoplastic agent, targeted therapy, immunotherapy, endocrine therapy, or hormonal therapy.

In some embodiments, any one of the herein described method comprises administering the herein described deuterated pyridopyrimidinone compounds via intravenous, intramuscular, intratumoral, intradermal, intraperitoneal, subcutaneous, oral, topical, intrathecal, inhalational, transdermal, or rectal administration.

In some embodiments, the herein described deuterated pyridopyrimidinone compounds used in any one of the herein described method is administered once daily.

In an embodiment, this disclosure provides uses of the herein described deuterated pyridopyrimidinone compounds for the treatment of a subject having a disease condition associated with overexpressing cyclin-dependent kinase 2 (CDK2), or cyclins modulated by CDK2 relative to an expression level in normal cells surrounding the cancer cells. In some embodiments, the disease condition associated with overexpressing CDK2, or cyclins modulated by CDK2 is selected from the group of breast cancer, triple negative breast cancer, ovarian cancer, neuroblastoma, glioblastoma, B-cell lymphoma, prostate cancer, liver cancer, acute myeloid leukemia, and melanoma.

### BRIEF DESCRIPTION OF THE DRAWINGS

FIG. 1 illustrates the CDKs, regulatory enzymes and cell cycle regulation.

FIG. 2 illustrates the treatment effects upon the body weights by the test compounds Comp. 4, Comp. 37, Comp. 5, Comp. 28, Comp. 163, and Comp. 164 evaluated on the growth of HCC1806 s.c. xenografts in BALB/c nude mice.

FIG. 3 illustrates the treatment effects upon the tumor volume by the test compounds Comp. 4, Comp. 37, Comp. 5, Comp. 28, Comp. 163, and Comp. 164 evaluated on the growth of HCC1806 s.c. xenografts in BALB/c nude mice.

FIG. 4 illustrates the treatment effects upon the relative tumor volume by the test compounds Comp. 4, Comp. 37, Comp. 5, Comp. 28, Comp. 163, and Comp. 164 evaluated on the growth of HCC1806 s.c. xenografts in BALB/c nude mice.

FIG. 5 illustrates the treatment effects upon the tumor weights by the test compounds Comp. 4, Comp. 37, Comp. 5, Comp. 28, Comp. 163, and Comp. 164 evaluated on the growth of HCC1806 s.c. xenografts in BALB/c nude mice.

### DETAILED DESCRIPTION OF THE INVENTION

### Definitions

As used in the preceding sections and throughout the rest of this specification, unless defined otherwise, all technical and scientific terms used herein have the same meaning as is commonly understood by one skilled in the art to which this invention belongs. All patents and publications referred to herein are incorporated by reference in their entireties.

All percentages, parts and ratios are based upon the total weight of the compounds or the composition of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” or “% w/w” herein.

As used herein, the term “alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range: e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, e.g., methyl (Me-), ethyl (Et-), n-propyl (Pr-), 1-methylethyl (isopropyl-), n-butyl (n-Bu-), n-pentyl, 1,1-dimethylethyl (t-butyl) and 3-methylhexyl.

Alkyl groups described herein as optionally substituted may be substituted by one or more substituent groups, which are selected independently unless otherwise indicated. The total number of substituent groups may equal the total number of hydrogen atoms on the alkyl moiety, to the extent such substitution makes chemical sense. Optionally substituted alklyl groups typically contain from 1 to 6 optional substituents, sometimes 1 to 5 optional substituents, preferably from 1 to 4 optional substituents, or more preferably from 1 to 3 optional substituents. Unless stated otherwise specifically in the specification, an alkyl group is optionally substituted by one or more of substituents which are independently selected from the group of heteroalkyl, alkenyl, alkynyl, cycloalkyl, heterocycloalkyl, aryl, arylalkyl, heteroaryl, heteroarylalkyl, hydroxy, thiol, halo, cyano (—CN), amino (—NH₂), oxo (=O), thiono (=S), trifluoromethyl, trifluoromethoxy, nitro (—NO₂), trimethylsilanyl, —OR^{a}, —SR^{a}, —S(O)ₜR^{a} (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or —PO₃(R^{a})₂, wherein each R^{a} is independently hydrogen, fluoroalkyl, cycloalkyl, carbocyclylalkyl, aryl, aralkyl, heterocycloalkyl, heteroaryl, or heteroarylalkyl. In some embodiments, the alkyl group is a C1-C4 alkyl group optionally substituted with one or more of deuterium or fluorine atom.

Typical substituent groups on alkyl include hydroxy, halo, C₁-C₄ alkoxy, cyano, C₆-C₁₂ aryl, 5-12 membered heteroaryl, 3-12 membered heterocycle, C₃-C₅ cycloalkyl, or —N(R^{a})₂, where each R^{a} is independently hydrogen, fluoroalkyl, cycloalkyl, carbocyclylalkyl, aryl, aralkyl, heterocycloalkyl, heteroaryl, or heteroarylalkyl. In some embodiments, alkyl is optionally substituted with 1 to 3 substituents which are independently selected from the group of hydroxy, halo, C₁-C₄ alkoxy, cyano, C₆-C₁₂ aryl, 5-12 membered heteroaryl, 3-12 membered heterocycle, C₃-C₈ cycloalkyl, and —N(R^{a})₂, where each R^{a} is independently hydrogen, fluoroalkyl, cycloalkyl, carbocyclylalkyl, aryl, aralkyl, heterocycloalkyl, heteroaryl, or heteroarylalkyl.

In some embodiments, substituted alkyl groups are named by reference to the substituent group, for example, “haloalkyl” refers to an alkyl group having the specific number of carbon atoms that is substituted by one or more halo substituents, and typically contain 1-6 carbon atoms, or preferably 1-4 carbon atoms or 1-2 carbon atoms and 1, 2, 3 halo atoms (e.g., C₁-C₆ haloalkyl, C₁-C₄ haloalkyl, or C₁-C₂ haloalkyl). More specifically, fluorinated alkyl groups may be specifically referred to as fluoroalkyl groups, e.g., C₁-C₆, C₁-C₄, or C₁-C₂ fluoroalkyl groups, which are typically substituted by 1, 2, or 3 fluoro atoms. Thus, a C₁-C₄ fluoroalkyl includes trifluoromethyl (—CF₃), difluoromethyl (—CF₂H), fluoromethyl (—CFH₂), difluoroethyl (—CH₂CF₂H), and the like.

As used herein, the term “alkenyl” refers to an alkyl group, as defined herein, consisting of at least two carbon atoms and at least one carbon-carbon double bond. Typically, alkenyl groups have 2 to 20 carbon atoms (“C2-C₂₀ alkenyl”), preferably 2 to 12 carbon atoms (“C2-C12 alkenyl”), more preferably 2 to 8 carbon atoms (“C2-C5 alkenyl”), or 2 to 6 carbon atoms (“C₂-C₆ alkenyl”), or 2 to 4 carbon atoms (“C₂-C₄ alkenyl”). Representative examples include, but are not limited to, ethenyl, 1-propenyl, 2-propenyl, 1-, 2-, or 3-butenyl, and the like. Alkenyl groups are unsubstituted or substituted by the same groups that are described herein as suitable for alkyl

As used herein, the term “alkynyl” refers to an alkyl group, as defined herein, consisting of at least two carbon atoms and at least one carbon-carbon triple bond. Alkynyl groups have 2 to 20 carbon atoms (C₂-C₂₀ alkynyl), preferably 2 to 12 carbon atoms (C₂ to C₁₂ alkynyl), more preferably 2 to 8 carbon atoms (C₂-C₈ alkynyl) or 2 to 6 carbon atoms (C₂-C₆ alkynyl), or 2 to 4 carbon atoms (C₂-C₄ alkynyl). Examples of alkynyl include, but are not limited to, ethynyl, 1-propynyl, 1-, 2-, or 3-butynyl, and the like. Alkynyl groups are unsubstituted or substituted by the same groups that are suitable for alkyl.

As used herein, the term “acyl” refers to a monovalent group —C(═O)-alkyl, wherein the alkyl portion has the specific number of carbon atoms (typically C₁-C₈, preferably C₁-C₆, or C₁-C₄) and are optionally substituted with the groups suitable for alkyl, e.g., F, OH or alkoxyl. The acyl group include both unsubstituted acyl groups such as —C(═O)—CH₃ (acetyl), as well as substituted acyl groups such as —C(═O)CF₃ (trifluoroacetyl), —C(═O)CH₂—OH (hydroxyacetyl), —C(═O)CH₂OCH₃ (methoxyacetyl), —C(═O)CF₂H (difluroracetyl) and the like.

As used herein, the term “aryl” refers to a benzene ring or a fused benzene ring system, for example anthracene, phenanthrene, or naphthalene ring systems. Examples of “aryl” groups include, but are not limited to, phenyl, biphenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl and the like. One preferred aryl group is phenyl. The aryl group is unsubstituted or substituted with those substituents as suitable for alkyl or cycloalkyl groups as described herein.

As used herein, the term “aralkyl” refers to an aryl group as described herein which is linked to the base molecule through an alkylene or similar linker. Arylarlkyl groups are described by the total number of carbon atoms in the ring and linkers. Thus a benzyl group is a C₇-arylalkyl group and a phenylethyl is a C₈-arylalkyl. Typically, arylalkyl group has 7-16 carbon atoms (C₇-C₁₆ arylalkyl), wherein the aryl portion has 6-12 carbon atoms and the alkylene portion has 1-4 carbon atoms. Such groups may also be represented as C₁-C₄ alkylene-C₆-C₁₂ aryl.

As used herein, the term “cycloalkyl” refers to a non-aromatic, monocyclic, spirocyclic, bridged or fused bicyclic, or polycyclic ring system that contains only carbon and hydrogen, and may be saturated, or partially unsaturated, and the ring system is connected to the base molecule through a carbon atom of the cycloalkyl ring. Cycloalkyl groups include groups having from 3 to 10 carbon atoms in the ring (i.e. (C₃₋₁₀) cycloalkyl or C₃₋₁₀ cycloalkyl). Typically, the cycloalkyl groups of the invention contains 3 to 12 carbon atoms, preferably 3 to 10 carbon atoms, more preferably 3 to 8 carbon atoms. Whenever it appears herein, a numerical range such as “3 to 10” refers to each integer in the given range, e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, cycloheptanyl, cycloheptenyl, octahydroindanyl, octahydropentalenyl, decahydronaphthalenyl, admantyl, bicycle-[1,1,1]pentanyl, bicycle-[2,2,1]-heptanyl, bicycle-[2,2,2]-octanyl, and the like. Unless stated otherwise specifically in the specification, a cycloalkyl group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, thiol, halo, cyano (—CN), amino (—NH₂), oxo (=O), thiono (=S), trifluoromethyl, trifluoromethoxy, nitro (—NO₂), trimethylsilanyl, —OR^{a}, —SR^{a}, —S(O)ₜR^{a} (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, wherein each R^{a} is independently hydrogen, alkyl, fluoroalkyl, cycloalkyl, cycloalkylalkyl, aryl, aralkyl, heterocycloalkyl, heteroaryl, or heteroarylalkyl. In some embodiments, cycloalkyl groups are unsubstituted or substituted by the same group as described herein as suitable for alkyl groups. In some embodiments, cycloalkyl groups used herein are unsubstituted or substituted cyclopentyl, or cyclohexyl.

As used herein, the term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

As used herein, the term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently selected from the group of alkyl, heteroalkyl, alkenyl, alkynyl, cycloalkyl, heterocycloalkyl, aryl, arylalkyl, heteroaryl, heteroarylalkyl, hydroxy, thiol, halo, cyano (—CN), amino (—NH₂), oxo (=O), thiono (=S), trifluoromethyl, trifluoromethoxy, nitro (—NO₂), trimethylsilanyl, —OR^{a}, —SR^{a}, —S(O)ₜR^{a} (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, wherein each R^{a} is independently hydrogen, alkyl, fluoroalkyl, cycloalkyl, cycloalkylalkyl, aryl, aralkyl, heterocycloalkyl, heteroaryl, or heteroarylalkyl.

As used herein, the term “amino” or “amine” refers to a —N(R^{a})₂ radical group, where each R^{a} is independently hydrogen, alkyl, fluoroalkyl, cycloalkyl, cycloalkylalkyl, aryl, aralkyl, heterocycloalkyl, heteroaryl or heteroarylalkyl, unless stated otherwise specifically in the specification. When a —N(R^{a})₂ group has two R^{a} substituents other than hydrogen, they can be combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, heteroalkyl, alkenyl, alkynyl, cycloalkyl, heterocycloalkyl, aryl, arylalkyl, heteroaryl, heteroarylalkyl, hydroxy, thiol, halo, cyano (—CN), amino (—NH₂), oxo (=O), thiono (=S), trifluoromethyl, trifluoromethoxy, nitro (—NO₂), trimethylsilanyl, —OR^{a}, —SR^{a}, —S(O)ₜR^{a} (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, wherein each R^{a} is independently hydrogen, alkyl, fluoroalkyl, cycloalkyl, cycloalkylalkyl, aryl, aralkyl, heterocycloalkyl, heteroaryl, or heteroarylalkyl.

As used herein, the term “substituted amino” also refers to N-oxides of the groups —NHR^{a}, and —NR^{a}R^{a} each as described above. N-oxides can be prepared by treatment of the corresponding amino group with, for example, hydrogen peroxide or m-chloroperoxybenzoic acid.

As used herein, the term “amide” or “amido” refers to a chemical moiety with formula —C(O)N(R)₂ or —NHC(O)R, where R is selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R₂ of —N(R)₂ of the amide may optionally be taken together with the nitrogen to which it is attached to form a 4-, 5-, 6- or 7-membered ring. Unless stated otherwise specifically in the specification, an amide or amido group is optionally substituted independently by one or more of the substituents as described herein for alkyl, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3rd Ed., John Wiley & Sons, New York, N.Y., 1999, which is incorporated herein by reference in its entirety.

As used herein, the term “cyano” refers to a —C—N group.

As used herein, the term “fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, —CH₂—CH₂—F, —CH₂—CF₃, and the like. The alkyl part of the fluoroalkyl radical may be optionally substituted as defined above for an alkyl group. As used herein, the term “substituted” means that the referenced group may have attached one or more additional groups, radicals or moieties individually and independently selected from the group of, for example, acyl, alkyl, alkylaryl, cycloalkyl, aralkyl, aryl, carbohydrate, carbonate (R^{a}—O—C(═O)—O—R^{a}), heteroaryl, heterocycloalkyl, hydroxy (—OH), alkoxy (Alkyl-O—), aryloxy (Ar—O—), mercapto (—SH), alkylthio (alkyl-S—), arylthio (Ar—S—), cyano, halo, carbonyl, ester, thiocarbonyl, isocyanato, thiocyanato, isothiocyanato, nitro (—NO₂), oxo, perhaloalkyl, perfluoroalkyl, phosphate, silyl, —S(O)ₜO_{w}R^{a} (where t is 1 or 2; w=0 or 1), —S(O)ₜN(R^{a})₂ (where t is 1 or 2), or PO₃(R^{a})₂, alkylsulfinyl, alkylsulfonyl, arylsulfinyl, arylsulfonyl, heteroarylsulfinyl, heteroarylsulfonyl, sulfonamidyl, sulfoxyl, sulfonate, urea, and amino, including mono- and di-substituted amino groups, and protected derivatives thereof; wherein each R^{a} is independently hydrogen, alkyl, fluoroalkyl, cycloalkyl, cycloalkylalkyl, aryl, aralkyl, heterocycloalkyl, heteroaryl, or heteroarylalkyl.

As used herein, the term “halo” or halogen refers to fluoro, chloro, bromo and iodo (F, Cl, Br, I). Preferably, halo refers to fluoro or chloro (F or Cl).

As used herein, the term “heteroaryl” refers to monocyclic or fused bicyclic or polycyclic ring systems having the well-known characteristics of aromaticity that contain the specific number of ring atoms and include at least one heteroatom selected from N, O and S as a ring member in an aromatic ring. The inclusion of a heteroatom permits aromaticity in 5-membered rings as well as 6-membered rings. Typically, heteroaryl groups contain 5 to 20 ring atoms (5-20 membered heteroaryl), preferably 5 to 14 ring atoms (5-14 membered heteroaryl), and more preferably 5 to 12 ring atoms (5-12 membered heteroaryl). Heteroaryl rings are attached to the base molecule via a ring atom of the heteroaromatic ring, such that aromaticity is maintained. Thus, 6-membered heteroaryl rings may be attached to the base molecule via a ring C or N atom. Heteroayl groups may also be fused to another aryl or heteroaryl ring, or fused to a saturated or partially unsaturated carbocyclic or heterocyclic ring, provided the point of attachment to the base molecule on such fused ring systems is an atom of the heteroaromatic portion of the ring system. Examples of unsubstituted heteroaryl groups often include, but are not limited to, pyrrolyl, furanyl, thiophenyl, pyrozolyl, imidazolyl, isoxazolyl, oxazolyl, isothiazolyl, thiazolyl, 1,2,3-triazoyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, benzofuranyl, benzothiophenyl, indolyl, benzimidazolyl, indazolyl, quinolonyl, isoquinolinyl, cinnolinyl, quinazolinyl, quinoxalinyl, phthalazinyl, 1,5-naphthyridinyl, 1,6-naphthyridinyl, 1,7-naphthyridinyl, 1,8-naphthyridinyl, 2,6-naphthyridinyl, 2,7-naphthyridinyl, purinyl, triazinyl, naphthryidinyl, carbazolyl, benzotrazolyl, pyrrolo-[2,3-b]-pyridinyl, pyrrolo-[2,3-c]-pyridinyl, pyrrolo-[3,2-c]-pyridinyl, pyrrolo-[3,2-b]-pyridinyl, imidazo-[4,5-b]-pyridinyl, imidazo-[4,5-c]-pyridinyl, pyrozolo-[4,3-d]-pyridinyl, pyrozolo-[4,3-c]-pyridinyl, pyrozolo-[3,4-c]-pyridinyl, pyrozolo-[3,4-b]-pyridinyl, isoindolyl, indolizinyl, imadazo-[1,2-a]-pyridinyl, imadazo-[1,5-a]-pyridinyl, pyrozolo-[1,5-a]-pyridinyl, pyrozolo-[1,2-b]-pyridinyl, pyrido-[3,2-d]-pyrimidinyl, pyrido-[4,3-d]-pyrimidinyl, pyrido-[2,3-d]-pyrimidinyl, pyrido-[2,3-b]-pyrazinyl, pyrido-[3,4-b]-pyrazinyl, pyrimido-[5,4-d]-pyrimidinyl, pyrazino-[2,3-b]-pyrazinyl, pyrimido-[4,5-d]-pyrimidinyl, and imadazo-[1,2-c]-pyridinyl. In some embodiments, 5- or 6-membered heteroaryl groups are selected from the group consisting of pyrrolyl, furanyl, thiophenyl, pyrazolyl, imidazoylyl, isoxazolyl, oxazolyl, isothiazolyl, thiazolyl, triazolyl, pyridinyl, and pyrimidinyl, pyrazinyl or pyridazinyl rings. The heteroaryl group is unsubstituted or substituted as further described herein.

Heteroayl moieties described herein as optionally substituted may be substituted by one or more substituent groups which are selected independently unless otherwise indicated. The total number of substituent groups may equal the total number of hydrogen atoms on the heteroaryl moiety, to the extent such substitution makes chemical sense and aromaticity is maintained in the case of aryl and heteroaryl rings.

Optionally substituted heteroaryl groups typically have from 1 to 5 optional substituents, preferably 1 to 4 optional substituents, preferably 1 to 3 optional substituents, or more preferably from 1-2 optional substituents. Optional groups suitable for heteroaryl rings include, but are not limited to: alkyl, heteroalkyl, alkenyl, alkynyl, cycloalkyl, heterocycloalkyl, aryl, arylalkyl, heteroaryl, heteroarylalkyl, hydroxy, thiol, halo, cyano (—CN), amino (—NH₂), oxo (=O), thiono (=S), trifluoromethyl, trifluoromethoxy, nitro (—NO₂), trimethylsilanyl, —OR^{a}, —SR^{a}, —S(O)ₜR^{a} (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, —N(R^{a})C(NR^{a})N(R^{a})₂, —N(R^{a})S(O)ₜR^{a} (where t is 1 or 2), —S(O)ₜOR^{a} (where t is 1 or 2), —S(O)ₜN(R^{a})₂ (where t is 1 or 2), or PO₃(R^{a})₂, wherein each R^{a} is independently hydrogen, alkyl, fluoroalkyl, cycloalkyl, cycloalkylalkyl, aryl, aralkyl, heterocycloalkyl, heteroaryl, or heteroarylalkyl.

As used herein, the term “heteroaralkyl” refers to a heteroaryl group as described herein that is attached to the base molecule through an alkylene linker, and differs from “arylalkyl” in that at least one ring atom of the aromatic moiety is a heteroatom selected from N, O and S. Heteroarylalkyl groups are sometimes described herein according to the total number of non-hydrogen atoms (e.g., C, N, S and O) in the ring and linker combined, excluding substituted groups. For example, pyridinylmethyl may be referred to as a “C₇”-heteroarylalkyl. Typically, unsubstituted heteroarylalkyl groups contain 6-20 non-hydrogen atoms (including C, N, S and O), wherein the heteroaryl portion typically has 5-12 atoms and the alkylene portion has 1-4 atoms, and it may expressed as —C₁-C₄ alkylene-5-12 membered heteroaryl. In some instances, heteroarylalkyl are described as -L-heteroarylalkyl, where the heteroarylalkyl group has the number of ring atoms indicated and -L- refers to an alkylene linker. It will be understood that when -L- is a bond, the group is heteroaryl.

As used herein, the term “carbohydrate” refers to any of the various neutral compounds of carbon, hydrogen, and oxygen such as sugars, starch, and cellulose. Carbohydrate may include monosaccharide, disaccharide, polysaccharide. Examples of carbohydrate may include, but not limited to, glucose, lactose, galactose, fructose, dextrose, maltose, maltotriose, maltooligosaccharides, sucrose, alpha-D-glucose, beta-D-glucose, starch, and modified starches.

As used herein, the term “cycloalkylalkyl” refers to a cycloalkyl group (e.g., C₃-C₈ cycloalkyl) connected to the base molecule through an alkylene linker, typically a C₁-C₄ alkylene. Cycloalkylalkyl groups are sometimes described by the total number of carbon atoms in the carbocyclic ring and linker, and typically contain from 4-12 carbon atoms (e.g., C₄-C₁₄ cycloalkylalkyl), for example, a cyclopropylmethyl group is a C₄-cycloalkylalkyl group and a cyclohexylethyl group is a C₈-cycloalkylalkyl group. Cycloalkylalkyl groups are unsubstituted or substituted on the cycloalkyl and or alkylene portions by the same groups that are described herein as suitable for alkyl groups.

As used herein, the term “heterocyclyl”, “heterocyclic” or “heteroalicyclic” are used interchangeably herein to refer to a non-aromatic, saturated or partially unsaturated ring system containing the specified number of ring atoms, including at least one heteroatom selected from N, O, and S as ring member, where ring S atoms are optionally substituted by one or two oxo groups (e.g., S(═O)ₓ, x is 0, 1 or 2) and where the heterocyclic ring is connected to the base molecule via ring atom, which may be C or N. Heterocyclic rings include rings which are spirocyclic, bridged, or fused to one or more other heterocyclic or carbocyclic rings, where such spirocyclic, bridged, or fused rings may themselves be saturated, partially unsaturated or aromatic to the extent unsaturation or aromaticity makes chemical sense, provided the point of attachment to the base molecule is an atom of the heterocyclic portion of the ring system. Preferably, heterocyclic ring contains 1 to 4 heteroatoms selected from N, O, and S(═O)ₓ as ring members, and more preferably 1 to 2 ring heteroatoms, provided that such heterocyclic rings do not contain two contiguous oxygen atoms. Heterocyclyl groups are unsubstituted or substituted with suitable substituent groups such as the same groups that are described herein as suitable for alkyl or cycloalkyl. Such substituents may be present on the heterocyclic ring attached to the base molecule or on a spirocyclic, bridged or fused ring attached thereto. In addition, ring nitrogen atoms are optionally substituted by groups suitable for an amine, e.g., alkyl, acyl, carbamoyl, sulfonyl, and the like.

Heterocycle typically includes 3-12 membered heterocyclyl groups, preferably 3-10 membered heterocyclyl groups, and more preferably 5-6 membered heterocyclyl groups, non-limited examples include: oriranyl, thiaranyl, aziridinyl, oxetanyl, thiatanyl, azetidinyl, tetrahydrofuranyl, tetrahydrothiophenyl, pyrrolidinyl, tetrahydropyranyl, tetrahydrothiopyranyl, piperidinyl, 1,4-dioxanyl, 1,4-oxathiaranyl, morpholinyl, 1,4-dithianyl, piperazinyl, thiomorpholinyl, oxepanyl, thiepanyl, azepanyl, 1,4-dioxepanyl, 1,4-oxathiepanyl, 1,4-oxaazepanyl, 1,4-thieazapanyl, 1,4-diazepanyl, or 1,4-dithiepanyl. Non-limiting example of partially unsaturated heterocycles include, but are not limited to 2H-pyranyl, 3,4-dihydro-2H-pyranyl, 5,6-dihydro-2H-pyranyl, 1,2,3,4-tetrahydropyridinyl, or 1,2,5,6-tetrahydropyridinyl. Non-limiting examples of bridged, fused and spiro heterocycles include: 2-oxa-5-azabicyclo-[2.2.1]-heptane, 3-oxa-8-azabicyclo-[3.2.1]-octane, 3-azabicylo-[3.1.0]-hexane, 8-azabicyclo-[2.2.1]-octane, 2-azabicyclo-[2.2.1]-heptane, 3-oxooctahydro-indolizine, and the like. In some embodiments, the heterocyclyl typically refers to heterocyclic groups contain 3-12 membered ring including both carbon and non-carbon heteroatoms, and preferably 4-7 membered rings. In some embodiments, the heterocyclyl groups containing 3-12 membered rings are selected from the group of azetidinyl, pyrrolidinyl, piperidinyl, piperazinyl, azepanyl, diazepanyl, tetrahydrofuranyl, tetrahydropyranyl, tetrahydrothiophenyl, tetrahydrothiopyranyl, morpholinyl and thiomorpholinyl rings and each of which are optionally substituted with particular substituents to the extent such substitution makes chemical sense.

As used herein, the term “heterocycloalkyl” may be used to describe a heterocyclic group of the specific size that is connected to the base molecule through an alkylene linker of the specific length. Typically, such group has an optionally substituted 3-12 membered heterocycle attached to the base molecule through a C₁-C₄ alkylene linker. The heterocycloalkyl may be optionally substituted with groups suitable for heterocyclic rings.

As used herein, the term “hydroxyl” refers to an “—OH” group.

As used herein, the term “oxo” refers to “=O” moiety.

The substituents themselves may be substituted, for example, a cycloalkyl substituent may itself have a halide substituent at one or more of its ring carbons. The term “optionally substituted” means optional substitution with the specified groups, radicals or moieties.

When any variable (e.g., R¹) occurs more than one time in any constituent for formula for a compound, its definition at each occurrence is independent of its definition at every other occurrence. For example, if a group is shown to be substituted with up to 0-2 R¹, then said group may optionally substituted with up to two R¹, the each R¹ occurrence is selected independently from the definition of R¹. When a substituent is listed without indicating the atom via which such substituent is bonded to the rest of the compound of a given formula, then such substituent may be bonded via any atom in such substituent. Combinations of substituents and/or variables are permissible only if such combinations results in a stable compound.

As used herein, the term “a”, “an”, or “the” generally is construed to cover both the singular and the plural forms.

As used herein, the term “about” as used herein, generally refers to a particular numeric value that includes variation and an acceptable error range as determined by one of ordinary skill in the art, which will depend in part on how the numeric value is measured or determined, i.e., the limitations of the measurement system. For example, “about” can mean zero variation, and a range of ±20%, ±10%, or ±5% of a given numeric value.

As used herein, the term “first-line therapy” refers to the first treatment given for a disease. It is often part of a standard set of treatments, such as surgery followed by chemotherapy and radiation. When used by itself, first-line therapy is the one accepted as the best treatment. If it doesn't cure the disease or it causes severe side effects, other treatment may be added or used instead. The “first-line therapy” is also known as induction therapy, primary therapy and primary treatment.

As used herein, the term “second-line therapy” refers to treatment that is given when initial treatment (first-line therapy) doesn't work, or stops working.

“Enantiomeric purity” (% ee) as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as at least 75% by weight, or such as at least 80% by weight. In some embodiments, the enrichment can be significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, or such as at least 95% by weight. The terms “enantiomerically pure” or “substantially enantiomerically pure” refers to a composition that comprises at least 98% of a single enantiomer and less than 2% of the opposite enantiomer.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; or enantiomers can be prepared by asymmetric syntheses. See, for example, Jacques et al. Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

As used herein, the term “elimination half-life” (also known as half-life, t_{1/2}) for a given compound measures the rate of change of the plasma concentration of the compound. The elimination half-life represents metabolism and excretion of the final fraction of the single dose, or the body residue after the period of multiple dosing, but it is controlled by the relatively slow efflux which occurs from tissue stores. A compound that is removed quickly from the systemic circulation and resides in small amounts within tissues will have a short half-life, translating to lower potential for side effects. Elimination half-life is defined by the equation t_{1/2}=ln(2)/k≈0.693/k. This indicates that elimination is 50% complete after one elimination half-life, 75% after two elimination half-lives, and about 99% complete after seven elimination half-lives. Since elimination represents the final removal of the compound from the body, the elimination half-life also serves as the determining factor of how much of the originally absorbed compound remains in the body, such that 50% of the absorbed compound remains after one elimination half-life, 25% remains after two elimination half-lives, and only about 1% remains after seven elimination half-lives. The elimination half-life is the half-life value reported in drug handbooks as an indication of how long a compound remains active in the body. With highly lipid-soluble compounds, it is usually measured as the half-life of plasma level decay at the very low levels which prevail in the time period 24-72 h after a single dose, or in approximately the same time period after the last dose of a series of multiple doses. In the first 24 h after a single dose, by any route of administration, and at virtually all times during long-term treatment, the most recent dose is in the process of reaching distribution equilibrium between tissues into which the compound penetrates slowly, and plasma water.

The term “carrier” refers to a diluent, adjuvant, excipient, or vehicle with which a compound is administered. Non-limiting examples of such pharmaceutical carriers include liquids, such as water and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Other examples of suitable pharmaceutical carriers are described in Remington: The Science and Practice of Pharmacy, 21st Edition (University of the Sciences in Philadelphia, ed., Lippincott Williams & Wilkins 2005). (hereby incorporated by reference in its entirety).

The term “isotopic variant” or “deuterium switch” as used herein means a compound obtained by substituting one or more hydrogen in a parent compound not comprising deuterium atoms by deuterium atoms. It is recognized that elements are present in natural isotopic abundances in most synthetic compounds, and result in inherent incorporation of deuterium. However, the natural isotopic abundance of hydrogen isotopes such as deuterium is immaterial (about 0.015%) relative to the degree of stable isotopic substitution of compounds indicated herein. Thus, as used herein, designation of an atom as deuterium at a position indicates that the abundance of deuterium is significantly greater than the natural abundance of deuterium. Any atom not designated as a particular isotope is intended to represent any stable isotope of that atom, as will be apparent to the ordinarily skilled artisan.

The term “in need thereof” refers to the need for symptomatic or asymptomatic relief from a condition such as, for example, breast cancer, triple negative breast cancer. The subject in need thereof may or may not be undergoing treatment for conditions related to, for example, breast cancer, triple negative breast cancer.

As used herein, the term “kinetic isotope effect” refers to an isotope effect caused by an isotopic substitution of one or more hydrogen atoms (H) by deuterium atoms (D) in a compound which may influence the reaction rate, e.g. metabolism of the compound. This is particularly the case when the isotopic replacement is in a chemical bond that is broken or formed in a rate limiting step.

As used herein, the term “metabolic switch” refers to changes in the metabolic profile of a particular drug due to deuterium incorporation, thus leading to different proportions of (or different) metabolites than observed with a non-deuterated analog of the same drug. The new metabolic profile may result in a distinct toxicological profile of the deuterated analog.

As used herein, the term “moiety” refers to a specific segment or functional group of a molecule. Chemical moieties are often recognized chemical entities embedded in or appended to a molecule.

As used herein, the term “oral administration” (P.O.) refers to a route of administration of drug where the drug is taken through the mouth. Per os abbreviated to P.O. is used as a direction for medication to be taken orally.

As used herein, the term “primary isotope effect” refers to the change in reaction rate, e.g. metabolism of the compound imparted by the isotopic replacement in a chemical bond that is broken or formed in a rate limiting step.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the term “Systemic Clearance” (CS) expressed as the volume of fluid cleared per unit time (L/h or m/min), is the sum of the clearance by various organs, primarily the liver where CSs are mostly cleared. The systemic clearance for most CSs such as BUD and FP (84 and 66 to 90 L/hr) approximates the maximum rate, at 90 L/hr determined by the hepatic blood flow, since the liver rapidly metabolizes the drug. However, for 17-BMP and des-CIC, which are converted metabolites, the apparent clearance values are much higher, suggesting extrahepatic metabolism. The faster the systemic clearance, the higher the therapeutic index and lower side-effect potential.

As used herein, the term “tautomers” refers to structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers can be reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1R)-one tautomers.

As used herein, the term “therapeutically effective amount” or “effective amount” refers to an amount of a compound that is sufficient to effect beneficial or desired results, including clinical results. As such, the effective amount may be sufficient, for example, to reduce or ameliorate the severity and/or duration of an affliction or condition, or one or more symptoms thereof, prevent the advancement of conditions related to an affliction or condition, prevent the recurrence, development, or onset of one or more symptoms associated with an affliction or condition, or enhance or otherwise improve the prophylactic or therapeutic effect(s) of another therapy. An effective amount also includes the amount of the compound that avoids or substantially attenuates undesirable side effects.

As used herein and as well understood in the art, “treatment” is an approach for obtaining beneficial or desired results, including clinical results. Beneficial or desired clinical results may include, but are not limited to, alleviation or amelioration of one or more symptoms or conditions, diminution of extent of disease, a stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

As used herein, the term “volume of distribution” (Vd) is a measure of drug distribution within tissues and is related to the lipophilicity of the drug. A large Vd does not necessarily imply greater potential for systemic effects, because CSs circulate primarily in an inactive protein-bound form. The free, unbound form is independent of the Vd; clearance and extent of protein binding are more important determinants.

### 1. Cyclin-Dependent Kinase-2 as Target for Cancer Therapy

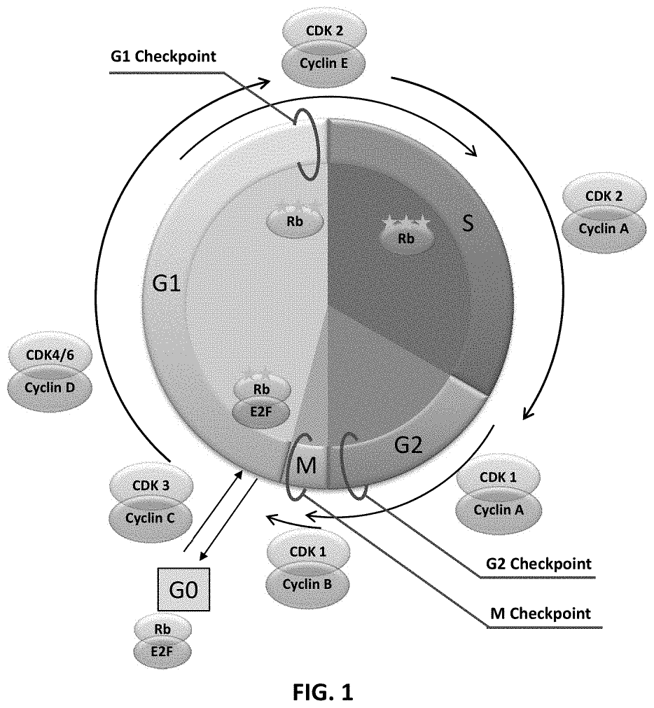

Cell-cycle progression is stimulated by protein kinase complexes, each of which consists of a cyclin and a cyclin-dependent kinase (CDK). The cell division cycle of the eukaryotic cells (see FIG. 1) involves four sequential phases including S-phase (DNA replication occurs), M-phase (chromosome segregation occurs and the cell divides into two daughter cells), S-phase and M-phase are separated by two gap phases known as G1 (cells undertake most of their growth and synthesize proteins, RNAs, and organelles needed for DNA synthesis), and G2 (the microtubules that will be used to mobilize the chromosomes in the M-phase are assembled). Quiescence (G0) represents an exit from the cell cycle either because of deprivation of mitogen or full differentiation of the cell (Schwartz, et al., Targeting the Cell Cycle: A New Approach to Cancer Therapy. J. Clin. Oncol. 2005, 23, pp. 9408-9421; Malumbres et al., Milestones in Cell Division: To Cycle or Not To Cycle: A Critical Decision in Cancer. Nat. Rev. Cancer 2001, 1, pp. 222-231).

Most adult cells are at G0 and the transcriptional activity of E2F transcription factors (E2Fs) is repressed by the retinoblastoma proteins (hereafter refers as Rb). When needed, these cells can go back into the cell division cycle. Under the action of the mitogenic stimuli, CDK3 forms complex with cyclin C and promotes cells at G0 to enter G1 (Ren et al., Cyclin C/CDK3 promotes Rb-dependent G0 exit, Cell, 2004, vol. 117, pp. 239-51). The complexes cyclin D/CDK4, cyclin D/CDK6, cyclin E/CDK2, and cyclin A/CDK2 regulate the progression from G1 phase to S phase. During G1-phase progression, cyclin D/CDK4 and cyclin E/CDK2 complexes are activated. These enzymes sequentially phosphorylate the Rb. Binding of hypophosphorylated Rb to E2F transcription factors arrests cells in the G0 phase. However, hyperphosphorylated pRb results in the dissociation of Rb and E2F and entry into S-phase. Cyclin A/CDK2 facilitates S/G2 transition, and cyclin A/CDK1 and cyclin B/CDK1 enable the commencement of mitosis and the progression through the M-phase, respectively. Progression from G2 to M phase is regulated by the cyclin B/CDK1 complex through mitosis. Cyclin A/CDK2, cyclin A/CDK1, and cyclin B/CDK1 sustain the phosphorylation of Rb ensuring cell-cycle progression. Finally, cyclin B is degraded, and Rb is dephosphorylated by two phosphatases, PP1 and PP2A, returning the cell to the G1 state (See FIG. 1; Malumbres, Physiological Relevance of Cell Cycle Kinases. Physiol. Rev. 2011, vol. 91, pp. 973-1007).

The cell cycle is controlled by checkpoints. The integrity of the DNA is assessed at the G1/S checkpoint. Proper chromosome replication is checked at the S and G2/M checkpoints (See FIG. 1; Ren et al., Cyclin C/CDK3 Promotes Rb-dependent G0 Exit. Cell 2004, vol. 117, pp. 239-251).

The CDKs belong to the serine/threonine-protein kinase family. In different phases of the cell division cycle, CDKs bind to a cyclin protein to form cyclin/CDK complex to promote transitions between different cell cycle phases, in which, cyclins are the regulatory subunits that control kinase activity and substrate specificity, and CDK functions as catalyst to promote phosphorylation of the substrate. CDKs family of proteins are involved in various aspects of cell biology including cell-cycle control (CDKs 1, 2, 3, 4 and 6), transcription (CDKs 7, 8, 9, 11, 12, 13 and 20), DNA damage repair (CDKs 1, 3 and 9), metabolism (CDKs 5 and 8), and cell differentiation in certain cell types (CDKs 1, 2, 4 and 16) (Malumbres, Cyclin-Dependent Kinases. Genome Biol. 2014, vol. 15, p. 122; Lim et al., CDKs, cyclins and CKIs: roles beyond cell cycle regulation, Development, 2013, vol. 140, pp. 3079-3093). Cyclin E1 and E2 (collectively refers to as cyclin E) drive proliferation by promoting the initiation of DNA replication and by activating CDK2. Cyclin E/CDK2, along with cyclin D/CDK4, phosphorylates Rb to activate E2F-responsive genes and promote progression into S-phase (Desmedt et al., Impact of cyclin E, neutrophil elastase and proteinase 3 expression levels on clinical outcome in primary breast cancer patients. Int. J. Cancer, 2006, vol. 119, pp. 2539-2545; Sieuwerts et al., Which cyclin E prevails as prognostic marker for breast cancer? Results from a retrospective study involving 635 lymph node-negative breast cancer patients. Clin Cancer Res., 2006, vol. 12, pp. 3319-3328).

CDK2 complexes with cyclin E plays a role in the phosphorylation of Rb and the G1 to S-phase transition of the cell cycle, as well as in assembly of the pre-replication complex in S-phase. CDK2 has broad substrates than those of CDK4/6 and plays important role in phosphorylation of proteins regulating cell cycle (e.g., p27KIP1, Rb), DNA replication (replication factors A and C), coactivator of histone transcription (e.g., protein NAPT), and centrosome replication-associated protein (e.g., nucleophosmin, NPM) (Asghar et al., The history and future of targeting cyclin-dependent kinases in cancer therapy, Nature Reviews Drug Discovery, 2015, vol. 14, pp. 130-146). CDK2 also binds cyclin A, forming a complex that is required to initiate DNA synthesis in S-phase and activate CDK1/cyclin B for the G2-M transition.

Loss of cell-cycle checkpoint is a hallmark of human cancers. Dysregulation of CDKs or its regulatory subunits cyclins has been reported to contribute to both cancer progression and aggressiveness in human cancer. Cell-cycle regulation by tumor suppressor protein Rb plays an integral role in preventing human tumors because oncogenic alterations in cyclins, CDKs and other upstream regulators of Rb occur in a variety of human tumors including breast cancer. For example, about 50% of the invasive breast cancers have elevated cyclin D expression in reference to surrounding normal breast epithelium. Amplification of genes encoding cyclin D1 has been identified in 29%-58% of breast cancers. Cyclin D1 expression is believed to drive aberrant phosphorylation and inactivation of the Rb, primarily in luminal A and luminal B. Overexpression of cyclin E has been described in tumor insensitive to CDK4/6 inhibitors as well as in ovarian and lung tumor types (See Sante et al. supra).

Breast cancer is the most common non-dermatological malignancy in women in many countries. Five distinct molecular subtypes of breast cancer were identified referred to as luminal A, luminal B, HER2-enriched, basal like and claudin-low, as well as a normal breast-like group. (Prat, et al., Deconstructing the molecular portraits of breast cancer, Molecular Oncology, 2011, vol. 5, pp. 5-23). The potential genetic targets for therapy include the estrogen receptor (ERα), progesterone receptor (PR), and/or human epidermal growth factor receptor 2 (HER2). Triple negative breast cancer (TNBC), which lacks ERα, PR and HER2, is the most deadly form of breast cancer (Sante et al., Recent advances with cyclin-dependent kinase inhibitors: therapeutic agents for breast cancer and their role in immune-oncology, Expert Review of Anticancer Therapy, 2019, vol. 19, pp. 569-587).

Ovarian carcinomas are a heterogeneous group of neoplasms. Recent discoveries have demonstrated that ovarian cancer is composed of multiple separate diseases. Epithelial ovarian cancer (EOC) remains the most lethal gynecological malignancy in the United States. Despite the recent advances in targeted therapy in different types of cancer, platinum based therapies such as cisplatin remain the standard of care for EOC patients. High-grade serous ovarian cancer (HGSOC) is the most common subtype (>70% of EOC cases) and accounts for the majority of EOC-associated mortalities (Cho et al., Ovarian Caner, Annu. Rev. Pathol., 2009, vol. 4, pp. 287-313).

The development of novel therapeutic strategies for triple negative breast cancer and ovarian cancer remains a major obstacle to overcome.

Precision medicine approaches have been deployed to reduce mortality from breast cancer by targeting specific abnormalities identified in the coding region of the genome. Treating HER2-positive breast cancer with anti-HER2 antibody (e.g., trastuzumab) has markedly improved the outcome of this disease. One major challenge to targeted therapy is acquired and primary resistance (Cheng et al., Diagnostic Pathology, 2013, vol. 8, pp. 129-138). The introduction of tamoxifen as a treatment for hormone receptor-positive breast cancer led to significant decrease in breast cancer mortality over the past 30 years. However, resistance to endocrine therapy occurs in more than 30% of patients and results in disease progression. A pervasive feature of tamoxifen-resistant breast cancer is increased expression of genes related to proliferation such as cyclin D1, and genes regulated by Myc proto-oncogene protein (MYC), E2F and Rb, suggesting a particularly important role of genes involved in the G1 phase to S-phase transition of cell cycle (Caldon et al., Cyclin E2 overexpression is associated with endocrine resistance but not insensitivity to CDK2 inhibition in human breast cancer cells, Mol. Cancer Ther., 2012, vol. 11, pp. 1488-1499). However, antiestrogens target these pathways through downregulation of cyclin D1 leading to loss of cyclin D1/CDK4 activity, downregulation of MYC leading to upregulation of p21 and p27. This leads to inhibition of both CDK2 and CDK4 activity and G1-S phase arrest (Garcia-Gutierrez et al., MYC Oncogene Contributions to Release of Cell Cycle Brakes, Genes, 2019, 10(3), p. 244; Doisneu-Sixou et al., Estrogen and antiestrogen regulation of cell cycle progression in breast cancer cells. Endocr Relat Cancer 2003; 10, pp. 179-186).

Recent advances in treatment of breast cancers using CDK inhibitors have focused on targeting cyclin D/CDK 4/6 for the treatment of HR-positive and HER2-negative breast cancer, with regulatory approvals of palcociclib, ribociclib and abemaciclib, and ongoing clinical development of lerociclib. Although CDK4/6 inhibitors are part of the established treatment regimens for certain form of breast cancer (BC), insensitivity to CDK4/6 inhibition has been found in primary resistance, such as forms of triple negative breast cancers (TNBC), or acquired resistance, by prior treatment with a CDK4/6 inhibitor in ER⁺ Her2⁻ breast cancer.

Inhibition of CDK2 gives a promising option of using CDK2 inhibitors to treat cancers with dysregulated cell-cycle. Cyclin E2 expression has been associated with poor outcome of ER-positive breast cancer. Cyclin E2 is induced in gene signatures that predicts disease progression in either tamoxifen-resistant breast cancer or metastatic breast cancer. Cyclin E1 overexpression can acutely reduce antiestrogen sensitivity in vitro. In addition, CDK2 activation is a possible mechanism of resistance to a CDK4 inhibitor that inhibits ER-positive breast cancer and can overcome acquired resistance to tamoxifen (Caldon et al. Cyclin E2 Overexpression Is Associated with Endocrine Resistance but not Insensitivity to CDK2 Inhibition in Human Breast Cancer Cells. Molecular Cancer Therapeutics, 2012, vol. 11, pp. 1488-1499). In Rb-deficient cancer cells, CDK4/6 signaling is redundant and the E2Fs are constitutively active. In Rb-positive cells, overexpression of cyclin E or loss of the CIP/KIP proteins might bypass CDK4/6 inhibition by activating CDK2. In triple-negative breast cancer (TNBC), where tumors often demonstrate loss of expression of the Rb protein or high expression of cyclin E, both of which would be expected to confer resistance to treatment with CDK4/6 inhibitors, and sensitizing CDK2 inhibitor (Pernas et al. CDK4/6 inhibition in breast cancer: current practice and future directions, Therapeutic Advances in Medical Oncology, 2018, 10:1758835918786451). In addition, in TNBC, CDK2 inhibition is synergistic with chemotherapeutic agent and radiotherapy, and restores chemo and radio-sensitivity in resistant cases.

In recent years, a number of CDK2 inhibitors have been developed and are under evaluation. Currently, dinaciclib is a regulatory approved orphan drug as a targeted treatment for chronic lymphocytic leukemia (CLL). Dinaciclib has inhibitory activity against CDK2, CDK5, CDK1 and CDK9. A few pharmacologic inhibitors of CDK2 are in ongoing clinical developments as anticancer agents. Phase II clinical trial has been completed for AT7519 (AT7519M, Astex Therapeutics Ltd). Patient recruiting is in progress for a phase II clinical trial for TG02 (Tragara Pharmaceuticals). Phase I clinical trial has been completed for roniciclib (BAY100394, Bayer). The phase I clinical trial for CYC065 (Cyclacel Pharmaceuticals) is ongoing.

Some CDK2 inhibitor clinical candidates have already been discontinued from clinical development because of promiscuity leading to off-target kinase inhibition and associated side effects as well as failure to achieve an acceptable clinical end point, for example, milciclib (PHA-848125, Tiziana Life Sciences) and seliciclib (CYC202, Cyclacel Pharmaceuticals) are discontinued from phase II clinical trial, and phase I trial for AG-024322 was stopped (https://www.clinicaltrials.gov).

In addition to treating breast cancer with CDK2 inhibitors, CDK2 inhibitors may also find application in the treatment of other types of cancer such as ovarian cancer, neuroblastoma, glioblastoma, B-cell lymphoma, prostate cancer, hepatocellular carcinoma, lung cancer, acute myeloid leukemia, glioma, colorectal cancers or melanoma. (Tadesse, et al. Cyclin Dependent Kinase 2 Inhibitors in Cancer Therapy: an Update, Journal of Medicinal Chemistry, 2019, vol. 62, 4233-4251). Beyond oncology, the potential application of CDK2 inhibitor based targeted therapy may be expanded into other diseases such as infectious diseases, proliferative diseases, chronic inflammation diseases, neurodegenerative diseases, or hearing loss.

Still, CDK family of proteins have high level of sequence homology and similarity in three dimensional structure at the active catalytic site, the available CDK2 inhibitors are not selective and possess on- and off-target side effects. Hence, there is a need to develop safe and selective inhibitors of CDK2 for the therapeutic management of cancer and associated disorders.

This disclosure provide deuterated pyridopyrimidinone compounds as selective CDK2 inhibitors with sub-nanomolar biochemical IC₅₀ values for CDK2 enzyme when complexed with cyclin A and cyclin E.

### 2. Site Selective Deuteration

The role of deuterium in drug design and development is expanding in recent decade as the industry learn more about deuterium kinetic isotope effect (DIE) and how to deploy it with greater sophistication. The judicious introduction of deuterium into a molecule can productively influence conformation (size and shape), physical property (e.g. hydrophilicity or hydrophobicity), metabolic pathway, or pharmacokinetic and pharmacodynamics properties.

Isotopically labeled compounds have long been applied for mechanistic studies and as tools in biochemical research. However, the introduction of deuterium in active pharmaceutical ingredients has only recently been recognized as potential path to better drugs. In contrast, due to the electronic properties and relatively small size of fluorine, fluorination has been explored extensively in drug design to modulate metabolism or off-target activity of a given drug (e.g., PF-06873600 supra; also See Albericio et al., The pharmaceutical industry in 2018, an analysis of FDA approvals from the perspective of molecules, Molecules, 2019, vol. 24, pii:E809). In the medicinal chemist's toolbox, the fluorination is the classic choice to modulate the metabolism of a given drug. For example, PF-06873600 ((−)-6-(difluoromethyl)-8-((1R,2R)-2-hydroxy-2-methylcyclopentyl)-2-((1-(methylsulfonyl)piperidin-4-yl)amino)pyrido[2,3-d]pyrimidin-7(8H)-one, Comp. 5) is a selective and orally bioavailable inhibitor of cyclin-dependent kinase 2/4/6 (CDK2/4/6) currently in Phase 1a/2 clinical development for the treatment of advanced breast cancer. The compound is described in e.g. WO 2018033815, US Patent Application No. 20180044344 and U.S. patent Ser. No. 10/233,188. A difluorinated methyl group at the 6-position of the pyrido[2,3-d]pyrimidin-7(8H)-one ring system was introduced to improve metabolic stability (See Table 6 for the t_{1/2} for Comp. 5 (t_{1/2}=2.76 hr) vs. non-fluorinated Comp. 4 (t_{1/2}=2.24 hr)).

View from the length and size of a C—F bond against a C—H bond, C—F bond (1.35 Å) is more closely aligned with a C═O bond (1.23 Å), and is shorter than a C—H (1.9 Å) or a C—OH bond (1.43 Å). The electronegativity of fluorine is closer to that of oxygen, which is reflected in the dipole moment of the C—F bond being larger and in the opposite direction of a C—H. Fluorine is modestly more lipophilic than a hydrogen atom and significantly more lipophilic than OH, C═O substituents. The replacement of a hydrogen atom by fluorine would be expected to modestly increase the lipophilicity of a molecule and significantly increase molecular weight (Meanwell, Fluorine and Fluorinated Motifs in the Design and Application of Bioisosteres for Drug Design, J. Med. Chem., 2018, vol. 61, pp. 5822-5880).

Deuterium substitution emerged in recent decades as an attractive means to improve pharmacokinetic, metabolic stability for drug molecules. Deutetrabenazine is the first FDA approved deuterated new chemical entity (NCE) in 2017 for the treatment of both chorea associated with Huntington's disease and tardive dyskinesia. There are more than 20 deuterated drugs are currently in clinical development with 6 of them (BMS-986165, AVP-786, RT001, ALK-001, donafenib, HC-1119) having reached Phase III clinical trials (Cargnin et al., A primer of deuterium in Drug Design, Future Med. Chem., 2019, vol. 11, pp. 2039-2042).

Strategic deuterium substitution on a given drug molecule provides an efficient and accelerated approach to creating significantly differentiated new medicine that address important unmet medical needs.

Deuterium can be represented by the symbol D or 2H and is also known as heavy hydrogen. It is a stable, non-radioactive and naturally-occurring isotope of hydrogen. The ordinary isotope of hydrogen, H, is known as Protium, the other two isotopes are Deuterium (a proton and a neutron) and Tritium (a proton and two neutrons). Hydrogen consists of one electron and one proton and has an atomic mass of approximately 1.0 atomic mass unit (AMU). Deuterium has a single electron and the nucleus contains one neutron and one proton resulting as atomic mass of approximately 2.0 AMU. With regard to the shape and size of a molecule, deuterium substitution for hydrogen yields a deuterated compound that is quite similar to the all-hydrogen compound (sterically similar). However, physical property changes have been observed in partially and fully deuterated compounds, for example, reduced hydrophobicity (Turowski et al., J. Am. Chem. Soc., 2003, vol. 125, p. 13836), decreased acidity of carboxylic acid and phenols (Perrin et al., J. Am. Chem. Soc., 2007, vol. 129, p. 4490), and increased basicity of amines (Perrin et al., J. Am. Chem. Soc., 2005, vol. 127, p. 9641). Thus, deuterium in the deuterated compound has similar electron clouds and polar surface to their hydrogen parents.

Carbon-hydrogen bond strength is directly proportional to the absolute value of the ground-state vibrational energy of the bond. This vibrational energy depends on the mass of the atoms that form the bond, and increases as the mass of one or both of the atoms making the bond increases. C-D bonds have a lower vibrational frequency and lower zero-point energy than a corresponding C—H bond. Cleavage of the carbon-deuterium (C-D) covalent bond requires greater energy than that of the carbon-hydrogen bond due to the greater atomic mass of deuterium. The lower zero-point energy results in a higher activation energy and a slower rate (k) for C-D bond cleavage. This rate effect is the primary deuterium isotope effect (DIE) and is expressed as the ratio of the rate of C—H bond cleavage to the rate of C-D bond cleavage (k_{H}/k_{D} ranges from about 1 (no isotope effect) to 50 (very large isotope effect)). Put in another way, when hydrogen is replaced by deuterium, the C—H bond cleavage step must be at least partially rate-limiting in order to observe a DIE.

In order to eliminate foreign substances such as therapeutic agents, the animal body expresses various enzymes, such as the cytochrome P450 enzymes (CYPs), esterases, proteases, reductases, dehydrogenases, aldehyde oxidase (AO), and monoamine oxidases (MAO), to react with and convert these foreign substances to more polar intermediates or metabolites for renal excretion. Such metabolic reactions frequently involve the oxidation of a carbon-hydrogen (C—H) bond to either a carbon-oxygen (C—O) or a carbon-carbon (C—C) π-bond. The resultant metabolites may be stable or unstable under physiological conditions, and can have substantially different pharmacokinetic, pharmacodynamics, and acute and long-term toxicity profiles relative to the parent compounds. For most drugs, such oxidations are generally rapid and ultimately lead to administration of multiple or high daily doses.

Some disorders are best treated when the subject is medicated around the clock or for an extended period of time. It is well-documented that a medicine with a longer half-life may result in greater efficacy and cost savings.

Deuterium substitution is the smallest possible structural change that can be made to a molecule. Substitution with heavier isotopes such as deuterium (2H, D) provides certain therapeutic advantages resulting from greater metabolic stability, for example, increased in vivo half-life, reduced clearance, reduced dosage requirements, improved safety, or improved efficacy (Harbeson et al., Deuerium Medicinal Chemistry: A New Approach to Drug Discovery and development, MEDCHEM News, 2014, pp. 8-22). Due to DIE, the carbon-deuterium bond is more stable than a carbon-hydrogen bond of an appropriately deuterated drug molecule, which leads to decreased kinetic rates of enzymatic metabolism of about 6 to 10 fold when bond breakage is the rate-limiting step, for example, when metabolic enzymes are CYP450 family of enzymes or other enzymes involved in metabolism such as monoamine oxidase (MAO) and aldehyde oxidase (AO).

Deuteration of pharmaceuticals to improve pharmacokinetics (PK), pharmacodynamics (PD), and toxicity profiles has been demonstrated previously with some classes of drugs. For example, the DIE was used to decrease the hepatotoxicity of halothane, presumably by limiting the production of reactive species such as trifluoroacetyl chloride (See Harbeson et al. supra).

In most cases, the difference in pharmacological effects resulted from deuteration tend to be negligible, for example, deuteration of a non-covalent drug has negligible effects upon biochemical potency or selectivity for relevant pharmacological targets. The effects of deuteration on drug stability toward CYP metabolism are complex and unpredictable. Many drugs are metabolized in complex patterns. The CYP catalysis is a highly complicated multiple-step process where a number of steps could be partially or wholly rate limiting, thereby “masking” the isotope effect. For example, isotope substitution upon some drugs may not give rise to significant isotope effects in metabolism or clearance. The lack of kinetic isotope effect may be due to the lack of contribution of C—H bond in the overall metabolism of the drug, or because of metabolic switching, which results in the potentiation of other metabolic pathways. The observed DIE (k_{H}/k_{D})_{obs} can be much smaller than k_{H}/k_{D}, or altogether absent in some cases.

Deuterium substitution does not solve issues for every compound and in every position. It is reported that less than 10% of all FDA-approved drugs are amendable to deuteration because of their chemical structure or because they are metabolized in a way that deuterium kinetic isotope effect would not be significant. If more than one oxidizable soft-spot is present in a given drug molecule, a judicious choice of both the molecule and the site of deuteration must be undertaken to exploit the metabolic fate and the enzymology of the given compound. For some drugs, there are many metabolic active sites (soft-spot) and it is difficult to predict which effect deuterium may have on a drug's metabolism in the absence of testing the metabolic properties of an actual deuterated compound.

Additionally, other non-CYP clearance mechanisms may predominate and blunt the observed DIE, e.g., aldehyde oxidase (AO) is known to oxidize aromatic nitrogen heterocycles and causes rapid drug metabolism. Nitrogen heterocyclic drug molecules such as carbazeran and zoniporide have been reported to have AO as clearance mechanism. The in vivo PK studies on the deuterated versions of carbazeran (1-[²H]-carbazeran) and zoniporide (2-[²H]-zoniporide) showed that while in vitro DIEs were significant (as high as 5), clearance mechanism are complex, and thus it is difficult to predict whether deuteration of an AO substrate may lead to improved pharmacokinetic parameters in vivo (See Goodnow Ed., Annual Reports In Medicinal Chemistry, vol. 50, Academic Press Elsevier, Cambridge, United States, 2017, Chapter 14. A decade of deuteration in medicinal chemistry, p. 519-538).

Furthermore, reducing CYP metabolism with deuterium at a known site of metabolism can “switch” metabolism to another site, resulting in no observed DIE (no change in clearance) or even an inverse DIS (faster clearance). Metabolic switching occurs when xenogens, sequestered by Phase I enzymes, bind transiently and re-bind in a variety of conformations prior to the chemical reaction (e.g., oxidation). Metabolic switching can lead to different proportions of known metabolites as well as altogether new metabolites. This new metabolic profile may impart more or less toxicity. Such pitfalls are non-obvious and are not predictable a priori for any drug class (Forster, Deuterium isotope effects in the metabolism of drugs and xenobiotic: implications for drug design, Adv. Drug Res. 1985, vol. 14, pp. 1-40).

Moreover, even incorporation of deuterium at know metabolic sites has an unpredictable impact on metabolite profile. For example, maraviroc (Selzentry®), a negative allosteric modulator of CCR5 receptor for treating HIV, has two major metabolic sites in human (pseudobenzylic methyl group and N-alkyl group). Stability studies in human microsomal incubations showed that deuteration at both sites is required to reduce in vitro microsomal clearance. Unexpectedly, deuteration only at the pseudobenzylic methyl group actually accelerated microsomal turnover compared with the protio drug (See declaration filed on Nov. 16, 2010 in the prosecution history of U.S. Pat. No. 7,932,235, 2011).

In some instances, deuteration on drug molecules has implications that go beyond mere increase of metabolic stability, e.g., deuterated compound BMS-986165 (an inhibitor of tyrosine kinase 2 for treatment of psoriasis) is more potent and selective compared to the parent drug on the market.

Lastly, the synthesis of deuterated compound at a selective position on a given drug molecule is far from trivial: on one hand, the deuterated pool strategy is limited by narrow scope and relatively high cost of deuterated substrates; whereas on the other the exchange approach might suffer from low selectivity and deuterium content.

The masked DIE, metabolic switch, the complexity of the CYP catalyzed metabolic reactions, together with alternative drug clearance mechanism or site of metabolism, the impact of deuterium substitution for hydrogen upon the metabolic reaction is highly unpredictable and will be dependent upon the specific compound and the deuterium substitution patterns on the molecule. Additional examples of studies indicating lack of predictability regarding deuterium incorporation include: U.S. Pat. Nos. 6,332,335, 7,678,914, 10,501,427; Fisher et al., Curr. Opin. Drug Discov. Devel., 2006, vol. 9, p. 101-109; J. Med. Chem. 1991, vol. 34, 2871-2876; Adv. Drug Res., 1985, vol. 14, pp. 1-40; Harbeson et al. supra).

### 3. Deuterated Pyridopyrimidinones as Selective CDK2 Inhibitors

In an embodiment, this disclosure provides a compound of Formula (2):

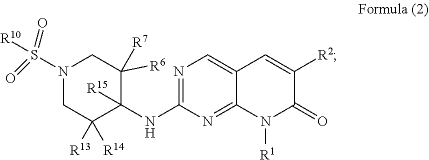

wherein,

R¹ is selected from the group of of

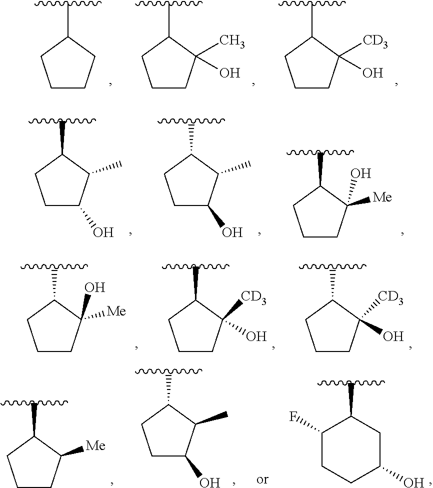

R² is a substituent selected from the group of H, D, F, Cl, Br, C1-C4 alkyl group optionally substituted with one or more D, —OH, —CN, Cl, —C(═O)—NH₂, CH₃—(CH₂)ₙ—O—, and C1-C4 fluoroalkoxyl group, or C1-C4 fluoroalkyl group optionally substituted with one or more D, —OH, —CN, —C(═O)—NH₂, CH₃—(CH₂)ₙ—O—, and C1-C4 fluoroalkoxyl group,

R⁶, R⁷, R¹³, R¹⁴ and R¹⁵ are each independently hydrogen or deuterium, and R⁶=R⁷=R¹³=R¹⁴;

R¹⁰ is a substituent selected from the group of —NHR¹⁶; C1-C2 alkyl group optionally substituted with one or more of D, F, CH₃—(CH₂)ₙ—O—, C3-C5 cycloalkyl group, and C1-C2 fluoroalkoxyl group; C1-C2 fluoroalkyl group; cyclopropyl group;

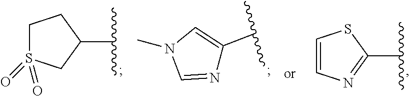

R¹⁶ is a substituent selected from the group of H, Me, C1-C3 fluoroalkoxyl group,

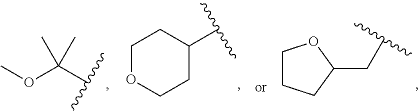

n is 0, 1, 2, or 3,

wherein the compound of Formula (2) is substituted with at least one deuterium atom, and

pharmaceutically acceptable salt, stereoisomer, cocrystal, prodrug, solvate, hydrate, or polymorph thereof.

In some embodiments, for the compounds of Formula (2), R¹ is

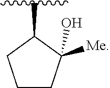 [SMILES: *[C@@H]1CCC[C@@]1(C)O]

In some embodiments, for the compounds of the Formula (2), R² is selected from the group of CH₃—, CF₂H—, CF₂D-, or CD₃-.

In some embodiments, for the compounds of the Formula (2), R¹⁰ is a substituent selected from the group of —CH₃ or -CD₃.

In some embodiments, for the compounds of the Formula (2), R⁶=R⁷=R¹³=R¹⁴=D.

In some embodiments, for the compounds of the Formula (2), R⁶=R⁷=R¹³=R¹⁴=R¹⁵=D.

In some embodiments, for the compounds of the Formula (2), R¹⁵=D.

In some embodiments, for the compounds of the Formula (2), R⁶=R⁷=R¹³=R¹⁴=D, and R¹⁰ is —CD₃ or —CH₃.

In some embodiments, for the compounds of the Formula (2), R⁶=R⁷=R¹³=R¹⁴=R¹⁵=D and R¹⁰ is —CD₃ or —CH₃.

In some embodiments, for the compounds of the Formula (2), R¹⁰ is —CD₃ or —CH₃ and R¹⁵=D.

In some embodiments, for the compounds of the Formula (2), R² is —CD₃ or —CH₃, and R¹⁶=R⁷=R¹³=R¹⁴=D.

In some embodiments, for the compounds of the Formula (2), R² is —CD₃ or —CH₃, and R¹⁶=R⁷=R¹³=R¹⁴=R¹⁵=D.

In some embodiments, for the compounds of the Formula (2), R² is —CD₃ or —CH₃, and R¹⁵=D.

In some embodiments, for the compounds of the Formula (2), R² is —CD₃ or —CH₃, R⁶=R⁷=R¹³=R¹⁴=D, and R¹⁰ is —CD₃ or —CH₃.

In some embodiments, for the compounds of the Formula (2), R² is —CD₃ or —CH₃, R⁶=R⁷=R¹³=R¹⁴=R¹⁵=D and R¹⁰ is —CD₃ or —CH₃.

In some embodiments, for the compounds of the Formula (2), R² is —CD₃ or —CH₃, R¹⁰ is —CD₃ or —CH₃, and R¹⁵=D.

In an embodiment, this disclosure provides an enantiomerically enriched compound of Formula (3):

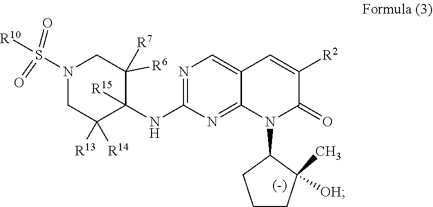

wherein,

R² is a substituent selected from the group of H; D; F; Cl; Br; C1-C4 alkyl group optionally substituted with one or more D, —OH, —CN, Cl, —C(═O)—NH₂, CH₃—(CH₂)ₙ—O—, and C1-C4 fluoroalkoxyl group; and C1-C4 fluoroalkyl group optionally substituted with one or more D, —OH, —CN, —C(═O)—NH₂, CH₃—(CH₂)ₙ—O—, and C1-C4 fluoroalkoxyl group,

R⁶, R⁷, R¹³, R¹⁴ and R¹⁵ are each independently hydrogen or deuterium, and R⁶=R⁷=R¹³=R¹⁴;

R¹⁰ is a substituent selected from the group of —NHR¹⁶; C1-C2 alkyl group optionally substituted with one or more of D, F, CH₃—(CH₂)ₙ—O—, C3-C5 cycloalkyl group, and C1-C2 fluoroalkoxyl group; C1-C2 fluoroalkyl group; cyclopropyl group;

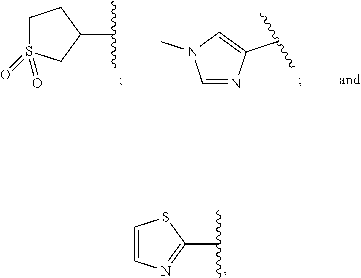

R¹⁶ is a substituent selected from the group of H, Me, C1-C3 fluoroalkoxyl group,

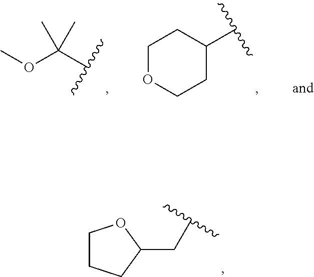

n is 0, 1, 2, or 3,

wherein the compound of Formula (3) is substituted with at least one deuterium atom, and

pharmaceutically acceptable acid addition salt, crystal, prodrug, solvate, and polymorph thereof.

In some embodiments, for the compounds of Formula (3), R² is selected from the group of CH₃—, CF₂H—, CF₂D-, and CD₃-.

In some embodiments, for the compounds of Formula (3), R¹⁰ is a substituent selected from the group of —CH₃, and —CD₃.

In some embodiments, for the compounds of Formula (3), R⁶=R⁷=R¹³=R¹⁴=D.

In some embodiments, for the compounds of Formula (3), R⁶=R⁷=R¹³=R¹⁴=R¹⁵=D.

In some embodiments, for the compounds of Formula (3), R¹⁵=D.

In some embodiments, for the compounds of Formula (3), R⁶=R⁷=R¹³=R¹⁴=D, and R¹⁰ is —CD₃ or —CH₃.

In some embodiments, for the compounds of Formula (3), R⁶=R⁷=R¹³=R¹⁴=R¹⁵=D and R¹⁰ is —CD₃ or —CH₃.

In some embodiments, for the compounds of Formula (3), R¹⁰ is —CD₃ or —CH₃, and R¹⁵=D.

In some embodiments, for the compounds of Formula (3), R² is —CD₃ or —CH₃, and R⁶=R⁷=R¹³=R¹⁴=D.

In some embodiments, for the compounds of Formula (3), R² is —CD₃ or —CH₃, and R⁶=R⁷=R¹³=R¹⁴=R¹⁵=D.

In some embodiments, for the compounds of Formula (3), R² is —CD₃ or —CH₃, R¹⁵=D.

In some embodiments, for the compounds of Formula (3), R² is —CD₃ or —CH₃, R⁶=R⁷=R¹³=R¹⁴=D, and R¹⁰ is —CD₃ or —CH₃.

In some embodiments, for the compounds of Formula (3), R² is —CD₃ or —CH₃, R⁶=R⁷=R¹³=R¹⁴=R⁵=D, and R¹⁰ is —CD₃ or —CH₃.

In some embodiments, for the compounds of Formula (3), R² is —CD₃ or —CH₃, R¹⁰ is —CD₃ or —CH₃, and R⁵=D.

In some embodiments, the deuterated pyridopyrimidinone compounds having Formula (3) is present as a single diastereomer having 95% ee or greater. In some embodiments, enantiomeric purity for the deuterated pyridopyrimidinone compounds having Formula (3) is selected from the group of about 95% ee, about 96% ee, about 97% ee, about 98% ee, and about 99.9% ee. In some embodiments, the pure enantiomeric isomer is essentially free of any other isomer. In some embodiments, the pure enantiomeric isomer having Formula (3) is present as a single (1R,2R)-(−)-isomer.

In an embodiment, this disclosure provides a compound of Formula (4):

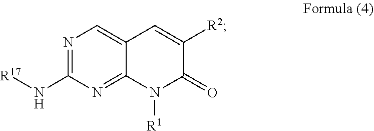

wherein,

R¹ is selected from the group of

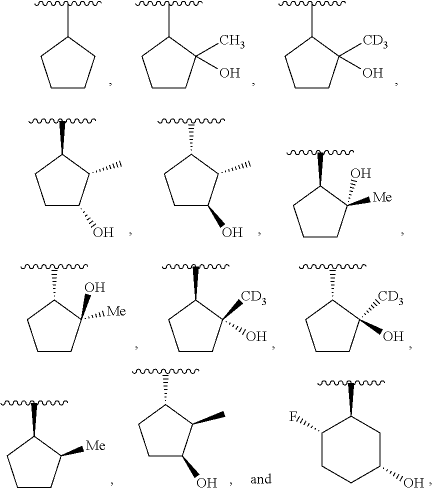

R² is selected from the group of H, F, Cl, Br, —CH₃, —CD₃, —CF₂H, or —CF₂D;

R¹⁷ is selected from the group of

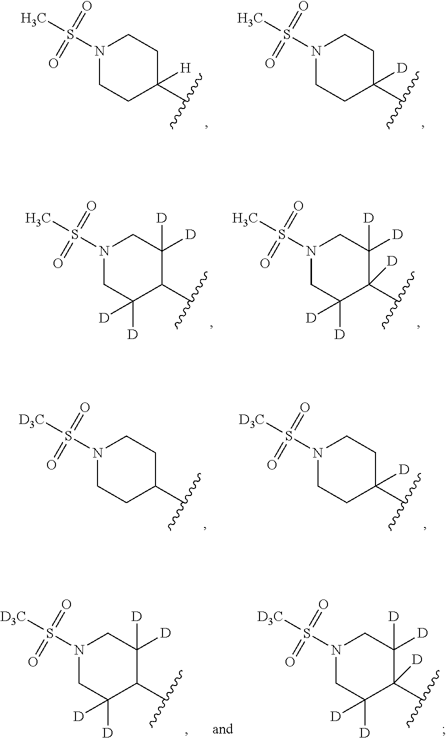

wherein the compound of Formula (4) is substituted with at least one deuterium atom; and

pharmaceutically acceptable salt, stereoisomer, cocrystal, prodrug, solvate, hydrate, and polymorph thereof.

In some embodiments, for the compounds of Formula (4), R¹ is

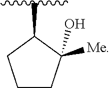 [SMILES: *[C@@H]1CCC[C@@]1(C)O]

In some embodiments, for the compounds of Formula (4), R¹ is

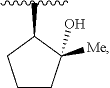 [SMILES: *[C@@H]1CCC[C@@]1(C)O]

and R² is —CH₃, or —CD₃.

In some embodiments, for the compounds of Formula (4), R¹ is

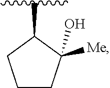 [SMILES: *[C@@H]1CCC[C@@]1(C)O]

and R² is —CD₃.

In some embodiments, for the compounds of Formula (4), R¹ is

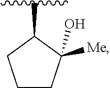 [SMILES: *[C@@H]1CCC[C@@]1(C)O]

and R² is —CF₂H, or —CF₂D.

In some embodiments, for the compounds of Formula (4), R¹ is

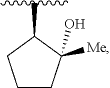 [SMILES: *[C@@H]1CCC[C@@]1(C)O]

R² is —CH₃, or —CD₃, and R¹⁷ is

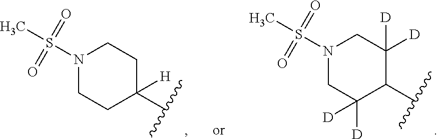 [SMILES: *C1CCN(S(C)(=O)=O)CC1 ; *C1C([2H])([2H])CN(S(C)(=O)=O)CC1([2H])[2H]]

In some embodiments, for the compounds of Formula (4), R² is —CH₃, or —CD₃, and R¹⁷ is

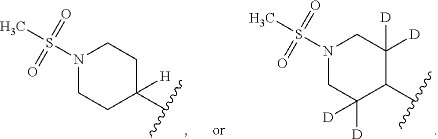 [SMILES: *C1CCN(S(C)(=O)=O)CC1 ; *C1C([2H])([2H])CN(S(C)(=O)=O)CC1([2H])[2H]]

In some embodiments, for the compounds of Formula (4), R² is —CD₃.

In an embodiment, this disclosure provides an enantiomerically enriched compound of Formula (5):

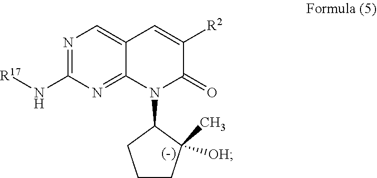

wherein,

R² is selected from the group of H, F, Cl, Br, —CH₃, —CD₃, —CF₂H, and —CF₂D;

R¹⁷ is selected from the group of

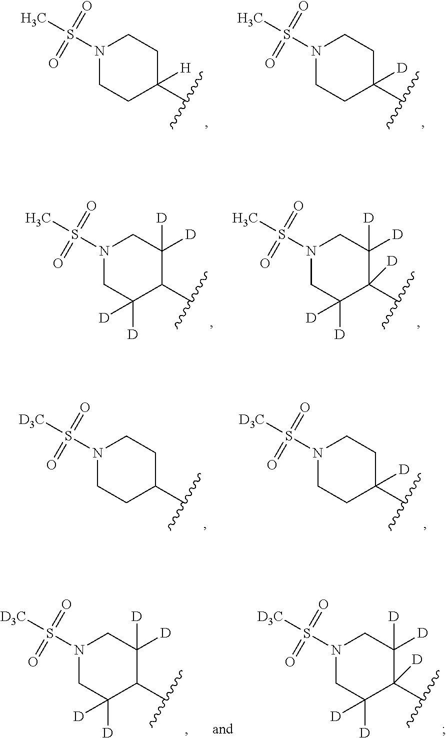

wherein the compound of Formula (5) is substituted with at least one deuterium atom; and

pharmaceutically acceptable salt, stereoisomer, cocrystal, prodrug, solvate, hydrate, and polymorph thereof.

In some embodiments, for the compounds of Formula (5), R² is selected from the group of H, —CH₃, —CD₃, —CF₂H, and —CF₂D. In some embodiments, for the compounds of Formula (5), R² is selected from the group of —CH₃, and —CD₃. In some embodiments, for the compounds of Formula (5), R² is —CD₃. In some embodiments, for the compound of Formula (5), R² is —CF₂H.

In some embodiments, for the compounds of Formula (5), R² is —CD₃, and R¹⁷ is selected from the group of

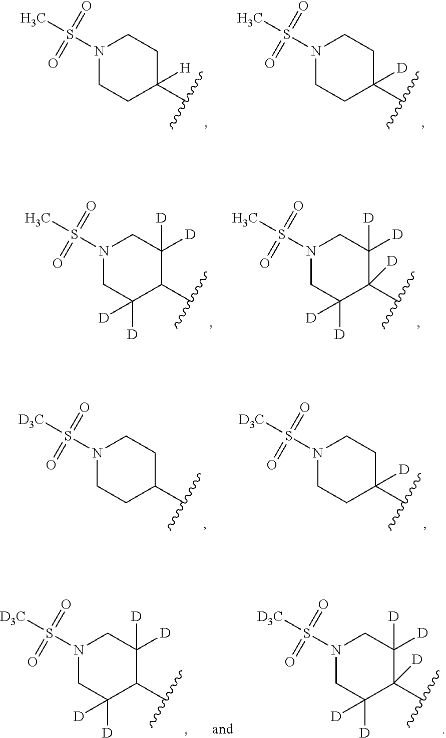

In some embodiments, for the compounds of Formula (5), R² is —CF₂H, and R¹⁷ is selected from the group of

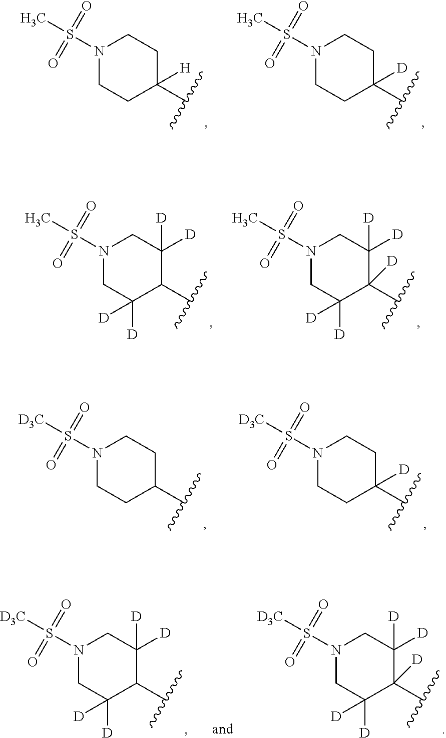

In some embodiments, for the compounds of Formula (5), R² is —CD₃, and R¹⁷ is selected from the group of

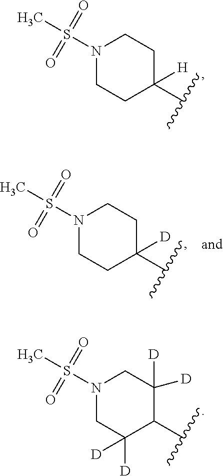 [SMILES: *C1CCN(S(C)(=O)=O)CC1 ; *C1([2H])CCN(S(C)(=O)=O)CC1 ; *C1C([2H])([2H])CN(S(C)(=O)=O)CC1([2H])[2H]]

In some embodiments, for the compounds of Formula (5), R² is —CF₂H, and R¹⁷ is selected from the group of

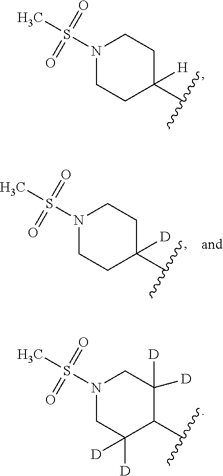 [SMILES: *C1CCN(S(C)(=O)=O)CC1 ; *C1([2H])CCN(S(C)(=O)=O)CC1 ; *C1C([2H])([2H])CN(S(C)(=O)=O)CC1([2H])[2H]]

In some embodiments, for the compounds of Formula (5), R¹⁷ is

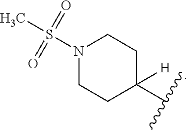 [SMILES: *C1CCN(S(C)(=O)=O)CC1]

In some embodiments, for the compounds of Formula (5), R¹⁷ is

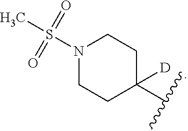 [SMILES: *C1([2H])CCN(S(C)(=O)=O)CC1]

In some embodiments, for the compounds of Formula (5), R⁷ is

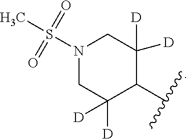 [SMILES: *C1C([2H])([2H])CN(S(C)(=O)=O)CC1([2H])[2H]]

In some embodiments, for the compounds of Formula (5), R⁷ is

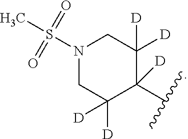 [SMILES: *C1([2H])C([2H])([2H])CN(S(C)(=O)=O)CC1([2H])[2H]]

In some embodiments, for the compounds of Formula (5), R¹⁷ is

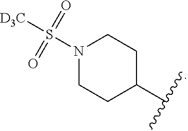 [SMILES: *C1CCN(S(=O)(=O)C([2H])([2H])[2H])CC1]

In some embodiments, for the compounds of Formula (5), R¹⁷ is

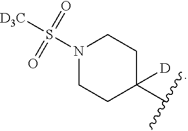 [SMILES: *C1([2H])CCN(S(=O)(=O)C([2H])([2H])[2H])CC1]

In some embodiments, for the compounds of Formula (5), R¹⁷ is

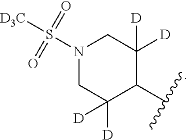 [SMILES: *C1C([2H])([2H])CN(S(=O)(=O)C([2H])([2H])[2H])CC1([2H])[2H]]

In some embodiments, for the compounds of Formula (5), R⁷ is

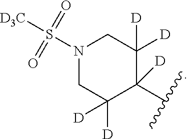 [SMILES: *C1([2H])C([2H])([2H])CN(S(=O)(=O)C([2H])([2H])[2H])CC1([2H])[2H]]

In some embodiments, the deuterated pyridopyrimidinone compounds having Formula (5) is present as a single diastereomer having 95% ee or greater. In some embodiments, enantiomeric purity for the deuterated pyridopyrimidinone compounds having Formula (5) is selected from the group of about 95% ee, about 96% ee, about 97% ee, about 98% ee, and about 99% ee. In some embodiments, the pure enantiomeric isomer is essentially free of any other isomer. In some embodiments, the pure enantiomeric isomer having Formula (5) is present as a single (1R,2R)-isomer.

In some embodiments, the deuterated pyridopyrimidinone compound of any one of the Formulae (1)-(5) is selected from one or more of the deuterated pyridopyrimidinone Comps. 7-213 disclosed in Table 1 below.

In some embodiments, the deuterated pyridopyrimidinone compounds of any one of the Formulae (1)-(5) is selected from the group of:
(±)-8-(2-hydroxy-2-methylcyclopentyl)-6-(methyl-d3)-2-((1-(methylsulfonyl)piperidin-4-yl)amino)pyrido[2,3-d]pyrimidin-7(8H)-one,
(±)-6-(difluoromethyl-d)-8-(2-hydroxy-2-methylcyclopentyl)-2-((1-(methylsulfonyl)piperidin-4-yl)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-yl)amino)pyrido[2,3-d]pyrimidin-7(8H)-one,
(−)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one,
(−)-8-((1R,2R)-2-hydroxy-2-methylcyclopentyl)-6-methyl-2-((1-(methylsulfonyl)piperidin-4-yl-3,3,5,5-d4)amino)pyrido[2,3-d]pyrimidin-7(8H)-one,
(−)-8-((1R,2R)-2-hydroxy-2-methylcyclopentyl)-6-methyl-2-((1-((methyl-d3)sulfonyl)piperidin-4-yl)amino)pyrido[2,3-d]pyrimidin-7(8H)-one,
(−)-8-((1R,2R)-2-hydroxy-2-methylcyclopentyl)-6-methyl-2-((1-(methylsulfonyl)piperidin-4-yl-4-d)amino)pyrido[2,3-d]pyrimidin-7(8H)-one,
(−)-8-((1R,2R)-2-hydroxy-2-methylcyclopentyl)-6-(methyl-d3)-2-((1-((methyl-d3)sulfonyl)piperidin-4-yl-3,3,4,5,5-d5)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-yl-3,3,4,5,5-d5)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-d-4-yl)amino)pyrido[2,3-d]pyrimidin-7(8H)-one,
(−)-6-(difluoromethyl-d)-8-((1R,2R)-2-hydroxy-2-methylcyclopentyl)-2-((1-(methylsulfonyl)piperidin-4-yl)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-6-(difluoromethyl)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-6-(difluoromethyl-d)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one,
(−)-6-(difluoromethyl)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl-3,3,5,5-d4)amino)pyrido[2,3-d]pyrimidin-7(8H)-one,
(−)-6-(difluoromethyl-d)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl-3,3,5,5-d4)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl-3,3,4,5,5-d5)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl-3,3,4,5,5-d5)amino)pyrido[2,3-d]pyrimidin-7(8H)-one,
(+)-8-((1S,2S)-2-hydroxy-2-methylcyclopentyl)-6-(methyl-d3)-2-((1-(methylsulfonyl)piperidin-4-yl)amino)pyrido[2,3-d]pyrimidin-7(8H)-one,
(+)-6-(difluoromethyl-d)-8-((1S,2S)-2-hydroxy-2-methylcyclopentyl)-2-((1-(methylsulfonyl)piperidin-4-yl)amino)pyrido[2,3-d]pyrimidin-7(8H)-one, and

pharmaceutically acceptable salt, stereoisomer, cocrystal, prodrug, solvate, hydrate, and polymorph thereof.

In some embodiments, the deuterated pyridopyrimidinone compounds of any one of the Formulae (1)-(5) is selected from the group of:
(−)-8-((1R,2R)-2-hydroxy-2-methylcyclopentyl)-6-(methyl-d3)-2-((1-(methylsulfonyl)piperidin-4-yl)amino)pyrido[2,3-d]pyrimidin-7(8H)-one,
(−)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one,
(−)-8-((1R,2R)-2-hydroxy-2-methylcyclopentyl)-6-methyl-2-((1-(methylsulfonyl)piperidin-4-yl-3,3,5,5-d4)amino)pyrido[2,3-d]pyrimidin-7(8H)-one,
(−)-8-((1R,2R)-2-hydroxy-2-methylcyclopentyl)-6-methyl-2-((1-((methyl-d3)sulfonyl)piperidin-4-yl)amino)pyrido[2,3-d]pyrimidin-7(8H)-one,
(−)-8-((1R,2R)-2-hydroxy-2-methylcyclopentyl)-6-methyl-2-((1-(methylsulfonyl)piperidin-4-yl-4-d)amino)pyrido[2,3-d]pyrimidin-7(8H)-one,
(−)-8-((1R,2R)-2-hydroxy-2-methylcyclopentyl)-6-(methyl-d3)-2-((1-((methyl-d3)sulfonyl)piperidin-4-yl-3,3,4,5,5-d5)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-yl-3,3,4,5,5-d5)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-d-4-yl)amino)pyrido[2,3-d]pyrimidin-7(8H)-one, and

pharmaceutically acceptable salt, stereoisomer, cocrystal, prodrug, solvate, hydrate, and polymorph thereof.

Any atom in the herein described deuterated pyridopyrimidinone compounds not specifically labelled as an isotope is meant to represent the given element at about its natural isotopic abundance. For example, H represents hydrogen (¹H) with a natural abundance of 99.985% and deuterium (²H) with a natural abundance of 0.015%.

While the natural isotopic abundance may vary in a synthesized compound based on the reagents used in the synthesis, the concentration of naturally abundant stable hydrogen isotopes such as deuterium is negligible compared to the concentration of stable isotopic substitution in the compounds of the disclosure. Thus, when a particular position of the herein described deuterated pyridopyrimidinone compound contains a deuterium atom, the concentration of deuterium at that position is substantially greater than the natural abundance of deuterium, which is 0.015%). In some embodiments, for the deuterated pyridopyrimidinone compounds of any one of the Formulae (1)-(5), a position containing a deuterium atom has a deuterium enrichment of at least 1%, of at least 5%, of at least 10%, of at least 15%, of at least 20%, of at least 25%, of at least 30%, of at least 35%, of at least 40%, of at least 45%, of at least 50%, of at least 55%, of at least 60%, of at least 65%, of at least 70%, of at least 75%, of at least 80%, of at least 85%, of at least 90%, of at least 91%, of at least 92%, of at least 93%, of at least 94%, of at least 95%, of at least 96%, of at least 97%, of at least 98%, or of at least 99%. In some embodiments, for the compounds of any one of the Formulae (1)-(5), a position containing a deuterium atom has a deuterium enrichment ranges from about 1% to about 99%. In some embodiments, for the compounds of any one of the Formulae (1)-(5), a position containing a deuterium atom has a deuterium enrichment ranges from about 95% to about 99%. In some embodiments, for the compounds of any one of the Formulae (1)-(5), a position containing a deuterium atom has a deuterium enrichment selected from the group of about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%. In some embodiments, for the deuterated pyridopyrimidinone compounds of any one of the Formulae (1)-(5), a position containing a deuterium atom has a deuterium enrichment selected from the group of about 90%, about 91%, about 92%, about 93%, about 94%, about 95%, about 96%, about 97%, about 98%, or about 99%. In some embodiments, for the deuterated pyridopyrimidinone compounds of any one of the Formulae (1)-(5), a position containing a deuterium atom has a deuterium enrichment selected from the group of about 95%, about 96%, about 97%, about 98%, or about 99%.

In some embodiments, the deuterium enrichment at the deuterated position is at least 50.1%. In some embodiments, the deuterium enrichment at the deuterated position is at least 60.0%. In some embodiments, the deuterium enrichment at the deuterated position is at least 67.5%. In some embodiments, the deuterium enrichment at the deuterated position is at least 72.5%. In some embodiments, the deuterium enrichment at the deuterated position is at least 75.0%. In some embodiments, the deuterium enrichment at the deuterated position is at least 77.5%. In some embodiments, the deuterium enrichment at the deuterated position is at least 80.0%. In some embodiments, the deuterium enrichment at the deuterated position is at least 82.5%. In some embodiments, the deuterium enrichment at the deuterated position is at least 85.0%. In some embodiments, the deuterium enrichment at the deuterated position is at least 87.5%. In some embodiments, the deuterium enrichment at the deuterated position is at least 90.0%. In some embodiments, the deuterium enrichment at the deuterated position is at least 92.5%. In some embodiments, the deuterium enrichment at the deuterated position is at least 95.0%. In some embodiments, the deuterium enrichment at the deuterated position is at least 97.5%. In some embodiments, the deuterium enrichment at the deuterated position is at least 99.0%. In some embodiments, the deuterium enrichment at the deuterated position is at least 99.5%.

As used herein, the term “deuterium enrichment” (also as deuterium incorporation or deuterium concentration) refers to the percentage of incorporation of deuterium at a given position of the herein described deuterated pyridopyrimidinone compounds in replacement of hydrogen than the natural abundance of deuterium.

In some embodiments, the herein described deuterated pyridopyrimidinone compounds may have one or more asymmetric centers. Unless otherwise indicated, all chiral (enantiomeric and diastereomeric) and racemic forms of compounds described herein are included. “Isomers” refer to different compounds that have the same molecular formula. “Stereoisomers” are isomers that differ only in the way the atoms are arranged in space, i.e., having a different stereochemical configuration. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon can be specified by either (R) or (S). Resolved compounds can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers can be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. In some embodiments, the deuterated pyridopyrimidinone compounds as described herein may exist in particular geometric or stereoisomeric forms including tautomers, cis- and trans-isomer, R- and S-enantiomers, diastereomers, (D)-isomers, (L)-isomers, the racemic mixtures thereof, and other mixtures thereof.

In some embodiments, the deuterated pyridopyrimidinone compounds of Formulae (1)-(5) may present as a mixture of diastereoisomers or as a single diastereomer. In some embodiments, the deuterated pyridopyrimidinone compounds of Formulae (1)-(5) may present as a racemic mixture of diastereoisomers.

In some embodiments, the deuterated pyridopyrimidinone compounds as described herein may be predominantly one enantiomer. In some embodiments, the deuterated pyridopyrimidinone compound of Formulae (1)-(5) comprises a mixture of about 90% or more by weight of the (−)-enantiomer and about 10% or less by weight of the (+)-enantiomer. In some embodiments, the deuterated pyridopyrimidinone compounds of Formulae (1)-(5) is present as a single diastereomer having 95% ee or greater. In some embodiments, the enantiomeric purity for the enantiomerically pure deuterated pyridopyrimidinone compounds of Formulae (1)-(5) is selected from the group of about 95% ee, about 96% ee, about 97% ee, about 98% ee, and about 99% ee. In some embodiments, the pure enantiomeric isomer is essentially free of any other isomer. In some embodiments, the pure enantiomeric isomer of Formulae (1)-(5) is present as a single (1R,2R)-(−)-isomer. In some embodiments, the pure enantiomeric isomer of Formulae (1)-(5) is present as a single (1S,2S)-(+)-isomer. In some embodiments, the pyridopyrimidinone compounds as described herein comprises an enantiomerically enriched mixture containing for example, at least 60 mole percent of one enantiomer. In some embodiments, enantiomerically enriched mixture may comprise at least 75, 90, 95 or 99 mole % of one enantiomer. In some embodiments, the compound enriched in one enantiomer is substantially free of the other enantiomer. In some embodiments, the term “substantially free of” as used herein refers to the amount of the substance at question makes up less than 10 mol. %, less than 5 mol. %, less than 4 mol. %, less than 3 mol. %, less than 2 mol. %, less than 1 mol. % as compared to the amount of the other enantiomer. In some embodiments, the deuterated pyridopyrimidinone compound as described herein may be enriched in one enantiomer. For example, a deuterated pyridopyrimidinone compound as described herein have greater than 30% ee, 40% ee, 50% ee, 60% ee, 70% ee, 80% ee, 90% ee, 95% ee, 97% ee, 99% ee, or greater % ee.

In some embodiments, the deuterated pyridopyrimidinone compounds as described herein may have more than one stereocenter. In some embodiments, the deuterated pyridopyrimidinone compounds as described herein may be enriched in one diastereoisomer. For example, the pyridopyrimidinone compounds of Formulas (1)-(5) may have greater than 30% de, 40% de, 50% de, 60% de, 70% de, 80% de, 90% de, 95% de, 99% de, or greater de. In some embodiments, the diastereomerically enriched mixture may comprise at least 60 mol percent of one diastereomer. In some embodiments, the diastereomerically enriched mixture may comprise at least 75, 90, 95, 99 mol percent of one diastereomer.

In some embodiments, the compound of Formulae (3) or (5) has an ee % greater than 95%. In some embodiments, the compound of Formulae (3) or (5) has an ee % greater than 97%. %. In some embodiments, the compound of Formula (3) or (5) has an ee % greater than 99%. In some embodiments, the compound of Formula (3) or (5) has an ee % ranges from about 95% to 100%. In some embodiments, the compound of Formula (5) has an ee % selected from the group of about 95%, about 95.5%, about 96%, about 96.5%, about 97%, about 97.5%, about 98%, about 98.5%, about 99%, about 99.5%, and 100%.

In some embodiments, the deuterated pyridopyrimidinone compounds as described herein also include crystalline and amorphous forms of those compounds, including, for example, polymorphs, pseudo polymorphs, solvates, hydrates, unsolvated polymorphs (including anhydrates), conformational polymorphs, and amorphous forms of the compounds, as well as mixtures thereof “Crystalline form” and “polymorph” are intended to include all crystalline and amorphous forms of the compound, including, for example, polymorphs, pseudo polymorphs, solvates, hydrates, unsolvated polymorphs (including anhydrates), conformational polymorphs, and amorphous forms, as well as mixtures thereof, unless a particular crystalline or amorphous form is referred to.

In some embodiments, the deuterated pyridopyrimidinone compounds as described herein also include prodrug thereof (or “pro-drug”). As used herein, the term “prodrug” refers to compounds that are transformed in vivo to yield a disclosed compound or a pharmaceutically acceptable form of the compound. In some embodiments, the prodrug for the deuterated pyridopyrimidinone compounds as described herein may be inactive when administered to a subject, but is converted in vivo to an active compound, for example, by hydrolysis {e.g., hydrolysis in blood). In some embodiments, the prodrug for the deuterated pyridopyrimidinone compounds as described herein has improved physical and/or delivery properties over the parent compound. In some embodiments, the prodrug for the deuterated pyridopyrimidinone compounds as described herein may increase the bioavailability of the compound when administered to a subject (e.g., by permitting enhanced absorption into the blood following oral administration) or which enhance delivery to a biological compartment of interest (e.g., the brain or lymphatic system) relative to the parent compound. In some embodiments, the prodrug for the deuterated pyridopyrimidinone compounds as described herein may include derivatives of a compound as described herein with enhanced aqueous solubility or active transport through the gut membrane, relative to the parent compound (e.g., amino acid derived prodrug).

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

In some embodiments, the deuterated pyridopyrimidinone compounds as described herein also include solvate thereof. As used herein, the term “solvate” refers to a compound in physical association with one or more molecules of a pharmaceutically acceptable solvent (e.g., a hydrate when the solvent is water). Solvate refers to compounds that further include a stoichiometric or non-stoichiometric amount of solvent bound by non-covalent intermolecular forces. The solvate can be of a compound of the invention or a pharmaceutically acceptable salt thereof. In some embodiments, the deuterated pyridopyrimidinone compounds as described herein may include pharmaceutically acceptable solvates and hydrates. For example, the solvate or hydrate for the herein described deuterated pyridopyrimidinone compounds may include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In some embodiments, the deuterated pyridopyrimidinone compounds as described herein also include pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art (Berge et al., J. Pharmaceutical Sciences, 1977, vol. 66, p. 1-19). Pharmaceutically acceptable salts of the compounds provided herein include those derived from suitable inorganic and organic acids and bases. In some embodiments, the salts can be prepared in situ during the isolation and purification of the herein described compounds, or separately, such as by reacting the free base or free acid of a parent compound with a suitable base or acid, respectively.

In some embodiments, the herein described deuterated pyridopyrimidinone compounds can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. In some embodiments, the compositions disclosed herein are in the form of a pharmaceutically acceptable salt.

In some embodiments, the pyridopyrimidinone compounds as described herein comprises nontoxic acid addition salts of an amino group formed with inorganic acids such as hydrochloric acid, hydro bromic acid, phosphoric acid, sulfuric acid, boric acid, and perchloric acid or with organic acids such as acetic acid, oxalic acid, maleic acid, tartaric acid, citric acid, succinic acid, malonic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, or valeric acid. In some embodiments, the pharmaceutically acceptable salts for any of the herein described deuterated pyridopyrimidinone compounds may include adipate, alginate, ascorbate, aspartate, benzenesulfonate, besylate, benzoate, bisulfate, borate, butyrate, camphorate, camphorsulfonate, citrate, cyclopentanepropionate, digluconate, dodecyl sulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. In some embodiments, the pharmaceutically acceptable acid addition salt for any of herein described deuterated pyridopyrimidinone compounds may be hydrochloride salt.

In some embodiments, the deuterated pyridopyrimidinone compounds as described herein comprises nontoxic salts of an acid group formed with appropriate bases such as alkali metal, alkaline earth metal, ammonium and N⁺(C1-4alkyl)₄ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, such as isopropylamine, trimethylamine, diethylamine, triethylamine, tripropylamine, ethanolamine, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, N-methyl-D-glucamine, 2-amino-2-(hydroxymethyl)-1,3-propanediol, and tromethamine. In some embodiments, the pharmaceutically acceptable base addition salt for any of herein described deuterated pyridopyrimidinone compounds may be chosen from ammonium, potassium, sodium, calcium, and magnesium salts.

### 4. Uses of Deuterated Pyridopyrimidinone Compounds

In an embodiment, this disclosure provides deuterated pyridopyrimidinone compounds as highly selective CDK2 inhibitors. The deuterated pyridopyrimidinone compounds as described herein modulate cyclin A, cyclin E, Rb and E2F responsive genes modulated cell cycle regulation, including gene transcription.

In an embodiment, this disclosure provides deuterated pyridopyrimidinone compounds, wherein hydrogen atom is replaced with deuterium atom at strategic locations of the molecule. Such strategic deuterium substitution leads to enhanced in vivo anti-tumor activities, CDK subtype selectivity and/or pharmacokinetic profile of the selected compounds.

In some embodiments, the herein described deuterated pyridopyrimidinone compound has an IC₅₀<1000 nM in CDK2 enzyme assays described below. In some embodiments, the deuterated pyridopyrimidinone compound has an IC₅₀<100 nM in CDK2 enzyme assays described below. In some embodiments, the herein described deuterated pyridopyrimidinone compound has an IC₅₀<50 nM in CDK2 enzyme assays described below. In some embodiments, the herein described deuterated pyridopyrimidinone compound has an IC₅₀<25 nM in CDK2 enzyme assays described below. In some embodiments, the herein described deuterated pyridopyrimidinone compound has an IC₅₀<10 nM in CDK2 enzyme assays described below. In some embodiments, the herein described deuterated pyridopyrimidinone compound has an IC₅₀<1 nM in CDK2 enzyme assays described below. In some embodiments, the herein described deuterated pyridopyrimidinone compound has an IC₅₀ value ranging from about 0.5 nM to about 1000 nM in CDK2 enzyme assays described below. In some embodiments, the herein described deuterated pyridopyrimidinone compound has an IC₅₀ value ranging from about 0.6 nM to about 100 nM in CDK2 enzyme assays described below. In some embodiments, the herein described deuterated pyridopyrimidinone compound has an IC₅₀ value ranging from about 0.6 nM to about 10 nM in CDK2 enzyme assays described below. In some embodiments, the herein described deuterated pyridopyrimidinone compound has an IC₅₀ value ranging from about 0.6 nM to about 5 nM in CDK2 enzyme assays described below. In some embodiments, the herein described deuterated pyridopyrimidinone compound has an IC₅₀ value ranging from about 0.6 nM to about 4 nM in CDK2 enzyme assays described below. In some embodiments, the herein described deuterated pyridopyrimidinone compound has an IC₅₀ value ranging from about 0.6 nM to about 3 nM in CDK2 enzyme assays described below. In some embodiments, the herein described deuterated pyridopyrimidinone compound has an IC₅₀ value ranging from about 0.6 nM to about 2 nM in CDK2 enzyme assays described below.

In some embodiments, the herein described the deuterated pyridopyrimidinone compounds exhibit high selectivity toward CDK2 over other CDK family enzymes (e.g., CDK1, CDK4, CDK6, CDK7, CDK9). In some embodiments, the herein described the deuterated pyridopyrimidinone compounds exhibit at least 10 fold selectivity toward CDK2 over other CDK family enzymes (e.g., CDK1, CDK4, CDK6, CDK7, CDK9): for example, the IC₅₀ value for CDK2 inhibition is 10 fold less than that of CDK4 and/or CDK6. In some embodiments, the herein described the deuterated pyridopyrimidinone compounds exhibit at least 25 fold selectivity toward CDK2 over other CDK family enzymes (e.g., CDK1, CDK4, CDK6, CDK7, CDK9). In some embodiments, the herein described the deuterated pyridopyrimidinone compounds exhibit at least 50 fold selectivity toward CDK2 over other CDK family enzymes (e.g., CDK1, CDK4, CDK6, CDK7, CDK9).

In some embodiments, the deuterated pyridopyrimidinone compounds have utilities in treating diseases associated with cell-cycle dysregulation by acting on check-point signaling pathway via CDK2 inhibition. In some embodiments, the deuterated pyridopyrimidinone compounds have utilities in treating conditions associated with the modulation of cyclin A, cyclin E, Rb and E2F responsive genes by acting on CDK2 to alter the growth phase or state within the cell cycle of the treated cells. In some embodiments, the deuterated pyridopyrimidinone compounds have utilities in treating diseases associated with cell-cycle dysregulation such as proliferative diseases, cancers, infectious diseases, chronic inflammation diseases, neurodegenerative diseases, or hearing loss.

In some embodiments, the deuterated pyridopyrimidinone compounds have utility in treating cancers such as breast cancer, ovarian cancer, neuroblastoma, glioblastoma, B-cell lymphoma, prostate cancer, liver cancer, lung, acute myeloid leukemia, glioma, colorectal cancers or melanoma. In some embodiments, the deuterated pyridopyrimidinone compounds have utility in treating breast cancer. In some embodiments, the deuterated pyridopyrimidinone compounds have utility in treating ovarian cancer.

In an embodiment, this disclosure provides a deuterated pyridopyrimidinone compound according to any one of Formulae (1)-(5) for use as active therapeutic agent. In some embodiments, the deuterated pyridopyrimidinone compounds of Formulae (1)-(5) possess CDK2 inhibitory activity and may be used in the treatment or prophylaxis of disorders in which CDK2 plays an active role.

In an embodiment, this disclosure provides a compound according to any one of Formulae (1)-(5) for use in the treatment of diseases associated with the modulation of cyclin A, cyclin E, Rb and E2F responsive genes to alter the growth phase or state within the cell cycle of treated cells. In an embodiment, this disclosure provides a compound of any one of Formulae (1)-(5) for use in the treatment of diseases associated with cell-cycle dysregulation. In an embodiment, this disclosure provides a compound of any one of Formulae (1)-(5) for use in the treatment of proliferative diseases. In an embodiment, this disclosure provides a compound of any one of Formulae (1)-(5) for use in the treatment of chronic inflammation diseases. In an embodiment, this disclosure provides a compound of any one of Formulae (1)-(5) for use in the treatment of infection. In an embodiment, this disclosure provides a compound of any one of Formulae (1)-(5) for use in the treatment of neurodegenerative diseases. In an embodiment, this disclosure provides a compound of any one of Formulae (1)-(5) for use in the treatment of cancer. In an embodiment, this disclosure provides a compound of any one of Formulae (1)-(5) for use in the treatment of breast cancer and ovarian cancer. In an embodiment, this disclosure provides a compound of any one of Formulae (1)-(5) for use in the treatment of advanced or metastatic breast cancer and ovarian cancer. In an embodiment, this disclosure provides a compound of any one of Formulae (1)-(5) for use in the treatment of breast cancer. In an embodiment, this disclosure provides a compound of any one of Formulae (1)-(5) for use in the treatment of ovarian cancer.

In an embodiment, this disclosure provides a method of treating diseases associated with the modulation of cyclin A, cyclin E, Rb and E2F responsive genes to alter the growth phase or state within the cell cycle of treated cells, comprising the administration to a subject in need of such treatment a therapeutically-effective amount of a deuterated pyridopyrimidinone compound of Formulae (1)-(5).

In some embodiments, this disclosure provides a method for treating proliferative diseases, chronic inflammation diseases, infectious diseases, neurodegenerative diseases, or hearing loss (or use of the herein described compounds of the present invention for the manufacture of a medicament for the treatment of these diseases), comprising administering to a subject in need of such treatment a therapeutically-effective amount of at least one of the deuterated pyridopyrimidinone compound of Formulae (1)-(5).

In some embodiments, this disclosure provides a method for treating a cancer (or use of the herein described compounds for the manufacture of a medicament for the treatment of these diseases), comprising administering to a subject in need of such treatment a therapeutically-effective amount of a deuterated pyridopyrimidinone compound of Formulae (1)-(5).

In an embodiment, this disclosure provides process and intermediates for making the compounds of the present invention. In some embodiments, the present invention provides the compounds of the present invention for use in therapy. In an embodiment, this disclosure provides the use of the herein described compounds for the treatment of disease conditions where CDK2 plays a role. In some embodiments, the disease conditions where CDK2 plays a role is selected from the group of breast cancer, triple negative breast cancer, ovarian cancer, neuroblastoma, glioblastoma, B-cell lymphoma, prostate cancer, liver cancer, acute myeloid leukemia, or melanoma.

In some embodiments, this disclosure provides a method for treating a disease (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5), wherein the disease is breast cancer, triple negative breast cancer, ovarian cancer, neuroblastoma, glioblastoma, B-cell lymphoma, chronic lymphocytic leukemia (CLL), prostate cancer, liver cancer, acute myeloid leukemia, epithelial ovarian cancer, triple negative breast cancer, HR-positive and HER2⁻ breast cancer, ER⁺ HER2⁻ breast cancer, tamoxifen resistant breast cancer, renal cancer, gastric cancer, lung cancer, esophageal carcinoma, non-Hodgkin's lymphoma, prostate cancer, colon cancer, cervical cancer, colorectal cancer, melanoma, non-small cell lung cancer (NSCLC), multiple myeloma, sarcoma, adenocarcinoma, renal epithelioid angiomyolipoma, 5-FU-resistant colony cancer, paclitaxel-resistant cervical cancer, Cushing's disease, thymoma, leukemia, HIV infection, Parkinson's disease, arthritis, herpes simplex infection, HIV, or cystic fibrosis.

In some embodiments, the present invention provides a method of treating a proliferation disease (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5), wherein the proliferation disease is selected from breast cancer, ovarian cancer, epithelial ovarian cancer, triple negative breast cancer, HR-positive and HER2 breast cancer, ER⁺ HER2⁻ breast cancer, or tamoxifen resistant breast cancer.

In some embodiments, the present invention provides a method of treating advanced or metastatic breast cancers (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5), wherein the advanced or metastatic breast cancer is selected from triple negative breast cancer, HR-positive and HER2⁻ breast cancer, ER⁺ HER2⁻ breast cancer, or tamoxifen resistant breast cancer.

In some embodiments, the present invention provides a method of treating ovarian cancers (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5), wherein the advanced/metastatic ovarian cancer, or epithelial ovarian cancer.

In some embodiments, the present invention provides a method of treating triple negative breast cancer (or use of the herein described deuterated pyridopyrimidinone compounds for the manufacture of a medicament for the treatment of this disease), comprising administering to a subject in need of such treatment a therapeutically-effective amount of a deuterated pyridopyrimidinone compound of Formulae (1)-(5).

In some embodiments, the present invention provides a method of treating HR-positive and HER2⁻ breast cancer (or use of the herein described deuterated pyridopyrimidinone compounds for the manufacture of a medicament for the treatment of this disease), comprising administering to a subject in need of such treatment a therapeutically-effective amount of a deuterated pyridopyrimidinone compound of Formulae (1)-(5).

In some embodiments, the present invention provides a method of treating ER⁺ HER2⁻ breast cancer (or use of the herein described deuterated pyridopyrimidinone compounds for the manufacture of a medicament for the treatment of this disease), comprising administering to a subject in need of such treatment a therapeutically-effective amount of a deuterated pyridopyrimidinone compound of Formulae (1)-(5).

In some embodiments, the present invention provides a method of treating tamoxifen resistant breast cancer (or use of the herein described deuterated pyridopyrimidinone compounds for the manufacture of a medicament for the treatment of this disease), comprising administering to a subject in need of such treatment a therapeutically-effective amount of a deuterated pyridopyrimidinone compound of Formulae (1)-(5).

In some embodiments, the present invention provides a method of treating epithelial ovarian cancer (or use of the herein described deuterated pyridopyrimidinone compounds for the manufacture of a medicament for the treatment of this disease), comprising administering to a subject in need of such treatment a therapeutically-effective amount of a deuterated pyridopyrimidinone compound of Formulae (1)-(5).

In some embodiments, the present invention provides a method of treating diseases associated with cell cycle deregulations (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5).

In some embodiments, the present invention provides a method of treating cycle regulating enzymes cyclin A, cyclin E, or Rb mediated diseases (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5).

In some embodiments, the present invention provides a method of treating cycle regulating enzymes cyclin A, cyclin E, or Rb mediated diseases (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5), wherein the cyclin A, cyclin E, or Rb mediated disease is selected from breast cancer, ovarian cancer, advanced or metastatic breast cancer, advanced or metastatic ovarian cancer, epithelial ovarian cancer, triple negative breast cancer, HR-positive and HER2 breast cancer, ER⁺ HER2⁻ breast cancer, or tamoxifen resistant breast cancer.

In some embodiments, the present invention provides a method of treating diseases (or use of the herein described deuterated pyridopyrimidinone compounds for the manufacture of a medicament for the treatment of these diseases), comprising administering to a subject in need of such treatment a therapeutically-effective amount of a deuterated pyridopyrimidinone compound of Formulae (1)-(5) in combination with an additional antineoplastic agent.

In some embodiments, this disclosure provides a method of treating a cancer in a subject in need thereof comprising administering to the subject a pharmaceutical composition comprising a therapeutically effective amount of a deuterated pyridopyrimidinone compound selected from one or more of Comps. 7-213 disclosed in Table 1.

In some embodiments, this disclosure provides a method of treating a cancer in a subject suffering from the cancer in which cancer cells overexpress cyclin-dependent kinase 2 (CDK2), or cyclins modulated by CDK2 (i.e., cyclin A, cyclin E including cyclin E1, cyclin E2) relative to an expression level in normal cells, the method comprising the step of administering to the subject a therapeutically effective dose of a deuterated pyridopyrimidinone compound selected from one or more of Comps. 7-213 disclosed in Table 1. The method for detecting overexpressed CDK2 proteins, or cyclins modulated by CDK2 in cancer patient is known to one skilled in the art, for example, the method for detecting overexpressed CDK2 has been described in U.S. Pat. No. 6,521,412, which is herein incorporated by reference in its entireties, and See Sante et al. and Caldon et al. supra for the methods of detecting overexpression of cyclin E1 and cyclin E2.

In some embodiments, for any of the herein described methods, the cancer in which cancer cells overexpress cyclin-dependent kinase 2 (CDK2), or cyclins modulated by CDK2 is selected from the group of breast cancer, metastatic breast cancer, triple negative breast cancer, tamoxifen resistant breast cancer, HR⁺ and HER2-negative breast cancer, ERα⁺ HER2⁻ breast cancer, ovarian cancer, epithelial ovarian cancer, neuroblastoma, glioblastoma, B-cell lymphoma, prostate cancer, liver cancer, acute myeloid leukemia, melanoma, lymphoma, esophageal squamous cell cancer, and uterine cancer.

In some embodiments, for any of the herein described methods, the cancer in which cancer cells overexpress CDK2, or cyclins modulated by CDK2 is selected from the group of breast cancer or ovarian cancer. In some embodiments, for any of the herein described methods, the cancer in which cancer cells overexpress CDK2, or cyclins modulated by CDK2 is breast cancer. In some embodiments, for any of the herein described methods, the cancer in which cancer cells overexpress CDK2, or cyclins modulated by CDK2 is ovarian cancer. In some embodiments, for any of the herein described methods, the beast cancer in which cancer cells overexpress CDK2, or cyclins modulated by CDK2 is selected from the group triple negative breast cancer, tamoxifen resistant breast cancer, HR⁺ and HER2-negative breast cancer, or ERα⁺ HER2⁻ breast cancer. In some embodiments, for any of the herein described methods, the beast cancer in which cancer cells overexpress CDK2, or cyclins modulated by CDK2 is triple negative breast cancer. In some embodiments, for any of the herein described methods, the beast cancer in which cancer cells overexpress CDK2, or cyclins modulated by CDK2 is tamoxifen resistant breast cancer. In some embodiments, for any of the herein described methods, the beast cancer in which cancer cells overexpress CDK2, or cyclins modulated by CDK2 is HR⁺ and HER2-negative breast cancer. In some embodiments, for any of the herein described methods, the beast cancer in which cancer cells overexpress CDK2, or cyclins modulated by CDK2 is ERα⁺ HER2⁻ breast cancer.

Efficacy of the compounds and combinations of compounds described herein in treating, preventing and/or managing the indicated diseases or disorders can be tested using various models known in the art, which provide guidance for treatment of human disease. For example, models for determining efficacy of treatments for ovarian cancer are described, e.g., in Mullany et al. Endocrinology 2012, 153, 1585-92; and Fong et al. J. Ovarian Res. 2009, 2, 12. Models for determining efficacy of treatments for breast cancer are described, e.g., in Fantozzi, Breast Cancer Res. 2006, 8, 212. Models for determining efficacy in B cell lymphomas, such as diffuse large B cell lymphoma (DLBCL), include the PiBCL1 murine model with BALB/c (haplotype H-2d) mice. Illidge et al. Cancer Biother. & Radiopharm. 2000, 15, 571-80.

In some embodiments, the method described herein finds application as first-line treatment for breast cancer. In some embodiments, the method described herein finds application as first-line treatment for postmenopausal women and premenopausal women with ERα⁺ HER2⁻ advanced or metastatic breast cancer. In some embodiments, the deuterated pyridopyrimidinone compound used in the method described herein finds application as first-line monotherapy for patients with previously untreated ERα⁺ HER2⁻ advanced or metastatic breast cancer. In some embodiments, the deuterated pyridopyrimidinone compound used in the method described herein finds application as first-line monotherapy for patients with ERα⁺ HER2⁻ advanced or metastatic breast cancer after previous endocrine therapy.

In some embodiments, the method described herein finds application as first-line treatment for ovarian cancer. In some embodiments, the method described herein finds application as first-line treatment for advanced or metastatic ovarian cancer.

In some embodiments, the deuterated pyridopyrimidinone compound used in any one of the herein described method finds application in combination therapy for patients with previously untreated ERα⁺ HER2⁻ advanced or metastatic breast cancer. In some embodiments, the deuterated pyridopyrimidinone compound used in any one of the herein described method finds application in combination therapy for patients with ERα⁺ HER2⁻ advanced or metastatic breast cancer after previous endocrine therapy.

In some embodiments, for any one of the methods described herein, the deuterated pyridopyrimidinone compounds of Formulae (1)-(5) may be administered by any conventional route of administration including, but not limited to, oral, pulmonary, intraperitoneal (ip), intravenous (iv), intramuscular (im), subcutaneous (sc), transdermal, buccal, nasal, sublingual, ocular, rectal, intranasal and the like. In some embodiments, for any one of the methods described herein, the medicament may be adapted to be administered by oral administration, injection (e.g., intravenous injection) or by other mode of administration (e.g., parenteral administration).

In some embodiments, the administration in any one of the herein described method or use results in the delivery of one or more deuterated pyridopyrimidinone compounds into the bloodstream (via enteral or parenteral administration), or alternatively, the one or more deuterated pyridopyrimidinone compounds are administered directly to the site of tumor.

Delivery of one or more of the herein described deuterated pyridopyrimidinone compounds may be via intravenous injection, or intravenous infusion. Devices and apparatuses for performing these delivery methods are well known in the art.

Delivery of one or more of the herein described deuterated pyridopyrimidinone compounds into the bloodstream may be achieved via intravenous injection or intravenous infusion.

Dosage forms suitable for parenteral administration include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

In some embodiments, delivery of the herein described deuterated pyridopyrimidinone compounds may be in a vesicle, in particular a liposome (see Langer, 1990, Science 249:1527-1533; Treat et al., in Liposomes in Therapy of Infectious Disease and Cancer, Lopez-Berestein and Fidler (eds.), Liss, New York, pp. 353-365 (1989).

In some embodiments, any one of the herein described deuterated pyridopyrimidinone compounds may be administered by a controlled-release or a sustained-release means or by delivery a device that is well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In other embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; Levy et al., 1985, Science 228:190; During et al., 1989, Ann. Neurol. 25:351; Howard et al., 1989, J. Neurosurg. 71:105).

In other embodiments, a controlled-release system can be placed in proximity of the target area to be treated, e.g., the tumor site, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533 may be used.

The dosage regimen utilizing any of herein described deuterated pyridopyrimidinone compounds can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed.

The dosage of the herein described deuterated pyridopyrimidinone compounds as well as the dosing schedule can depend on various parameters and factors, including, but not limited to, the specific deuterated pyridopyrimidinone compounds, the disease being treated, the severity of the condition, whether the condition is to be treated or prevented, the subject's age, weight, and general health, and the administering physician's discretion. Additionally, pharmacogenomics (the effect of genotype on the pharmacokinetic, pharmacodynamics or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

In some embodiments, the herein described deuterated pyridopyrimidinone compounds may be administered at a dose of about 0.01 mg/kg to about 30 mg/kg. In some embodiments, the herein described deuterated pyridopyrimidinone compounds may be administered at a dose of about 1.0 mg/kg to about 30 mg/kg. In some embodiments, the herein described deuterated pyridopyrimidinone compounds may be administered at a dose of about 0.01 mg/kg to about 20 mg/kg. In some embodiments, the herein described deuterated pyridopyrimidinone compounds may be administered at a dose of about 1.0 mg/kg to about 20 mg/kg. In some embodiments, the herein described deuterated pyridopyrimidinone compounds may be administered at a dose of about 0.01 mg/kg to about 7.5 mg/kg. In some embodiments, the herein described deuterated pyridopyrimidinone compounds may be administered at a dose of about 0.01 mg/kg to about 5.0 mg/kg. In some embodiments, the herein described deuterated pyridopyrimidinone compounds may be administered at a dose of about 1 mg/kg to about 2.5 mg/kg. In some embodiments, the herein described deuterated pyridopyrimidinone compounds may be administered at a dose of about 1 mg/kg to about 2.0 mg/kg. In some embodiments, the herein described deuterated pyridopyrimidinone compounds may be administered at a dose of about 0.5 mg/kg to about 2.0 mg/kg. In some embodiments, the herein described deuterated pyridopyrimidinone compounds may be administered at a dose of about 1 mg/kg to about 5 mg/kg. In some embodiments, the herein described deuterated pyridopyrimidinone compounds may be administered at a dose of about 2 mg/kg to about 7 mg/kg. In some embodiments, the herein described deuterated pyridopyrimidinone compounds may be administered at a dose of about 3 mg/kg to about 8 mg/kg. In some embodiments, the herein described deuterated pyridopyrimidinone compounds may be administered at a dose of about 4 mg/kg to about 9 mg/kg. In some embodiments, the herein described deuterated pyridopyrimidinone compounds may be administered at a dose of about 5 mg/kg to about 10 mg/kg. In some embodiments, the herein described deuterated pyridopyrimidinone compounds may be administered at a dose of about 6 mg/kg to about 11 mg/kg. In some embodiments, the herein described deuterated pyridopyrimidinone compounds may be administered at a dose of about 7 mg/kg to about 12 mg/kg. In some embodiments, the herein described deuterated pyridopyrimidinone compounds may be administered at a dose of about 8 mg/kg to about 13 mg/kg. In some embodiments, the herein described deuterated pyridopyrimidinone compounds may be administered at a dose of about 9 mg/kg to about 14 mg/kg. In some embodiments, the herein described deuterated pyridopyrimidinone compounds may be administered at a dose of about 10 mg/kg to about 15 mg/kg. In some embodiments, the herein described deuterated pyridopyrimidinone compounds may be administered at a dose of about 11 mg/kg to about 16 mg/kg. In some embodiments, the herein described deuterated pyridopyrimidinone compounds may be administered at a dose of about 12 mg/kg to about 17 mg/kg. In some embodiments, the herein described deuterated pyridopyrimidinone compounds may be administered at a dose of about 13 mg/kg to about 18 mg/kg. In some embodiments, the herein described deuterated pyridopyrimidinone compounds may be administered at a dose of about 14 mg/kg to about 19 mg/kg. In some embodiments, the herein described deuterated pyridopyrimidinone compounds may be administered at a dose of about 15 mg/kg to about 20 mg/kg. In some embodiments, the herein described deuterated pyridopyrimidinone compounds may be administered at a dose selected from the group of about 0.01 mg/kg, about 0.1 mg/kg, about 0.5 mg/kg, about 1.0 mg/kg, about 2.0 mg/kg, about 3.0 mg/kg, about 4.0 mg/kg, about 5.0 mg·kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, about 25.0 mg/kg, about 26.0 mg/kg, about 27.0 mg/kg, about 28.0 mg/kg, about 29.0 mg/kg, or about 30.0 mg/kg.

In some embodiments, the herein described deuterated pyridopyrimidinone compounds may be administered in a single daily dose (also known as QD, qd or q.d.), or the total daily dosage can be administered in divided doses of twice daily (also known as BID, bid, or b.i.d.), three times daily (also known as TID, tid, or t.i.d.), or four times daily (also known as QID, qid, or q.i.d.). In some embodiments, any one of herein described deuterated pyridopyrimidinone compounds may be administered once daily. In some embodiments, the herein described deuterated pyridopyrimidinone compounds may be administered twice daily. Furthermore, the herein described deuterated pyridopyrimidinone compounds may be administered continuously or intermittently throughout the dosage regimen. Furthermore, the herein described deuterated pyridopyrimidinone compounds may be administered continuously throughout the dosage regimen. Furthermore, the herein described deuterated pyridopyrimidinone compounds may be administered intermittently throughout the dosage regimen.

In some embodiments, this disclosure provides a method of assaying for cancer cell sensitivity to the deuterated pyridopyrimidinone compound of Formulae (1)-(5) as described herein comprising: (a) providing cancer cells; (b) contacting the cancer cells with the deuterated pyridopyrimidinone compound of Formulae (1)-(5) as described herein; (c) analyzing the cells for inhibition of growth; and (d) comparing the inhibition of growth in the cancer cell from step (c) with the inhibition of growth in the cancer in the absence of the deuterated pyridopyrimidinone compound of Formulae (1)-(5) as described herein, wherein growth inhibition indications that said cancer cell is susceptible to the deuterated pyridopyrimidinone compound of Formulae (1)-(5) described herein. In some embodiments, the cancer cells used in any of herein described method may include human ovarian carcinoma OVCAR3 cells (ATCC HTB-161™) or human breast squamous HCC1806 cells. In some embodiments, the cancer cells used in any of herein described method may include human OVCAR3 cells. In some embodiments, the cancer cells used in any of herein described method may include human breast squamous HCC1806 cells.

### 5. Pharmaceutical Compositions and Formulations

In an embodiment, this disclosure provides a pharmaceutical composition comprising one or more of the herein described deuterated pyridopyrimidinone compounds, or pharmaceutically acceptable salt, stereoisomer, cocrystal, prodrug, solvate, hydrate, and polymorph thereof. In an embodiment, this disclosure provides a pharmaceutical composition comprising the compound of any one of the deuterated pyridopyrimidinone compounds of Formulae (1)-(5), or pharmaceutically acceptable salt, stereoisomer, cocrystal, prodrug, solvate, hydrate, and polymorph thereof, and at least one pharmaceutically acceptable carrier, diluent or excipient. In some embodiments, this disclosure provides a pharmaceutical composition comprising one or more of the deuterated pyridopyrimidinone compounds selected from Comps. 7-213 disclosed in Table 1 below, or pharmaceutically acceptable salt, stereoisomer, cocrystal, prodrug, solvate, hydrate, and polymorph thereof, and at least one pharmaceutically acceptable carrier, diluent or excipient.

In an embodiment, this disclosure provides a pharmaceutical composition useful for treating diseases associated with the modulation of cyclin A, cyclin E, Rb and E2F responsive genes by acting on CDK2 to alter the growth phase or state within the cell cycle of treated cells, comprising the compound of one or more of the herein described deuterated pyridopyrimidinone compounds, or pharmaceutically acceptable salt, stereoisomer, cocrystal, prodrug, solvate, hydrate, and polymorph thereof, and at least one pharmaceutically acceptable carrier or diluent.

In some embodiments, the pharmaceutical composition further comprises an additional antineoplastic agent. In some embodiments, the additional antineoplastic agent is selected from the group of aromatase inhibitor, hormonal therapy, selective estrogen receptor degrader, cytotoxic agents, PD-1 antagonist, PD-L1 antagonist, AR inhibitor, inhibitor of glutaminase, CDK4/6 inhibitor, CDK9 inhibitor, or Akt inhibitor. In some embodiments, the additional antineoplastic agent is tamoxifen, docetaxel, paclitaxel, cisplatin, capecitabine, gemcitabine, vinorelbine, exemestane, letrozole, fulvestrant, anastrozole, or trastuzumab.

In some embodiments, the pharmaceutical composition further comprises an additional antineoplastic agent to form a fixed combination dose. In some embodiments, the any one of the herein described pharmaceutical formulations may be presented in unitary dose forms containing a predetermined amount of the any one of the herein described deuterated pyridopyrimidinone compounds per unit dose. Such a unit may contain, as a non-limiting example, 0.5 mg to 2500 mg of a deuterated pyridopyrimidinone compound described herein, depending on the condition being treated, the route of administration, and the age, weight, and condition of the patient. In some embodiments, the unitary dose form may contain 0.5 mg to 1000 mg of a deuterated pyridopyrimidinone compound described herein. In some embodiments, the unitary dose form may contain 1.0 mg to 750 mg of a deuterated pyridopyrimidinone compound described herein. In some embodiments, the unitary dose form may contain 1.0 mg to 500 mg of a deuterated pyridopyrimidinone compound described herein. In some embodiments, the unitary dose form may contain 1.0 mg to 250 mg of a deuterated pyridopyrimidinone compound described herein. In some embodiments, the unitary dose form may contain 1.0 mg to 200 mg of a deuterated pyridopyrimidinone compound described herein. In some embodiments, the unitary dose form may contain 1.0 mg to 150 mg of a deuterated pyridopyrimidinone compound described herein. In some embodiments, the unitary dose form may contain 1.0 mg to 100 mg of a deuterated pyridopyrimidinone compound described herein. In some embodiments, the unitary dose form may contain 1.0 mg to 50 mg of a deuterated pyridopyrimidinone compound described herein. In some embodiments, the unitary dose form may contain 1.0 mg to 25 mg of a deuterated pyridopyrimidinone compound described herein. In some embodiments, the unitary dose form may contain 1.0 mg to 10 mg of a deuterated pyridopyrimidinone compound described herein. In some embodiments, the unitary dose form may contain 1.0 mg to 5 mg of a deuterated pyridopyrimidinone compound described herein. In some embodiments, the unitary dose form may contain about 0.5 mg, about 1.0 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2100 mg, about 2200 mg, about 2300 mg, about 2400 mg, or about 2500 mg of a deuterated pyridopyrimidinone compound described herein, or pharmaceutically acceptable salt, stereoisomer, cocrystal, prodrug, solvate, hydrate, and polymorph thereof. In some embodiments, the herein described unitary dosage forms contains about 10 mg of a deuterated pyridopyrimidinone compound described herein. In some embodiments, the herein described unitary dosage forms contains about 20 mg of a deuterated pyridopyrimidinone compound described herein. In some embodiments, the herein described unitary dosage forms contains about 30 mg of a deuterated pyridopyrimidinone compound described herein. In some embodiments, the herein described unitary dosage forms contains about 50 mg of a deuterated pyridopyrimidinone compound described herein. In some embodiments, the herein described unitary dosage forms contains about 75 mg of a deuterated pyridopyrimidinone compound described herein. In some embodiments, the herein described unitary dosage forms contains about 100 mg of a deuterated pyridopyrimidinone compound described herein. In some embodiments, the herein described unitary dosage forms contains about 150 mg of a deuterated pyridopyrimidinone compound described herein. In some embodiments, the herein described unitary dosage forms contains about 200 mg of a deuterated pyridopyrimidinone compound described herein. In some embodiments, the herein described unitary dosage forms contains about 250 mg of a deuterated pyridopyrimidinone compound described herein. In some embodiments, the herein described unitary dosage forms contains about 350 mg of a deuterated pyridopyrimidinone compound described herein. In some embodiments, the herein described unitary dosage forms contains about 500 mg of a deuterated pyridopyrimidinone compound described herein. In some embodiments, the herein described unitary dosage forms contains about 750 mg of a deuterated pyridopyrimidinone compound described herein. In some embodiments, the herein described unitary dosage forms contains about 1000 mg of a deuterated pyridopyrimidinone compound described herein.

In some embodiments, the any one of the herein described unitary dosage forms contains a daily dose or sub-dose, as herein described, or an appropriate fraction thereof, of a deuterated pyridopyrimidinone compounds described herein, or pharmaceutically acceptable salt, stereoisomer, cocrystal, prodrug, solvate, hydrate, and polymorph thereof. Such pharmaceutical formulations may be prepared by any of the methods well known in the pharmacy art.

In some embodiments, the any one of the herein described pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

In some embodiments, the pharmaceutical formulation may additionally comprises a pharmaceutically acceptable excipient, carrier, diluent, or vehicle, such as a liquid or solid filler, diluent, excipient, solvent or encapsulating material, involved in carrying or transporting of biologically active agents to animals, in particular, mammals. In some embodiments, the pharmaceutically acceptable carriers are formulated according to a number of factors well within the purview of those of ordinary skill in the art. These include without limitation the type and nature of the active agent being formulated; the subject to which the agent containing composition to be administered, the intended route of administration of the composition, and the therapeutic indication being targeted. In some embodiments, the pharmaceutically acceptable carriers include both the aqueous and non-aqueous liquid media as well as a variety of solid and semi-solid dosage forms. Such carriers may include a number of different ingredients and additives in addition to the active agent. Each carrier must be “acceptable” in the sense of being compatible with the other ingredients of the formulation and not injurious to the patient.

In some embodiments, the pharmaceutically acceptable excipient may include solvents, dispersion media, diluents, or other liquid vehicles, dispersion or suspension aids, surface active agents, isotonic agents, thickening or emulsifying agents, preservatives, solid binders, lubricants, antimicrobial preservatives, antioxidants and other excipients such as dispersing, suspending, thickening, emulsifying, buffering, wetting, solubilizing, stabilizing, flavoring and sweetening agents. Liquid vehicle may include PBS buffer, saline, sucrose or a suitable polyhydric alcohol or alcohols and which optionally contain ethanol, an elixir or linctus.

In some embodiments, the pharmaceutically acceptable excipient may include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. In some embodiments, the pharmaceutically acceptable excipient may further include wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants.

In some embodiments, any one of the herein described pharmaceutical formulation adapted for administration by any appropriate route is selected from the group of a capsule, a tablet, a buccal tablet, an oral disintegrating tablet, a mucoadhesive tablet, a liquid formulation, a dispersion, an injection preparation, powder for injection, and suppository.

In some embodiments, the any one of the herein described pharmaceutical composition comprises an oral dosage selected from tablets, capsules, troches, pellets, granules, powders, solutions, syrups, elixirs, suspensions, or dispersions.

In some embodiments, the pharmaceutically acceptable excipients may be used in the manufacture of solid oral dosage include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, and flavoring can be present in the composition, according to the judgment of the formulator. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, the any one of the herein described pharmaceutical formulations adapted for oral administration may be presented as discrete units such as capsules or tablets; powders or granules; solutions or suspensions, each with aqueous or non-aqueous liquids; edible foams or whips; or emulsions including oil-in-water emulsions or water-in-oil emulsions. For Example, for oral administration in the form of a tablet or capsule, the deuterated pyridopyrimidinone compounds as described herein may be combined with an oral, non-toxic pharmaceutically acceptable inert carrier such as ethanol, glycerol, water, and the like. Generally, granules or powders are prepared by comminuting the deuterated pyridopyrimidinone compounds to a suitable fine size and mixing with an appropriate pharmaceutical carrier such as an edible carbohydrate, as, for example, starch or mannitol. Flavorings, preservatives, dispersing agents, and coloring agents may also be present.

In some embodiments, the any one of the herein described pharmaceutical formulations adapted for oral administration may be capsules made by preparing a powder, liquid, or suspension mixture and encapsulating with gelatin or some other appropriate shell material (e.g., pullulan polysaccharide). Glidants and lubricants such as colloidal silica, talc, magnesium stearate, calcium stearate, or solid polyethylene glycol may be added to the mixture before the encapsulation. A disintegrating or solubilizing agent such as agar-agar, calcium carbonate or sodium carbonate may also be added to improve the availability of the medicament when the capsule is ingested. Moreover, when desired or necessary, suitable binders, lubricants, disintegrating agents, and coloring agents may also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these capsule dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

In some embodiments, the any one of the herein described pharmaceutical formulations adapted for oral administration may be tablets formulated by preparing a powder mixture, granulating or slugging, adding a lubricant and disintegrant, and pressing into tablets. A powder mixture may be prepared by mixing the deuterated pyridopyrimidinone CDK2 inhibitor as described herein, suitably comminuted, with one or more diluents or excipients. In some embodiments, the tablets as described herein may comprise optional ingredients including binders such as carboxymethylcellulose, alginates, gelatins, or polyvinyl pyrrolidone, solution retardants such as paraffin, resorption accelerators such as a quaternary salt, and/or absorption agents such as bentonite, kaolin, or dicalcium phosphate. In some embodiments, the powder mixture may be wet-granulated with a binder such as syrup, starch paste, acadia mucilage or solutions of cellulosic or polymeric materials, and forcing through a screen. In some embodiments, the granules may be lubricated to prevent sticking to the tablet forming dies by means of the addition of stearic acid, a stearate salt, talc or mineral oil. The lubricated mixture is then compressed into tablets. In some embodiments, the compounds of the present invention may also be combined with a free flowing inert carrier and compressed into tablets directly without going through the granulating or slugging steps. In some embodiments, a clear or opaque protective coating consisting of a sealing coat of shellac, a coating of sugar or polymeric material, and a polish coating of wax may be provided. In some embodiments, dyestuffs may be added to these coatings to distinguish different unit dosages.

In some embodiments, the liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such Cremophor®, alcohols, oils, modified oils, glycols, polysorbates, cyclodextrins, polymers, and/or combinations thereof.

In some embodiments, the unitary dosage forms for oral administration may be microencapsulated. In some embodiments, the formulation may also be prepared to prolong or sustain the release as for example by coating or embedding particulate material in polymers, wax or the like.

In some embodiments, the intravenous injection or infusion for systemic administration of the herein described pharmaceutical formulations may be employed. In some embodiments, the pharmaceutically acceptable excipients suitable for liquid formulation include solvents, dispersion media, diluents, or other liquid vehicles, dispersion or suspension aids, surface active agents, isotonic agents, thickening or emulsifying agents, preservatives, antimicrobial preservatives, antioxidants and other excipients such as dispersing, suspending, thickening, emulsifying, buffering, wetting, or stabilizing agents. In some embodiments, the liquid vehicle may include PBS buffer, saline, sucrose or a suitable polyhydric alcohol or alcohols and which optionally contain ethanol, an elixir or linctus.

In some embodiments, the liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions are mixed with Cremophor®, alcohols, oils, modified oils, glycols, polysorbates, cyclodextrins, polymers, and/or combinations thereof.

In some embodiments, the injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectable compositions.

In some embodiments, the deuterated pyridopyrimidinone compounds in any of the herein described pharmaceutical composition comprises a mixture of about 90% or more by weight of the (−)-enantiomer and about 10% or less by weight of the (+)-enantiomer.

Further, the deuterated pyridopyrimidinone compounds disclosed herein may be administered to a subject as a component of a composition, e.g., pharmaceutical composition that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In some embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The deuterated pyridopyrimidinone compounds disclosed herein, if desired, can also formulated with wetting or emulsifying agents, or pH buffering agents. Other examples of suitable pharmaceutical excipients are described in Remington's Pharmaceutical Sciences 1447-1676 (Alfonso R. Gennaro eds., 19th ed. 1995), incorporated herein by reference.

In some embodiments, the compositions, e.g., pharmaceutical compositions, disclosed herein are suspended in a saline buffer (including, without limitation TBS, PBS, and the like).

In some embodiments, the pharmaceutical composition for the deuterated pyridopyrimidinone compounds disclosed herein can take the form of solutions, suspensions, emulsion, drops, tablets, pills, pellets, capsules, capsules containing liquids, powders, sustained-release formulations, emulsions, aerosols, sprays, suspensions, or any other form suitable for use. Where necessary, the pharmaceutical compositions comprising the deuterated pyridopyrimidinone compounds described herein can also include a solubilizing agent. Also, the agents can be delivered with a suitable vehicle or delivery device as known in the art.

In some embodiments, the pharmaceutical composition for the deuterated pyridopyrimidinone compounds disclosed herein may optionally include a local anesthetic such as, for example, lignocaine to lessen pain at the site of the injection.

In some embodiments, the herein described pharmaceutical composition comprises a liquid composition having about 0.0001% (w/v) to about 50% (w/v), about 0.01% (w/v) to about 20% (w/v), about 0.01% to about 10% (w/v), about 1% (w/v) to about 3% (w/v), about 3% (w/v) to about 5% (w/v), about 5% (w/v) to about 7% (w/v), about 5% (w/v) to about 15% (w/v), about 7% (w/v) to about 10% (w/v), about 10% (w/v) to about 15% (w/v), about 15% (w/v) to about 20% (w/v), about 20% (w/v) to about 30% (w/v), about 30% (w/v) to about 40% (w/v), or about 40% (w/v) to about 50% (w/v) of a deuterated pyridopyrimidinone compound as described herein by the total weight of the pharmaceutical composition.

In some embodiments, the pharmaceutical composition comprises a liquid dosage form containing about 10 mg to about 2000 mg, about 10 mg to about 1500 mg, about 10 mg to about 1000 mg, about 50 mg to about 1000 mg, about 10 mg to about 50 mg, about 50 mg to about 100 mg, about 40 mg to about 90 mg, about 200 mg to about 300 mg, about 70 mg to about 95 mg, about 100 mg to about 200 mg, about 105 mg to about 200 mg, about 110 mg to about 140 mg, about 180 mg to about 220 mg, about 280 mg to about 320 mg, about 200 mg, about 150 mg, or about 300 mg of the deuterated pyridopyrimidinone compound as described herein, or any amount of the deuterated pyridopyrimidinone compounds in a range bounded by, or between, any of these values.

In some embodiments, the pharmaceutical composition comprises a solid composition having at least about 5% (w/w), at least about 10% (w/w), at least about 20% (w/w), at least about 50% (w/w), at least about 70% (w/w), at least about 80%, about 10% (w/w) to about 30% (w/w), about 10% (w/w) to about 20% (w/w), about 20% (w/w) to about 30% (w/w), about 30% (w/w) to about 50% (w/w), about 30% (w/w) to about 40% (w/w), about 40% (w/w) to about 50% (w/w), about 50% (w/w) to about 80% (w/w), about 50% (w/w) to about 60% (w/w), about 70% (w/w) to about 80% (w/w), or about 80% (w/w) to about 90% (w/w) of the deuterated pyridopyrimidinone compound as described herein by the total weight of the pharmaceutical composition.

In some embodiments, the pharmaceutical composition comprises a solid dosage form containing about 10 mg to about 2000 mg, about 10 mg to about 1500 mg, about 10 mg to about 1000 mg, about 50 mg to about 1000 mg, about 10 mg to about 50 mg, about 50 mg to about 100 mg, about 40 mg to about 90 mg, about 200 mg to about 300 mg, about 70 mg to about 95 mg, about 100 mg to about 200 mg, about 105 mg to about 200 mg, about 110 mg to about 140 mg, about 50 mg to about 150 mg, about 180 mg to about 220 mg, about 280 mg to about 320 mg, about 200 mg, about 150 mg, or about 300 mg of the deuterated pyridopyrimidinone compound as described herein.

### 6. Combination Therapy

In some embodiments, the deuterated pyridopyrimidinone compounds as described herein may be used as monotherapy for the treatment of disorders associated with cell cycle deregulations caused by cyclin A and/or cyclin E by acting on CDK2. In some embodiments, the deuterated pyridopyrimidinone compound as described herein may be used as a first-line or second-line monotherapy for the treatment of disorders associated with cell cycle deregulations caused by cyclin A and/or cyclin E by acting on CDK2. In some embodiments, the deuterated pyridopyrimidinone compound as described herein may also be combined or used in combination with therapeutic agents useful in the treatment of disorders associated with cell cycle deregulations caused by cyclin A and/or cyclin E by acting on CDK2. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced).

In some embodiments, the herein described pharmaceutical composition further comprises an additional antineoplastic agent for use in the treatment of disorders associated with cell cycle deregulations caused by cyclin A and/or cyclin E by acting on CDK2. In some embodiments, the present disclosure provides a pharmaceutical composition comprising the combination of a deuterated pyridopyrimidinone compound selected from Comps. 7-213 disclosed in Table 1, or pharmaceutically acceptable salt, stereoisomer, cocrystal, prodrug, solvate, hydrate, and polymorph thereof, with one or more of an additional antineoplastic agent for the treatment of a disease or disorder selected from the group consisting of breast cancer, metastatic breast cancer, triple negative breast cancer, tamoxifen resistant breast cancer, HR⁺ and HER2-negative breast cancer, ERα⁺ HER2⁻ breast cancer, ovarian cancer, epithelial ovarian cancer, neuroblastoma, glioblastoma, B-cell lymphoma, prostate cancer, liver cancer, acute myeloid leukemia, melanoma.

As used herein, the term “combination” or “pharmaceutical combination” refers to the combined administration of the herein described deuterated pyridopyrimidinone compounds with one or more of additional antineoplastic agents. The combination therapies described herein overcome drug resistant issues associated with CDK4/6 inhibitor monotherapy.

In some embodiments, the herein described deuterated pyridopyrimidinone compound may be combined with radiation, surgery, additional antineoplastic agents, aromatase inhibitor, mitotic inhibitors, hormonal therapy, selective estrogen receptor degrader (SERD, e.g., fulvestrant), cytotoxic agents, microtubule stabilizing agent, alkylating agents, topoisomerase inhibitors, targeted cancer therapy, cancer immunotherapy monoclonal antibodies, immunoregulator such as PD-1 antagonist, thalidomide, anticancer agents, endocrine therapy, and the like. In some embodiments, the targeted therapy used in any of the herein described combination therapy is selected from the group of IMiDs (immunomodulatory drugs), protease inhibitor, HDAC (histone deacetylase) inhibitor, IKK (IκB kinase) inhibitor, PI3K inhibitor, mTOR inhibitor, Akt inhibitor, poly(ADP-ribose) (PARP) inhibitor, IDO (indoleamine 2,3-dioxygenase) inhibitor, TDO (tryptophan 2,3 dioxygenase) inhibitor, ALK (anaplastic lymphoma kinase) inhibitor, ROS (reactive oxygen species) inhibitor, MEK (mitogen-activated protein kinase) inhibitor, VEGF (vascular endothelial growth factor) inhibitor, FLT3 (FMS-like receptor tyrosine kinase-3) inhibitor, AXL (AXL receptor tyrosine kinase) inhibitor, ROR2 (orphan receptor tyrosine kinase-like receptor 2) inhibitor, EGFR (epidermal growth factor receptor) inhibitor, FGFR (fibroblast growth factor receptor) inhibitor, AR (androgen receptor) inhibitor, Src/Abl (proto-oncogene tyrosine-protein kinase/Abelson tyrosine kinase) inhibitor, PRK/Ras (phosphoribulokinase/Ras) inhibitor, Myc inhibitor, Raf inhibitor, PGDF (platelet-derived growth factor receptor) inhibitor, c-Kit (c-Kit receptor tyrosine kinase) inhibitor, ErbB2 (HER2) inhibitor, CDK4/6 inhibitor, CDK5 inhibitor, CDK7 inhibitor, CDK9 inhibitor, SMO (smoothened) inhibitor, CXCR4 inhibitor, GLS1 inhibitor, EZH2 (enhancer of zeste homolog 2) inhibitor, Hsp90 inhibitor, immunoreglutory agent such as PD-1, PD-L1 antagonist, OX40 agonist or 4-1BB agonist.

In some embodiments, the additional antineoplastic agent used in any of the herein described combination therapy is selected from aromatase inhibitor, endocrine therapy, hormonal therapy, selective estrogen receptor degrader, cytotoxic agents, PD-1 antagonist, PD-L1 antagonist, AR inhibitor, inhibitor of glutaminase, CDK4/6 inhibitor, CDK9 inhibitor, or Akt inhibitor. In some embodiments, the additional antineoplastic agent is selected from the group of PI3K inhibitor, mTOR inhibitor, and Akt inhibitor.

In some embodiments, the additional antineoplastic agent used in any of the herein described combination therapy is tamoxifen, docetaxel, paclitaxel, cisplatin, capecitabine, gemcitabine, vinorelbine, exemestane, letrozole, fulvestrant, anastrozole, or trastuzumab.

In some embodiments, the herein described deuterated pyridopyrimidinone compound may be combined with surgical removal of a cancer. In some embodiments, the herein described deuterated pyridopyrimidinone compound may be combined with radiation therapy. In some embodiments, the herein described deuterated pyridopyrimidinone compound may be combined with endocrine therapy.

In some embodiments, the additional antineoplastic agent used in any of the herein described combination therapy acts in a phase of the cell cycle other than G2-M phase selected from thymidylate synthase inhibitor, DNA cross linking agent, topoisomerase I or II inhibitor, DNA alkylating agent, a ribonuclease inhibitor, a cytotoxic agent, a growth factor inhibitor, and combinations thereof.

In some embodiments, the deuterated pyridopyrimidinone compound as described herein may be combined with one or more DNA alkylating agents including, but not limited to, chlorambucil, chlormethine, cyclophosphamide, ifosfamide, melphalan, carmustine, fotemustine, lomustine, streptozocin, carboplatin, cisplatin, oxaliplatin, satraplatin, busulfan, dacarbazine, procarbazine, temozolomide, thioTEPA, and uramustine.

In some embodiments, the deuterated pyridopyrimidinone compound as described herein may be combined with one or more mitotic inhibitors including, but not limited to, docetaxel, paclitaxel, vinblastine, vincristine, vindesine, and vinorelbine. In some embodiments, the deuterated compounds described herein can be combined with one or more microtubule stabilizing agent including, but not limited to, docetaxel, paclitaxel, and epothilones A-F.

In some embodiments, the deuterated pyridopyrimidinone compound as described herein may be combined with one or more topoisomerase I or II inhibitors, including, but not limited to, etoposide, etoposide phosphate, teniposide, camptothecin, topotecan, and irinotecan.

In some embodiments, the deuterated pyridopyrimidinone compound as described herein may be combined with one or more cancer immunotherapy monoclonal antibodies, including, but not limited to, rituximab, alemtuzumab, bevacizumab, cetuximab, gemtuzumab, panitumumab, tositumomab, and trastuzumab.

In some embodiments, the deuterated pyridopyrimidinone compound as described herein may be combined with one or more anticancer agents including, but not limited to, daunorubicin, doxorubicin, epirubicin, idarubicin, mitoxantrone, valrubicin, actinomycin, bleomycin, mitomycin, plicamycin, bortezomib, and hydroxyurea.

In some embodiments, the additional antineoplastic agent used in the herein described combination therapy comprises immune checkpoint inhibitor, AR inhibitor, inhibitor of glutaminase, or Akt inhibitor.

In some embodiments, the additional antineoplastic agent used in the herein described combination therapy is a small molecule compound selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, darinaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, cabazitaxel, capecitabine, crizotinib, dabrafenib, dasatinib, N1,N11-bis(ethyl)norspermine (BENSM), ibrutinib, idelalisib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipotecan, GSK-461364, and combinations thereof.

In some embodiments, the additional antineoplastic agent used in the herein described combination therapy is a PI3K inhibitor selected from the group consisting of wortmannin, temsirolimus, everolimus, buparlisib (BMK-120), 5-(2,6-dimorpholinopyrimidin-4-yl)-4-(trifluoromethyl)pyridin-2-amine), pictilisib, gedatolisib, apitolisib, pilaralisib, copanlisib, alpelisib, taselisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), LY294002 (2-Morpholin-4-yl-8-phenylchromen-4-one), dactolisib (2-Methyl-2-{4-[3-methyl-2-oxo-8-(quinolin-3-yl)-2,3-dihydro-1H-imidazo[4,5-c]quinolin-1-yl]phenyl}propanenitrile), omipalisib (2,4-difluoro-N-(2-methoxy-5-(4-(pyridazin-4-yl)quinolin-6-yl)pyridin-3-yl)benzenesulfonamide), bimiralisib (5-(4,6-dimorpholin-4-yl-1,3,5-triazin-2-yl)-4-(trifluoromethyl)pyridin-2-amine), serabelisib (5-(4-amino-1-propan-2-ylpyrazolo[3,4-d]pyrimidin-3-yl)-1,3-benzoxazol-2-amine), GSK2636771 (2-methyl-1-(2-methyl-3-(trifluoromethyl)benzyl)-6-morpholino-1H-benzo[d]imidazole-4-carboxylic acid), AZD8186 (8-[(1R)-1-(3,5-difluoroanilino)ethyl]-N,N-dimethyl-2-morpholin-4-yl-4-oxochromene-6-carboxamide), SAR260301 (2-[2-[(2S)-2,3-dihydro-2-methyl-1H-indol-1-yl]-2-oxoethyl]-6-(4-morpholinyl)-4(3H)-pyrimidinone), IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide), and combinations thereof.

In some embodiments, the one or more of the additional antineoplastic agents used in the herein described combination therapy may include tamoxifen, docetaxel, paclitaxel, cisplatin, capecitabine, gemcitabine, vinorelbine, exemestane, letrozole, fulvestrant, anastrozole, or trastuzumab.

In some embodiments, the combination of the deuterated pyridopyrimidinone compound as described herein with one or more of the additional antineoplastic agents may be formulated as fixed dose combination or co-packaged discrete dosages. In some embodiments, the fixed dose combination therapy of the deuterated pyridopyrimidinone compound as described herein comprises bilayer tablet, triple layer tablet, multilayered tablet, or capsule having plurality populations of particles of deuterated pyridopyrimidinone. In some embodiments, the combination of deuterated pyridopyrimidinone compound with one or more of the additional antineoplastic agents may be administered to a subject in need thereof, e.g., intermittently, concurrently, or sequentially.

In some embodiments, the deuterated pyridopyrimidinone compound in the combination therapy as described herein comprising a single (R,R)-(−) optically pure isomer may be administered intravenously before the additional antineoplastic agent. In some embodiments, the deuterated pyridopyrimidinone compound in the combination therapy as described herein comprising a single (R,R)-(−) optically pure isomer may be administered intravenously after the additional antineoplastic agent. In some embodiments, the deuterated pyridopyrimidinone compound in the combination therapy as described herein comprising a single (R,R)-(−) optically pure isomer may be administered orally before the additional antineoplastic agent. In some embodiments, the deuterated pyridopyrimidinone compound in the combination therapy as described herein comprising a single (R,R)-(−) optically pure isomer may be administered orally after the additional antineoplastic agent.

In some embodiments, the deuterated pyridopyrimidinone compound in the combination therapy as described herein comprising a single (R,R)-(−) optically pure isomer may be administered intravenously before the radiation therapy. In some embodiments, the deuterated pyridopyrimidinone compound in the combination therapy as described herein comprising a single (R,R)-(−) optically pure isomer may be administered intravenously after the radiation therapy. In some embodiments, the deuterated pyridopyrimidinone compound in the combination therapy as described herein comprising a single (R,R)-(−) optically pure isomer may be administered orally before the radiation therapy. In some embodiments, the deuterated pyridopyrimidinone compound in the combination therapy as described herein comprising a single (R,R)-(−) optically pure isomer may be administered orally after the radiation therapy.

In some embodiments, the deuterated pyridopyrimidinone compound in the combination therapy as described herein comprising a single (R,R)-(−) optically pure isomer may be administered intravenously before the surgical removal. In some embodiments, the deuterated pyridopyrimidinone compound in the combination therapy as described herein comprising a single (R,R)-(−) optically pure isomer may be administered intravenously after the surgical removal. In some embodiments, the deuterated pyridopyrimidinone compound in the combination therapy as described herein comprising a single (R,R)-(−) optically pure isomer may be administered orally before the surgical removal. In some embodiments, the deuterated pyridopyrimidinone compound in the combination therapy as described herein comprising a single (R,R)-(−) optically pure isomer may be administered orally after the surgical removal.

In some embodiments, the combination therapies of deuterated pyridopyrimidinone compound as described herein give synergistic effects on inhibiting tumor cell proliferations in a subject. The term “synergistic,” or “synergistic effect” or “synergism” as used herein, generally refers to an effect such that the one or more effects of the combination of compositions is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination (Chou, Cancer Res. 2010, vol. 70, pp. 440-446). When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations administered simultaneously or sequentially.

### EQUIVALENCE

The present invention may be embodied in other specific forms without departing from the spirit or essential attributes thereof. This invention encompasses all combinations of preferred aspects and/or embodiments of the inventions described herein. It is understood that any and all embodiments of the present invention may be taken in conjunction with any other embodiment or embodiments to describe additional more preferred embodiments. It is also to be understood that each individual element of the preferred embodiments is its own independent preferred embodiment. Furthermore, any element of an embodiment is meant to be combined with any and all other elements from any embodiment to describe an additional embodiment.

### EXAMPLES

The following working examples are used to provide descriptions of the present invention, but does not limit the present invention in any way.

### Abbreviations

As used herein the symbols and conventions used in these processes, schemes and examples are consistent with those used in the contemporary scientific literature, for example, the Journal of the American Chemical Society or the Journal of Biological Chemistry. Specifically, the following abbreviations may be used in the examples and throughout the specification:

g (grams); mg (milligrams);
L (liters); mL (milliliters);
μL (microliters); psi (pounds per square inch);
M (molar); mM (millimolar);
Hz (Hertz); MHz (megahertz);
mol (moles); mmol (millimoles);
RT/rt (room temperature); min (minute);
h (hour); TLC (thin layer chromatography);
CH₂Cl₂ (methylene chloride); THF (tetrahydrofuran);
CDCl₃ (deuterated chloroform); CD₃OD (deuterated methanol);
SiO₂ (silica); DMSO (dimethylsulfoxide);
EtOAc (ethyl acetate); atm (atmosphere);
HCl (hydrochloric acid); NaOMe (sodium methoxide);
DMF (N,N-dimethylformamide); Ac (acetyl);
Cs₂CO₃ (cesium carbonate); Me (methyl);
Et (ethyl); EtOH (ethanol);
MeOH (methanol); t-Bu (tert-butyl);
Et₂O (diethyl ether); NaBH₄ (sodium borohydride);
Boc₂O (di-tert-butyl dicarbonate); PhthK (sodium phthalimide);
Ms (methylsulfonyl); MsCl (methylsulfonyl chloride);
Cbz (benzyl); DIPEA (diisopropylethylamine);
D/d (deuterium); TBHP (tert-butyl hydroperoxide);
FeCl₂ (ferrous chloride); EDTA (tetrasodium ethylenediaminetetraacetate);
Boc (tert-butyloxycarbonyl); Zn (zinc);
DMA (N,N-dimethylacetamide); ACN (acetonitrile);
Hex (n-hexane); Bn (Benzyl);
TEA (trimethylamine).

Unless otherwise indicated, all temperatures are expressed in ° C. (degrees Centigrade). All reactions conducted under an inert atmosphere at room temperature unless otherwise noted. Reagents employed without synthetic details are commercially available or made according to literature procedures.

¹H NMR spectra were recorded on a Bruker ASCEND™ 400 equipped with a PABBO 400S1 BBF-H-D-05 Z plus probe. Chemical shifts are expressed in parts per million (ppm, 6 units). Coupling constants are in units of hertz (Hz). Splitting patterns describe apparent multiplicities and are designated as s (singlet), d (doublet), t (triplet), q (quartet), m (multiplet), or b (broad).

Mass spectra were obtained on Agilent 1290-6120B from Agilent Technologies, Inc. Detection is by MS, UV at 254 nM using Electrospray Ionization (ESI).

Compounds of Formula (1) where variables as defined herein can be prepared according to Scheme 1 below.

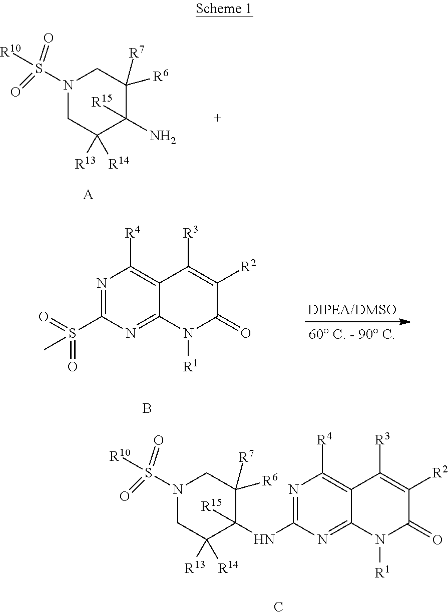

The enantiomerically enriched compound of Formula (3) where variables as defined herein can be prepared according to Scheme 2 below.

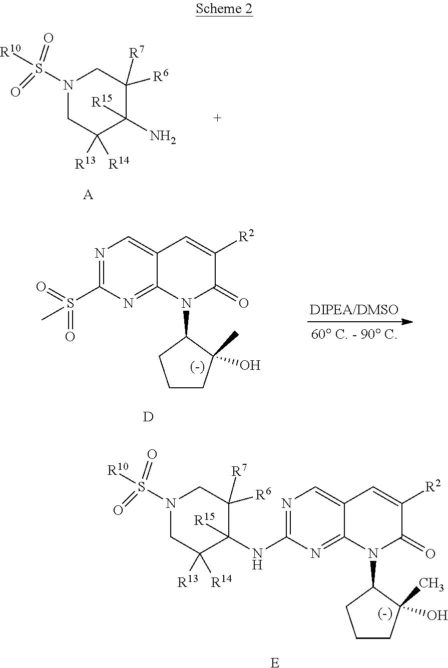

Some of the enantiomerically enriched compound of Formula (5) where variables as defined herein can be prepared according to Scheme 3 below.

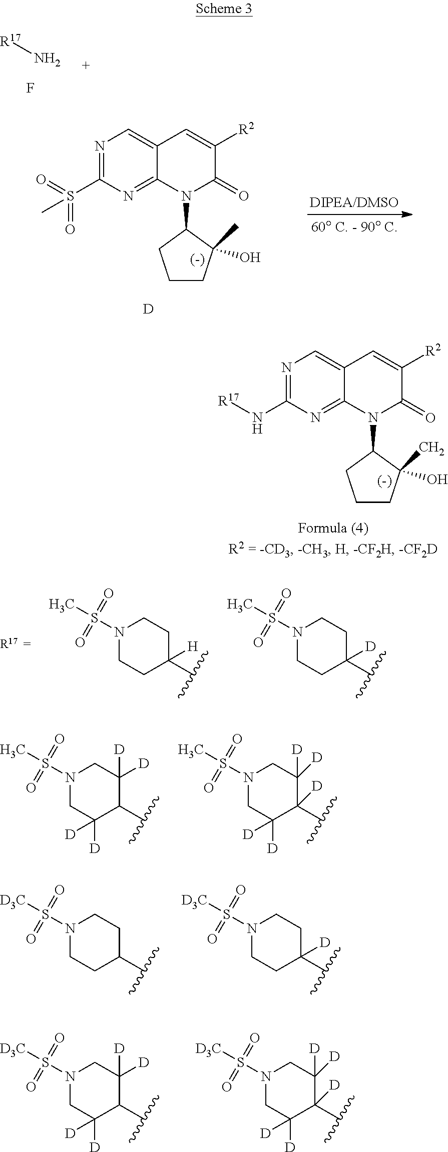

Some of the compound of Formula (4) where R² variable is —CD₃ can be prepared according to Scheme 4 below.

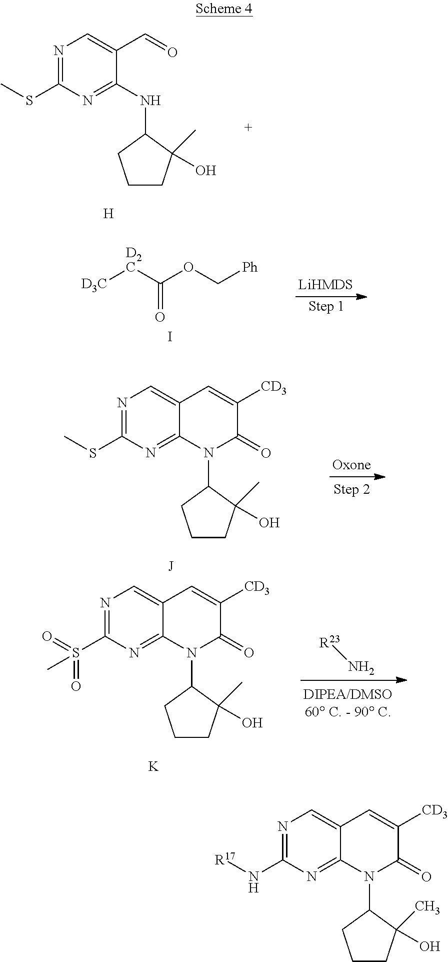

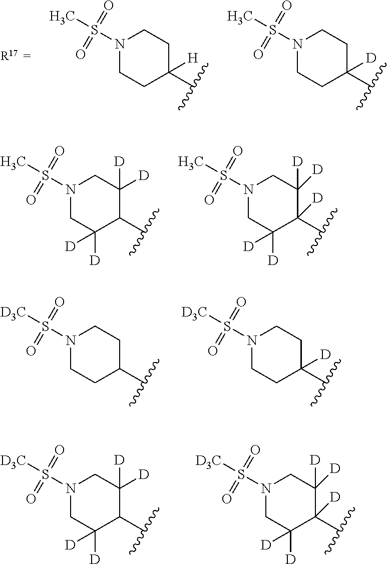

Some of the compounds of Formula (4) where R² variable is —CHF₂ or —CDF₂ can be prepared according to Scheme 5 below.

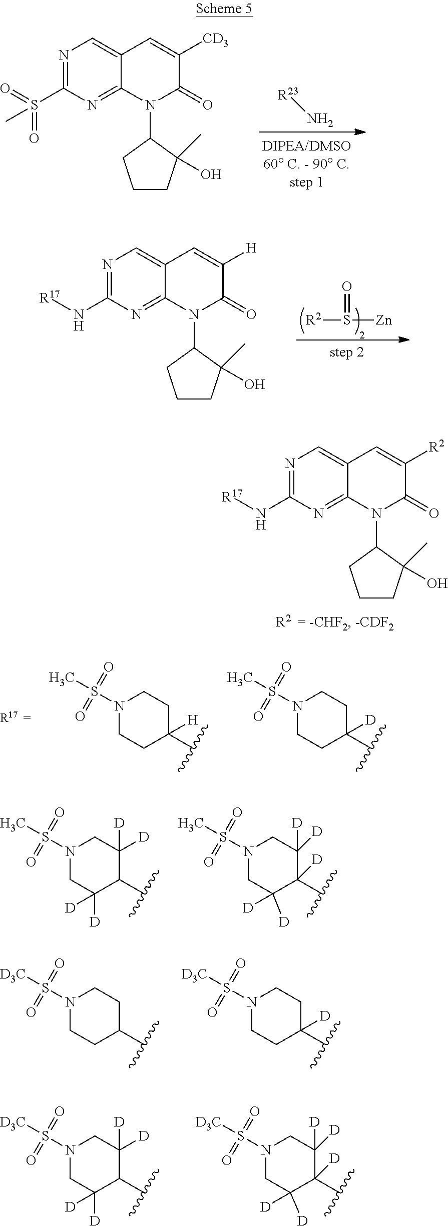

### Intermediate 1. 1-(methylsulfonyl)-piperidin-4-d-4-amine hydrochloride

Synthetic route for intermediate 1 is illustrated in the Scheme 6 below.

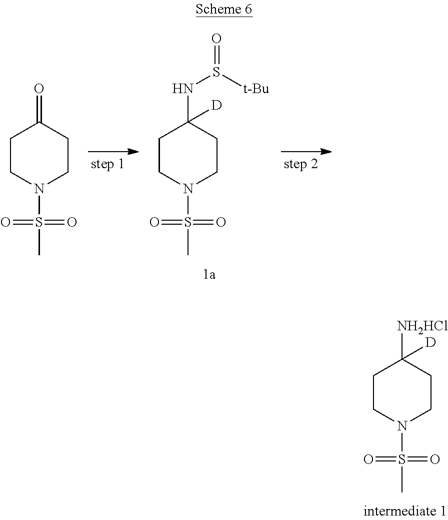

Step 1: To a solution of 1-(methylsulfonyl)-piperidin-4-one (500 mg, 2.82 mmol) dissolved in THF, tert-butyl-sulfinamide (348 mg, 2.87 mmol) and titanium isopropoxide (800 mg, 2.8 mmol) were added to form a mixture. The mixture was heated at 80° C. overnight. Then the reaction mixture was cooled to ambient temperature and sodium borodeuteride (236 mg, 5.64 mmol) was added. The resulting mixture was allowed to react at room temperature overnight. The crude reaction mixture was concentrated and purified by column chromatography using silica gel to provide 660 mg of the product 1a. 2-methyl-N-(1-(methylsulfonyl)-piperidin-4-yl-4-d)propane-2-sulfinamide. [M+H]⁺=284.1

Step 2: To a solution of 2-methyl-N-(1-(methylsulfonyl)-piperidin-4-yl-4-d)propane-2-sulfinamide (1a., 300 mg, 1.06 mmol) dissolved in dichloromethane (10 mL), 50 mL of solution of hydrogen chloride in ethyl acetate was added to form a mixture. The mixture was allowed to react at room temperature overnight. The crude reaction mixture was concentrated purified by column chromatography using silica gel to provide 165 mg of desired intermediate 1:1-(methylsulfonyl)-piperidin-4-d-4-amine hydrochloride. [M⁺ H]⁺=180.1.

### Intermediate 2. 1-((methyl-d₃)sulfonyl)piperidin-4-amine

Synthetic route for intermediate 2 is illustrated in the Scheme 7 below.

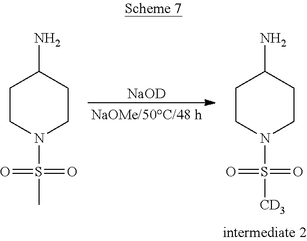

To a solution of 1-(methylsulfonyl)-piperidin-4-one (250 mg, 1.4 mmol) dissolved in deuterated methanol (5 mL), sodium methoxide (302 mg, 5.6 mmol) was added to form a mixture. The mixture was heated at 50° C. for two days. The crude reaction mixture was concentrated purified by column chromatography using silica gel to provide 200 mg of the desired intermediate 2: 1-((methyl-d3)sulfonyl)piperidin-4-amine. [M+H]⁺=182.1.

### Intermediate 3. 1-(methylsulfonyl)piperidin-3,3,5,5-d4-4-amine

Synthetic route for intermediate 3 is illustrated in the Scheme 8 below.

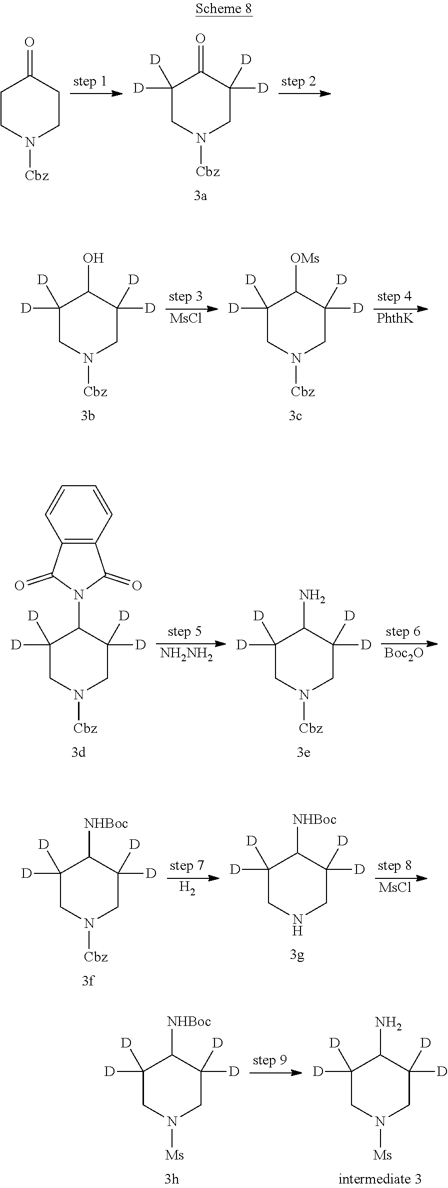

Step 1: To a solution of benzyl 4-oxopiperidine-1-carboxylate (4.3 mg, 18.4 mmol) dissolved in deuterated chloroform (500 mL), catalytic amount of 1,5,7-triazabicyclo[4.4.0]-dec-5-ene was added to form a mixture. The mixture was reacted at room temperature for 1 hour. The reaction mixture was dried over a rotatory evaporator to provide 4.2 g of the desired 3a: benzyl 4-oxopiperidine-1-carboxylate-3,3,5,5-d₄. [M+H]⁺=238.1.

Step 2: To a solution of benzyl 4-oxopiperidine-1-carboxylate-3,3,5,5-d₄ (3a, 4.2 g, 17.7 mmol) dissolved in THF (200 mL), sodium borohydride (1.34 g, 35.4 mmol) was added to form a mixture. The mixture was reacted at room temperature. The reaction mixture was concentrated and purified by column chromatography using silica gel to provide 4.1 g of the desired 3b: benzyl 4-hydroxypiperidine-1-carboxylate-3,3,5,5-d₄. [M+H]⁺=240.1.

Step 3: To a solution of benzyl 4-hydroxypiperidine-1-carboxylate-3,3,5,5-d₄ (3b, 4.0 g, 16.7 mmol) dissolved in DCM (200 mL), diisopropylethylamine (2.58 g, 20 mmol) and methyl sulfonyl chloride (1.95 g, 17 mmol) were added to form a mixture. The mixture was allowed to react at room temperature overnight. The reaction mixture was concentrated and purified by column chromatography using silica gel to provide 3.8 g of the desired 3c: benzyl 4-((methylsulfonyl)oxy)piperidine-1-carboxylate-3,3,5,5-d₄. [M+H]⁺=318.1.

Step 4: To a solution of benzyl 4-((methylsulfonyl)oxy)piperidine-1-carboxylate-3,3,5,5-d₄ (3c, 3.5 g, 11 mmol) dissolved in DMF (100 mL), Potassium phthalimide (2.22 g, 12 mmol) was added to form a mixture. The mixture was heated at 90° C. overnight. The reaction mixture was concentrated and purified by column chromatography using silica gel to provide 4.2 g of the desired 3d: benzyl 4-(1,3-dioxoisoindolin-2-yl)piperidine-1-carboxylate-3,3,5,5-d₄. [M+H]⁺=369.2.

Step 5: Benzyl 4-(1,3-dioxoisoindolin-2-yl)piperidine-1-carboxylate-3,3,5,5-d₄ (3d, 4.2 g, 11.4 mmol) was dissolved in 200 mL of hydrazine hydrate to form a reaction mixture. The reaction mixture was allowed to react at room temperature overnight. The reaction mixture was concentrated and purified by column chromatography using silica gel to provide 2.3 g of the desired 3e: benzyl 4-aminopiperidine-1-carboxylate-3,3,5,5-d₄. [M+H]⁺=239.2.

Step 6: To a solution of benzyl 4-aminopiperidine-1-carboxylate-3,3,5,5-d₄ (3e, 2.3 g, 9.6 mmol) dissolved in DCM (100 mL), diisopropylethylamine (1.29 g, 10 mmol) and Boc anhydride (2.18 g, 10 mmol) were added to form a mixture. The mixture was allowed to react at room temperature overnight. The reaction mixture was concentrated and purified by column chromatography using silica gel to provide 700 mg of the desired 3f: benzyl 4-((tert-butoxycarbonyl)amino)piperidine-1-carboxylate-3,3,5,5-d₄. [M+H]⁺=339.2.

Step 7: To a solution of benzyl 4-((tert-butoxycarbonyl)amino)piperidine-1-carboxylate-3,3,5,5-d₄ (3f, 700 mg, 2 mmol) dissolved in ethyl acetate (50 mL), palladium carbon black was added. The mixture was allowed to react under H₂ atmosphere at room temperature overnight. The reaction mixture was filtered to collect the filtrate. The filtrate was concentrated to provide 410 mg of the desired 3g: tert-butyl (piperidin-4-yl-3,3,5,5-d₄)carbamate. [M+H]⁺=205.2.

Step 8: To a solution of tert-butyl (piperidin-4-yl-3,3,5,5-d₄)carbamate (3g, 410 mg, 1.45 mmol) dissolved in DCM (50 mL), diisopropylethylamine (258 mg, 2 mmol) and methyl sulfonyl chloride (229 mg, 2 mmol) to form a mixture. The mixture was allowed to react at room temperature overnight. The crude mixture was concentrated and purified by column chromatography using silica gel to provide 510 mg of the desired 3h: tert-butyl (1-(methylsulfonyl)piperidin-4-yl-3,3,5,5-d4)carbamate. [M+H]⁺=283.2.

Step 9: To a solution of tert-butyl (1-(methylsulfonyl)piperidin-4-yl-3,3,5,5-d4)carbamate (3h, 510 mg, 1.8 mmol) dissolved in ethyl acetate (10 mL), 50 mL of 1M hydrogen chloride-ethyl acetate was added to form a mixture. The mixture was allowed to react at room temperature overnight. The reaction mixture was concentrated and purified by column chromatography using silica gel to provide 390 mg of the intermediate 3: 1-(methylsulfonyl)piperidin-3,3,5,5-d₄-4-amine. [M+H]⁺=183.2.

### Intermediate 4. 1-((methyl-d3)sulfonyl)piperidin-4-d-4-amine

Synthetic route for intermediate 4 is illustrated in the Scheme 9 below.

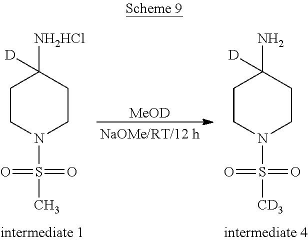

To a solution of 1-(methylsulfonyl)piperidin-4-d-4-amine hydrochloride intermediate 1 (600 mg, 2.78 mmol) dissolved in deuterated methanol (50 mL), sodium methoxide (162 mg, 3 mmol) was added to form a mixture. The mixture was allowed to react at room temperature overnight. The crude mixture was concentrated and purified by column chromatography using silica gel to provide 400 mg of the desired intermediate 4: 1-((methyl-d₃)sulfonyl)piperidin-4-d-4-amine. [M+H]⁺=183.1.

### Intermediate 5. 1-((methyl-d3)sulfonyl)piperidin-3,3,5,5-d4-4-amine

Synthetic route for intermediate 5 is illustrated in the Scheme 10 below.

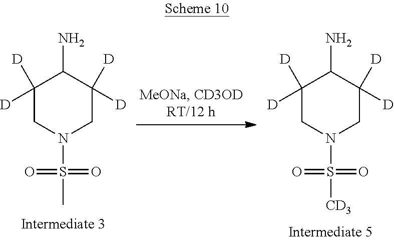 [SMILES: [2H]C1([2H])CN(S(C)(=O)=O)CC([2H])([2H])C1N ; [2H]C1([2H])CN(S(=O)(=O)C([2H])([2H])[2H])CC([2H])([2H])C1N]

Intermediate 5 was synthesized according to the same procedure of Intermediate 4. [M+H]⁺=186.1.

### Intermediate 6. 1-(methylsulfonyl)piperidin-3,3,4,5,5-d5-4-amine

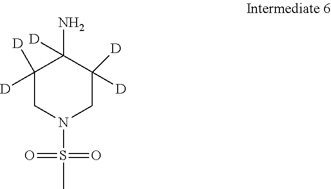 [SMILES: [2H]C1([2H])CN(S(C)(=O)=O)CC([2H])([2H])C1([2H])N]

Intermediate 6 was synthesized according to the same synthetic route for Intermediate 3, using sodium borodeuteride instead of sodium borohydride in step 2. [M+H]⁺=184.1.

### Intermediate 7. 1-((methyl-d3)sulfonyl)piperidin-3,3,4,5,5-d5-4-amine

Synthetic route for intermediate 7 is illustrated in the Scheme 11 below.

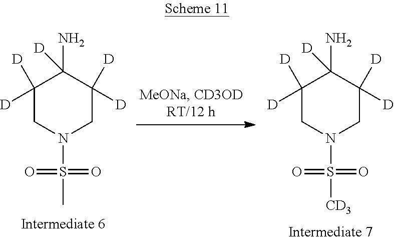 [SMILES: [2H]C1([2H])CN(S(C)(=O)=O)CC([2H])([2H])C1([2H])N ; [2H]C1([2H])CN(S(=O)(=O)C([2H])([2H])[2H])CC([2H])([2H])C1([2H])N]

Intermediate 7 was synthesized according to the same procedure of Intermediate 4. [M+H]⁺=187.1.

### Intermediate 8, 9 and 10. trans-8-(-2-hydroxy-2-methylcyclopentyl)-6-(methyl-d3)-2-(methylsulfonyl)pyrido[2,3-d]pyrimidin-7(8H)-one

Synthetic route for intermediate 8, 9 and 10 is illustrated in the Scheme 12 below.

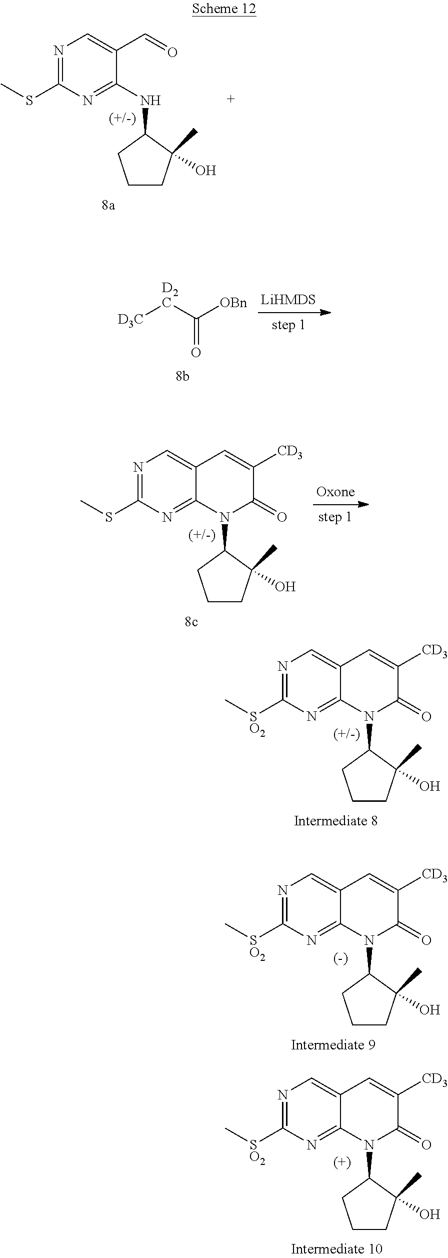

Step 1: To a solution of (+/−)-trans-4-(2-hydroxy-2-methylcyclopentyl)amino)-2-(methylthio)pyrimidine-5-carbaldehyde 8a (200 mg, 0.75 mmol, prepared as WO2018033815) dissolved in THF (8 mL), benzyl propanoate-d₅ 8b (316 mg, 1.87 mmol, prepared by condensation of d6-propionic acid and benzyl alcohol) was added to form a mixture. The mixture was cooled to −5° C. and 1M solution of LiHMDS in toluene was added dropwise. The cooled mixture was then warmed to ambient temperature and stayed at this temperature overnight. The reaction mixture was diluted with ethyl acetate and the aqueous layer was separated from the organic phase. The organic phase was collected and concentrated to remove solvent to provide a crude product. The crude product was purified by column chromatography (eluted with CH₂Cl₂/MeOH 20/1) using silica gel to provide 210 mg of a yellow oil compound 8c: (+/−)-trans-8-(2-hydroxy-2-methylcyclopentyl)-6-(methyl-d3)-2-(methylthio)pyrido[2,3-d]pyrimidin-7(8H)-one. [M+H]⁺=309.1.

Step 2: To a solution of (+/−)-trans-8-(2-hydroxy-2-methylcyclopentyl)-6-(methyl-d3)-2-(methylthio)pyrido[2,3-d]pyrimidin-7(8H)-one 8c (180 mg, 0.58 mmol) dissolved in 2-methyl tetrahydrofuran (10 mL) and water (2 mL), Oxone (900 mg, 1.46 mmol) was added to form a mixture. The mixture as stirred at ambient temperature overnight. The reaction mixture was diluted with ethyl acetate and the aqueous layer was separated from the organic phase. The organic phase was collected and concentrated to remove solvent to provide a crude product. The crude product was purified by column chromatography (eluted with CH₂Cl₂/MeOH 20/1) using silica gel to provide 65 mg of white solid Intermediate 8: (+/−)-trans-8-(-2-hydroxy-2-methylcyclopentyl)-6-(methyl-d3)-2-(methylsulfonyl)pyrido[2,3-d]pyrimidin-7(8H)-one. [M+H]⁺=341.1.

Intermediate 9 and Intermediate 10 was synthesized according to the same route, using chiral 8a (WO2018033815) instead of racemic 8a.

### Intermediate 11, 12 and 13. trans-8-(-2-hydroxy-2-methylcyclopentyl)-6-methyl-2-(methylsulfonyl)pyrido[2,3-d]pyrimidin-7(8H)-one

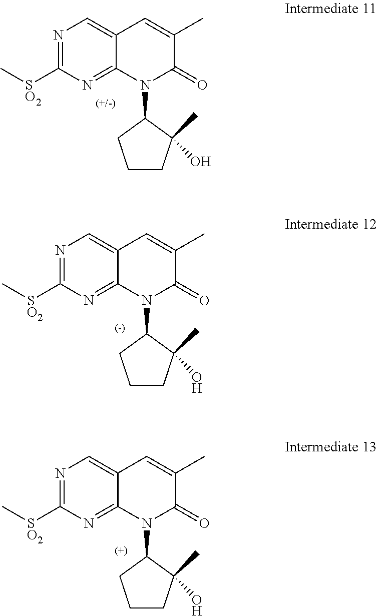 [SMILES: Cc1cc2cnc(S(C)(=O)=O)nc2n([C@@H]2CCC[C@@]2(C)O)c1=O ; Cc1cc2cnc(S(C)(=O)=O)nc2n([C@@H]2CCC[C@@]2(C)O)c1=O ; Cc1cc2cnc(S(C)(=O)=O)nc2n([C@@H]2CCC[C@@]2(C)O)c1=O]

Intermediate 11, 12 and 13 was synthesized according to the same route for Intermediate 8, 9 and 10, using ethyl propionate instead of 8b. [M+H]⁺=338.1.

### Intermediate 14, 15 and 16. trans-8-(-2-hydroxy-2-methylcyclopentyl)-2-(methylsulfonyl)pyrido[2,3-d]pyrimidin-7(8H)-one

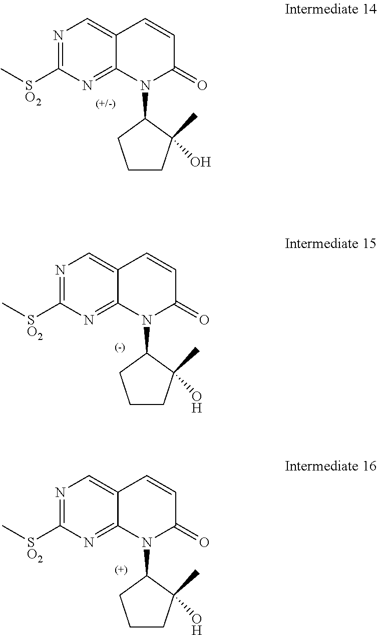 [SMILES: C[C@@]1(O)CCC[C@H]1n1c(=O)ccc2cnc(S(C)(=O)=O)nc21 ; C[C@@]1(O)CCC[C@H]1n1c(=O)ccc2cnc(S(C)(=O)=O)nc21 ; C[C@@]1(O)CCC[C@H]1n1c(=O)ccc2cnc(S(C)(=O)=O)nc21]

Intermediate 14, 15 and 16 was synthesized according to the same route for Intermediate 8, 9 and 10, using ethyl acetate instead of 8b. [M+H]⁺=324.1.

### Example 1. (±)-8-(2-hydroxy-2-methylcyclopentyl)-2-((1-(methylsulfonyl)piperidin-4-yl)amino)pyrido[2,3-d]pyrimidin-7(8H)-one (Comp. 1)

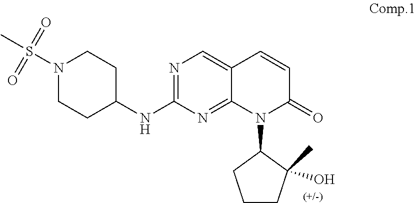 [SMILES: C[C@@]1(O)CCC[C@H]1n1c(=O)ccc2cnc(NC3CCN(S(C)(=O)=O)CC3)nc21]

Compound 1 was synthesized according to WO2018033815. [M+H]⁺=422.2.

### Example 2. (±)-8-(2-hydroxy-2-methylcyclopentyl)-6-difluoromethyl-2-((1-(methylsulfonyl)piperidin-4-yl)amino)pyrido[2,3-d]pyrimidin-7(8H)-one (Comp. 2)

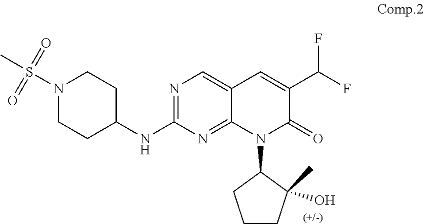 [SMILES: C[C@@]1(O)CCC[C@H]1n1c(=O)c(C(F)F)cc2cnc(NC3CCN(S(C)(=O)=O)CC3)nc21]

Compound 2 was synthesized according to WO2018033815. [M+H]⁺=472.2.

### Example 3. (±)-8-(2-hydroxy-2-methylcyclopentyl)-6-methyl-2-((1-(methylsulfonyl)piperidin-4-yl)amino)pyrido[2,3-d]pyrimidin-7(8H)-one (Comp. 3)

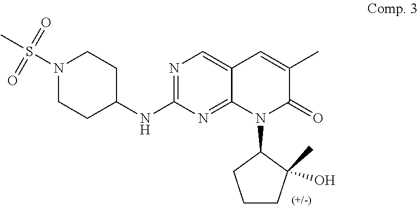 [SMILES: Cc1cc2cnc(NC3CCN(S(C)(=O)=O)CC3)nc2n([C@@H]2CCC[C@@]2(C)O)c1=O]

Compound 3 was synthesized according to WO2018033815. [M+H]⁺=436.2.

### Example 4. (−)-8-(2-hydroxy-2-methylcyclopentyl)-6-methyl-2-((1-(methylsulfonyl)piperidin-4-yl)amino)pyrido[2,3-d]pyrimidin-7(8H)-one (Comp. 4)

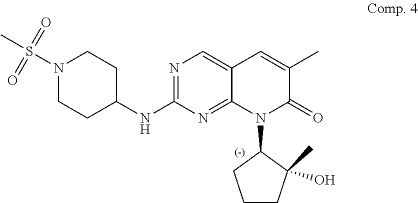 [SMILES: Cc1cc2cnc(NC3CCN(S(C)(=O)=O)CC3)nc2n([C@@H]2CCC[C@@]2(C)O)c1=O]

Compound 4 was synthesized according to WO2018033815. [M+H]⁺=436.2.

### Example 5. (−)-8-(2-hydroxy-2-methylcyclopentyl)-6-difluoromethyl-2-((1-(methylsulfonyl)piperidin-4-yl)amino)pyrido[2,3-d]pyrimidin-7(8H)-one (Comp. 5)

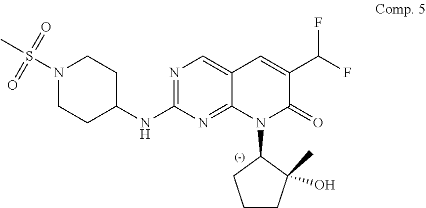 [SMILES: C[C@@]1(O)CCC[C@H]1n1c(=O)c(C(F)F)cc2cnc(NC3CCN(S(C)(=O)=O)CC3)nc21]

Compound 5 was synthesized according to WO2018033815. [M+H]⁺=472.2.

### Example 6. (−)-8-(2-hydroxy-2-methylcyclopentyl)-2-((1-(methylsulfonyl)piperidin-4-yl)amino)pyrido[2,3-d]pyrimidin-7(8H)-one (Comp. 6)

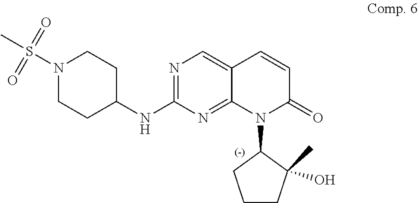 [SMILES: C[C@@]1(O)CCC[C@H]1n1c(=O)ccc2cnc(NC3CCN(S(C)(=O)=O)CC3)nc21]

Compound 6 was synthesized according to WO2018033815. [M+H]⁺=422.2.

### Example 7. racemic (±)-8-(2-hydroxy-2-methylcyclopentyl)-6-(methyl-d3)-2-((1-(methylsulfonyl)piperidin-4-yl)amino)pyrido[2,3-d]pyrimidin-7(8H)-one (Comp. 11)

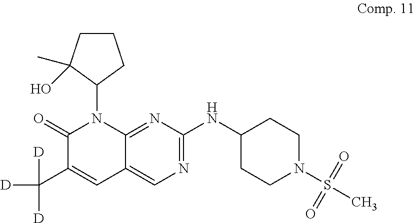 [SMILES: [2H]C([2H])([2H])c1cc2cnc(NC3CCN(S(C)(=O)=O)CC3)nc2n(C2CCCC2(C)O)c1=O]

Synthetic route for Comp. 11 is illustrated in the Scheme 13 below.

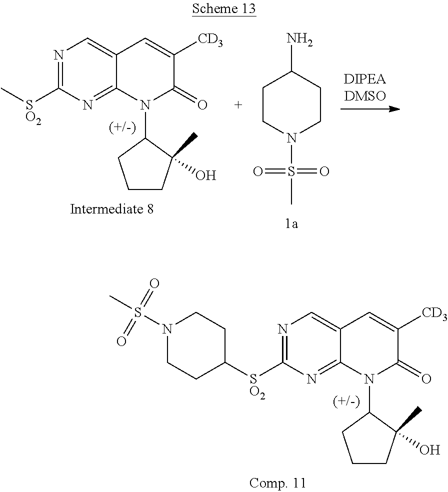 [SMILES: [2H]C([2H])([2H])c1cc2cnc(S(C)(=O)=O)nc2n(C2CCC[C@@]2(C)O)c1=O ; CS(=O)(=O)N1CCC(N)CC1 ; [2H]C([2H])([2H])c1cc2cnc(S(=O)(=O)C3CCN(S(C)(=O)=O)CC3)nc2n(C2CCC[C@@]2(C)O)c1=O]

To a solution of (+/−)-trans-8-(-2-hydroxy-2-methylcyclopentyl)-6-(methyl-d3)-2-(methylsulfonyl)pyrido[2,3-d]pyrimidin-7(8H)-one (Intermediate 8, 65 mg, 0.19 mmol) dissolved in dimethylsulfoxide (DMSO, 5 mL), 1-(methylsulfonyl)-piperidin-4-amine (1a, 68 mg, 0.38 mmol) and diisopropylethylamine (74 mg, 0.57 mmol) were added to form a mixture. The mixture was heated at 70° C. for 9 hours. The reaction mixture was cooled to ambient temperature, diluted with water and extracted with DCM/MeOH (10:1). The organic solvent was removed with rotatory evaporator. The crude product was purified by column chromatography (eluted with CH₂Cl₂/MeOH 20/1) using silica gel to provide 36 mg white solid, Comp. 11, (+/−)-trans-8-(-2-hydroxy-2-methylcyclopentyl)-6-(methyl-d3)-2-((1-(methylsulfonyl)piperidin-4-yl)amino)pyrido[2,3-d]pyrimidin-7(8H)-one. ¹HNMR (400 MHz, CDCl₃) δ 8.40 (s, 1H), 7.35 (s, 1H), 5.78-5.73 (m, 1H), 5.46 (brs, 1H), 4.01 (brs, 1H), 3.86-3.80 (m, 1H), 2.98-2.85 (m, 6H), 2.32-2.23 (m, 2H), 2.06-1.84 (m, 4H), 1.72-1.67 (m, 4H). [M+H]⁺=439.2.

### Example 8. racemic (±)-6-(difluoromethyl-d)-8-(2-hydroxy-2-methylcyclopentyl)-2-((1-(methylsulfonyl)piperidin-4-yl)amino)pyrido[2,3-d]pyrimidin-7(8H)-one (Comp. 20)

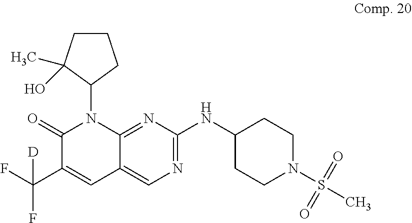 [SMILES: [2H]C(F)(F)c1cc2cnc(NC3CCN(S(C)(=O)=O)CC3)nc2n(C2CCCC2(C)O)c1=O]

Synthetic route for Comp. 20 is illustrated in the Scheme 14 below.

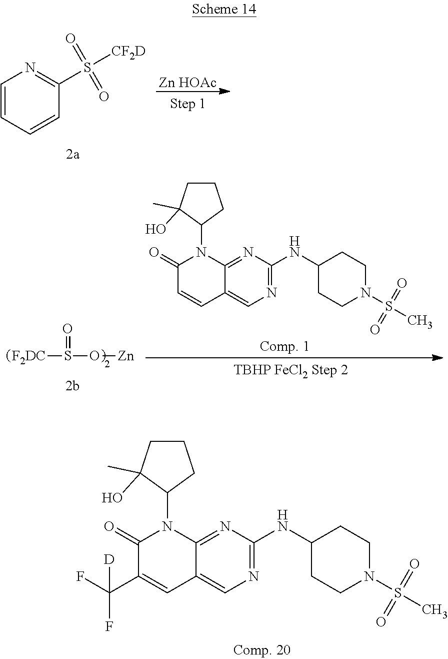

Step 1: To a solution of 2-((difluoromethyl-d)sulfonyl)pyridine (6 g, 31 mmol) (prepared according to J. Am. Chem. Soc. 2018, 140, 880-883) dissolved in methanol (200 mL) and acetic acid (20 mL), zinc powder (8 g, 124 mmol) was added to form a mixture. The mixture was stirred at ambient temperature for 4 hours. The crude reaction mixture was filtered and the solid was washed with methanol. The methanol solution of the product was dried with rotatory evaporator to provide 7 g of the solid crude product (difluoromethyl-d)-sulfonyl zinc. The solid crude product was used in the next step reaction without further purification.

Step 2: To a solution of (+/−)-trans-8-(-2-hydroxy-2-methylcyclopentyl)-2-((1-(methylsulfonyl)piperidin-4-yl)amino)pyrido[2,3-d]pyrimidin-7(8H)-one (Comp. 1, 4.5 g, 10.7 mmol) dissolved in DMSO (100 mL), trifluoroacetic acid (1 g, 10.7 mmol), the crude (difluoromethyl-d)-sulfonyl zinc of Step 1 (7 g, 23.5 mmol), and a solution of FeCl₂ (678 mg, 5.35 mmol) in 20 mL water were added to form a mixture. Then tert-butyl hydroperoxide solution 70% in H₂O (3.85 g, 42.8 mmol) was added dropwise to the mixture. The reaction mixture was stayed at ambient temperature overnight. The reaction mixture was poured into cold 10% aqueous solution of tetrasodium ethylenediaminetetraacetate (EDTA tetrasodium salt) in water. The EDTA tetrasodium worked up reaction mixture was extracted with ethyl acetate. The organic phase was washed with 10% aqueous solution of EDTA tetrasodium salt and concentrated. The crude product was purified by column chromatography using silica gel and liquid phase chromatography to provide 700 mg of white solid a the desired racemic Comp. 20, (±)-6-(difluoromethyl-d)-8-(2-hydroxy-2-methylcyclopentyl)-2-((1-(methylsulfonyl)piperidin-4-yl)amino)pyrido[2,3-d]pyrimidin-7(8H)-one. ¹HNMR (400 MHz, CDCl₃) δ 8.52 (s, 1H), 7.82 (s, 1H), 5.78-5.50 (m, 2H), 4.11-3.83 (m, 3H), 2.96-2.88 (m, 2H), 2.83 (s, 3H), 2.77 (brs, 1H), 2.31-1.65 (m, 10H), 1.17 (s, 3H); [M+H]⁺=473.2.

The degree of deuterium enrichment is >95% as compared the ¹H NMR spectrum of the deuterated and non-deuterated compound (Comp. 2 disclosed in WO 2018033815) as below.

¹HNMR (400 MHz, CDCl₃) δ 8.55 (s, 1H), 7.85 (s, 1H), 6.94-6.66 (m, 1H, the deuterium substitution), 5.80-5.60 (m, 2H), 4.14-4.00 (m, 1H), 3.88-3.86 (m, 2H), 2.96-2.86 (m, 6H), 2.45-1.84 (m, 10H), 1.19 (s, 3H); [M+H]⁺=472.2.

### Example 9. (−)-6-(difluoromethyl-d)-8-((1R,2R)-2-hydroxy-2-methylcyclopentyl)-2-((1-(methylsulfonyl)piperidin-4-yl)amino)pyrido[2,3-d]pyrimidin-7(8H)-one (Comp. 28)

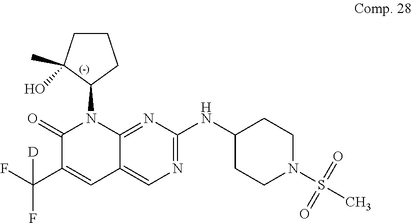 [SMILES: [2H]C(F)(F)c1cc2cnc(NC3CCN(S(C)(=O)=O)CC3)nc2n([C@@H]2CCC[C@@]2(C)O)c1=O]

Compound 28, white solid, was synthesized according to the similar synthetic route for compound 20 using Compound 6 instead of Compound 1. ¹HNMR (400 MHz, d₆-DMSO) δ 8.81-8.76 (m, 1H), 8.21-7.95 (m, 2H), 5.89-5.85 (m, 1H), 4.43-4.38 (m, 1H), 4.08-3.90 (m, 1H), 3.63-3.55 (m, 2H), 2.90-2.83 (m, 5H), 2.27-2.18 (m, 2H), 2.00-1.45 (m, 8H), 1.01 (d, J=13.2 Hz, 3H). [M+H]⁺=472.2. ee %=99.75%. The degree of deuterium enrichment is >95%.

### Example 10. (−)-8-((1R,2R)-2-hydroxy-2-methylcyclopentyl)-6-(methyl-d3)-2-((1-(methylsulfonyl)piperidin-4-yl)amino)pyrido[2,3-d]pyrimidin-7(8H)-one (Comp. 37)

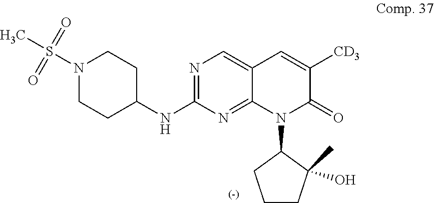 [SMILES: [2H]C([2H])([2H])c1cc2cnc(NC3CCN(S(C)(=O)=O)CC3)nc2n([C@@H]2CCC[C@@]2(C)O)c1=O]

Compound 37, white solid, was synthesized according to the similar synthetic route for compound Comp. 11 using Intermediate 9 instead of Intermediate 8. ¹HNMR (400 MHz, CDCl₃) δ 8.40 (s, 1H), 7.35 (s, 1H), 5.78-5.74 (m, 1H), 5.43 (brs, 1H), 4.01 (brs, 1H), 3.86-3.81 (m, 2H), 2.98-2.90 (m, 2H), 2.85 (s, 4H), 2.32-2.23 (m, 3H), 2.09-1.84 (m, 4H), 1.71-1.62 (m, 3H), 1.18 (s, 3H). [M+H]⁺=439.2. ee %=99.75%. The degree of deuterium enrichment is >95%.

### Example 11. (−)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one (Comp. 47)

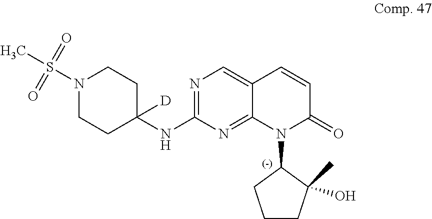 [SMILES: [2H]C1(Nc2ncc3ccc(=O)n([C@@H]4CCC[C@@]4(C)O)c3n2)CCN(S(C)(=O)=O)CC1]

Synthetic route for Compound 47 is illustrated in the Scheme 15 below.

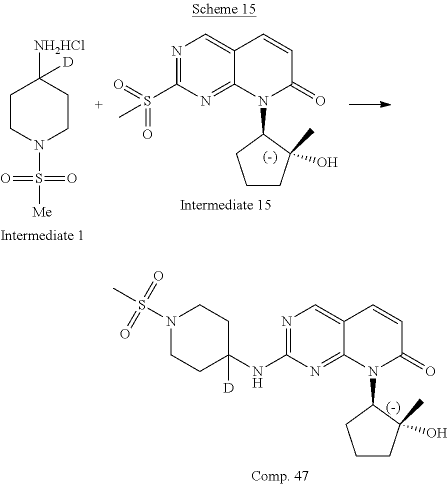

To a solution of 1-(methylsulfonyl)-piperidin-4-d-4-amine hydrochloride (Intermediate 1, 67 mg, 0.31 mmol) dissolved in DMSO (5 mL), (−)-8-((1R,2R)-2-hydroxy-2-methylcyclopentyl)-2-(methylsulfonyl)pyrido[2,3-d]pyrimidin-7(8H)-one (Intermediate 15, 50 mg, 0.15 mmol) and diisopropylethylamine (129 mg, 1 mmol) were added to form a mixture. The mixture was heated at 80° C. overnight. The crude reaction mixture was concentrated purified by preparative thin layer chromatography to provide 20 mg of the desired product Comp. 47, (−)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one. ¹HNMR (400 MHz, CDCl₃) δ 8.45 (s, 1H), 7.47 (d, J=9.2 Hz, 1H), 6.38 (d, J=9.2 Hz, 1H), 5.77-5.73 (m, 1H), 3.86-3.81 (m, 2H), 2.99-2.91 (m, 2H), 2.85 (s, 4H), 2.31-2.22 (m, 3H), 2.05-1.84 (m, 4H), 1.75-1.68 (m, 2H), 1.20 (s, 3H). [M+H]⁺=423.2. ee %=99.7%. The degree of deuterium enrichment is >95%.

### Example 12. (−)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one (Comp. 55)

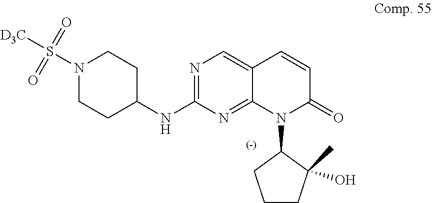 [SMILES: [2H]C([2H])([2H])S(=O)(=O)N1CCC(Nc2ncc3ccc(=O)n([C@@H]4CCC[C@@]4(C)O)c3n2)CC1]

Synthetic route for Compound CDK-1700275 is illustrated in the Scheme 16 below:

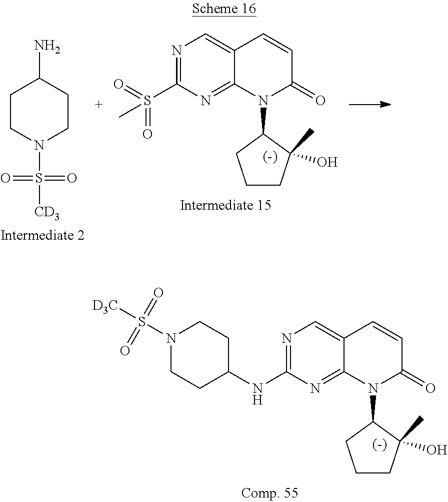 [SMILES: [2H]C([2H])([2H])S(=O)(=O)N1CCC(N)CC1 ; C[C@@]1(O)CCC[C@H]1n1c(=O)ccc2cnc(S(C)(=O)=O)nc21 ; [2H]C([2H])([2H])S(=O)(=O)N1CCC(Nc2ncc3ccc(=O)n([C@@H]4CCC[C@@]4(C)O)c3n2)CC1]

To solution of 1-((methyl-d3)sulfonyl)piperidin-4-amine (Intermediate 2, 200 mg, 1.1 mmol) dissolved in DMSO (5 mL), (−)-8-((1R,2R)-2-hydroxy-2-methylcyclopentyl)-2-(methylsulfonyl)pyrido[2,3-d]pyrimidin-7(8H)-one (Intermediate 15, 356 mg, 1.1 mmol) and diisopropylethylamine (258 mg, 2 mmol) were added to form a mixture. The mixture was heated at 60° C. The crude reaction mixture was concentrated purified by preparative thin layer chromatography to provide 180 mg of the desired product Comp. 55, (−)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one. ¹HNMR (400 MHz, CDCl₃) δ 8.44 (s, 1H), 7.47 (d, J=9.6 Hz, 1H), 6.37 (d, J=9.2 Hz, 1H), 5.77-5.72 (m, 1H), 5.54 (brs, 1H), 4.02 (brs, 1H), 3.86-3.81 (m, 2H), 2.98-2.82 (m, 3H), 2.30-2.23 (m, 3H), 2.03-1.87 (m, 4H), 1.70-1.63 (m, 2H), 1.19 (s, 3H). [M+H]⁺=425.2. ee %=99.7%. The degree of deuterium enrichment is 90%.

### Example 13. (−)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one (Comp. 61)

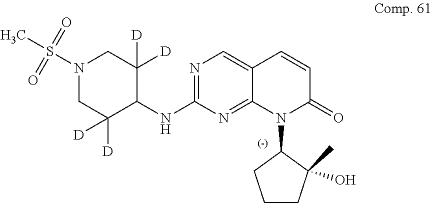 [SMILES: [2H]C1([2H])CN(S(C)(=O)=O)CC([2H])([2H])C1Nc1ncc2ccc(=O)n([C@@H]3CCC[C@@]3(C)O)c2n1]

Synthetic route for Compound 61 is illustrated in the Scheme 17 below.

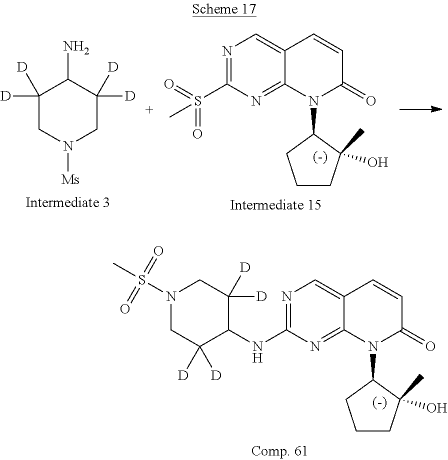

To a solution of 1-(methylsulfonyl)piperidin-3,3,5,5-d₄-4-amine (Intermediate 3, 244 mg, 1.34 mmol) dissolved in DMSO, diisopropylethylamine (258 mg, 2 mmol) and (−)-8-((1R,2R)-2-hydroxy-2-methylcyclopentyl)-2-(methylsulfonyl)pyrido[2,3-d]pyrimidin-7(8H)-one (Intermediate 15, 485 mg, 1.5 mmol) were added to form a mixture. The mixture was heated at 70° C. overnight. The crude mixture was concentrated and purified by column chromatography using silica gel to provide 112 mg of the desired product Comp. 61, (−)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one. ¹HNMR (400 MHz, CDCl₃) δ 8.45 (s, 1H), 7.47 (d, J=9.2 Hz, 1H), 6.37 (d, J=9.2 Hz, 1H), 5.77-5.73 (m, 1H), 5.53 (brs, 1H), 4.00 (brs, 1H), 3.85-3.80 (m, 2H), 2.96-2.91 (m, 2H), 2.85 (s, 4H), 2.30-2.22 (m, 2H), 2.05-1.84 (m, 4H), 1.19 (s, 3H). [M+H]⁺=426.2. ee %=99.7%. The degree of deuterium enrichment is 85%.

### Example 14. (−)-8-((1R,2R)-2-hydroxy-2-methylcyclopentyl)-6-methyl-2-((1-(methylsulfonyl)piperidin-4-yl-3,3,5,5-d4)amino)pyrido[2,3-d]pyrimidin-7(8H)-one (Comp. 67)

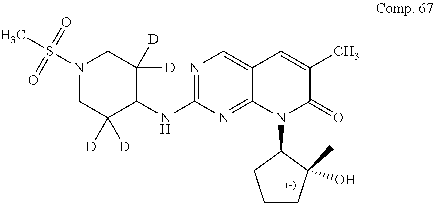 [SMILES: [2H]C1([2H])CN(S(C)(=O)=O)CC([2H])([2H])C1Nc1ncc2cc(C)c(=O)n([C@@H]3CCC[C@@]3(C)O)c2n1]

Compound 67 was synthesized according to the similar synthetic route for Comp. 61, using Intermediate 12 instead of Intermediate 15. ¹HNMR (400 MHz, CDCl₃) δ 8.39 (s, 1H), 7.35 (s, 1H), 5.78-5.73 (m, 1H), 5.53 (brs, 1H), 3.99 (brs, 1H), 3.83-3.78 (m, 2H), 2.95-2.90 (m, 2H), 2.84 (s, 4H), 2.31-2.19 (m, 2H), 2.16 (s, 3H), 2.08-1.88 (m, 4H), 1.17 (s, 3H). [M+H]⁺=440.2. ee %=99.7%. The degree of deuterium enrichment is 85%.

### Example 15. (−)-8-((1R,2R)-2-hydroxy-2-methylcyclopentyl)-6-methyl-2-((1-((methyl-d3)sulfonyl)piperidin-4-yl)amino)pyrido[2,3-d]pyrimidin-7(8H)-one (Comp. 74)

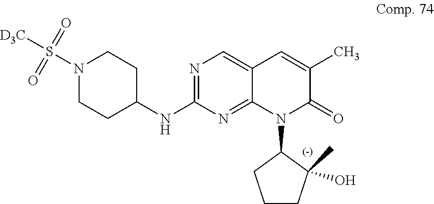 [SMILES: [2H]C([2H])([2H])S(=O)(=O)N1CCC(Nc2ncc3cc(C)c(=O)n([C@@H]4CCC[C@@]4(C)O)c3n2)CC1]

Compound 74 was synthesized according to the similar synthetic route for Comp. 55, using Intermediate 12 instead of Intermediate 15. ¹HNMR (400 MHz, CDCl₃) δ 8.40 (s, 1H), 7.35 (s, 1H), 5.78-5.74 (m, 1H), 5.42 (brs, 1H), 4.00 (brs, 1H), 3.86-3.80 (m, 2H), 2.99-2.82 (m, 3H), 2.23-2.33 (m, 3H), 2.17 (s, 3H), 2.09-1.84 (m, 4H), 1.72-1.62 (m, 3H), 1.18 (s, 3H). [M+H]⁺=439.2. ee %=99.7%. The degree of deuterium enrichment is 90%.

### Example 16. (−)-8-((1R,2R)-2-hydroxy-2-methylcyclopentyl)-6-methyl-2-((1-(methylsulfonyl)piperidin-4-yl-4-d)amino)pyrido[2,3-d]pyrimidin-7(8H)-one (Comp. 80)

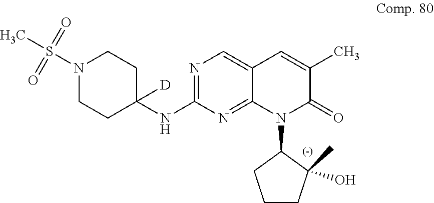 [SMILES: [2H]C1(Nc2ncc3cc(C)c(=O)n([C@@H]4CCC[C@@]4(C)O)c3n2)CCN(S(C)(=O)=O)CC1]

Compound 80 was synthesized according to the similar synthetic route for Comp. 47, using Intermediate 12 instead of Intermediate 15. ¹HNMR (400 MHz, CDCl₃) δ 8.4 (s, 1H), 7.35 (s, 1H), 5.78-5.74 (m, 1H), 5.39 (brs, 1H), 3.85-3.80 (m, 2H), 2.99-2.91 (m, 2H), 2.85 (s, 4H), 2.34-2.21 (m, 3H), 2.17 (s, 3H), 2.07-1.84 (m, 4H), 1.74-1.64 (m, 3H), 1.18 (s, 3H). [M+H]⁺=437.2. ee %=99.7%. The degree of deuterium enrichment is >95%.

### Example 17. (−)-6-(difluoromethyl)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one (Comp. 85)

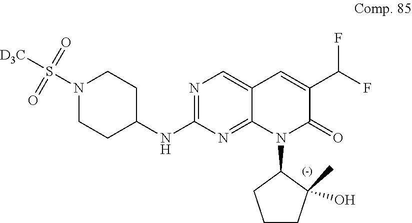 [SMILES: [2H]C([2H])([2H])S(=O)(=O)N1CCC(Nc2ncc3cc(C(F)F)c(=O)n([C@@H]4CCC[C@@]4(C)O)c3n2)CC1]

Compound 85 was synthesized according to the same procedure as step 2 of Comp. 20, using Comp. 55 instead of Comp. 1, commercially available (difluoromethyl)-sulfinate zinc instead of custom made 2b. ¹HNMR (400 MHz, CDCl₃) δ 8.55 (s, 1H), 7.85 (s, 1H), 6.93-6.66 (m, 1H), 5.78-5.69 (m, 2H), 4.17-3.99 (m, 1H), 3.88-3.85 (m, 2H), 2.96-2.91 (m, 2H), 2.83-2.75 (m, 1H), 2.33-2.14 (m, 3H), 2.07-1.82 (m, 4H), 1.19 (s, 3H). [M+H]⁺=475.2. ee %=99.7%. The degree of deuterium enrichment is 90%.

### Example 18. (−)-6-(difluoromethyl-d)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one (Comp. 92)

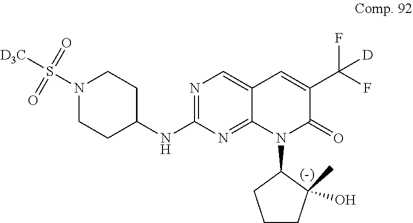 [SMILES: [2H]C(F)(F)c1cc2cnc(NC3CCN(S(=O)(=O)C([2H])([2H])[2H])CC3)nc2n([C@@H]2CCC[C@@]2(C)O)c1=O]

Comp. 92 was synthesized according to the similar synthetic route for Comp. 20, using Comp. 55 instead of Comp. 1. ¹HNMR (400 MHz, CDCl₃) δ 8.56 (s, 1H), 7.85 (s, 1H), 6.22 (brs, 0.5H), 5.80 (s, 1H), 5.65 (brs, 0.5H), 4.12-4.02 (m, 1H), 3.86-3.78 (m, 2H), 3.01-2.95 (m, 2H), 2.83-2.74 (m, 1.5H), 2.35-2.21 (m, 3.5H), 2.09-1.94 (m, 4H), 1.87-1.82 (m, 2H), 1.19 (s, 3H). [M+H]⁺=476.2. ee %=99.7%. The degree of deuterium enrichment is 90%.

### Example 19. (−)-6-(difluoromethyl)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one (Comp. 99)

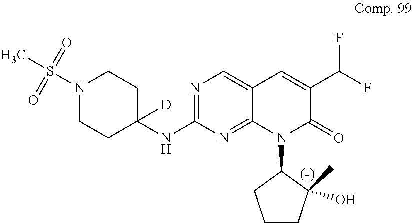 [SMILES: [2H]C1(Nc2ncc3cc(C(F)F)c(=O)n([C@@H]4CCC[C@@]4(C)O)c3n2)CCN(S(C)(=O)=O)CC1]

Compound 99 was synthesized according to the similar synthetic route for Comp. 20, using Comp. 47 instead of Comp. 1, commercially available (difluoromethyl)-sulfinate zinc was used instead of custom made 2b. ¹HNMR (400 MHz, CDCl₃) δ 8.55 (s, 1H), 7.84 (s, 1H), 6.93-6.66 (m, 1H), 5.80-5.62 (m, 2H), 3.88-3.82 (m, 2H), 2.97-2.91 (m, 2H), 2.85 (s, 4H), 2.33-2.21 (m, 3H), 2.09-1.91 (m, 3H), 1.87-1.82 (m, 1H), 1.78-1.69 (m, 2H), 1.19 (s, 3H). [M+H]⁺=473.2. ee %=99.7%. The degree of deuterium enrichment is >95%.

### Example 20. (−)-6-(difluoromethyl-d)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one (Comp. 108)

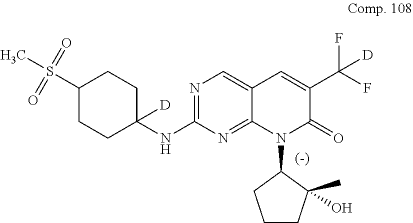 [SMILES: [2H]C1(Nc2ncc3cc(C([2H])(F)F)c(=O)n([C@@H]4CCC[C@@]4(C)O)c3n2)CCC(S(C)(=O)=O)CC1]

Compound 108 was synthesized according to the similar synthetic route for Comp. 20, using comp. 47 instead of Comp. 1. ¹HNMR (400 MHz, CDCl₃) δ 8.56 (s, 1H), 7.84 (s, 1H), 6.41 (brs, 0.5H), 5.80 (brs, 1.5H), 3.85-3.77 (m, 2H), 3.02-2.96 (m, 2H), 2.86 (s, 3H), 2.79-2.74 (m, 1H), 2.30-2.20 (m, 4H), 2.07-2.02 (m, 3H), 1.86-1.82 (m, 2H), 1.75-1.68 (m, 1H), 1.18 (s, 3H). [M+H]⁺=474.2. ee %=99.7%. The degree of deuterium enrichment is 95%.

### Example 21. (−)-6-(difluoromethyl)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one (Comp. 121)

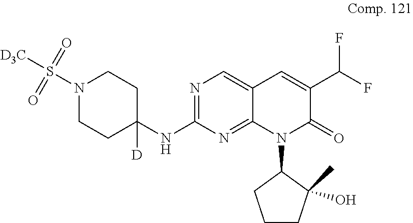 [SMILES: [2H]C1(Nc2ncc3cc(C(F)F)c(=O)n([C@@H]4CCC[C@@]4(C)O)c3n2)CCN(S(=O)(=O)C([2H])([2H])[2H])CC1]

Synthetic route for Comp. 121 is illustrated in the Scheme 18 below:

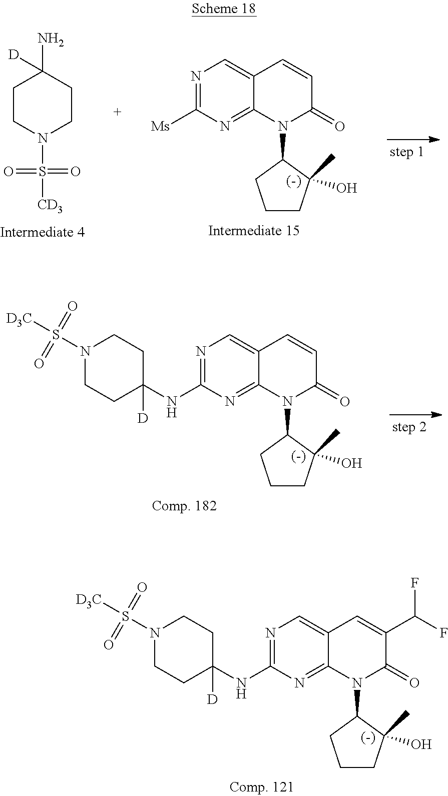 [SMILES: [2H]C1(N)CCN(S(=O)(=O)C([2H])([2H])[2H])CC1 ; C[C@@]1(O)CCC[C@H]1n1c(=O)ccc2cnc(S(C)(=O)=O)nc21 ; [2H]C1(Nc2ncc3ccc(=O)n([C@@H]4CCC[C@@]4(C)O)c3n2)CCN(S(=O)(=O)C([2H])([2H])[2H])CC1 ; [2H]C1(Nc2ncc3cc(C(F)F)c(=O)n([C@@H]4CCC[C@@]4(C)O)c3n2)CCN(S(=O)(=O)C([2H])([2H])[2H])CC1]

Step 1: To a solution of 1-((methyl-d₃)sulfonyl)piperidin-4-d-4-amine (Intermediate 4, 400 mg, 2.2 mmol) dissolved in DMSO (20 mL), diisopropylethylamine (517 mg, 4 mmol) and (−)-8-((1R,2R)-2-hydroxy-2-methylcyclopentyl)-2-(methylsulfonyl)pyrido[2,3-d]pyrimidin-7(8H)-one (Intermediate 15, 711 mg, 2.2 mmol) to form a mixture. The mixture was heated at 70° C. overnight. The crude mixture was concentrated and purified by column chromatography using silica gel to provide 300 mg of Comp. 182, (−)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one. [M+H]⁺=426.2.

Step 2: To a solution of (−)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one (Comp. 182, 22 mg, 0.19 mmol) dissolved in DMSO (5 mL), trifluoroacetic acid (22 mg, 0.19 mmol), (difluoromethyl)-sulfonyl zinc (118 mg, 0.4 mmol) and 1 mL of aqueous solution of FeCl₂ (12.7 mg, 0.1 mmol) were added to form a mixture. Then tert-butyl hydroperoxide solution 70% in H₂O (3.85 g, 42.8 mmol) was added dropwise to the mixture. The reaction mixture was stayed at ambient temperature overnight. The reaction mixture was poured into cold 10% aqueous solution of tetrasodium ethylenediaminetetraacetate (EDTA tetrasodium salt) in water. The EDTA tetrasodium worked up reaction mixture was extracted with ethyl acetate. The organic phase was washed with 10% aqueous solution of EDTA tetrasodium salt and concentrated. The crude product was purified by column chromatography using silica gel and liquid phase chromatography to provide 38 mg of the desired product Comp. 121, (−)-6-(difluoromethyl)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one. ¹HNMR (400 MHz, CDCl₃) δ 8.55 (s, 1H), 7.85 (s, 1H), 6.93-6.66 (m, 1H), 5.81-5.63 (m, 2H), 3.87-3.81 (m, 2H), 2.97-2.91 (m, 2H), 2.83-2.72 (m, 1H), 2.33-2.21 (m, 3H), 2.07-1.82 (m, 5H), 1.79-1.69 (m, 2H), 1.19 (s, 3H). [M+H]⁺=476.2. ee %=99.7%. The degree of deuterium enrichment is 90%.

### Example 22. (−)-6-(difluoromethyl-d)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one (Comp. 127)

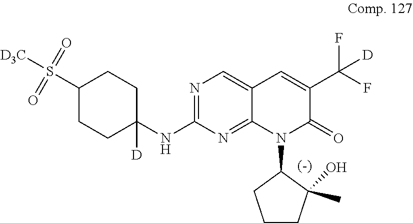 [SMILES: [2H]C1(Nc2ncc3cc(C([2H])(F)F)c(=O)n([C@@H]4CCC[C@@]4(C)O)c3n2)CCC(S(=O)(=O)C([2H])([2H])[2H])CC1]

Compound 127 was synthesized according to the similar synthetic route for compound Comp. 121, using reagent 2b from Example 8 instead of (difluoromethyl)-sulfinate zinc. ¹HNMR (400 MHz, CDCl₃) δ 8.56 (s, 1H), 7.85 (s, 1H), 6.34 (brs, 0.5H), 5.80 (brs, 1.5H), 3.85-3.77 (m, 2H), 3.01-2.95 (m, 2H), 2.83-2.74 (s, 1H), 2.29-2.20 (m, 3H), 2.04-1.67 (m, 8H), 1.18 (s, 3H). [M+H]⁺=477.2. ee %=99.7%. The degree of deuterium enrichment is 90%.

### Example 23. 6-(difluoromethyl)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one (Comp. 143)

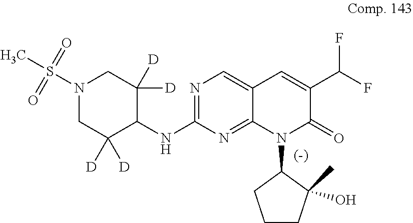 [SMILES: [2H]C1([2H])CN(S(C)(=O)=O)CC([2H])([2H])C1Nc1ncc2cc(C(F)F)c(=O)n([C@@H]3CCC[C@@]3(C)O)c2n1]

Compound 143 was synthesized according to the similar synthetic route for Comp. 121 using Intermediate 3 instead of Intermediate 4. The commercially available (difluoromethyl)-sulfonyl zinc was used instead of custom made (difluoromethyl-d)-sulfonyl zinc. ¹HNMR (400 MHz, CDCl₃) δ 8.55 (s, 1H), 7.84 (s, 1H), 6.93-6.66 (m, 1H), 5.79-5.68 (m, 2H), 4.14-3.96 (m, 1H), 3.86-3.80 (m, 2H), 2.98-2.91 (m, 2H), 2.85 (s, 4H), 2.33-2.25 (m, 1H), 2.07-1.82 (m, 5H), 1.19 (s, 3H). [M+H]⁺=476.2. ee %=99.7%. The degree of deuterium enrichment is 85%.

### Example 24. (−)-6-(difluoromethyl)-8-((1R,2R)-2-hydroxy-2-methylcyclopentyl)-2-((1-((methyl-d3)sulfonyl)piperidin-4-yl-3,3,5,5-d4)amino)pyrido[2,3-d]pyrimidin-7(8H)-one (Comp. 146)

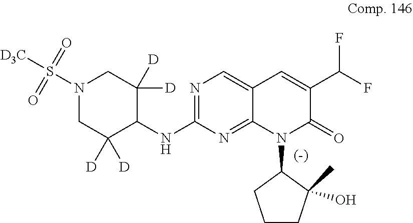 [SMILES: [2H]C1([2H])CN(S(=O)(=O)C([2H])([2H])[2H])CC([2H])([2H])C1Nc1ncc2cc(C(F)F)c(=O)n([C@@H]3CCC[C@@]3(C)O)c2n1]

Compound 146 was synthesized according to the similar synthetic route for Comp. 121, using Intermediate 5 instead of Intermediate 4. The commercially available (difluoromethyl)-sulfonyl zinc was used instead of custom made (difluoromethyl-d)-sulfonyl zinc. ¹HNMR (400 MHz, CDCl₃) δ 8.55 (s, 1H), 7.85 (s, 1H), 6.93-6.66 (m, 1H), 5.80-5.69 (m, 2H), 4.11-3.99 (m, 1H), 3.86-3.80 (m, 2H), 2.94-2.91 (m, 2H), 2.83-2.71 (m, 1H), 2.33-2.25 (m, 1H), 2.07-1.82 (m, 5H), 1.19 (s, 3H). [M+H]⁺=479.2. ee %=99.7%. The degree of deuterium enrichment is 80%.

### Example 25. (−)-6-(difluoromethyl-d)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one (Comp. 149)

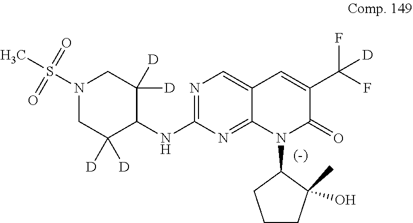 [SMILES: [2H]C(F)(F)c1cc2cnc(NC3C([2H])([2H])CN(S(C)(=O)=O)CC3([2H])[2H])nc2n([C@@H]2CCC[C@@]2(C)O)c1=O]

Compound 149 was synthesized according to the similar synthetic route for Comp. 20, using Comp. 61 instead of Comp. 1. ¹HNMR (400 MHz, CDCl₃) δ 8.56 (s, 1H), 7.85 (s, 1H), 5.79 (brs, 1H), 4.14-3.99 (m, 2H), 3.86-3.79 (m, 2H), 2.95-2.92 (m, 2H), 2.86 (s, 4H), 2.34-2.26 (m, 1H), 2.09-1.82 (m, 5H), 1.20 (s, 3H). [M+H]⁺=477.2. ee %=99.7%. The degree of deuterium enrichment is 85%.

### Example 26. (−)-6-(difluoromethyl-d)-8-((1R,2R)-2-hydroxy-2-methylcyclopentyl)-2-((1-((methyl-d3)sulfonyl)piperidin-4-yl-3,3,5,5-d4)amino)pyrido[2,3-d]pyrimidin-7(8H)-one (Comp. 152)

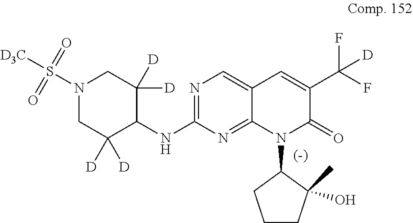 [SMILES: [2H]C(F)(F)c1cc2cnc(NC3C([2H])([2H])CN(S(=O)(=O)C([2H])([2H])[2H])CC3([2H])[2H])nc2n([C@@H]2CCC[C@@]2(C)O)c1=O]

Synthetic route for Comp. 152 is illustrated in the Scheme 19 below:

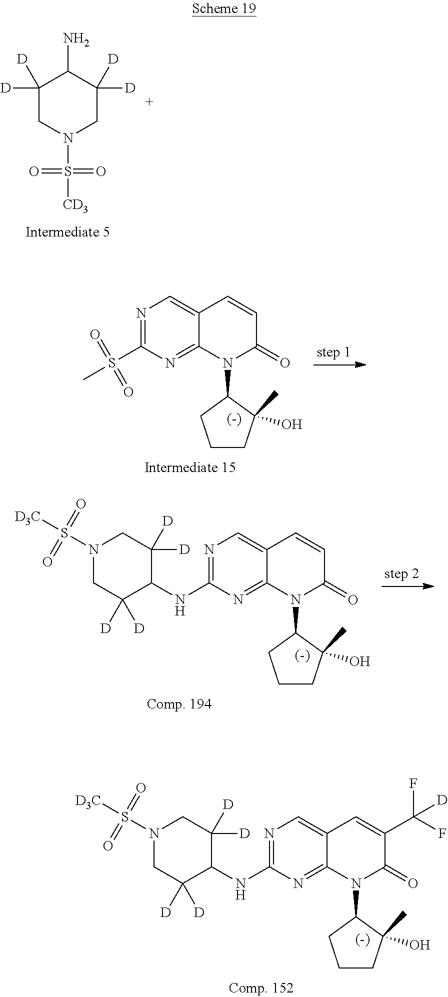 [SMILES: [2H]C1([2H])CN(S(=O)(=O)C([2H])([2H])[2H])CC([2H])([2H])C1N ; C[C@@]1(O)CCC[C@H]1n1c(=O)ccc2cnc(S(C)(=O)=O)nc21 ; [2H]C1([2H])CN(S(=O)(=O)C([2H])([2H])[2H])CC([2H])([2H])C1Nc1ncc2ccc(=O)n([C@@H]3CCC[C@@]3(C)O)c2n1 ; [2H]C(F)(F)c1cc2cnc(NC3C([2H])([2H])CN(S(=O)(=O)C([2H])([2H])[2H])CC3([2H])[2H])nc2n([C@@H]2CCC[C@@]2(C)O)c1=O]

Step 1: The procedure was the same as Comp. 11 to provide Comp. 194, 8-((1R,2R)-2-hydroxy-2-methylcyclopentyl)-2-((1-((methyl-d3)sulfonyl)piperidin-3,3,5,5-d4-4-yl)amino)pyrido[2,3-d]pyrimidin-7(8H)-one. [M+H]⁺=429.2.

Step 2: The procedure was the same as the step 2 of Comp. 20 to provide Comp. 152. ¹HNMR (400 MHz, CDCl₃) δ 8.56 (s, 1H), 7.85 (s, 1H), 5.80 (br s, 2H), 4.11-3.98 (m, 2H), 3.02-2.92 (m, 2H), 2.83-2.70 (m, 1H, 2.34-2.26 (m, 1H), 2.09-1.82 (m, 5H), 1.20 (s, 3H). [M+H]⁺=480.2. ee %=99.7%. The degree of deuterium enrichment is 80%.

### Example 27. (−)-6-(difluoromethyl)-8-((1R,2R)-2-hydroxy-2-methylcyclopentyl)-2-((1-((methyl-d3)sulfonyl)piperidin-4-yl-3,3,4,5,5-d5)amino)pyrido[2,3-d]pyrimidin-7(8H)-one (Comp. 156)

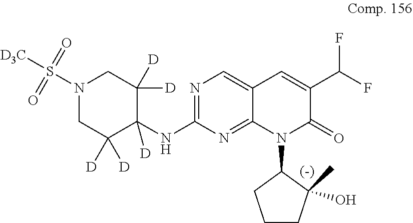 [SMILES: [2H]C1([2H])CN(S(=O)(=O)C([2H])([2H])[2H])CC([2H])([2H])C1([2H])Nc1ncc2cc(C(F)F)c(=O)n([C@@H]3CCC[C@@]3(C)O)c2n1]

Compound 156 was synthesized according to the similar synthetic route for Comp. 121, using Intermediate 7 instead of Intermediate 4. ¹HNMR (400 MHz, CDCl₃) δ 8.55 (s, 1H), 7.84 (s, 1H), 5.81-5.59 (m, 2H), 3.86-3.81 (m, 2H), 2.96-2.91 (m, 2H), 2.83-2.71 (m, 1H), 2.33-2.26 (m, 1H), 2.09-1.82 (m, 5H), 1.19 (s, 3H). [M+H]⁺=480.2. ee %=99.7%. The degree of deuterium enrichment is 88.8%.

### Example 28. (−)-6-(difluoromethyl-d)-8-((1R,2R)-2-hydroxy-2-methylcyclopentyl)-2-((1-((methyl-d3)sulfonyl)piperidin-4-yl-3,3,4,5,5-d5)amino)pyrido[2,3-d]pyrimidin-7(8H)-one (Comp. 159)

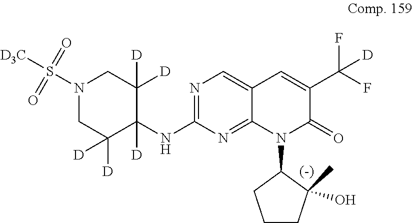 [SMILES: [2H]C(F)(F)c1cc2cnc(NC3([2H])C([2H])([2H])CN(S(=O)(=O)C([2H])([2H])[2H])CC3([2H])[2H])nc2n([C@@H]2CCC[C@@]2(C)O)c1=O]

Compound 159, white solid, was synthesized according to the similar synthetic route for Comp. 152, using Intermediate 7 instead of Intermediate 5. ¹HNMR (400 MHz, CDCl₃) δ 8.55 (s, 1H), 7.84 (s, 1H), 6.03 (brs, 0.5H), 5.78 (brs, 1.5H), 3.85-3.78 (m, 2H), 2.98-2.92 (m, 2H), 2.83-2.75 (m, 1H), 2.31-2.25 (m, 1H), 2.08-2.01 (m, 3H), 1.86-1.82 (m, 2H), [M+H]⁺=481.2. ee %=99.7%. The degree of deuterium enrichment is 88.8%.

### Example 29. (−)-8-((1R,2R)-2-hydroxy-2-methylcyclopentyl)-6-(methyl-d3)-2-((1-((methyl-d3)sulfonyl)piperidin-4-yl-3,3,4,5,5-d5)amino)pyrido[2,3-d]pyrimidin-7(8H)-one (Comp. 163)

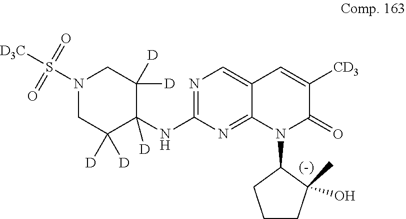 [SMILES: [2H]C([2H])([2H])c1cc2cnc(NC3([2H])C([2H])([2H])CN(S(=O)(=O)C([2H])([2H])[2H])CC3([2H])[2H])nc2n([C@@H]2CCC[C@@]2(C)O)c1=O]

Compound 163, white solid, was synthesized according to the similar synthetic route for Comp. 11, using Intermediate 7 instead of 1-(methylsulfonyl)-piperidin-4-amine, Intermediate 9 instead of Intermediate 8. ¹HNMR (400 MHz, CDCl₃) δ 8.39 (s, 1H), 7.35 (s, 1H), 5.77-5.73 (m, 1H), 3.84-3.79 (m, 2H), 2.94-2.82 (m, 3H), 2.29-2.24 (m, 1H), 2.05-1.83 (m, 5H), 1.16 (s, 3H). [M+H]⁺=447.2. ee %=99.7%. The degree of deuterium enrichment is 83%.

### Example 30. (−)-8-((1R,2R)-2-hydroxy-2-methylcyclopentyl)-6-(methyl-d3)-2-((1-(methylsulfonyl)piperidin-4-yl-3,3,4,5,5-d5)amino)pyrido[2,3-d]pyrimidin-7(8H)-one (Comp. 164)

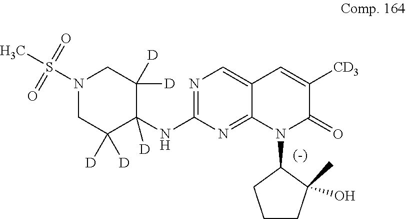 [SMILES: [2H]C([2H])([2H])c1cc2cnc(NC3([2H])C([2H])([2H])CN(S(C)(=O)=O)CC3([2H])[2H])nc2n([C@@H]2CCC[C@@]2(C)O)c1=O]

Compound 164, light yellow solid, was synthesized according to the similar synthetic route for Comp. 11, using Intermediate 6 instead of 1-(methylsulfonyl)-piperidin-4-amine, Intermediate 9 instead of Intermediate 8. ¹HNMR (400 MHz, CDCl₃) δ 8.40 (s, 1H), 7.35 (s, 1H), 5.78-5.74 (m, 1H), 5.48 (brs, 1H), 3.84-3.79 (m, 2H), 2.95-2.90 (m, 2H), 2.84 (s, 4H), 2.32-2.24 (m, 1H), 2.06-1.84 (m, 5H), 1.17 (s, 3H). [M+H]⁺=444.2. ee %=99.7%. The degree of deuterium enrichment is 85%.

### Example 31. (−) 8-((1R,2R)-2-hydroxy-2-methylcyclopentyl)-2-((1-(methylsulfonyl)piperidin-4-yl)amino)pyrido[2,3-d]pyrimidin-7(8H)-one (Comp. 169)

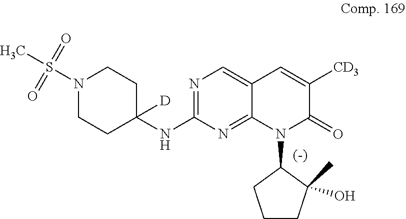 [SMILES: [2H]C1(Nc2ncc3cc(C([2H])([2H])[2H])c(=O)n([C@@H]4CCC[C@@]4(C)O)c3n2)CCN(S(C)(=O)=O)CC1]

Compound 169 was synthesized according to the similar synthetic route for Comp. 11, using Intermediate 1 instead of 1-(methylsulfonyl)-piperidin-4-amine, Intermediate 9 instead of Intermediate 8. ¹HNMR (400 MHz, CDCl₃) δ 8.40 (s, 1H), 7.34 (s, 1H), 5.78-5.74 (m, 1H), 5.41 (brs, 1H), 3.85-3.80 (m, 2H), 2.99-2.91 (m, 2H), 2.84 (s, 4H), 2.30-2.21 (m, 3H), 2.06-1.85 (m, 4.5H), 1.74-1.64 (m, 5H), 1.18 (s, 3H). [M+H]⁺=440.2. ee %=99.7%. The degree of deuterium enrichment is 99%.

### Example 32. (+)-8-((1S,2S)-2-hydroxy-2-methylcyclopentyl)-6-methyl-2-((1-(methylsulfonyl)piperidin-4-yl)amino)pyrido[2,3-d]pyrimidin-7(8H)-one (Comp. 182)

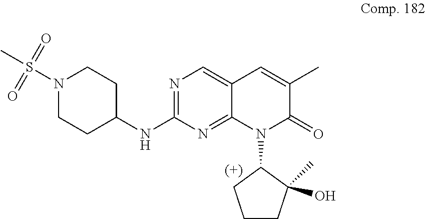 [SMILES: Cc1cc2cnc(NC3CCN(S(C)(=O)=O)CC3)nc2n([C@H]2CCC[C@]2(C)O)c1=O]

Compound 182 was synthesized according to WO2018033815. [M+H]⁺=436.2.

### Example 33. (+)-8-((1S,2S)-2-hydroxy-2-methylcyclopentyl)-6-(methyl-d3)-2-((1-(methylsulfonyl)piperidin-4-yl)amino)pyrido[2,3-d]pyrimidin-7(8H)-one (Comp. 189)

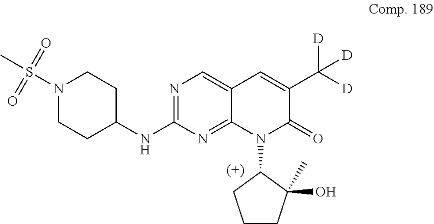 [SMILES: [2H]C([2H])([2H])c1cc2cnc(NC3CCN(S(C)(=O)=O)CC3)nc2n([C@H]2CCC[C@]2(C)O)c1=O]

Comp. 189 was synthesized according to the same route for Comp. 37. [M+H]⁺=439.2.

### Example 34. (+)-6-(difluoromethyl)-8-((1S,2S)-2-hydroxy-2-methylcyclopentyl)-2-((1-(methylsulfonyl)piperidin-4-yl)amino)pyrido[2,3-d]pyrimidin-7(8H)-one (Comp. 194)

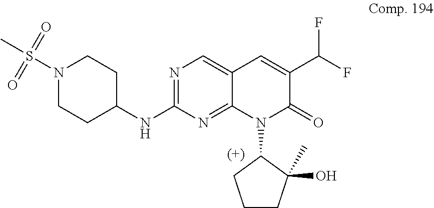 [SMILES: C[C@]1(O)CCC[C@@H]1n1c(=O)c(C(F)F)cc2cnc(NC3CCN(S(C)(=O)=O)CC3)nc21]

Comp. 194 was synthesized according to WO2018033815. [M+H]⁺=472.2.

### Example 35. (+)-6-(difluoromethyl-d)-8-((1S,2S)-2-hydroxy-2-methylcyclopentyl)-2-((1-(methylsulfonyl)piperidin-4-yl)amino)pyrido[2,3-d]pyrimidin-7(8H)-one (Comp. 205)

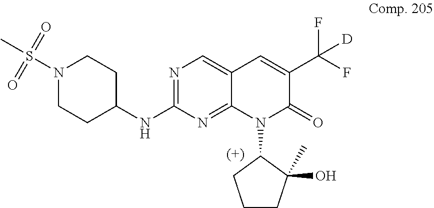 [SMILES: [2H]C(F)(F)c1cc2cnc(NC3CCN(S(C)(=O)=O)CC3)nc2n([C@H]2CCC[C@]2(C)O)c1=O]

Comp. 205 was synthesized according to the same route for Comp. 28. [M+H]⁺=473.2.

TABLE 1
Deuterated Pyridopyrimidinone CDK2 inhibitors (a. D % stands for degree of
deuterium enrichment; ee % stands for enantiomeric purity)

| | | | m/z |
|---|---|---|---|
| | | | [M + H]⁺/ |
| | | | D % ^{a}/ |
| Compound | Chemical Structure | Chemical Name | ee % |
| Comp. 1 | 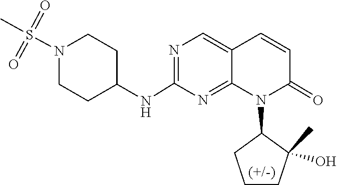 [SMILES: C[C@@]1(O)CCC[C@H]1n1c(=O)ccc2cnc(NC3CCN(S(C)(=O)=O)CC3)nc21] | (±)-8-(trans-2-hydroxy-2- methylcyclopentyl)-2-((1- (methylsulfonyl)piperidin- 4-yl)amino)pyrido[2,3- d]pyrimidin-7(8H)-one | 422.2 |
| Comp. 2 | 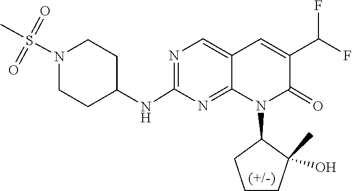 [SMILES: C[C@@]1(O)CCC[C@H]1n1c(=O)c(C(F)F)cc2cnc(NC3CCN(S(C)(=O)=O)CC3)nc21] | (±)-6-(difluoromethyl)-8- (trans-2-hydroxy-2- methylcyclopentyl)-2-((1- (methylsulfonyl)piperidin- 4-yl)amino)pyrido[2,3- d]pyrimidin-7(8H)-one | 472.2 |
| Comp. 3 | 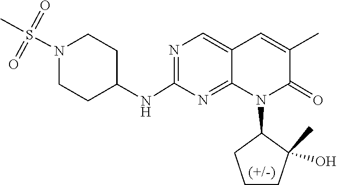 [SMILES: Cc1cc2cnc(NC3CCN(S(C)(=O)=O)CC3)nc2n([C@@H]2CCC[C@@]2(C)O)c1=O] | (±)-8-(trans-2-hydroxy-2- methylcyclopentyl)-6- methyl-2-((1- (methylsulfonyl)piperidin- 4-yl)amino)pyrido[2,3- d]pyrimidin-7(8H)-one | 436.2 |
| Comp. 4 | 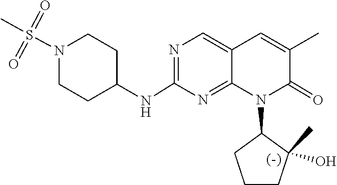 [SMILES: Cc1cc2cnc(NC3CCN(S(C)(=O)=O)CC3)nc2n([C@@H]2CCC[C@@]2(C)O)c1=O] | (−)-8-((1R,2R)-2-hydroxy- 2-methylcyclopentyl)-6- methyl-2-((1- (methylsulfonyl)piperidin- 4-yl)amino)pyrido[2,3- d]pyrimidin-7(8H)-one | 436.2 |
| Comp. 5 | 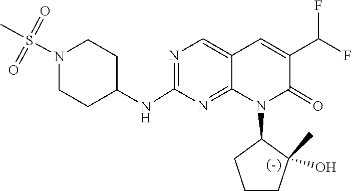 [SMILES: C[C@@]1(O)CCC[C@H]1n1c(=O)c(C(F)F)cc2cnc(NC3CCN(S(C)(=O)=O)CC3)nc21] | (−)-6-(difluoromethyl)-8- ((1R,2R)-2-hydroxy-2- methylcyclopentyl)-2-((1- (methylsulfonyl)piperidin- 4-yl)amino)pyrido[2,3- d]pyrimidin-7(8H)-one | 472.2 |
| Comp. 6 | 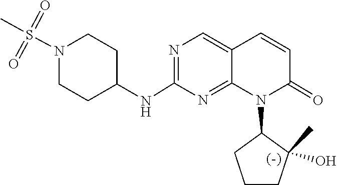 [SMILES: C[C@@]1(O)CCC[C@H]1n1c(=O)ccc2cnc(NC3CCN(S(C)(=O)=O)CC3)nc21] | (−)-8-((1R,2R)-2-hydroxy- 2-methylcyclopentyl)-2- ((1- (methylsulfonyl)piperidin- 4-yl)amino)pyrido[2,3- d]pyrimidin-7(8H)-one | 422.2 |
| Comp. 7 | 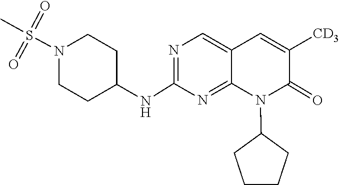 [SMILES: [2H]C([2H])([2H])c1cc2cnc(NC3CCN(S(C)(=O)=O)CC3)nc2n(C2CCCC2)c1=O] | 8-cyclopentyl-6-(methyl- d3)-2-((1- (methylsulfonyl)piperidin- 4-yl)amino)pyrido[2,3- d]pyrimidin-7(8H)-one | 409.2 |
| Comp. 8 | 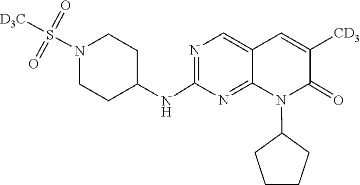 [SMILES: [2H]C([2H])([2H])c1cc2cnc(NC3CCN(S(=O)(=O)C([2H])([2H])[2H])CC3)nc2n(C2CCCC2)c1=O] | 8-cyclopentyl-6-(methyl- d3)-2-((1-((methyl-d3)- sulfonyl)piperidin-4- yl)amino)pyrido[2,3- d]pyrimidin-7(8H)-one | 412.2 |
| Comp. 9 | 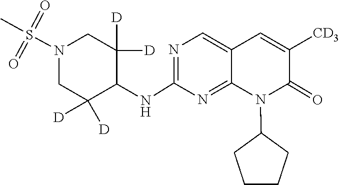 [SMILES: [2H]C([2H])([2H])c1cc2cnc(NC3C([2H])([2H])CN(S(C)(=O)=O)CC3([2H])[2H])nc2n(C2CCCC2)c1=O] | 8-cyclopentyl-6-(methyl- d3)-2-((1- (methylsulfonyl)piperidin- 4-yl-3,3,5,5-d4)- amino)pyrido[2,3- d]pyrimidin-7(8H)-one | 413.2 |
| Comp. 10 | 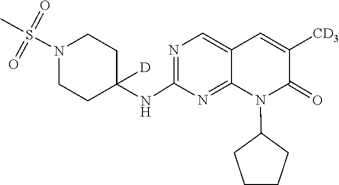 [SMILES: [2H]C1(Nc2ncc3cc(C([2H])([2H])[2H])c(=O)n(C4CCCC4)c3n2)CCN(S(C)(=O)=O)CC1] | 8-cyclopentyl-6-(methyl- d3)-2-((1- (methylsulfonyl)piperidin- 4-yl-4-d)- amino)pyrido[2,3- d]pyrimidin-7(8H)-one | 410.2 |
| Comp. 11 | 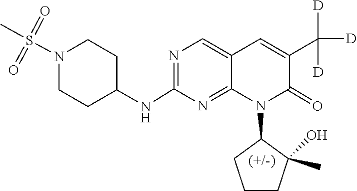 [SMILES: [2H]C([2H])([2H])c1cc2cnc(NC3CCN(S(C)(=O)=O)CC3)nc2n([C@@H]2CCC[C@@]2(C)O)c1=O] | (±)-8-(trans-2-hydroxy-2- methylcyclopentyl)-6- (methyl-d3)-2-((1- (methylsulfonyl)piperidin- 4-yl)amino)pyrido[2,3- d]pyrimidin-7(8H)-one | 439.2 |
| Comp. 12 | 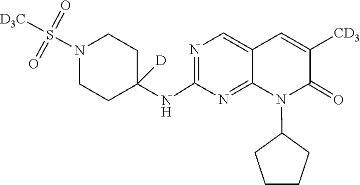 [SMILES: [2H]C1(Nc2ncc3cc(C([2H])([2H])[2H])c(=O)n(C4CCCC4)c3n2)CCN(S(=O)(=O)C([2H])([2H])[2H])CC1] | 8-cyclopentyl-6-(methyl- d3)-2-((1-((methyl- d3)sulfonyl)piperidin-4- yl-4-d)-amino)pyrido[2,3- d]pyrimidin-7(8H)-one | 413.2 |
| Comp. 13 | 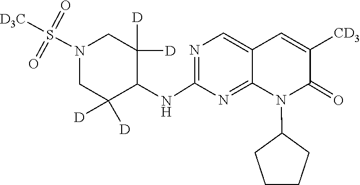 [SMILES: [2H]C([2H])([2H])c1cc2cnc(NC3C([2H])([2H])CN(S(=O)(=O)C([2H])([2H])[2H])CC3([2H])[2H])nc2n(C2CCCC2)c1=O] | 8-cyclopentyl-6-(methyl- d3)-2-((1-((methyl- d3)sulfonyl)piperidin-4- yl-3,3,5,5-d4)- amino)pyrido[2,3- d]pyrimidin-7(8H)-one | 416.2 |
| Comp. 14 | 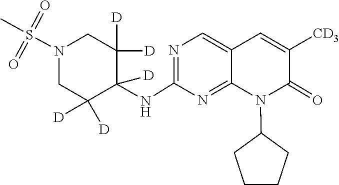 [SMILES: [2H]C([2H])([2H])c1cc2cnc(NC3([2H])C([2H])([2H])CN(S(C)(=O)=O)CC3([2H])[2H])nc2n(C2CCCC2)c1=O] | 8-cyclopentyl-6-(methyl- d3)-2-((1- (methylsulfonyl)piperidin- 4-yl-3,3,4,5,5-d5)- amino)pyrido[2,3- d]pyrimidin-7(8H)-one | 414.2 |
| Comp. 15 | 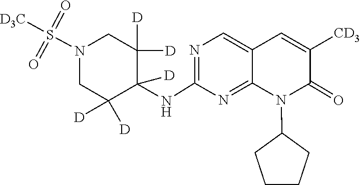 [SMILES: [2H]C([2H])([2H])c1cc2cnc(NC3([2H])C([2H])([2H])CN(S(=O)(=O)C([2H])([2H])[2H])CC3([2H])[2H])nc2n(C2CCCC2)c1=O] | 8-cyclopentyl-6-(methyl- d3)-2-((1-((methyl-d3)- sulfonyl)piperidin-4-yl- 3,3,4,5,5-d5)- amino)pyrido[2,3- d]pyrimidin-7(8H)-one | 417.2 |
| Comp. 16 | 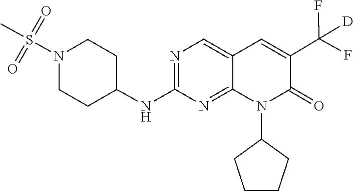 [SMILES: [2H]C(F)(F)c1cc2cnc(NC3CCN(S(C)(=O)=O)CC3)nc2n(C2CCCC2)c1=O] | 8-cyclopentyl-6- (difluoromethyl-d)-2-((1- (methylsulfonyl)piperidin- 4-yl)amino)pyrido[2,3- d]pyrimidin-7(8H)-one | 443.2 |
| Comp. 17 | 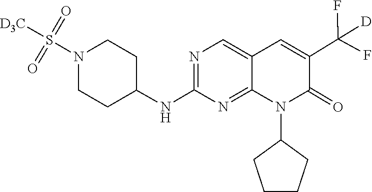 [SMILES: [2H]C(F)(F)c1cc2cnc(NC3CCN(S(=O)(=O)C([2H])([2H])[2H])CC3)nc2n(C2CCCC2)c1=O] | 8-cyclopentyl-6- (difluoromethyl-d)-2-((1- ((methyl- d3)sulfonyl)piperidin-4- yl)amino)pyrido[2,3- d]pyrimidin-7(8H)-one | 446.2 |
| Comp. 18 | 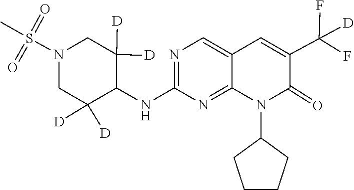 [SMILES: [2H]C(F)(F)c1cc2cnc(NC3C([2H])([2H])CN(S(C)(=O)=O)CC3([2H])[2H])nc2n(C2CCCC2)c1=O] | 8-cyclopentyl-6- (difluoromethyl-d)-2-((1- (methylsulfonyl)piperidin- 4-yl-3,3,5,5-d4)- amino)pyrido[2,3- d]pyrimidin-7(8H)-one | 447.2 |
| Comp. 19 | 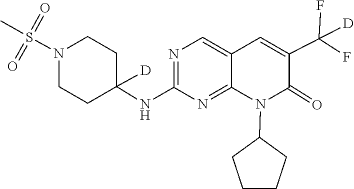 [SMILES: [2H]C1(Nc2ncc3cc(C([2H])(F)F)c(=O)n(C4CCCC4)c3n2)CCN(S(C)(=O)=O)CC1] | 8-cyclopentyl-6- (difluoromethyl-d)-2-((1- (methylsulfonyl)piperidin- 4-yl-4-d)- amino)pyrido[2,3- d]pyrimidin-7(8H)-one | 444.2 |
| Comp. 20 | 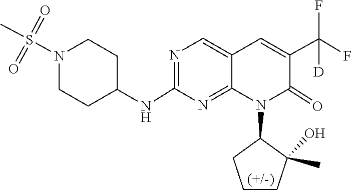 [SMILES: [2H]C(F)(F)c1cc2cnc(NC3CCN(S(C)(=O)=O)CC3)nc2n([C@@H]2CCC[C@@]2(C)O)c1=O] | (±)-6-(difluoromethyl-d)- 8-(trans-2-hydroxy-2- methylcyclopentyl)-2-((1- (methylsulfonyl)piperidin- 4-yl)amino)pyrido[2,3- d]pyrimidin-7(8H)-one | 473.2 |
| Comp. 21 | 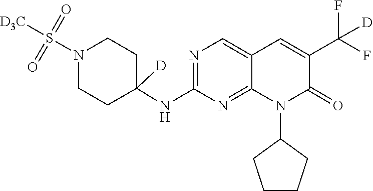 [SMILES: [2H]C1(Nc2ncc3cc(C([2H])(F)F)c(=O)n(C4CCCC4)c3n2)CCN(S(=O)(=O)C([2H])([2H])[2H])CC1] | 8-cyclopentyl-6- (difluoromethyl-d)-2-((1- ((methyl- d3)sulfonyl)piperidin-4- yl-4-d)-amino)pyrido[2,3- d]pyrimidin-7(8H)-one | 447.2 |
| Comp. 22 | 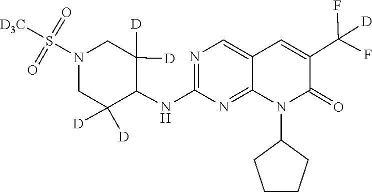 [SMILES: [2H]C(F)(F)c1cc2cnc(NC3C([2H])([2H])CN(S(=O)(=O)C([2H])([2H])[2H])CC3([2H])[2H])nc2n(C2CCCC2)c1=O] | 8-cyclopentyl-6- (difluoromethyl-d)-2-((1- ((methyl- d3)sulfonyl)piperidin-4- yl-3,3,5,5-d4)- amino)pyrido[2,3- d]pyrimidin-7(8H)-one | 450.2 |
| Comp. 23 | 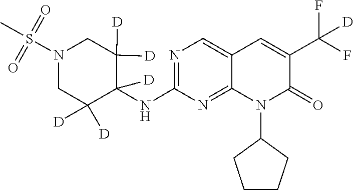 [SMILES: [2H]C(F)(F)c1cc2cnc(NC3([2H])C([2H])([2H])CN(S(C)(=O)=O)CC3([2H])[2H])nc2n(C2CCCC2)c1=O] | 8-cyclopentyl-6- (difluoromethyl-d)-2-((1- (methylsulfonyl)piperidin- 4-yl-3,3,4,5,5-d5)- amino)pyrido[2,3- d]pyrimidin-7(8H)-one | 448.2 |
| Comp. 24 | 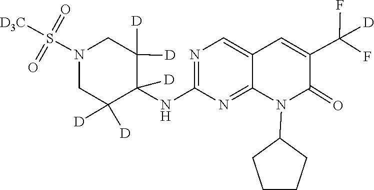 [SMILES: [2H]C(F)(F)c1cc2cnc(NC3([2H])C([2H])([2H])CN(S(=O)(=O)C([2H])([2H])[2H])CC3([2H])[2H])nc2n(C2CCCC2)c1=O] | 8-cyclopentyl-6- (difluoromethyl-d)-2-((1- ((methyl- d3)sulfonyl)piperidin-4- yl-3,3,4,5,5-d5)- amino)pyrido[2,3- d]pyrimidin-7(8H)-one | 451.2 |
| Comp. 25 | 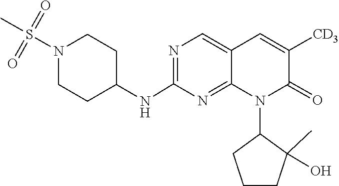 [SMILES: [2H]C([2H])([2H])c1cc2cnc(NC3CCN(S(C)(=O)=O)CC3)nc2n(C2CCCC2(C)O)c1=O] | (±)-8-(2-hydroxy-2- methylcyclopentyl)-6- (methyl-d3)-2-((1- (methylsulfonyl)piperidin- 4-yl)amino)pyrido[2,3- d]pyrimidin-7(8H)-one | 439.2 |
| Comp. 26 | 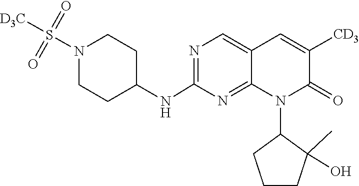 [SMILES: [2H]C([2H])([2H])c1cc2cnc(NC3CCN(S(=O)(=O)C([2H])([2H])[2H])CC3)nc2n(C2CCCC2(C)O)c1=O] | (±)-8-(2-hydroxy-2- methylcyclopentyl)-6- (methyl-d3)-2-((1- ((methyl- d3)sulfonyl)piperidin-4- yl)amino)pyrido[2,3- d]pyrimidin-7(8H)-one | 442.2 |
| Comp. 27 | 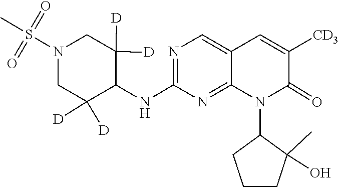 [SMILES: [2H]C([2H])([2H])c1cc2cnc(NC3C([2H])([2H])CN(S(C)(=O)=O)CC3([2H])[2H])nc2n(C2CCCC2(C)O)c1=O] | (±)-8-(2-hydroxy-2- methylcyclopentyl)-6- (methyl-d3)-2-((1- (methylsulfonyl)piperidin- 4-yl-3,3,5,5-d4)- amino)pyrido[2,3- d]pyrimidin-7(8H)-one | 443.2 |
| Comp. 28 | 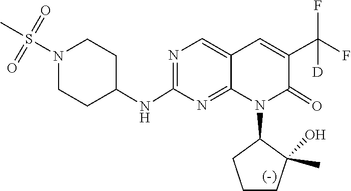 [SMILES: [2H]C(F)(F)c1cc2cnc(NC3CCN(S(C)(=O)=O)CC3)nc2n([C@@H]2CCC[C@@]2(C)O)c1=O] | (−)-6-(difluoromethyl-d)- 8-((1R,2R)-2-hydroxy-2- methylcyclopentyl)-2-((1- (methylsulfonyl)piperidin- 4-yl)amino)pyrido[2,3- d]pyrimidin-7(8H)-one | 472.2/ >95% D/ 99.75% ee |
| Comp. 29 | 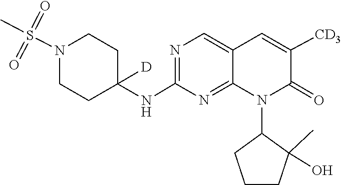 [SMILES: [2H]C1(Nc2ncc3cc(C([2H])([2H])[2H])c(=O)n(C4CCCC4(C)O)c3n2)CCN(S(C)(=O)=O)CC1] | (±)-8-(2-hydroxy-2- methylcyclopentyl)-6- (methyl-d3)-2-((1- (methylsulfonyl)piperidin- 4-yl-4-d)- amino)pyrido[2,3- d]pyrimidin-7(8H)-one | 440.2 |
| Comp. 30 | 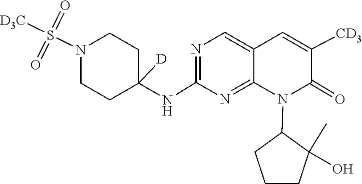 [SMILES: [2H]C1(Nc2ncc3cc(C([2H])([2H])[2H])c(=O)n(C4CCCC4(C)O)c3n2)CCN(S(=O)(=O)C([2H])([2H])[2H])CC1] | (±)-8-(2-hydroxy-2- methylcyclopentyl)-6- (methyl-d3)-2-((1- ((methyl- d3)sulfonyl)piperidin-4- yl-4-d)-amino)pyrido[2,3- d]pyrimidin-7(8H)-one | 443.2 |
| Comp. 31 | 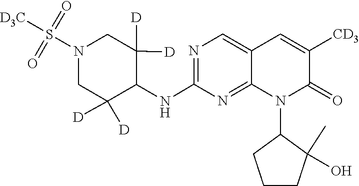 [SMILES: [2H]C([2H])([2H])c1cc2cnc(NC3C([2H])([2H])CN(S(=O)(=O)C([2H])([2H])[2H])CC3([2H])[2H])nc2n(C2CCCC2(C)O)c1=O] | (±)-8-(2-hydroxy-2- methylcyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 446.3 |
| Comp. 32 | 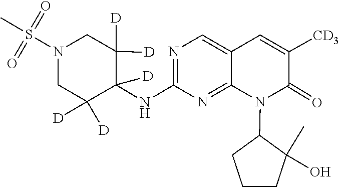 [SMILES: [2H]C([2H])([2H])c1cc2cnc(NC3([2H])C([2H])([2H])CN(S(C)(=O)=O)CC3([2H])[2H])nc2n(C2CCCC2(C)O)c1=O] | (±)-8-(2-hydroxy-2- methylcyclopentyl)-6- (methyl-d3)-2-((1- (methylsulfonyl)piperidin- 4-yl-3,3,4,5,5-d5)- amino)pyrido[2,3- d]pyrimidin-7(8H)-one | 444.2 |
| Comp. 33 | 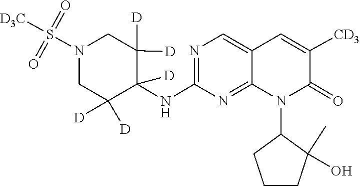 [SMILES: [2H]C([2H])([2H])c1cc2cnc(NC3([2H])C([2H])([2H])CN(S(=O)(=O)C([2H])([2H])[2H])CC3([2H])[2H])nc2n(C2CCCC2(C)O)c1=O] | (±)-8-(2-hydroxy-2- methylcyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 447.3 |
| Comp. 34 | 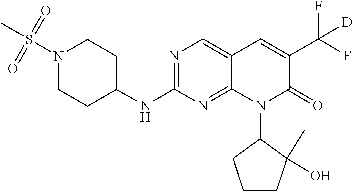 [SMILES: [2H]C(F)(F)c1cc2cnc(NC3CCN(S(C)(=O)=O)CC3)nc2n(C2CCCC2(C)O)c1=O] | (±)-8-(2-hydroxy-2- methylcyclopentyl)-6- (difluoromethyl-d)-2-((1- (methylsulfonyl)piperidin- 4-yl)amino)pyrido[2,3- d]pyrimidin-7(8H)-one | 473.2 |
| Comp. 35 | 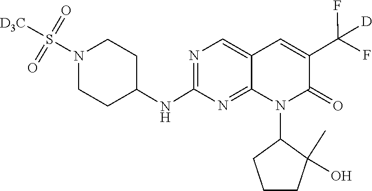 [SMILES: [2H]C(F)(F)c1cc2cnc(NC3CCN(S(=O)(=O)C([2H])([2H])[2H])CC3)nc2n(C2CCCC2(C)O)c1=O] | (±)-8-(2-hydroxy-2- methylcyclopentyl)-6- (difluoromethyl-d)-2-((1- ((methyl- d3)sulfonyl)piperidin-4- yl)amino)pyrido[2,3- d]pyrimidin-7(8H)-one | 476.2 |
| Comp. 36 | 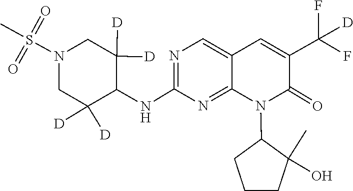 [SMILES: [2H]C(F)(F)c1cc2cnc(NC3C([2H])([2H])CN(S(C)(=O)=O)CC3([2H])[2H])nc2n(C2CCCC2(C)O)c1=O] | (±)-8-(2-hydroxy-2- methylcyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 477.2 |
| Comp. 37 | 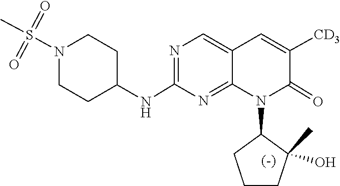 [SMILES: [2H]C([2H])([2H])c1cc2cnc(NC3CCN(S(C)(=O)=O)CC3)nc2n([C@@H]2CCC[C@@]2(C)O)c1=O] | (−)-8-((1R,2R)-2-hydroxy- 2-methylcyclopentyl)-6- (methyl-d3)-2-((1- (methylsulfonyl)piperidin- 4-yl)amino)pyrido[2,3- d]pyrimidin-7(8H)-one | 439.2/ >95% D/ 99.75% ee |
| Comp. 38 | 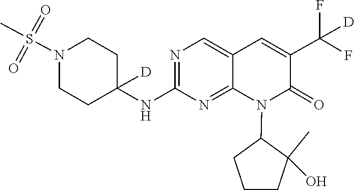 [SMILES: [2H]C1(Nc2ncc3cc(C([2H])(F)F)c(=O)n(C4CCCC4(C)O)c3n2)CCN(S(C)(=O)=O)CC1] | (±)-8-(2-hydroxy-2- methylcyclopentyl)-6- (difluoromethyl-d)-2-((1- (methylsulfonyl)piperidin- 4-yl-4-d)- amino)pyrido[2,3- d]pyrimidin-7(8H)-one | 474.2 |
| Comp. 39 | 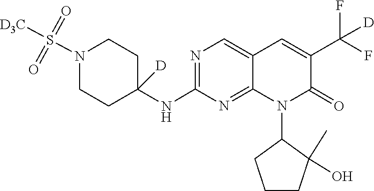 [SMILES: [2H]C1(Nc2ncc3cc(C([2H])(F)F)c(=O)n(C4CCCC4(C)O)c3n2)CCN(S(=O)(=O)C([2H])([2H])[2H])CC1] | (±)-8-(2-hydroxy-2- methylcyclopentyl)-6- (difluoromethyl-d)-2-((1- ((methyl- d3)sulfonyl)piperidin-4- yl-4-d)-amino)pyrido[2,3- d]pyrimidin-7(8H)-one | 477.2 |
| Comp. 40 | 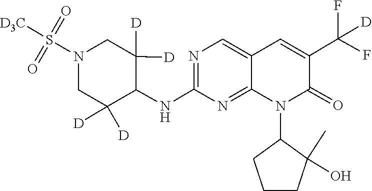 [SMILES: [2H]C(F)(F)c1cc2cnc(NC3C([2H])([2H])CN(S(=O)(=O)C([2H])([2H])[2H])CC3([2H])[2H])nc2n(C2CCCC2(C)O)c1=O] | (±)-8-(2-hydroxy-2- methylcyclopentyl)-6- (difluoromethyl-d)-2-((1- ((methyl- d3)sulfonyl)piperidin-4- yl-3,3,5,5-d4)- amino)pyrido[2,3- d]pyrimidin-7(8H)-one | 480.2 |
| Comp. 41 | 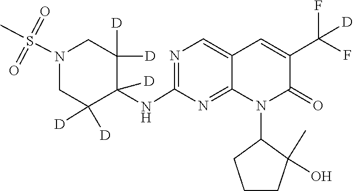 [SMILES: [2H]C(F)(F)c1cc2cnc(NC3([2H])C([2H])([2H])CN(S(C)(=O)=O)CC3([2H])[2H])nc2n(C2CCCC2(C)O)c1=O] | (±)-8-(2-hydroxy-2- methylcyclopentyl)-6- (difluoromethyl-d)-2-((1- (methylsulfonyl)piperidin- 4-yl-3,3,4,5,5-d5)- amino)pyrido[2,3- d]pyrimidin-7(8H)-one | 478.2 |
| Comp. 42 | 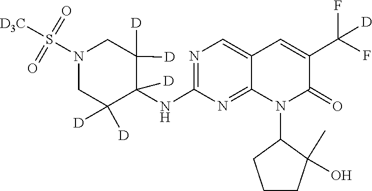 [SMILES: [2H]C(F)(F)c1cc2cnc(NC3([2H])C([2H])([2H])CN(S(=O)(=O)C([2H])([2H])[2H])CC3([2H])[2H])nc2n(C2CCCC2(C)O)c1=O] | (±)-8-(2-hydroxy-2- methylcyclopentyl)-6- (difluoromethyl-d)-2-((1- ((methyl- d3)sulfonyl)piperidin-4- yl-3, 3,4,5,5-d5)- amino)pyrido[2,3- d]pyrimidin-7(8H)-one | 481.2 |
| Comp. 43 | 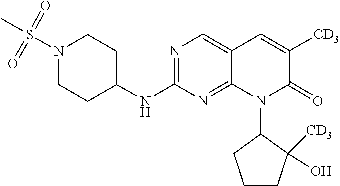 [SMILES: [2H]C([2H])([2H])c1cc2cnc(NC3CCN(S(C)(=O)=O)CC3)nc2n(C2CCCC2(O)C([2H])([2H])[2H])c1=O] | (±)-8-(2-hydroxy-2- (methyl-d3)cyclopentyl)- 6-(methyl-d3)-2-((1- (methylsulfonyl)piperidin- 4-yl)amino)pyrido[2,3- d]pyrimidin-7(8H)-one | 442.2 |
| Comp. 44 | 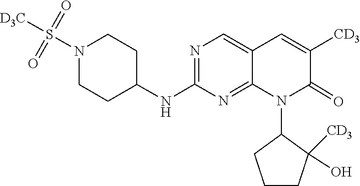 [SMILES: [2H]C([2H])([2H])c1cc2cnc(NC3CCN(S(=O)(=O)C([2H])([2H])[2H])CC3)nc2n(C2CCCC2(O)C([2H])([2H])[2H])c1=O] | (±)-8-(2-hydroxy-2- (methyl-d3)cyclopentyl)- 6-(methyl-d3)-2-((1- ((methyl- d3)sulfonyl)piperidin-4- yl)amino)pyrido[2,3- d]pyrimidin-7(8H)-one | 445.2 |
| Comp. 45 | 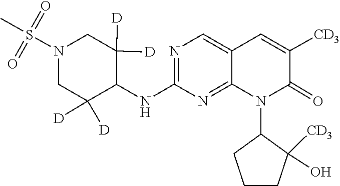 [SMILES: [2H]C([2H])([2H])c1cc2cnc(NC3C([2H])([2H])CN(S(C)(=O)=O)CC3([2H])[2H])nc2n(C2CCCC2(O)C([2H])([2H])[2H])c1=O] | (±)-8-(2-hydroxy-2- (methyl-d3)cyclopentyl)- 6-(methyl-d3)-2-((1- (methylsulfonyl)piperidin- 4-yl-3,3,5,5-d4)- amino)pyrido[2,3- d]pyrimidin-7(8H)-one | 446.3 |
| Comp. 46 | 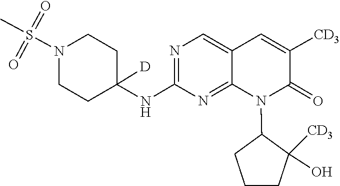 [SMILES: [2H]C1(Nc2ncc3cc(C([2H])([2H])[2H])c(=O)n(C4CCCC4(O)C([2H])([2H])[2H])c3n2)CCN(S(C)(=O)=O)CC1] | (±)-8-(2-hydroxy-2- (methyl-d3)cyclopentyl)- 6-(methyl-d3)-2-((1- (methylsulfonyl)piperidin- 4-yl-4-d)- amino)pyrido[2,3- d]pyrimidin-7(8H)-one | 443.2 |
| Comp. 47 | 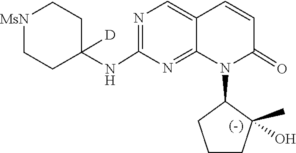 | (−)-8-((1R,2R)-2-hydroxy- 2-methylcyclopentyl)-2- ((1- (methylsulfonyl)piperidin- 4-yl-4- d)amino)pyrido[2,3- d]pyrimidin-7(8H)-one | 423.2/ >95% D/ 99.7% ee |
| Comp. 48 | 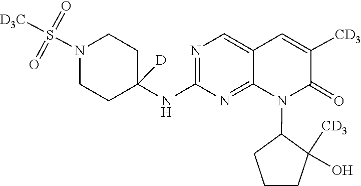 [SMILES: [2H]C1(Nc2ncc3cc(C([2H])([2H])[2H])c(=O)n(C4CCCC4(O)C([2H])([2H])[2H])c3n2)CCN(S(=O)(=O)C([2H])([2H])[2H])CC1] | (±)-8-(2-hydroxy-2- (methyl-d3)cyclopentyl)- 6-(methyl-d3)-2-((1- ((methyl- d3)sulfonyl)piperidin-4- yl-4-d)-amino)pyrido[2,3- d]pyrimidin-7(8H)-one | 446.3 |
| Comp. 49 | 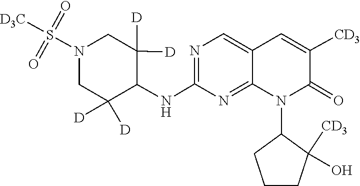 [SMILES: [2H]C([2H])([2H])c1cc2cnc(NC3C([2H])([2H])CN(S(=O)(=O)C([2H])([2H])[2H])CC3([2H])[2H])nc2n(C2CCCC2(O)C([2H])([2H])[2H])c1=O] | (±)-8-(2-hydroxy-2- (methyl-d3)cyclopentyl)- 6-(methyl-d3)-2-((1- ((methyl- d3)sulfonyl)piperidin-4- yl-3,3,5,5-d4)- amino)pyrido[2,3- d]pyrimidin-7(8H)-one | 449.3 |
| Comp. 50 | 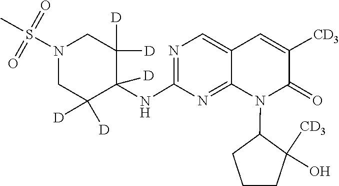 [SMILES: [2H]C([2H])([2H])c1cc2cnc(NC3([2H])C([2H])([2H])CN(S(C)(=O)=O)CC3([2H])[2H])nc2n(C2CCCC2(O)C([2H])([2H])[2H])c1=O] | (±)-8-(2-hydroxy-2- (methyl-d3)cyclopentyl)- 6-(methyl-d3)-2-((1- (methylsulfonyl)piperidin- 4-yl-3,3,4,5,5-d5)- amino)pyrido[2,3- d]pyrimidin-7(8H)-one | 447.3 |
| Comp. 51 | 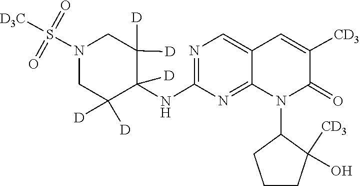 [SMILES: [2H]C([2H])([2H])c1cc2cnc(NC3([2H])C([2H])([2H])CN(S(=O)(=O)C([2H])([2H])[2H])CC3([2H])[2H])nc2n(C2CCCC2(O)C([2H])([2H])[2H])c1=O] | (±)-8-(2-hydroxy-2- (methyl-d3)cyclopentyl)- 6-(methyl-d3)-2-((1- ((methyl- d3)sulfonyl)piperidin-4- yl-3,3,4,5,5-d5)- amino)pyrido[2,3- d]pyrimidin-7(8H)-one | 450.3 |
| Comp. 52 | 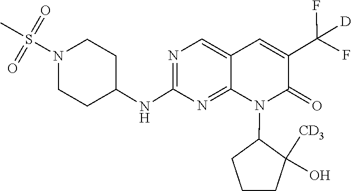 [SMILES: [2H]C(F)(F)c1cc2cnc(NC3CCN(S(C)(=O)=O)CC3)nc2n(C2CCCC2(O)C([2H])([2H])[2H])c1=O] | (±)-8-(2-hydroxy-2- (methyl-d3)cyclopentyl)- 6-(difluoromethyl-d)-2- ((1- (methylsulfonyl)piperidin- 4-yl)amino)pyrido[2,3- d]pyrimidin-7(8H)-one | 476.2 |
| Comp. 53 | 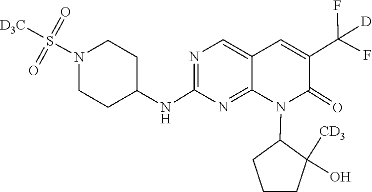 [SMILES: [2H]C(F)(F)c1cc2cnc(NC3CCN(S(=O)(=O)C([2H])([2H])[2H])CC3)nc2n(C2CCCC2(O)C([2H])([2H])[2H])c1=O] | (±)-8-(2-hydroxy-2- (methyl-d3)cyclopentyl)- 6-(difluoromethyl-d)-2- ((1-((methyl- d3)sulfonyl)piperidin-4- yl)amino)pyrido[2,3- d]pyrimidin-7(8H)-one | 479.2 |
| Comp. 54 | 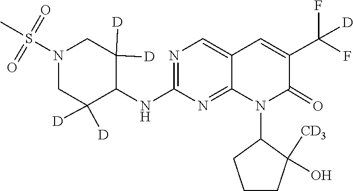 [SMILES: [2H]C(F)(F)c1cc2cnc(NC3C([2H])([2H])CN(S(C)(=O)=O)CC3([2H])[2H])nc2n(C2CCCC2(O)C([2H])([2H])[2H])c1=O] | (±)-8-(2-hydroxy-2- (methyl-d3)cyclopentyl)- 6-(difluoromethyl-d)-2- ((1- (methylsulfonyl)piperidin- 4-yl-3,3,5,5-d4)- amino)pyrido[2,3- d]pyrimidin-7(8H)-one | 480.2 |
| Comp. 55 | 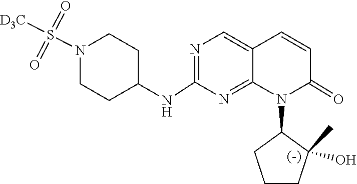 [SMILES: [2H]C([2H])([2H])S(=O)(=O)N1CCC(Nc2ncc3ccc(=O)n([C@@H]4CCC[C@@]4(C)O)c3n2)CC1] | (−)-8-((1R,2R)-2-hydroxy- 2-methylcyclopentyl)-2- ((1-((methyl- d3)sulfonyl)piperidin-4- yl)amino)pyrido[2,3- d]pyrimidin-7(8H)-one | 425.2/ 90% D/ 99.7% ee |
| Comp. 56 | 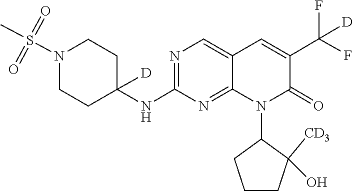 [SMILES: [2H]C1(Nc2ncc3cc(C([2H])(F)F)c(=O)n(C4CCCC4(O)C([2H])([2H])[2H])c3n2)CCN(S(C)(=O)=O)CC1] | (±)-8-(2-hydroxy-2- (methyl-d3)cyclopentyl)- 6-(difluoromethyl-d)-2- ((1- (methylsulfonyl)piperidin- 4-yl-4-d)- amino)pyrido[2,3- d]pyrimidin-7(8H)-one | 477.2 |
| Comp. 57 | 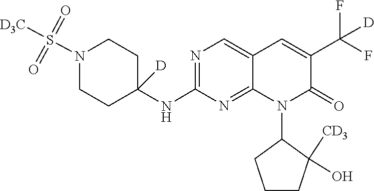 [SMILES: [2H]C1(Nc2ncc3cc(C([2H])(F)F)c(=O)n(C4CCCC4(O)C([2H])([2H])[2H])c3n2)CCN(S(=O)(=O)C([2H])([2H])[2H])CC1] | (±)-8-(2-hydroxy-2- (methyl-d3)cyclopentyl)- 6-(difluoromethyl-d)-2- ((1-((methyl- d3)sulfonyl)piperidin-4- yl-4-d)-amino)pyrido[2,3- d]pyrimidin-7(8H)-one | 480.2 |
| Comp. 58 | 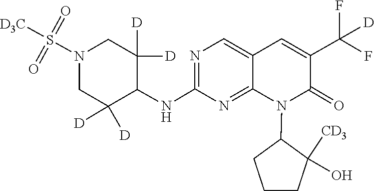 [SMILES: [2H]C(F)(F)c1cc2cnc(NC3C([2H])([2H])CN(S(=O)(=O)C([2H])([2H])[2H])CC3([2H])[2H])nc2n(C2CCCC2(O)C([2H])([2H])[2H])c1=O] | (±)-8-(2-hydroxy-2- (methyl-d3)cyclopentyl)- 6-(difluoromethyl-d)-2- ((1-((methyl- d3)sulfonyl)piperidin-4- yl-3,3,5,5-d4)- amino)pyrido[2,3- d]pyrimidin-7(8H)-one | 483.2 |
| Comp. 59 | 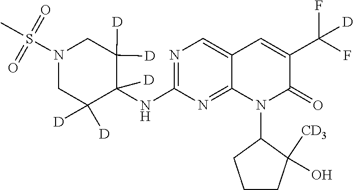 [SMILES: [2H]C(F)(F)c1cc2cnc(NC3([2H])C([2H])([2H])CN(S(C)(=O)=O)CC3([2H])[2H])nc2n(C2CCCC2(O)C([2H])([2H])[2H])c1=O] | (±)-8-(2-hydroxy-2- (methyl-d3)cyclopentyl)- 6-(difluoromethyl-d)-2- ((1- (methylsulfonyl)piperidin- 4-yl-3,3,4,5,5-d5)- amino)pyrido[2,3- d]pyrimidin-7(8H)-one | 481.2 |
| Comp. 60 | 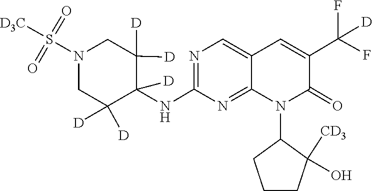 [SMILES: [2H]C(F)(F)c1cc2cnc(NC3([2H])C([2H])([2H])CN(S(=O)(=O)C([2H])([2H])[2H])CC3([2H])[2H])nc2n(C2CCCC2(O)C([2H])([2H])[2H])c1=O] | (±)-8-(2-hydroxy-2- (methyl-d3)cyclopentyl)- 6-(difluoromethyl-d)-2- ((1-((methyl- d3)sulfonyl)piperidin-4- yl-3,3,4,5,5-d5)- amino)pyrido[2,3- d]pyrimidin-7(8H)-one | 484.2 |
| Comp. 61 | 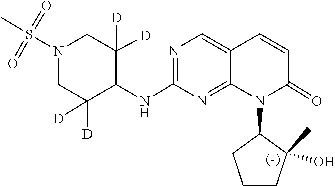 [SMILES: [2H]C1([2H])CN(S(C)(=O)=O)CC([2H])([2H])C1Nc1ncc2ccc(=O)n([C@@H]3CCC[C@@]3(C)O)c2n1] | (−)-8-((1R,2R)-2-hydroxy- 2-methylcyclopentyl)-2- ((1- (methylsulfonyl)piperidin- 4-yl-3,3,5,5- d4)amino)pyrido[2,3- d]pyrimidin-7(8H)-one | 426.2/ 85% D/ 99.7% ee |
| Comp. 62 | 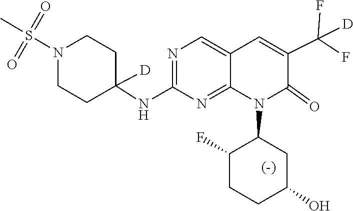 [SMILES: [2H]C1(Nc2ncc3cc(C([2H])(F)F)c(=O)n([C@H]4C[C@H](O)CC[C@@H]4F)c3n2)CCN(S(C)(=O)=O)CC1] | (−)-6-(difluoromethyl-d)- 8-((1S,2S,5R)-2-fluoro-5- hydroxycyclohexyl)-2-((1- (methylsulfonyl)piperidin- 4-yl-4-d)- amino)pyrido[2,3- d]pyrimidin-7(8H)-one | 492.2 |
| Comp. 63 | 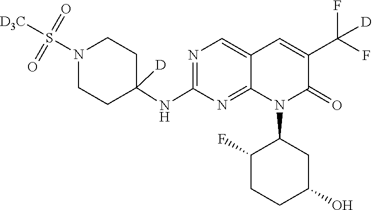 [SMILES: [2H]C1(Nc2ncc3cc(C([2H])(F)F)c(=O)n([C@H]4C[C@H](O)CC[C@@H]4F)c3n2)CCN(S(=O)(=O)C([2H])([2H])[2H])CC1] | (−)-6-(difluoromethyl-d)- 8-((1S,2S,5R)-2-fluoro-5- hydroxycyclohexyl)-2-((1- ((methyl- d3)sulfonyl)piperidin-4- yl-4-d)-amino)pyrido[2,3- d]pyrimidin-7(8H)-one | 495.2 |
| Comp. 64 | 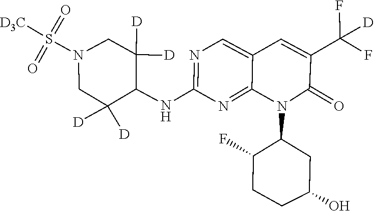 [SMILES: [2H]C(F)(F)c1cc2cnc(NC3C([2H])([2H])CN(S(=O)(=O)C([2H])([2H])[2H])CC3([2H])[2H])nc2n([C@H]2C[C@H](O)CC[C@@H]2F)c1=O] | (−)-6-(difluoromethyl-d)- 8-((1S,2S,5R)-2-fluoro-5- hydroxycyclohexyl)-2-((1- ((methyl- d3)sulfonyl)piperidin-4- yl-3,3,5,5-d4)- amino)pyrido[2,3- d]pyrimidin-7(8H)-one | 498.2 |
| Comp. 65 | 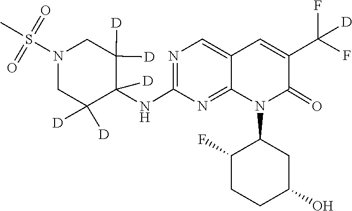 [SMILES: [2H]C(F)(F)c1cc2cnc(NC3([2H])C([2H])([2H])CN(S(C)(=O)=O)CC3([2H])[2H])nc2n([C@H]2C[C@H](O)CC[C@@H]2F)c1=O] | (−)-6-(difluoromethyl-d)- 8-((1S,2S,5R)-2-fluoro-5- hydroxycyclohexyl)-2-((1- (methylsulfonyl)piperidin- 4-yl-3,3,4,5,5-d5)- amino)pyrido[2,3- d]pyrimidin-7(8H)-one | 496.2 |
| Comp. 66 | 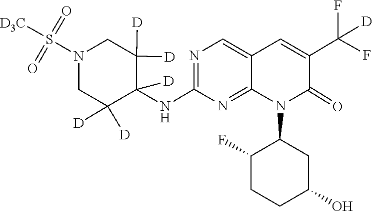 [SMILES: [2H]C(F)(F)c1cc2cnc(NC3([2H])C([2H])([2H])CN(S(=O)(=O)C([2H])([2H])[2H])CC3([2H])[2H])nc2n([C@H]2C[C@H](O)CC[C@@H]2F)c1=O] | (−)-6-(difluoromethyl-d)- 8-((1S,2S,5R)-2-fluoro-5- hydroxycyclohexyl)-2-((1- ((methyl- d3)sulfonyl)piperidin-4- yl-3, 3,4,5,5-d5)- amino)pyrido[2,3- d]pyrimidin-7(8H)-one | 499.2 |
| Comp. 67 | 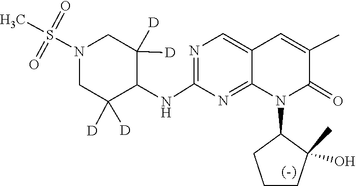 [SMILES: [2H]C1([2H])CN(S(C)(=O)=O)CC([2H])([2H])C1Nc1ncc2cc(C)c(=O)n([C@@H]3CCC[C@@]3(C)O)c2n1] | (−)-8-((1R,2R)-2-hydroxy- 2-methylcyclopentyl)-6- methyl-2-((1- (methylsulfonyl)piperidin- 4-yl-3,3,5,5- d4)amino)pyrido[2,3- d]pyrimidin-7(8H)-one | 440.2/ 85% D/ 99.7% ee |
| Comp. 68 | 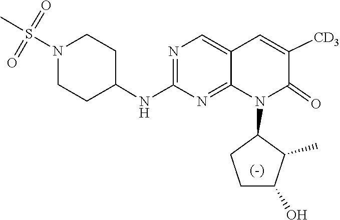 [SMILES: [2H]C([2H])([2H])c1cc2cnc(NC3CCN(S(C)(=O)=O)CC3)nc2n([C@@H]2CC[C@@H](O)[C@H]2C)c1=O] | (−)-8-((1R,2S,3R)-3- hydroxy-2- methylcyclopentyl)-6- (methyl-d3)-2-((1- (methylsulfonyl)piperidin- 4-yl)amino)pyrido[2,3- d]pyrimidin-7(8H)-one | 439.2 |
| Comp. 69 | 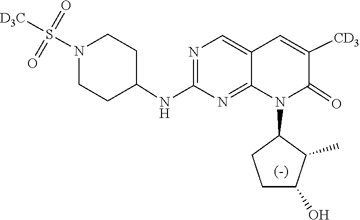 [SMILES: [2H]C([2H])([2H])c1cc2cnc(NC3CCN(S(=O)(=O)C([2H])([2H])[2H])CC3)nc2n([C@@H]2CC[C@@H](O)[C@H]2C)c1=O] | (−)-8-((1R,2S,3R)-3- hydroxy-2- methylcyclopentyl)-6- (methyl-d3)-2-((1- ((methyl- d3)sulfonyl)piperidin-4- yl)amino)pyrido[2,3- d]pyrimidin-7(8H)-one | 442.2 |
| Comp. 70 | 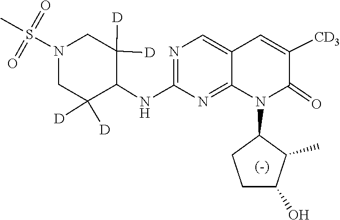 [SMILES: [2H]C([2H])([2H])c1cc2cnc(NC3C([2H])([2H])CN(S(C)(=O)=O)CC3([2H])[2H])nc2n([C@@H]2CC[C@@H](O)[C@H]2C)c1=O] | (−)-8-((1R,2S,3R)-3- hydroxy-2- methylcyclopentyl)-6- (methyl-d3)-2-((1- (methylsulfonyl)piperidin- 4-yl-3,3,5,5-d4)- amino)pyrido[2,3- d]pyrimidin-7(8H)-one | 443.2 |
| Comp. 71 | 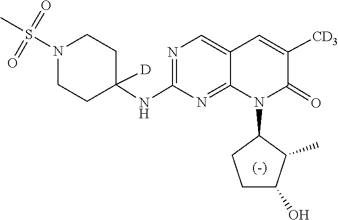 | (−)-8-((1R,2S,3R)-3- hydroxy-2- methylcyclopentyl)-6- (methyl-d3)-2-((1- (methylsulfonyl)piperidin- 4-yl-4-d)- amino)pyrido[2,3- d]pyrimidin-7(8H)-one | 440.2 |
| Comp. 72 | 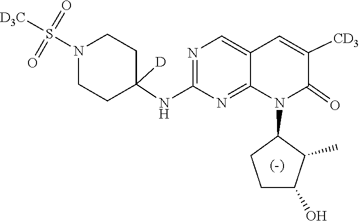 [SMILES: [2H]C1(Nc2ncc3cc(C([2H])([2H])[2H])c(=O)n([C@@H]4CC[C@@H](O)[C@H]4C)c3n2)CCN(S(=O)(=O)C([2H])([2H])[2H])CC1] | (−)-8-((1R,2S,3R)-3- hydroxy-2- methylcyclopentyl)-6- (methyl-d3)-2-((1- ((methyl- d3)sulfonyl)piperidin-4- yl-4-d)-amino)pyrido[2,3- d]pyrimidin-7(8H)-one | 443.2 |
| Comp. 73 | 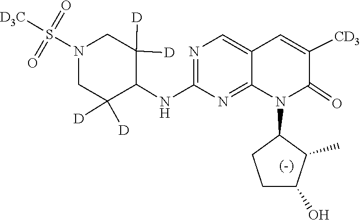 [SMILES: [2H]C([2H])([2H])c1cc2cnc(NC3C([2H])([2H])CN(S(=O)(=O)C([2H])([2H])[2H])CC3([2H])[2H])nc2n([C@@H]2CC[C@@H](O)[C@H]2C)c1=O] | (−)-8-((1R,2S,3R)-3- hydroxy-2- methylcyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 446.3 |
| Comp. 74 | 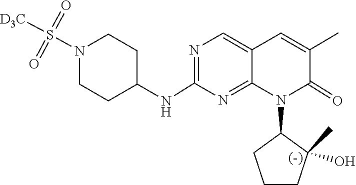 [SMILES: [2H]C([2H])([2H])S(=O)(=O)N1CCC(Nc2ncc3cc(C)c(=O)n([C@@H]4CCC[C@@]4(C)O)c3n2)CC1] | (−)-8-((1R,2R)-2-hydroxy- 2-methylcyclopentyl)-6- methyl-2-((1-((methyl- d3)sulfonyl)piperidin-4- yl)amino)pyrido[2,3- d]pyrimidin-7(8H)-one | 439.2/ 90% D/ 99.7% ee |
| Comp. 75 | 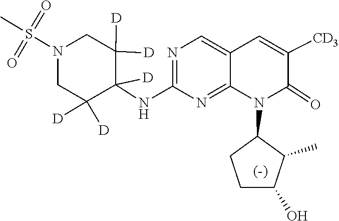 [SMILES: [2H]C([2H])([2H])c1cc2cnc(NC3([2H])C([2H])([2H])CN(S(C)(=O)=O)CC3([2H])[2H])nc2n([C@@H]2CC[C@@H](O)[C@H]2C)c1=O] | (−)-8-((1R,2S,3R)-3- hydroxy-2- methylcyclopentyl)-6- (methyl-d3)-2-((1- (methylsulfonyl)piperidin- 4-yl-3,3,4,5,5-d5)- amino)pyrido[2,3- d]pyrimidin-7(8H)-one | 444.2 |
| Comp. 76 | 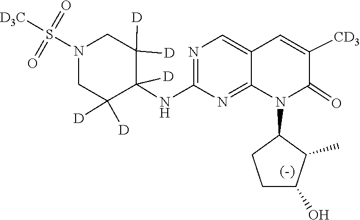 [SMILES: [2H]C([2H])([2H])c1cc2cnc(NC3([2H])C([2H])([2H])CN(S(=O)(=O)C([2H])([2H])[2H])CC3([2H])[2H])nc2n([C@@H]2CC[C@@H](O)[C@H]2C)c1=O] | (−)-8-((1R,2S,3R)-3- hydroxy-2- methylcyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 447.3 |
| Comp. 77 | 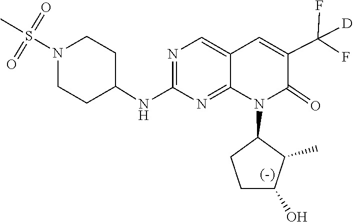 [SMILES: [2H]C(F)(F)c1cc2cnc(NC3CCN(S(C)(=O)=O)CC3)nc2n([C@@H]2CC[C@@H](O)[C@H]2C)c1=O] | (−)-8-((1R,2S,3R)-3- hydroxy-2- methylcyclopentyl)-6- (difluoromethyl-d)-2-((1- (methylsulfonyl)piperidin- 4-yl)amino)pyrido[2,3- d]pyrimidin-7(8H)-one | 473.2 |
| Comp. 78 | 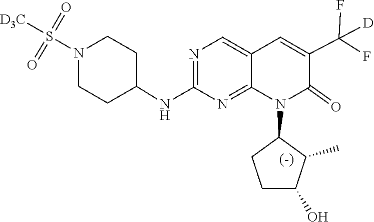 [SMILES: [2H]C(F)(F)c1cc2cnc(NC3CCN(S(=O)(=O)C([2H])([2H])[2H])CC3)nc2n([C@@H]2CC[C@@H](O)[C@H]2C)c1=O] | (−)-8-((1R,2S,3R)-3- hydroxy-2- methylcyclopentyl)-6- (difluoromethyl-d)-2-((1- ((methyl- d3)sulfonyl)piperidin-4- yl)amino)pyrido[2,3- d]pyrimidin-7(8H)-one | 476.2 |
| Comp. 79 | 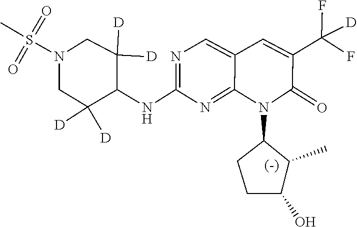 [SMILES: [2H]C(F)(F)c1cc2cnc(NC3C([2H])([2H])CN(S(C)(=O)=O)CC3([2H])[2H])nc2n([C@@H]2CC[C@@H](O)[C@H]2C)c1=O] | (−)-8-((1R,2S,3R)-3- hydroxy-2- methylcyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 477.2 |
| Comp. 80 | 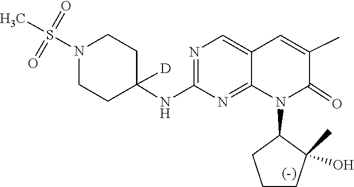 | (−)-8-((1R,2R)-2-hydroxy- 2-methylcyclopentyl)-6- methyl-2-((1- (methylsulfonyl)piperidin- 4-yl-4- d)amino)pyrido[2,3- d]pyrimidin-7(8H)-one | 472.2/ |
| Comp. 81 | 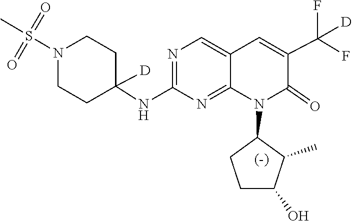 [SMILES: [2H]C1(Nc2ncc3cc(C([2H])(F)F)c(=O)n([C@@H]4CC[C@@H](O)[C@H]4C)c3n2)CCN(S(C)(=O)=O)CC1] | (−)-8-((1R,2S,3R)-3- hydroxy-2- methylcyclopentyl)-6- (difluoromethyl-d)-2-((1- (methylsulfonyl)piperidin- 4-yl-4-d)- amino)pyrido[2,3- d]pyrimidin-7(8H)-one | 474.2/ >95% D/ 99.7% ee |
| Comp. 82 | 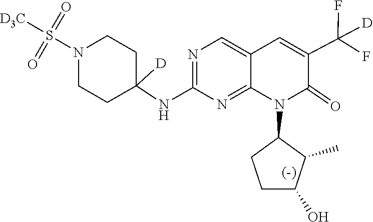 [SMILES: [2H]C1(Nc2ncc3cc(C([2H])(F)F)c(=O)n([C@@H]4CC[C@@H](O)[C@H]4C)c3n2)CCN(S(=O)(=O)C([2H])([2H])[2H])CC1] | (−)-8-((1R,2S,3R)-3- hydroxy-2- methylcyclopentyl)-6- (difluoromethyl-d)-2-((1- ((methyl- d3)sulfonyl)piperidin-4- yl-4-d)-amino)pyrido[2,3- d]pyrimidin-7(8H)-one | 477.2 |
| Comp. 83 | 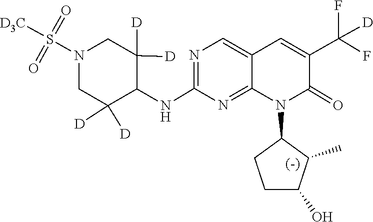 [SMILES: [2H]C(F)(F)c1cc2cnc(NC3C([2H])([2H])CN(S(=O)(=O)C([2H])([2H])[2H])CC3([2H])[2H])nc2n([C@@H]2CC[C@@H](O)[C@H]2C)c1=O] | (−)-8-((1R,2S,3R)-3- hydroxy-2- methylcyclopentyl)-6- (difluoromethyl-d)-2-((1- ((methyl- d3)sulfonyl)piperidin-4- yl-3,3,5,5-d4)- amino)pyrido[2,3- d]pyrimidin-7(8H)-one | 480.2 |
| Comp. 84 | 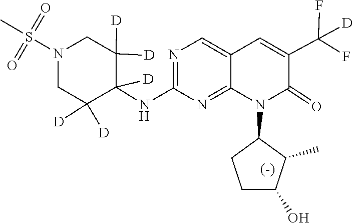 [SMILES: [2H]C(F)(F)c1cc2cnc(NC3([2H])C([2H])([2H])CN(S(C)(=O)=O)CC3([2H])[2H])nc2n([C@@H]2CC[C@@H](O)[C@H]2C)c1=O] | (−)-8-((1R,2S,3R)-3- hydroxy-2- methylcyclopentyl)-6- (difluoromethyl-d)-2-((1- (methylsulfonyl)piperidin- 4-yl-3,3,4,5,5-d5)- amino)pyrido[2,3- d]pyrimidin-7(8H)-one | 478.2 |
| Comp. 85 | 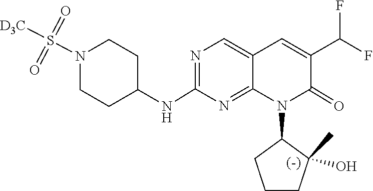 [SMILES: [2H]C([2H])([2H])S(=O)(=O)N1CCC(Nc2ncc3cc(C(F)F)c(=O)n([C@@H]4CCC[C@@]4(C)O)c3n2)CC1] | (−)-6-(difluoromethyl)-8- ((1R,2R)-2-hydroxy-2- methylcyclopentyl)-2-((1- ((methyl- d3)sulfonyl)piperidin-4- yl)amino)pyrido[2,3- d]pyrimidin-7(8H)-one | 475.2/ 90% D/ 99.7% ee |
| Comp. 86 | 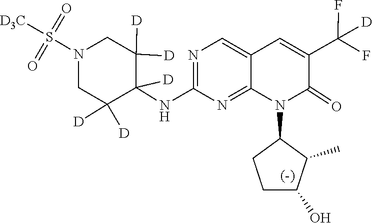 [SMILES: [2H]C(F)(F)c1cc2cnc(NC3([2H])C([2H])([2H])CN(S(=O)(=O)C([2H])([2H])[2H])CC3([2H])[2H])nc2n([C@@H]2CC[C@@H](O)[C@H]2C)c1=O] | (−)-8-((1R,2S,3R)-3- hydroxy-2- methylcyclopentyl)-6- (difluoromethyl-d)-2-((1- ((methyl- d3)sulfonyl)piperidin-4- yl-3,3,4,5,5-d5)- amino)pyrido[2,3- d]pyrimidin-7(8H)-one | 481.2 |
| Comp. 87 | 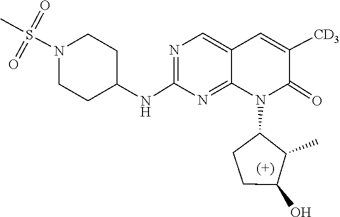 [SMILES: [2H]C([2H])([2H])c1cc2cnc(NC3CCN(S(C)(=O)=O)CC3)nc2n([C@H]2CC[C@H](O)[C@H]2C)c1=O] | (+)-8-((1S,2S,3S)-3- hydroxy-2- methylcyclopentyl)-6- (methyl-d3)-2-((1- (methylsulfonyl)piperidin- 4-yl)amino)pyrido[2,3- d]pyrimidin-7(8H)-one | 439.2 |
| Comp. 88 | 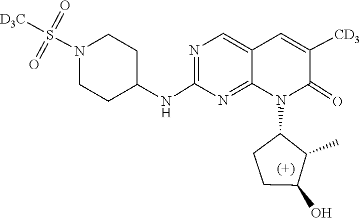 [SMILES: [2H]C([2H])([2H])c1cc2cnc(NC3CCN(S(=O)(=O)C([2H])([2H])[2H])CC3)nc2n([C@H]2CC[C@H](O)[C@H]2C)c1=O] | (+)-8-((1S,2S,3S)-3- hydroxy-2- methylcyclopentyl)-6- (methyl-d3)-2-((1- ((methyl- d3)sulfonyl)piperidin-4- yl)amino)pyrido[2,3- d]pyrimidin-7(8H)-one | 442.2 |
| Comp. 89 | 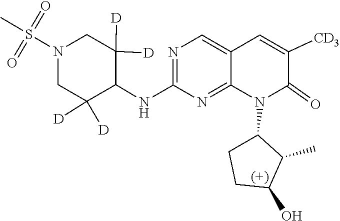 [SMILES: [2H]C([2H])([2H])c1cc2cnc(NC3C([2H])([2H])CN(S(C)(=O)=O)CC3([2H])[2H])nc2n([C@H]2CC[C@H](O)[C@H]2C)c1=O] | (+)-8-((1S,2S,3S)-3- hydroxy-2- methylcyclopentyl)-6- (methyl-d3)-2-((1- (methylsulfonyl)piperidin- 4-yl-3,3,5,5-d4)- amino)pyrido[2,3- d]pyrimidin-7(8H)-one | 443.2 |
| Comp. 90 | 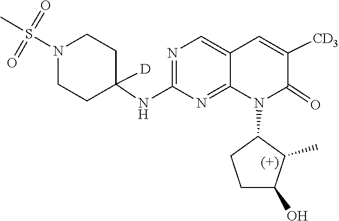 [SMILES: [2H]C1(Nc2ncc3cc(C([2H])([2H])[2H])c(=O)n([C@H]4CC[C@H](O)[C@H]4C)c3n2)CCN(S(C)(=O)=O)CC1] | (+)-8-((1S,2S,3S)-3- hydroxy-2- methylcyclopentyl)-6- (methyl-d3)-2-((1- (methylsulfonyl)piperidin- 4-yl-4-d)- amino)pyrido[2,3- d]pyrimidin-7(8H)-one | 440.2 |
| Comp. 91 | 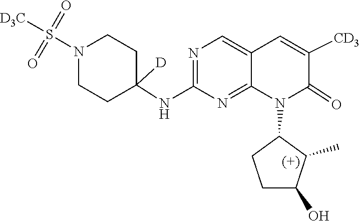 [SMILES: [2H]C1(Nc2ncc3cc(C([2H])([2H])[2H])c(=O)n([C@H]4CC[C@H](O)[C@H]4C)c3n2)CCN(S(=O)(=O)C([2H])([2H])[2H])CC1] | (+)-8-((1S,2S,3S)-3- hydroxy-2- methylcyclopentyl)-6- (methyl-d3)-2-((1- ((methyl- d3)sulfonyl)piperidin-4- yl-4-d)-amino)pyrido[2,3- d]pyrimidin-7(8H)-one | 443.2 |
| Comp. 92 | 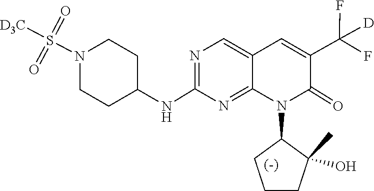 [SMILES: [2H]C(F)(F)c1cc2cnc(NC3CCN(S(=O)(=O)C([2H])([2H])[2H])CC3)nc2n([C@@H]2CCC[C@@]2(C)O)c1=O] | (−)-6-(difluoromethyl-d)- 8-((1R,2R)-2-hydroxy-2- methylcyclopentyl)-2-((1- ((methyl- d3)sulfonyl)piperidin-4- yl)amino)pyrido[2,3- d]pyrimidin-7(8H)-one | 476.2/ 90% D/ 99.7% ee |
| Comp. 93 | 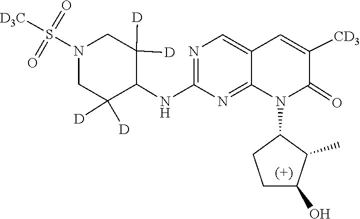 [SMILES: [2H]C([2H])([2H])c1cc2cnc(NC3C([2H])([2H])CN(S(=O)(=O)C([2H])([2H])[2H])CC3([2H])[2H])nc2n([C@H]2CC[C@H](O)[C@H]2C)c1=O] | (+)-8-((1S,2S,3S)-3- hydroxy-2- methylcyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 446.3 |
| Comp. 94 | 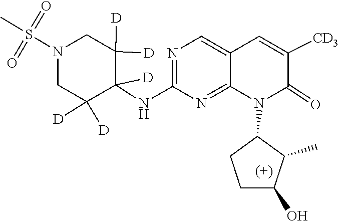 [SMILES: [2H]C([2H])([2H])c1cc2cnc(NC3([2H])C([2H])([2H])CN(S(C)(=O)=O)CC3([2H])[2H])nc2n([C@H]2CC[C@H](O)[C@H]2C)c1=O] | (+)-8-((1S,2S,3S)-3- hydroxy-2- methylcyclopentyl)-6- (methyl-d3)-2-((1- (methylsulfonyl)piperidin- 4-yl-3,3,4,5,5-d5)- amino)pyrido[2,3- d]pyrimidin-7(8H)-one | 444.2 |
| Comp. 95 | 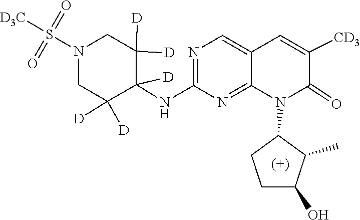 [SMILES: [2H]C([2H])([2H])c1cc2cnc(NC3([2H])C([2H])([2H])CN(S(=O)(=O)C([2H])([2H])[2H])CC3([2H])[2H])nc2n([C@H]2CC[C@H](O)[C@H]2C)c1=O] | (+)-8-((1S,2S,3S)-3- hydroxy-2- methylcyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 447.3 |
| Comp. 96 | 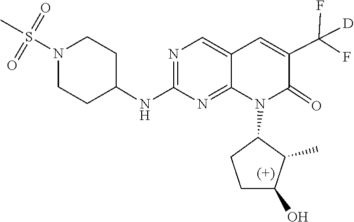 [SMILES: [2H]C(F)(F)c1cc2cnc(NC3CCN(S(C)(=O)=O)CC3)nc2n([C@H]2CC[C@H](O)[C@H]2C)c1=O] | (+)-8-((1S,2S,3S)-3- hydroxy-2- methylcyclopentyl)-6- (difluoromethyl-d)-2-((1- (methylsulfonyl)piperidin- 4-yl)amino)pyrido[2,3- d]pyrimidin-7(8H)-one | 473.2 |
| Comp. 97 | 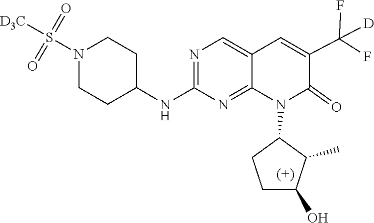 [SMILES: [2H]C(F)(F)c1cc2cnc(NC3CCN(S(=O)(=O)C([2H])([2H])[2H])CC3)nc2n([C@H]2CC[C@H](O)[C@H]2C)c1=O] | (+)-8-((1S,2S,3S)-3- hydroxy-2- methylcyclopentyl)-6- (difluoromethyl-d)-2-((1- ((methyl- d3)sulfonyl)piperidin-4- yl)amino)pyrido[2,3- d]pyrimidin-7(8H)-one | 476.2 |
| Comp. 98 | 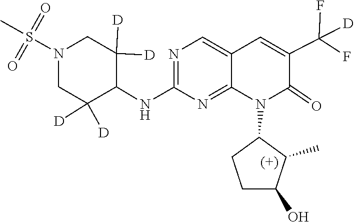 [SMILES: [2H]C(F)(F)c1cc2cnc(NC3C([2H])([2H])CN(S(C)(=O)=O)CC3([2H])[2H])nc2n([C@H]2CC[C@H](O)[C@H]2C)c1=O] | (+)-8-((1S,2S,3S)-3- hydroxy-2- methylcyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 477.2 |
| Comp. 99 | 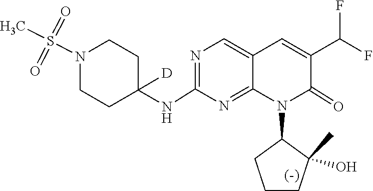 [SMILES: [2H]C1(Nc2ncc3cc(C(F)F)c(=O)n([C@@H]4CCC[C@@]4(C)O)c3n2)CCN(S(C)(=O)=O)CC1] | (−)-6-(difluoromethyl)-8- ((1R,2R)-2-hydroxy-2- methylcyclopentyl)-2-((1- (methylsulfonyl)piperidin- 4-yl-4- d)amino)pyrido[2,3- d]pyrimidin-7(8H)-one | 473.2/ >95% D/ 99.7% ee |
| Comp. 100 | 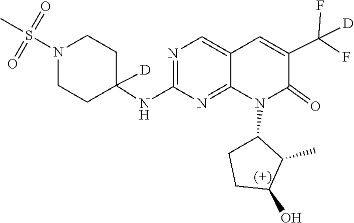 [SMILES: [2H]C1(Nc2ncc3cc(C([2H])(F)F)c(=O)n([C@H]4CC[C@H](O)[C@H]4C)c3n2)CCN(S(C)(=O)=O)CC1] | (+)-8-((1S,2S,3S)-3- hydroxy-2- methylcyclopentyl)-6- (difluoromethyl-d)-2-((1- (methylsulfonyl)piperidin- 4-yl-4-d)- amino)pyrido[2,3- d]pyrimidin-7(8H)-one | 474.2 |
| Comp. 101 | 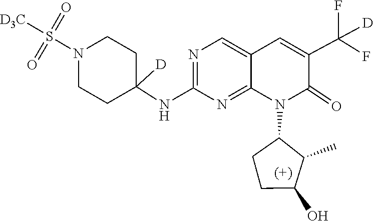 [SMILES: [2H]C1(Nc2ncc3cc(C([2H])(F)F)c(=O)n([C@H]4CC[C@H](O)[C@H]4C)c3n2)CCN(S(=O)(=O)C([2H])([2H])[2H])CC1] | (+)-8-((1S,2S,3S)-3- hydroxy-2- methylcyclopentyl)-6- (difluoromethyl-d)-2-((1- ((methyl- d3)sulfonyl)piperidin-4- yl-4-d)-amino)pyrido[2,3- d]pyrimidin-7(8H)-one | 477.2 |
| Comp. 102 | 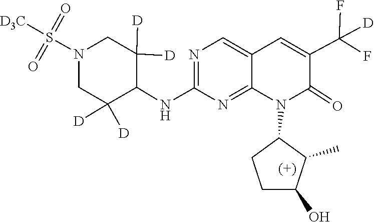 [SMILES: [2H]C(F)(F)c1cc2cnc(NC3C([2H])([2H])CN(S(=O)(=O)C([2H])([2H])[2H])CC3([2H])[2H])nc2n([C@H]2CC[C@H](O)[C@H]2C)c1=O] | (+)-8-((1S,2S,3S)-3- hydroxy-2- methylcyclopentyl)-6- (difluoromethyl-d)-2-((1- ((methyl- d3)sulfonyl)piperidin-4- yl-3,3,5,5-d4)- amino)pyrido[2,3- d]pyrimidin-7(8H)-one | 480.2 |
| Comp. 103 | 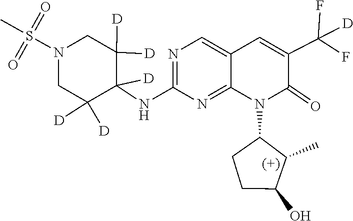 [SMILES: [2H]C(F)(F)c1cc2cnc(NC3([2H])C([2H])([2H])CN(S(C)(=O)=O)CC3([2H])[2H])nc2n([C@H]2CC[C@H](O)[C@H]2C)c1=O] | (+)-8-((1S,2S,3S)-3- hydroxy-2- methylcyclopentyl)-6- (difluoromethyl-d)-2-((1- (methylsulfonyl)piperidin- 4-yl-3,3,4,5,5-d5)- amino)pyrido[2,3- d]pyrimidin-7(8H)-one | 478.2 |
| Comp. 104 | 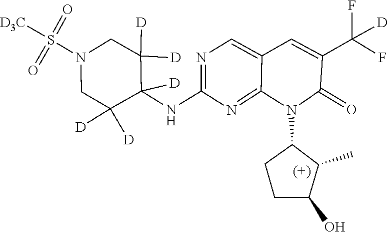 [SMILES: [2H]C(F)(F)c1cc2cnc(NC3([2H])C([2H])([2H])CN(S(=O)(=O)C([2H])([2H])[2H])CC3([2H])[2H])nc2n([C@H]2CC[C@H](O)[C@H]2C)c1=O] | (+)-8-((1S,2S,3S)-3- hydroxy-2- methylcyclopentyl)-6- (difluoromethyl-d)-2-((1- ((methyl- d3)sulfonyl)piperidin-4- yl-3,3,4,5,5-d5)- amino)pyrido[2,3- d]pyrimidin-7(8H)-one | 481.2 |
| Comp. 105 | 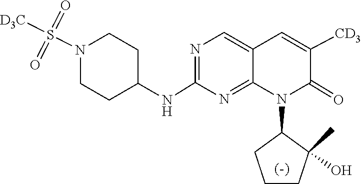 [SMILES: [2H]C([2H])([2H])c1cc2cnc(NC3CCN(S(=O)(=O)C([2H])([2H])[2H])CC3)nc2n([C@@H]2CCC[C@@]2(C)O)c1=O] | (−)-8-((1R,2R)-2-hydroxy- 2-methylcyclopentyl)-6- (methyl-d3)-2-((1- ((methyl- d3)sulfonyl)piperidin-4- yl)amino)pyrido[2,3- d]pyrimidin-7(8H)-one | 442.2 |
| Comp. 106 | 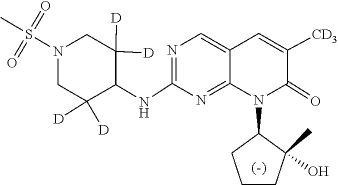 [SMILES: [2H]C([2H])([2H])c1cc2cnc(NC3C([2H])([2H])CN(S(C)(=O)=O)CC3([2H])[2H])nc2n([C@@H]2CCC[C@@]2(C)O)c1=O] | (−)-8-((1R,2R)-2-hydroxy- 2-methylcyclopentyl)-6- (methyl-d3)-2-((1- (methylsulfonyl)piperidin- 4-yl-3,3,5,5-4d)- amino)pyrido[2,3- d]pyrimidin-7(8H)-one | 443.2 |
| Comp. 107 | 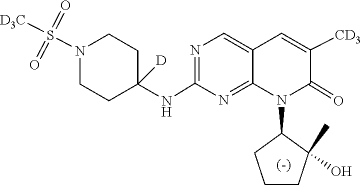 [SMILES: [2H]C1(Nc2ncc3cc(C([2H])([2H])[2H])c(=O)n([C@@H]4CCC[C@@]4(C)O)c3n2)CCN(S(=O)(=O)C([2H])([2H])[2H])CC1] | (−)-8-((1R,2R)-2-hydroxy- 2-methylcyclopentyl)-6- (methyl-d3)-2-((1- ((methyl- d3)sulfonyl)piperidin-4- yl-4-d)-amino)pyrido[2,3- d]pyrimidin-7(8H)-one | 443.2 |
| Comp. 108 | 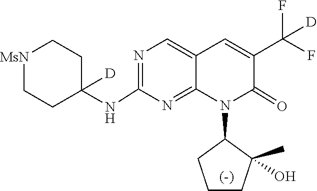 | (−)-6-(difluoromethyl-d)- 8-((1R,2R)-2-hydroxy-2- methylcyclopentyl)-2-((1- (methylsulfonyl)piperidin- 4-yl-4- d)amino)pyrido[2,3- d]pyrimidin-7(8H)-one | 474.2/ >95% D/ 99.7% ee |
| Comp. 109 | 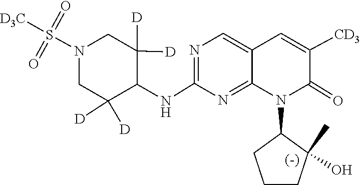 [SMILES: [2H]C([2H])([2H])c1cc2cnc(NC3C([2H])([2H])CN(S(=O)(=O)C([2H])([2H])[2H])CC3([2H])[2H])nc2n([C@@H]2CCC[C@@]2(C)O)c1=O] | (−)-8-((1R,2R)-2-hydroxy- 2-methylcyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 446.3 |
| Comp. 110 | 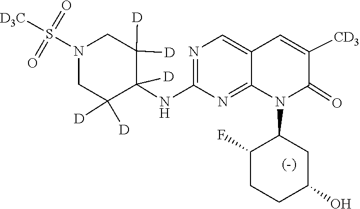 [SMILES: [2H]C([2H])([2H])c1cc2cnc(NC3([2H])C([2H])([2H])CN(S(=O)(=O)C([2H])([2H])[2H])CC3([2H])[2H])nc2n([C@H]2C[C@H](O)CC[C@@H]2F)c1=O] | (−)-8-((1S,2S,5R)-2- fluoro-5- hydroxycyclohexyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 465.2 |
| Comp. 111 | 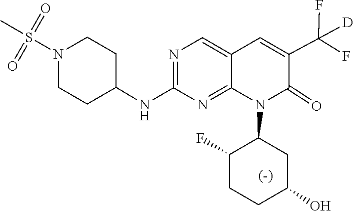 [SMILES: [2H]C(F)(F)c1cc2cnc(NC3CCN(S(C)(=O)=O)CC3)nc2n([C@H]2C[C@H](O)CC[C@@H]2F)c1=O] | (−)-8-((1S,2S,5R)-2- fluoro-5- hydroxycyclohexyl)-6- (difluoromethyl-d)-2-((1- (methylsulfonyl)piperidin- 4-yl)amino)pyrido[2,3- d]pyrimidin-7(8H)-one | 491.2 |
| Comp. 112 | 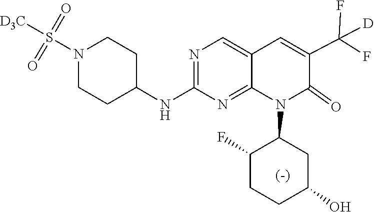 [SMILES: [2H]C(F)(F)c1cc2cnc(NC3CCN(S(=O)(=O)C([2H])([2H])[2H])CC3)nc2n([C@H]2C[C@H](O)CC[C@@H]2F)c1=O] | (−)-8-((1S,2S,5R)-2- fluoro-5- hydroxycyclohexyl)-6- (difluoromethyl-d)-2-((1- ((methyl- d3)sulfonyl)piperidin-4- yl)amino)pyrido[2,3- d]pyrimidin-7(8H)-one | 494.2 |
| Comp. 113 | 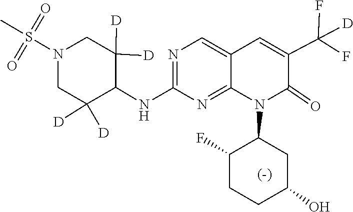 [SMILES: [2H]C(F)(F)c1cc2cnc(NC3C([2H])([2H])CN(S(C)(=O)=O)CC3([2H])[2H])nc2n([C@H]2C[C@H](O)CC[C@@H]2F)c1=O] | (−)-8-((1S,2S,5R)-2- fluoro-5- hydroxycyclohexyl)-6- (difluoromethyl-d)-2-((1- (methylsulfonyl)piperidin- 4-yl-3,3,5,5-d4)- amino)pyrido[2,3- d]pyrimidin-7(8H)-one | 495.2 |
| Comp. 114 | 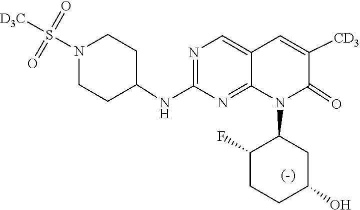 [SMILES: [2H]C([2H])([2H])c1cc2cnc(NC3CCN(S(=O)(=O)C([2H])([2H])[2H])CC3)nc2n([C@H]2C[C@H](O)CC[C@@H]2F)c1=O] | (−)-8-((1S,2S,5R)-2- fluoro-5- hydroxycyclohexyl)-6- (methyl-d3)-2-((1- ((methyl- d3)sulfonyl)piperidin-4- yl)amino)pyrido[2,3- d]pyrimidin-7(8H)-one | 460.2 |
| Comp. 115 | 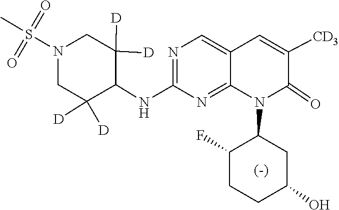 [SMILES: [2H]C([2H])([2H])c1cc2cnc(NC3C([2H])([2H])CN(S(C)(=O)=O)CC3([2H])[2H])nc2n([C@H]2C[C@H](O)CC[C@@H]2F)c1=O] | (−)-8-((1S,2S,5R)-2- fluoro-5- hydroxycyclohexyl)-6- (methyl-d3)-2-((1- (methylsulfonyl)piperidin- 4-yl-3,3,5,5-d4)- amino)pyrido[2,3- d]pyrimidin-7(8H)-one | 461.2 |
| Comp. 116 | 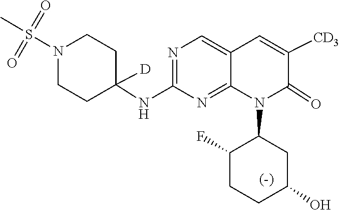 [SMILES: [2H]C1(Nc2ncc3cc(C([2H])([2H])[2H])c(=O)n([C@H]4C[C@H](O)CC[C@@H]4F)c3n2)CCN(S(C)(=O)=O)CC1] | (−)-8-((1S,2S,5R)-2- fluoro-5- hydroxycyclohexyl)-6- (methyl-d3)-2-((1- (methylsulfonyl)piperidin- 4-yl-4-d)- amino)pyrido[2,3- d]pyrimidin-7(8H)-one | 458.2 |
| Comp. 117 | 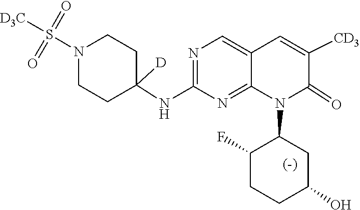 [SMILES: [2H]C1(Nc2ncc3cc(C([2H])([2H])[2H])c(=O)n([C@H]4C[C@H](O)CC[C@@H]4F)c3n2)CCN(S(=O)(=O)C([2H])([2H])[2H])CC1] | (−)-8-((1S,2S,5R)-2- fluoro-5- hydroxycyclohexyl)-6- (methyl-d3)-2-((1- ((methyl- d3)sulfonyl)piperidin-4- yl-4-d)-amino)pyrido[2,3- d]pyrimidin-7(8H)-one | 461.2 |
| Comp. 118 | 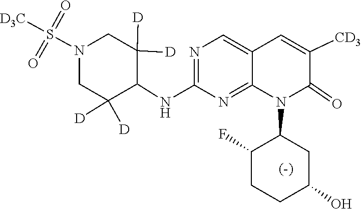 [SMILES: [2H]C([2H])([2H])c1cc2cnc(NC3C([2H])([2H])CN(S(=O)(=O)C([2H])([2H])[2H])CC3([2H])[2H])nc2n([C@H]2C[C@H](O)CC[C@@H]2F)c1=O] | (−)-8-((1S,2S,5R)-2- fluoro-5- hydroxycyclohexyl)-6- (methyl-d3)-2-((1- ((methyl- d3)sulfonyl)piperidin-4- yl-3,3,5,5-d4)- amino)pyrido[2,3- d]pyrimidin-7(8H)-one | 464.2 |
| Comp. 119 | 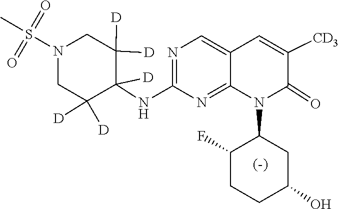 [SMILES: [2H]C([2H])([2H])c1cc2cnc(NC3([2H])C([2H])([2H])CN(S(C)(=O)=O)CC3([2H])[2H])nc2n([C@H]2C[C@H](O)CC[C@@H]2F)c1=O] | (−)-8-((1S,2S,5R)-2- fluoro-5- hydroxycyclohexyl)-6- (methyl-d3)-2-((1- (methylsulfonyl)piperidin- 4-yl-3,3,4,5,5-d5)- amino)pyrido[2,3- d]pyrimidin-7(8H)-one | 462.2 |
| Comp. 120 | 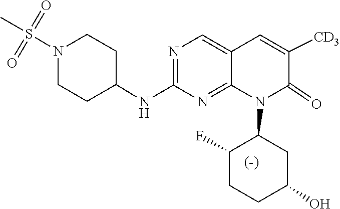 [SMILES: [2H]C([2H])([2H])c1cc2cnc(NC3CCN(S(C)(=O)=O)CC3)nc2n([C@H]2C[C@H](O)CC[C@@H]2F)c1=O] | (−)-8-((1S,2S,5R)-2- fluoro-5- hydroxycyclohexyl)-6- (methyl-d3)-2-((1- (methylsulfonyl)piperidin- 4-yl)-amino)pyrido[2,3- d]pyrimidin-7(8H)-one | 457.2 |
| Comp. 121 | 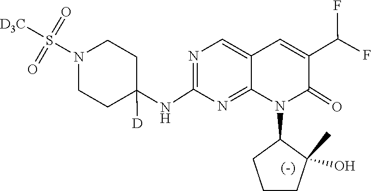 [SMILES: [2H]C1(Nc2ncc3cc(C(F)F)c(=O)n([C@@H]4CCC[C@@]4(C)O)c3n2)CCN(S(=O)(=O)C([2H])([2H])[2H])CC1] | (−)-6-(difluoromethyl)-8- ((1R,2R)-2-hydroxy-2- methylcyclopentyl)-2-((1- ((methyl- d3)sulfonyl)piperidin-4- yl-4-d)amino)pyrido[2,3- d]pyrimidin-7(8H)-one | 476.2/ 90% D/ 99.7% ee |
| Comp. 122 | 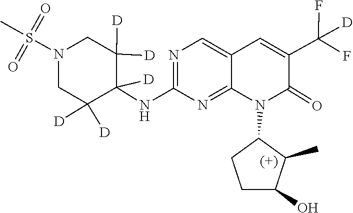 [SMILES: [2H]C(F)(F)c1cc2cnc(NC3([2H])C([2H])([2H])CN(S(C)(=O)=O)CC3([2H])[2H])nc2n([C@H]2CC[C@H](O)[C@@H]2C)c1=O] | (+)-6-(difluoromethyl-d)- 8-((1S,2R,3S)-3-hydroxy- 2-methylcyclopentyl)-2- ((1- (methylsulfonyl)piperidin- 4-yl-3,3,4,5,5-d5)- amino)pyrido[2,3- d]pyrimidin-7(8H)-one | 478.2 |
| Comp. 123 | 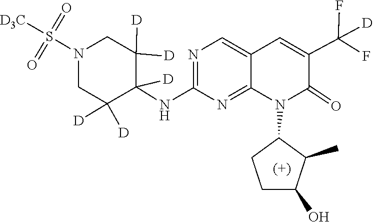 [SMILES: [2H]C(F)(F)c1cc2cnc(NC3([2H])C([2H])([2H])CN(S(=O)(=O)C([2H])([2H])[2H])CC3([2H])[2H])nc2n([C@H]2CC[C@H](O)[C@@H]2C)c1=O] | (+)-6-(difluoromethyl-d) 8-((1S,2R,3S)-3-hydroxy- 2-methylcyclopentyl)-2- ((1-((methyl- d3)sulfonyl)piperidin-4- yl-3,3,4,5,5-d5)- amino)pyrido[2,3- d]pyrimidin-7(8H)-one | 481.2 |
| Comp. 124 | 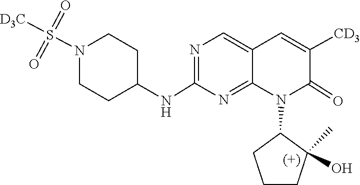 [SMILES: [2H]C([2H])([2H])c1cc2cnc(NC3CCN(S(=O)(=O)C([2H])([2H])[2H])CC3)nc2n([C@H]2CCC[C@]2(C)O)c1=O] | (+)-8-((1S,2S)-2-hydroxy- 2-methylcyclopentyl)-6- (methyl-d3)-2-((1- ((methyl- d3)sulfonyl)piperidin-4- yl)amino)pyrido[2,3- d]pyrimidin-7(8H)-one | 442.2 |
| Comp. 125 | 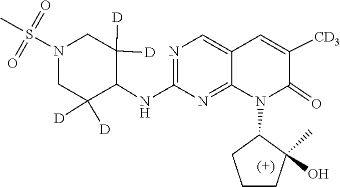 [SMILES: [2H]C([2H])([2H])c1cc2cnc(NC3C([2H])([2H])CN(S(C)(=O)=O)CC3([2H])[2H])nc2n([C@H]2CCC[C@]2(C)O)c1=O] | (+)-8-((1S,2S)-2-hydroxy- 2-methylcyclopentyl)-6- (methyl-d3)-2-((1- (methylsulfonyl)piperidin- 4-yl-3,3,5,5-d4)- amino)pyrido[2,3- d]pyrimidin-7(8H)-one | 443.2 |
| Comp. 126 | 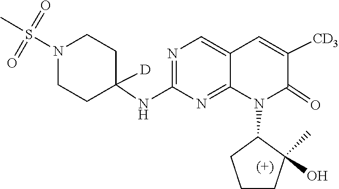 [SMILES: [2H]C1(Nc2ncc3cc(C([2H])([2H])[2H])c(=O)n([C@H]4CCC[C@]4(C)O)c3n2)CCN(S(C)(=O)=O)CC1] | (+)-8-((1S,2S)-2-hydroxy- 2-methylcyclopentyl)-6- (methyl-d3)-2-((1- (methylsulfonyl)piperidin- 4-yl-4-d)- amino)pyrido[2,3- d]pyrimidin-7(8H)-one | 440.2 |
| Comp. 127 | 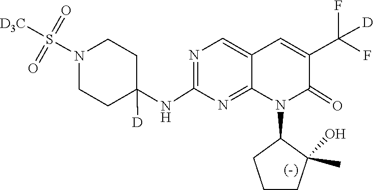 [SMILES: [2H]C1(Nc2ncc3cc(C([2H])(F)F)c(=O)n([C@@H]4CCC[C@@]4(C)O)c3n2)CCN(S(=O)(=O)C([2H])([2H])[2H])CC1] | (−)-6-(difluoromethyl-d)- 8-((1R,2R)-2-hydroxy-2- methylcyclopentyl)-2-((1- ((methyl- d3)sulfonyl)piperidin-4- yl-4-d)amino)pyrido[2,3- d]pyrimidin-7(8H)-one | 477.2/ 90% D/ 99.7% ee |
| Comp. 128 | 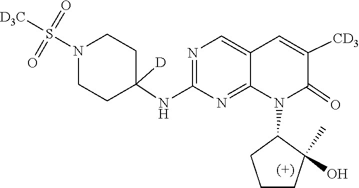 [SMILES: [2H]C1(Nc2ncc3cc(C([2H])([2H])[2H])c(=O)n([C@H]4CCC[C@]4(C)O)c3n2)CCN(S(=O)(=O)C([2H])([2H])[2H])CC1] | (+)-8-((1S,2S)-2-hydroxy- 2-methylcyclopentyl)-6- (methyl-d3)-2-((1- ((methyl- d3)sulfonyl)piperidin-4- yl-4-d)-amino)pyrido[2,3- d]pyrimidin-7(8H)-one | 443.2 |
| Comp. 129 | 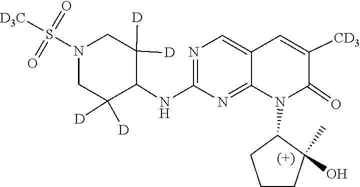 [SMILES: [2H]C([2H])([2H])c1cc2cnc(NC3C([2H])([2H])CN(S(=O)(=O)C([2H])([2H])[2H])CC3([2H])[2H])nc2n([C@H]2CCC[C@]2(C)O)c1=O] | (+)-8-((1S,2S)-2-hydroxy- 2-methylcyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 446.3 |
| Comp. 130 | 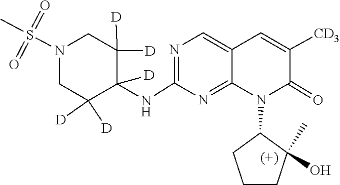 [SMILES: [2H]C([2H])([2H])c1cc2cnc(NC3([2H])C([2H])([2H])CN(S(C)(=O)=O)CC3([2H])[2H])nc2n([C@H]2CCC[C@]2(C)O)c1=O] | (+)-8-((1S,2S)-2-hydroxy- 2-methylcyclopentyl)-6- (methyl-d3)-2-((1- (methylsulfonyl)piperidin- 4-yl-3,3,4,5,5-d5)- amino)pyrido[2,3- d]pyrimidin-7(8H)-one | 444.2 |
| Comp. 131 | 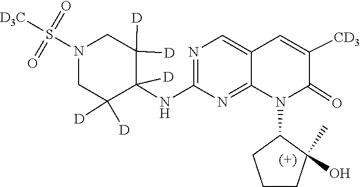 [SMILES: [2H]C([2H])([2H])c1cc2cnc(NC3([2H])C([2H])([2H])CN(S(=O)(=O)C([2H])([2H])[2H])CC3([2H])[2H])nc2n([C@H]2CCC[C@]2(C)O)c1=O] | (+)-8-((1S,2S)-2-hydroxy- 2-methylcyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 447.3 |
| Comp. 132 | 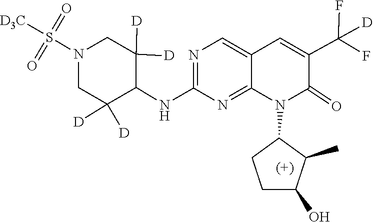 [SMILES: [2H]C(F)(F)c1cc2cnc(NC3C([2H])([2H])CN(S(=O)(=O)C([2H])([2H])[2H])CC3([2H])[2H])nc2n([C@H]2CC[C@H](O)[C@@H]2C)c1=O] | (+)-6-(difluoromethyl-d)- 8-((1S,2R,3S)-3-hydroxy- 2-methylcyclopentyl)-2- ((1-((methyl- d3)sulfonyl)piperidin-4- yl-3,3,5,5-d4)- amino)pyrido[2,3- d]pyrimidin-7(8H)-one | 480.2 |
| Comp. 133 | 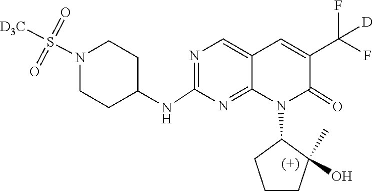 [SMILES: [2H]C(F)(F)c1cc2cnc(NC3CCN(S(=O)(=O)C([2H])([2H])[2H])CC3)nc2n([C@H]2CCC[C@]2(C)O)c1=O] | (+)-8-((1S,2S)-2-hydroxy- 2-methylcyclopentyl)-6- (difluoromethyl-d)-2-((1- ((methyl- d3)sulfonyl)piperidin-4- yl)amino)pyrido[2,3- d]pyrimidin-7(8H)-one | 476.2 |
| Comp. 134 | 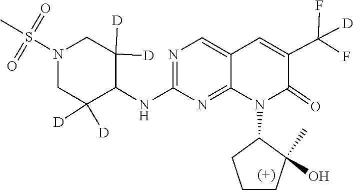 [SMILES: [2H]C(F)(F)c1cc2cnc(NC3C([2H])([2H])CN(S(C)(=O)=O)CC3([2H])[2H])nc2n([C@H]2CCC[C@]2(C)O)c1=O] | (+)-8-((1S,2S)-2-hydroxy- 2-methylcyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 477.2 |
| Comp. 135 | 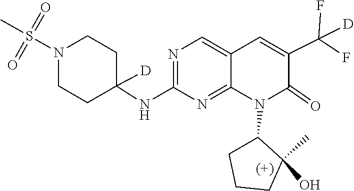 [SMILES: [2H]C1(Nc2ncc3cc(C([2H])(F)F)c(=O)n([C@H]4CCC[C@]4(C)O)c3n2)CCN(S(C)(=O)=O)CC1] | (+)-8-((1S,2S)-2-hydroxy- 2-methylcyclopentyl)-6- (difluoromethyl-d)-2-((1- (methylsulfonyl)piperidin- 4-yl-4-d)- amino)pyrido[2,3- d]pyrimidin-7(8H)-one | 474.2 |
| Comp. 136 | 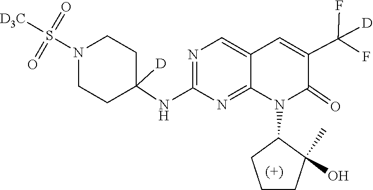 [SMILES: [2H]C1(Nc2ncc3cc(C([2H])(F)F)c(=O)n([C@H]4CCC[C@]4(C)O)c3n2)CCN(S(=O)(=O)C([2H])([2H])[2H])CC1] | (+)-8-((1S,2S)-2-hydroxy- 2-methylcyclopentyl)-6- (difluoromethyl-d)-2-((1- ((methyl- d3)sulfonyl)piperidin-4- yl-4-d)-amino)pyrido[2,3- d]pyrimidin-7(8H)-one | 477.2 |
| Comp. 137 | 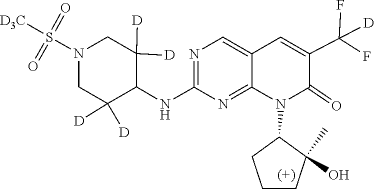 [SMILES: [2H]C(F)(F)c1cc2cnc(NC3C([2H])([2H])CN(S(=O)(=O)C([2H])([2H])[2H])CC3([2H])[2H])nc2n([C@H]2CCC[C@]2(C)O)c1=O] | (+)-8-((1S,2S)-2-hydroxy- 2-methylcyclopentyl)-6- (difluoromethyl-d)-2-((1- ((methyl- d3)sulfonyl)piperidin-4- yl-3,3,5,5-d4)- amino)pyrido[2,3- d]pyrimidin-7(8H)-one | 480.2 |
| Comp. 138 | 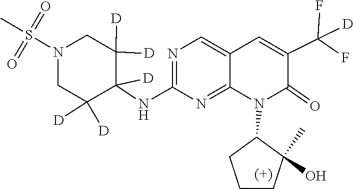 [SMILES: [2H]C(F)(F)c1cc2cnc(NC3([2H])C([2H])([2H])CN(S(C)(=O)=O)CC3([2H])[2H])nc2n([C@H]2CCC[C@]2(C)O)c1=O] | (+)-8-((1S,2S)-2-hydroxy- 2-methylcyclopentyl)-6- (difluoromethyl-d)-2-((1- (methylsulfonyl)piperidin- 4-yl-3,3,4,5,5-d5)- amino)pyrido[2,3- d]pyrimidin-7(8H)-one | 478.2 |
| Comp. 139 | 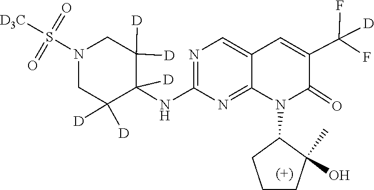 [SMILES: [2H]C(F)(F)c1cc2cnc(NC3([2H])C([2H])([2H])CN(S(=O)(=O)C([2H])([2H])[2H])CC3([2H])[2H])nc2n([C@H]2CCC[C@]2(C)O)c1=O] | (+)-8-((1S,2S)-2-hydroxy- 2-methylcyclopentyl)-6- (difluoromethyl-d)-2-((1- ((methyl- d3)sulfonyl)piperidin-4- yl-3,3,4,5,5-d5)- amino)pyrido[2,3- d]pyrimidin-7(8H)-one | 481.2 |
| Comp. 140 | 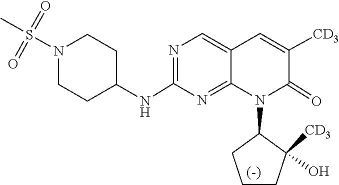 [SMILES: [2H]C([2H])([2H])c1cc2cnc(NC3CCN(S(C)(=O)=O)CC3)nc2n([C@@H]2CCC[C@]2(O)C([2H])([2H])[2H])c1=O] | (−)-8-((1R,2R)-2-hydroxy- 2-(methyl- d3)cyclopentyl)-6- (methyl-d3)-2-((1- (methylsulfonyl)piperidin- 4-yl)amino)pyrido[2,3- d]pyrimidin-7(8H)-one | 442.2 |
| Comp. 141 | 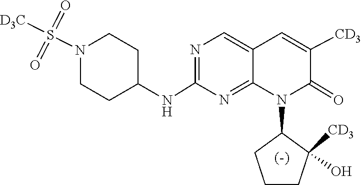 [SMILES: [2H]C([2H])([2H])c1cc2cnc(NC3CCN(S(=O)(=O)C([2H])([2H])[2H])CC3)nc2n([C@@H]2CCC[C@]2(O)C([2H])([2H])[2H])c1=O] | (−)-8-((1R,2R)-2-hydroxy- 2-(methyl- d3)cyclopentyl)-6- (methyl-d3)-2-((1- ((methyl- d3)sulfonyl)piperidin-4- yl)amino)pyrido[2,3- d]pyrimidin-7(8H)-one | 445.2 |
| Comp. 142 | 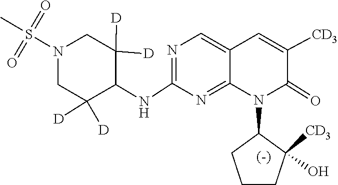 [SMILES: [2H]C([2H])([2H])c1cc2cnc(NC3C([2H])([2H])CN(S(C)(=O)=O)CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (methyl-d3)-2-((1- (methylsulfonyl)piperidin- 4-yl-3,3,5,5-d4)- amino)pyrido[2,3- d]pyrimidin-7(8H)-one | 446.3 |
| Comp. 143 | 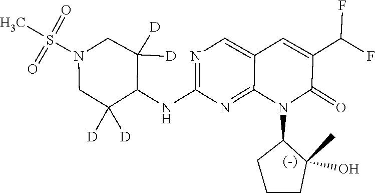 [SMILES: [2H]C1([2H])CN(S(C)(=O)=O)CC([2H])([2H])C1Nc1ncc2cc(C(F)F)c(=O)n([C@@H]3CCC[C@@]3(C)O)c2n1] | (−)-6-(difluoromethyl)-8- ((1R,2R)-2-hydroxy-2- methylcyclopentyl)-2-((1- (methylsulfonyl)piperidin- 4-yl-3,3,5,5- d4)amino)pyrido[2,3- d]pyrimidin-7(8H)-one | 476.2/ 85% D/ 99.7% ee |
| Comp. 144 | 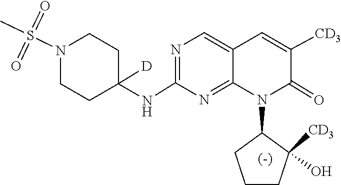 [SMILES: [2H]C1(Nc2ncc3cc(C([2H])([2H])[2H])c(=O)n([C@@H]4CCC[C@]4(O)C([2H])([2H])[2H])c3n2)CCN(S(C)(=O)=O)CC1] | (−)-8-((1R,2R)-2-hydroxy- 2-(methyl- d3)cyclopentyl)-6- (methyl-d3)-2-((1- (methylsulfonyl)piperidin- 4-yl-4-d)- amino)pyrido[2,3- d]pyrimidin-7(8H)-one | 443.2 |
| Comp. 145 | 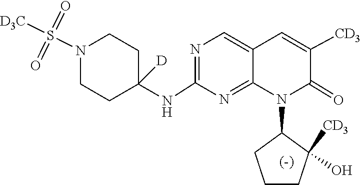 [SMILES: [2H]C1(Nc2ncc3cc(C([2H])([2H])[2H])c(=O)n([C@@H]4CCC[C@]4(O)C([2H])([2H])[2H])c3n2)CCN(S(=O)(=O)C([2H])([2H])[2H])CC1] | (−)-8-((1R,2R)-2-hydroxy- 2-(methyl- d3)cyclopentyl)-6- (methyl-d3)-2-((1- ((methyl- d3)sulfonyl)piperidin-4- yl-4-d)-amino)pyrido[2,3- d]pyrimidin-7(8H)-one | 446.3 |
| Comp. 146 | 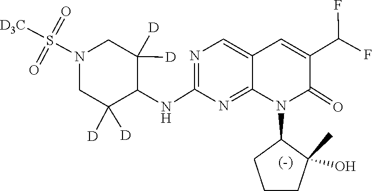 [SMILES: [2H]C1([2H])CN(S(=O)(=O)C([2H])([2H])[2H])CC([2H])([2H])C1Nc1ncc2cc(C(F)F)c(=O)n([C@@H]3CCC[C@@]3(C)O)c2n1] | (−)-6-(difluoromethyl)-8- ((1R,2R)-2-hydroxy-2- methylcyclopentyl)-2-((1- ((methyl- d3)sulfonyl)piperidin-4- yl-3,3,5,5- d4)amino)pyrido[2,3- d]pyrimidin-7(8H)-one | 479.2/ 80% D/ 99.7% ee |
| Comp. 147 | 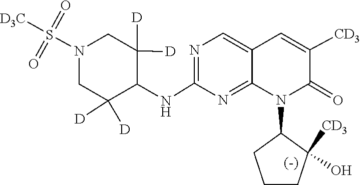 [SMILES: [2H]C([2H])([2H])c1cc2cnc(NC3C([2H])([2H])CN(S(=O)(=O)C([2H])([2H])[2H])CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 449.3 |
| Comp. 148 | 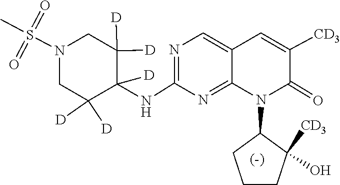 [SMILES: [2H]C([2H])([2H])c1cc2cnc(NC3([2H])C([2H])([2H])CN(S(C)(=O)=O)CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (methyl-d3)-2-((1- (methylsulfonyl)piperidin- 4-yl-3,3,4,5,5-d5)- amino)pyrido[2,3- d]pyrimidin-7(8H)-one | 447.3 |
| Comp. 149 | 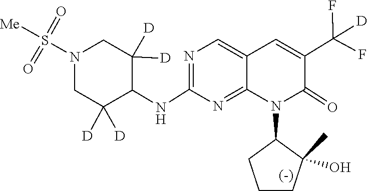 [SMILES: [2H]C(F)(F)c1cc2cnc(NC3C([2H])([2H])CN(S(C)(=O)=O)CC3([2H])[2H])nc2n([C@@H]2CCC[C@@]2(C)O)c1=O] | (−)-6-(difluoromethyl-d)- 8-((1R,2R)-2-hydroxy-2- methylcyclopentyl)-2-((1- (methylsulfonyl)piperidin- 4-yl-3,3,5,5- d4)amino)pyrido[2,3- d]pyrimidin-7(8H)-one | 477.2/ 85% D/ 99.7% ee |
| Comp. 150 | 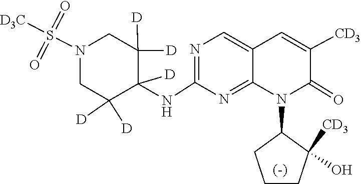 [SMILES: [2H]C([2H])([2H])c1cc2cnc(NC3([2H])C([2H])([2H])CN(S(=O)(=O)C([2H])([2H])[2H])CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 450.3 |
| Comp. 151 | 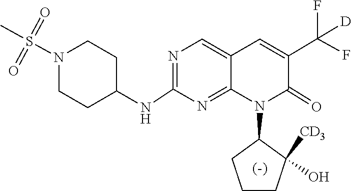 [SMILES: [2H]C(F)(F)c1cc2cnc(NC3CCN(S(C)(=O)=O)CC3)nc2n([C@@H]2CCC[C@]2(O)C([2H])([2H])[2H])c1=O] | (−)-8-((1R,2R)-2-hydroxy- 2-(methyl- d3)cyclopentyl)-6- (difluoromethyl-d)-2-((1- (methylsulfonyl)piperidin- 4-yl)amino)pyrido[2,3- d]pyrimidin-7(8H)-one | 476.2 |
| Comp. 152 | 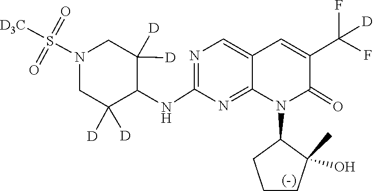 [SMILES: [2H]C(F)(F)c1cc2cnc(NC3C([2H])([2H])CN(S(=O)(=O)C([2H])([2H])[2H])CC3([2H])[2H])nc2n([C@@H]2CCC[C@@]2(C)O)c1=O] | (−)-6-(difluoromethyl-d)- 8-((1R,2R)-2-hydroxy-2- methylcyclopentyl)-2-((1- ((methyl- d3)sulfonyl)piperidin-4- yl-3,3,5,5- d4)amino)pyrido[2,3- d]pyrimidin-7(8H)-one | 480.2/ 80% D/ 99.7% ee |
| Comp. 153 | 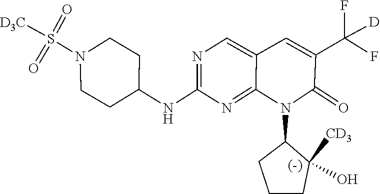 [SMILES: [2H]C(F)(F)c1cc2cnc(NC3CCN(S(=O)(=O)C([2H])([2H])[2H])CC3)nc2n([C@@H]2CCC[C@]2(O)C([2H])([2H])[2H])c1=O] | (−)-8-((1R,2R)-2-hydroxy- 2-(methyl- d3)cyclopentyl)-6- (difluoromethyl-d)-2-((1- ((methyl- d3)sulfonyl)piperidin-4- yl)amino)pyrido[2,3- d]pyrimidin-7(8H)-one | 479.2 |
| Comp. 154 | 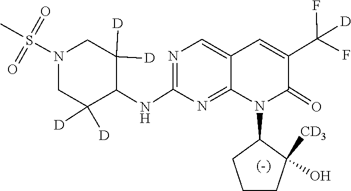 [SMILES: [2H]C(F)(F)c1cc2cnc(NC3C([2H])([2H])CN(S(C)(=O)=O)CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 480.2 |
| Comp. 155 | 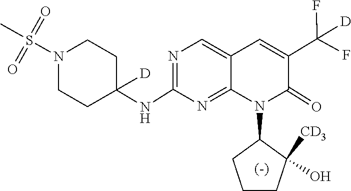 [SMILES: [2H]C1(Nc2ncc3cc(C([2H])(F)F)c(=O)n([C@@H]4CCC[C@]4(O)C([2H])([2H])[2H])c3n2)CCN(S(C)(=O)=O)CC1] | (−)-8-((1R,2R)-2-hydroxy- 2-(methyl- d3)cyclopentyl)-6- (difluoromethyl-d)-2-((1- (methylsulfonyl)piperidin- 4-yl-4-d)- amino)pyrido[2,3- d]pyrimidin-7(8H)-one | 477.2 |
| Comp. 156 | 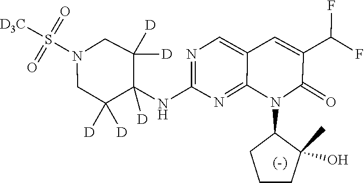 [SMILES: [2H]C1([2H])CN(S(=O)(=O)C([2H])([2H])[2H])CC([2H])([2H])C1([2H])Nc1ncc2cc(C(F)F)c(=O)n([C@@H]3CCC[C@@]3(C)O)c2n1] | (−)-6-(difluoromethyl)-8- ((1R,2R)-2-hydroxy-2- methylcyclopentyl)-2-((1- ((methyl- d3)sulfonyl)piperidin-4- yl-3,3,4,5,5- d5)amino)pyrido[2,3- d]pyrimidin-7(8H)-one | 480.2/ 88.8% D/ 99.7% ee |
| Comp. 157 | 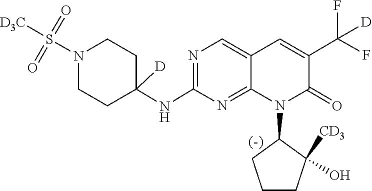 [SMILES: [2H]C1(Nc2ncc3cc(C([2H])(F)F)c(=O)n([C@@H]4CCC[C@]4(O)C([2H])([2H])[2H])c3n2)CCN(S(=O)(=O)C([2H])([2H])[2H])CC1] | (−)-8-((1R,2R)-2-hydroxy- 2-(methyl- d3)cyclopentyl)-6- (difluoromethyl-d)-2-((1- ((methyl- d3)sulfonyl)piperidin-4- yl-4-d)-amino)pyrido[2,3- d]pyrimidin-7(8H)-one | 480.2 |
| Comp. 158 | 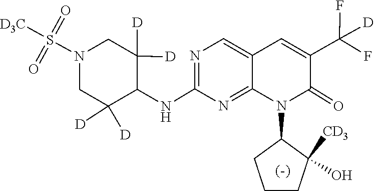 [SMILES: [2H]C(F)(F)c1cc2cnc(NC3C([2H])([2H])CN(S(=O)(=O)C([2H])([2H])[2H])CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (difluoromethyl-d)-2-((1- ((methyl- d3)sulfonyl)piperidin-4- yl-3,3,5,5-d4)- amino)pyrido[2,3- d]pyrimidin-7(8H)-one | 483.2 |
| Comp. 159 | 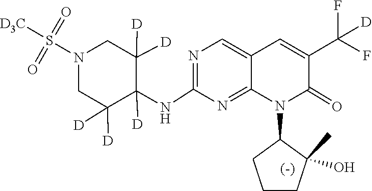 [SMILES: [2H]C(F)(F)c1cc2cnc(NC3([2H])C([2H])([2H])CN(S(=O)(=O)C([2H])([2H])[2H])CC3([2H])[2H])nc2n([C@@H]2CCC[C@@]2(C)O)c1=O] | (−)-6-(difluoromethyl-d)- 8-((1R,2R)-2-hydroxy-2- methylcyclopentyl)-2-((1- ((methyl- d3)sulfonyl)piperidin-4- yl-3,3,4,5,5- d5)amino)pyrido[2,3- d]pyrimidin-7(8H)-one | 481.2/ 88.8 D/ 99.7% ee |
| Comp. 160 | 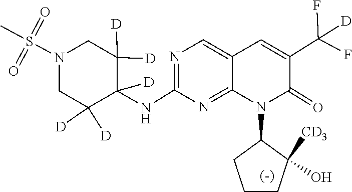 [SMILES: [2H]C(F)(F)c1cc2cnc(NC3([2H])C([2H])([2H])CN(S(C)(=O)=O)CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (difluoromethyl-d)-2-((1- (methylsulfonyl)piperidin- 4-yl-3,3,4,5,5-d5)- amino)pyrido[2,3- d]pyrimidin-7(8H)-one | 481.2 |
| Comp. 161 | 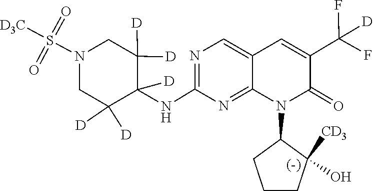 [SMILES: [2H]C(F)(F)c1cc2cnc(NC3([2H])C([2H])([2H])CN(S(=O)(=O)C([2H])([2H])[2H])CC3([2H])[2H])nc2n([C@@H]2CCC[C@]2(O)C([2H])([2H])[2H])c1=O] | (−)-8-((1R,2R)-2-hydroxy- 2-(methyl- d3)cyclopentyl)-6- (difluoromethyl-d)-2-((1- ((methyl- d3)sulfonyl)piperidin-4- yl-3,3,4,5,5-d5)- amino)pyrido[2,3- d]pyrimidin-7(8H)-one | 484.2 |
| Comp. 162 | 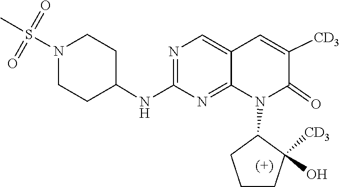 [SMILES: [2H]C([2H])([2H])c1cc2cnc(NC3CCN(S(C)(=O)=O)CC3)nc2n([C@H]2CCC[C@@]2(O)C([2H])([2H])[2H])c1=O] | (+)-8-((1S,2S)-2-hydroxy- 2-(methyl- d3)cyclopentyl)-6- (methyl-d3)-2-((1- (methylsulfonyl)piperidin- 4-yl)amino)pyrido[2,3- d]pyrimidin-7(8H)-one | 442.2 |
| Comp. 163 | 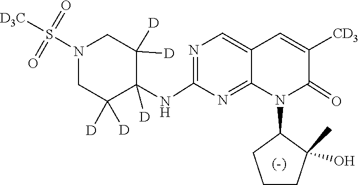 [SMILES: [2H]C([2H])([2H])c1cc2cnc(NC3([2H])C([2H])([2H])CN(S(=O)(=O)C([2H])([2H])[2H])CC3([2H])[2H])nc2n([C@@H]2CCC[C@@]2(C)O)c1=O] | (−)-8-((1R,2R)-2-hydroxy- 2-methylcyclopentyl)-6- (methyl-d3)-2-((1- ((methyl- d3)sulfonyl)piperidin-4- yl-3,3,4,5,5- d5)amino)pyrido[2,3- d]pyrimidin-7(8H)-one | 447.2/ 83% D/ 99.7% ee |
| Comp. 164 | 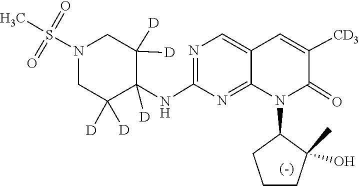 [SMILES: [2H]C([2H])([2H])c1cc2cnc(NC3([2H])C([2H])([2H])CN(S(C)(=O)=O)CC3([2H])[2H])nc2n([C@@H]2CCC[C@@]2(C)O)c1=O] | (−)-8-((1R,2R)-2-hydroxy- 2-methylcyclopentyl)-6- (methyl-d3)-2-((1- (methylsulfonyl)piperidin- 4-yl-3,3,4,5,5- d5)amino)pyrido[2,3- d]pyrimidin-7(8H)-one | 444.2/ 85% D/ 99.7% ee |
| Comp. 165 | 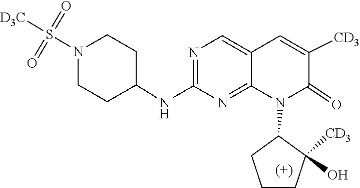 [SMILES: [2H]C([2H])([2H])c1cc2cnc(NC3CCN(S(=O)(=O)C([2H])([2H])[2H])CC3)nc2n([C@H]2CCC[C@@]2(O)C([2H])([2H])[2H])c1=O] | (+)-8-((1S,2S)-2-hydroxy- 2-(methyl- d3)cyclopentyl)-6- (methyl-d3)-2-((1- ((methy- d3)lsulfonyl)piperidin-4- yl)amino)pyrido[2,3- d]pyrimidin-7(8H)-one | 445.2 |
| Comp. 166 | 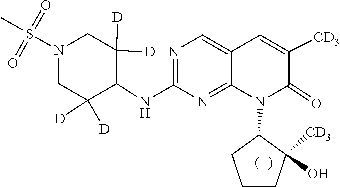 [SMILES: [2H]C([2H])([2H])c1cc2cnc(NC3C([2H])([2H])CN(S(C)(=O)=O)CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (methyl-d3)-2-((1- (methylsulfonyl)piperidin- 4-yl-3,3,5,5-d4)- amino)pyrido[2,3- | 446.3 |
| Comp. 167 | 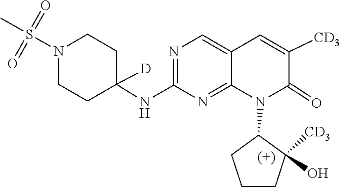 [SMILES: [2H]C1(Nc2ncc3cc(C([2H])([2H])[2H])c(=O)n([C@H]4CCC[C@@]4(O)C([2H])([2H])[2H])c3n2)CCN(S(C)(=O)=O)CC1] | (+)-8-((1S,2S)-2-hydroxy- 2-(methyl- d3)cyclopentyl)-6- (methyl-d3)-2-((1- (methylsulfonyl)piperidin- 4-yl-4-d)- amino)pyrido[2,3- d]pyrimidin-7(8H)-one | 443.2 |
| Comp. 168 | 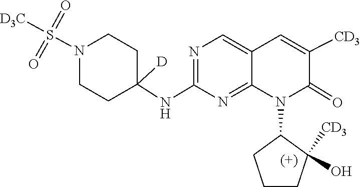 [SMILES: [2H]C1(Nc2ncc3cc(C([2H])([2H])[2H])c(=O)n([C@H]4CCC[C@@]4(O)C([2H])([2H])[2H])c3n2)CCN(S(=O)(=O)C([2H])([2H])[2H])CC1] | (+)-8-((1S,2S)-2-hydroxy- 2-(methyl- d3)cyclopentyl)-6- (methyl-d3)-2-((1- ((methyl- d3)sulfonyl)piperidin-4- yl-4-d)-amino)pyrido[2,3- d]pyrimidin-7(8H)-one | 446.3 |
| Comp. 169 | 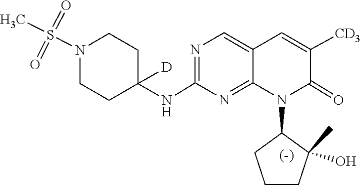 [SMILES: [2H]C1(Nc2ncc3cc(C([2H])([2H])[2H])c(=O)n([C@@H]4CCC[C@@]4(C)O)c3n2)CCN(S(C)(=O)=O)CC1] | (−) 8-((1R,2R)-2-hydroxy- 2-methylcyclopentyl)-6- (methyl-d3)-2-((1- (methylsulfonyl)piperidin- 4-d-4- yl)amino)pyrido[2,3- d]pyrimidin-7(8H)-one | 440.2/ 99% D/ 99.7% ee |
| Comp. 170 | 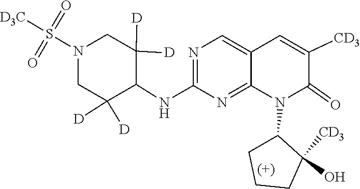 [SMILES: [2H]C([2H])([2H])c1cc2cnc(NC3C([2H])([2H])CN(S(=O)(=O)C([2H])([2H])[2H])CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 449.3 |
| Comp. 171 | 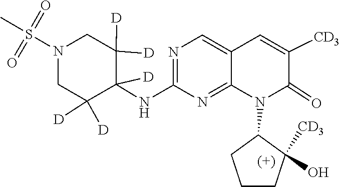 [SMILES: [2H]C([2H])([2H])c1cc2cnc(NC3([2H])C([2H])([2H])CN(S(C)(=O)=O)CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (methyl-d3)-2-((1- (methylsulfonyl)piperidin- 4-yl-3,3,4,5,5-d5)- amino)pyrido[2,3- d]pyrimidin-7(8H)-one | 447.3 |
| Comp. 172 | 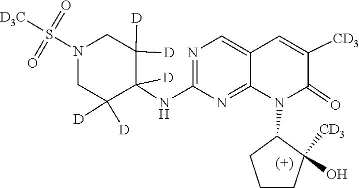 [SMILES: [2H]C([2H])([2H])c1cc2cnc(NC3([2H])C([2H])([2H])CN(S(=O)(=O)C([2H])([2H])[2H])CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 450.3 |
| Comp. 173 | 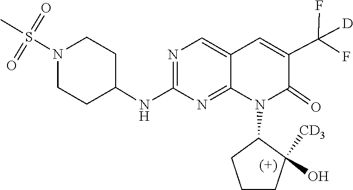 [SMILES: [2H]C(F)(F)c1cc2cnc(NC3CCN(S(C)(=O)=O)CC3)nc2n([C@H]2CCC[C@@]2(O)C([2H])([2H])[2H])c1=O] | (+)-8-((1S,2S)-2-hydroxy- 2-(methyl- d3)cyclopentyl)-6- (difluoromethyl-d)-2-((1- (methylsulfonyl)piperidin- 4-yl)amino)pyrido[2,3- d]pyrimidin-7(8H)-one | 476.2 |
| Comp. 174 | 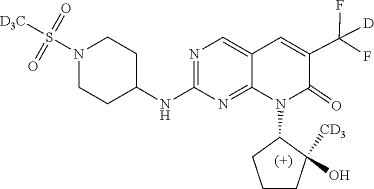 [SMILES: [2H]C(F)(F)c1cc2cnc(NC3CCN(S(=O)(=O)C([2H])([2H])[2H])CC3)nc2n([C@H]2CCC[C@@]2(O)C([2H])([2H])[2H])c1=O] | (+)-8-((1S,2S)-2-hydroxy- 2-(methyl- d3)cyclopentyl)-6- (difluoromethyl-d)-2-((1- ((methyl- d3)sulfonyl)piperidin-4- yl)amino)pyrido[2,3- d]pyrimidin-7(8H)-one | 479.2 |
| Comp. 175 | 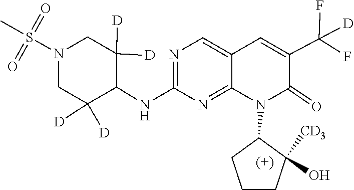 [SMILES: [2H]C(F)(F)c1cc2cnc(NC3C([2H])([2H])CN(S(C)(=O)=O)CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 480.2 |
| Comp. 176 | 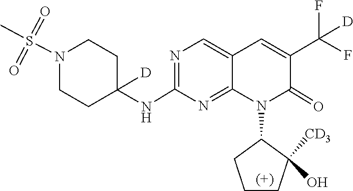 [SMILES: [2H]C1(Nc2ncc3cc(C([2H])(F)F)c(=O)n([C@H]4CCC[C@@]4(O)C([2H])([2H])[2H])c3n2)CCN(S(C)(=O)=O)CC1] | (+)-8-((1S,2S)-2-hydroxy- 2-(methyl- d3)cyclopentyl)-6- (difluoromethyl-d)-2-((1- (methylsulfonyl)piperidin- 4-yl-4-d)- amino)pyrido[2,3- d]pyrimidin-7(8H)-one | 477.2 |
| Comp. 177 | 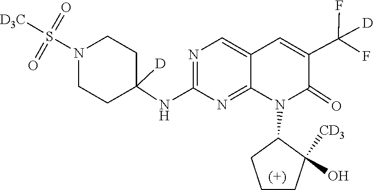 [SMILES: [2H]C1(Nc2ncc3cc(C([2H])(F)F)c(=O)n([C@H]4CCC[C@@]4(O)C([2H])([2H])[2H])c3n2)CCN(S(=O)(=O)C([2H])([2H])[2H])CC1] | (+)-8-((1S,2S)-2-hydroxy- 2-(methyl- d3)cyclopentyl)-6- (difluoromethyl-d)-2-((1- ((methyl- d3)sulfonyl)piperidin-4- yl-4-d)-amino)pyrido[2,3- d]pyrimidin-7(8H)-one | 480.2 |
| Comp. 178 | 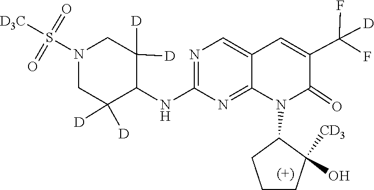 [SMILES: [2H]C(F)(F)c1cc2cnc(NC3C([2H])([2H])CN(S(=O)(=O)C([2H])([2H])[2H])CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (difluoromethyl-d)-2-((1- ((methyl- d3)sulfonyl)piperidin-4- yl-3,3,5,5-d4)- amino)pyrido[2,3- d]pyrimidin-7(8H)-one | 483.2 |
| Comp. 179 | 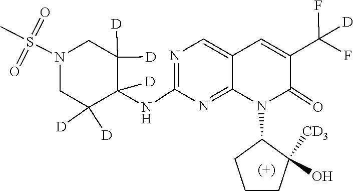 [SMILES: [2H]C(F)(F)c1cc2cnc(NC3([2H])C([2H])([2H])CN(S(C)(=O)=O)CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (difluoromethyl-d)-2-((1- (methylsulfonyl)piperidin- 4-yl-3,3,4,5,5-d5)- amino)pyrido[2,3- d]pyrimidin-7(8H)-one | 481.2 |
| Comp. 180 | 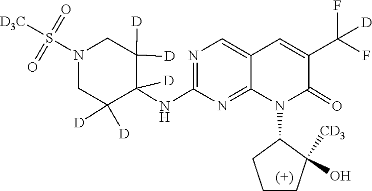 [SMILES: [2H]C(F)(F)c1cc2cnc(NC3([2H])C([2H])([2H])CN(S(=O)(=O)C([2H])([2H])[2H])CC3([2H])[2H])nc2n([C@H]2CCC[C@@]2(O)C([2H])([2H])[2H])c1=O] | (+)-8-((1S,2S)-2-hydroxy- 2-(methyl- d3)cyclopentyl)-6- (difluoromethyl-d)-2-((1- ((methyl- d3)sulfonyl)piperidin-4- yl-3,3,4,5,5-d5)- amino)pyrido[2,3- d]pyrimidin-7(8H)-one | 484.2 |
| Comp. 181 | 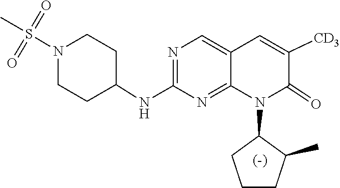 [SMILES: [2H]C([2H])([2H])c1cc2cnc(NC3CCN(S(C)(=O)=O)CC3)nc2n([C@@H]2CCC[C@@H]2C)c1=O] | (−)-6-(methyl-d3)-8- ((1R,2S)-2- methylcyclopentyl)-2-((1- (methylsulfonyl)piperidin- 4-yl)amino)pyrido[2,3- d]pyrimidin-7(8H)-one | 423.2 |
| Comp. 182 | 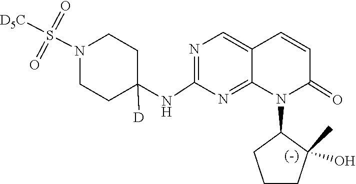 | (−)-8-((1R,2R)-2-hydroxy- 2-methylcyclopentyl)-2- ((1-((methyl- d3)sulfonyl)piperidin-4- yl-4-d)amino)pyrido[2,3- d]pyrimidin-7(8H)-one | 425.20 |
| Comp. 183 | 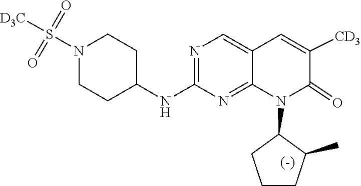 [SMILES: [2H]C([2H])([2H])c1cc2cnc(NC3CCN(S(=O)(=O)C([2H])([2H])[2H])CC3)nc2n([C@@H]2CCC[C@@H]2C)c1=O] | (−)-6-(methyl-d3)-8- ((1R,2S)-2- methylcyclopentyl)-2-((1- ((methyl- d3)sulfonyl)piperidin-4- yl)amino)pyrido[2,3- d]pyrimidin-7(8H)-one | 426.2 |
| Comp. 184 | 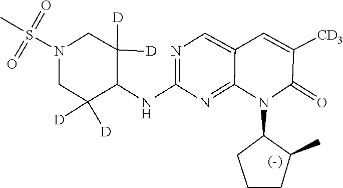 [SMILES: [2H]C([2H])([2H])c1cc2cnc(NC3C([2H])([2H])CN(S(C)(=O)=O)CC3([2H])[2H])nc2n([C@@H]2CCC[C@@H]2C)c1=O] | (−)-6-(methyl-d3)-8- ((1R,2S)-2- methylcyclopentyl)-2-((1- (methylsulfonyl)piperidin- 4-yl-3,3,5,5-d4)- amino)pyrido[2,3- d]pyrimidin-7(8H)-one | 427.2 |
| Comp. 185 | 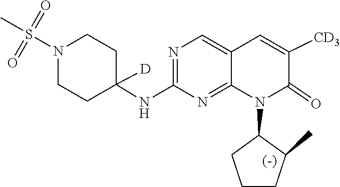 [SMILES: [2H]C1(Nc2ncc3cc(C([2H])([2H])[2H])c(=O)n([C@@H]4CCC[C@@H]4C)c3n2)CCN(S(C)(=O)=O)CC1] | (−)-6-(methyl-d3)-8- ((1R,2S)-2- methylcyclopentyl)-2-((1- (methylsulfonyl)piperidin- 4-yl-4-d)- amino)pyrido[2,3- d]pyrimidin-7(8H)-one | 424.2 |
| Comp. 186 | 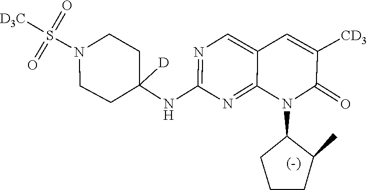 [SMILES: [2H]C1(Nc2ncc3cc(C([2H])([2H])[2H])c(=O)n([C@@H]4CCC[C@@H]4C)c3n2)CCN(S(=O)(=O)C([2H])([2H])[2H])CC1] | (−)-6-(methyl-d3)-8- ((1R,2S)-2- methylcyclopentyl)-2-((1- ((methyl- d3)sulfonyl)piperidin-4- yl-4-d)-amino)pyrido[2,3- d]pyrimidin-7(8H)-one | 427.2 |
| Comp. 187 | 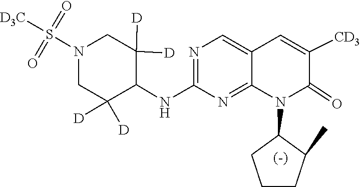 [SMILES: [2H]C([2H])([2H])c1cc2cnc(NC3C([2H])([2H])CN(S(=O)(=O)C([2H])([2H])[2H])CC3([2H])[2H])nc2n([C@@H]2CCC[C@@H]2C)c1=O] | (−)-6-(methyl-d3)-8- ((1R,2S)-2- methylcyclopentyl)-2-((1- ((methyl- d3)sulfonyl)piperidin-4- yl-3,3,5,5-d4)- amino)pyrido[2,3- d]pyrimidin-7(8H)-one | 430.3 |
| Comp. 188 | 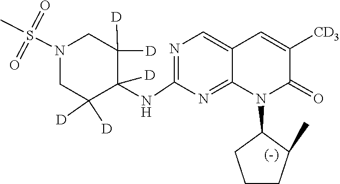 [SMILES: [2H]C([2H])([2H])c1cc2cnc(NC3([2H])C([2H])([2H])CN(S(C)(=O)=O)CC3([2H])[2H])nc2n([C@@H]2CCC[C@@H]2C)c1=O] | (−)-6-(methyl-d3)-8- ((1R,2S)-2- methylcyclopentyl)-2-((1- (methylsulfonyl)piperidin- 4-yl-3,3,4,5,5-d5)- amino)pyrido[2,3- d]pyrimidin-7(8H)-one | 428.2 |
| Comp. 189 | 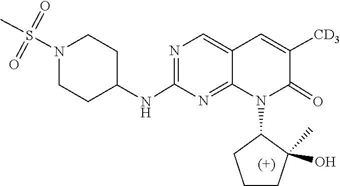 [SMILES: [2H]C([2H])([2H])c1cc2cnc(NC3CCN(S(C)(=O)=O)CC3)nc2n([C@H]2CCC[C@]2(C)O)c1=O] | (+)-8-((1S,2S)-2-hydroxy- 2-methylcyclopentyl)-6- (methyl-d3)-2-((1- (methylsulfonyl)piperidin- 4-yl)amino)pyrido[2,3- d]pyrimidin-7(8H)-one | 439.2 |
| Comp. 190 | 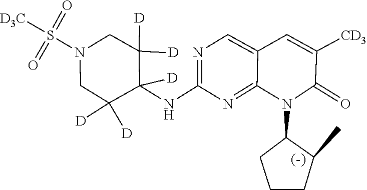 [SMILES: [2H]C([2H])([2H])c1cc2cnc(NC3([2H])C([2H])([2H])CN(S(=O)(=O)C([2H])([2H])[2H])CC3([2H])[2H])nc2n([C@@H]2CCC[C@@H]2C)c1=O] | (−)-6-(methyl-d3)-8- ((1R,2S)-2- methylcyclopentyl)-2-((1- ((methyl- d3)sulfonyl)piperidin-4- yl-3,3,4,5,5-d5)- amino)pyrido[2,3- d]pyrimidin-7(8H)-one | 431.3 |
| Comp. 191 | 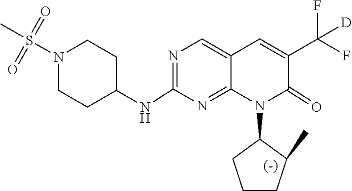 [SMILES: [2H]C(F)(F)c1cc2cnc(NC3CCN(S(C)(=O)=O)CC3)nc2n([C@@H]2CCC[C@@H]2C)c1=O] | (−)-6-(difluoromethyl-d)- 8-((1R,2S)-2- methylcyclopentyl)-2-((1- (methylsulfonyl)piperidin- 4-yl)amino)pyrido[2,3- d]pyrimidin-7(8H)-one | 457.2 |
| Comp. 192 | 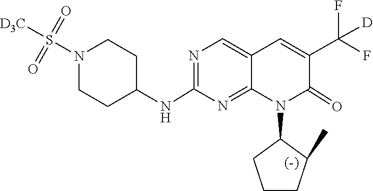 [SMILES: [2H]C(F)(F)c1cc2cnc(NC3CCN(S(=O)(=O)C([2H])([2H])[2H])CC3)nc2n([C@@H]2CCC[C@@H]2C)c1=O] | (−)-6-(difluoromethyl-d)- 8-((1R,2S)-2- methylcyclopentyl)-2-((1- ((methyl- d3)sulfonyl)piperidin-4- yl)amino)pyrido[2,3- d]pyrimidin-7(8H)-one | 460.2 |
| Comp. 193 | 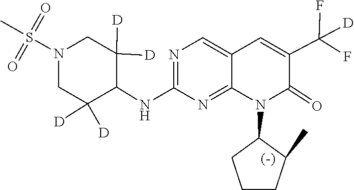 [SMILES: [2H]C(F)(F)c1cc2cnc(NC3C([2H])([2H])CN(S(C)(=O)=O)CC3([2H])[2H])nc2n([C@@H]2CCC[C@@H]2C)c1=O] | (−)-6-(difluoromethyl-d)- 8-((1R,2S)-2- methylcyclopentyl)-2-((1- (methylsulfonyl)piperidin- 4-yl-3,3,5,5-d4)- amino)pyrido[2,3- d]pyrimidin-7(8H)-one | 461.2 |
| Comp. 194 | 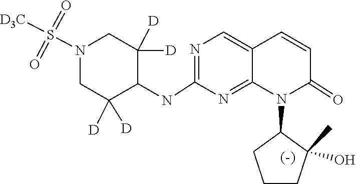 | (−)-8-((1R,2R)-2-hydroxy- 2-methylcyclopentyl)-2- ((1-((methyl- d3)sulfonyl)piperidin-4- yl-3,3,5,5- d4)amino)pyrido[2,3- d]pyrimidin-7(8H)-one | 428.22 |
| Comp. 195 | 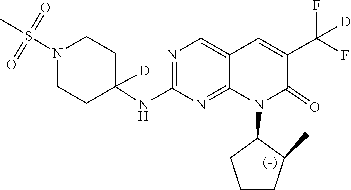 [SMILES: [2H]C1(Nc2ncc3cc(C([2H])(F)F)c(=O)n([C@@H]4CCC[C@@H]4C)c3n2)CCN(S(C)(=O)=O)CC1] | (−)-6-(difluoromethyl-d)- 8-((1R,2S)-2- methylcyclopentyl)-2-((1- (methylsulfonyl)piperidin- 4-yl-4-d)- amino)pyrido[2,3- d]pyrimidin-7(8H)-one | 458.2 |
| Comp. 196 | 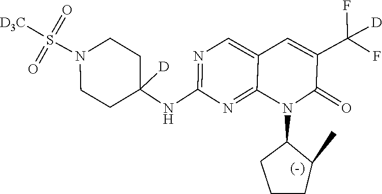 [SMILES: [2H]C1(Nc2ncc3cc(C([2H])(F)F)c(=O)n([C@@H]4CCC[C@@H]4C)c3n2)CCN(S(=O)(=O)C([2H])([2H])[2H])CC1] | (−)-6-(difluoromethyl-d)- 8-((1R,2S)-2- methylcyclopentyl)-2-((1- ((methyl- d3)sulfonyl)piperidin-4- yl-4-d)-amino)pyrido[2,3- d]pyrimidin-7(8H)-one | 461.2 |
| Comp. 197 | 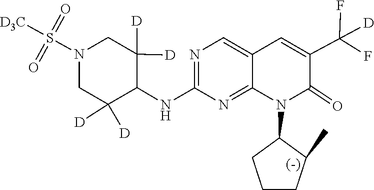 [SMILES: [2H]C(F)(F)c1cc2cnc(NC3C([2H])([2H])CN(S(=O)(=O)C([2H])([2H])[2H])CC3([2H])[2H])nc2n([C@@H]2CCC[C@@H]2C)c1=O] | (−)-6-(difluoromethyl-d)- 8-((1R,2S)-2- methylcyclopentyl)-2-((1- ((methyl- d3)sulfonyl)piperidin-4- yl-3,3,5,5-d4)- amino)pyrido[2,3- d]pyrimidin-7(8H)-one | 464.2 |
| Comp. 198 | 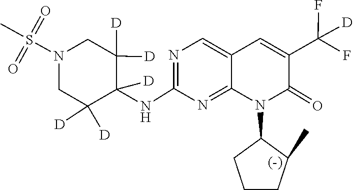 [SMILES: [2H]C(F)(F)c1cc2cnc(NC3([2H])C([2H])([2H])CN(S(C)(=O)=O)CC3([2H])[2H])nc2n([C@@H]2CCC[C@@H]2C)c1=O] | (−)-6-(difluoromethyl-d)- 8-((1R,2S)-2- methylcyclopentyl)-2-((1- (methylsulfonyl)piperidin- 4-yl-3,3,4,5,5-d5)- amino)pyrido[2,3- d]pyrimidin-7(8H)-one | 462.2 |
| Comp. 199 | 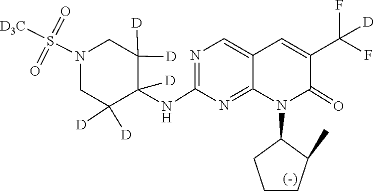 [SMILES: [2H]C(F)(F)c1cc2cnc(NC3([2H])C([2H])([2H])CN(S(=O)(=O)C([2H])([2H])[2H])CC3([2H])[2H])nc2n([C@@H]2CCC[C@@H]2C)c1=O] | (−)-6-(difluoromethyl-d)- 8-((1R,2S)-2- methylcyclopentyl)-2-((1- ((methyl- d3)sulfonyl)piperidin-4- yl-3,3,4,5,5-d5)- amino)pyrido[2,3- d]pyrimidin-7(8H)-one | 465.2 |
| Comp. 200 | 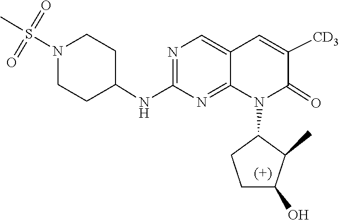 [SMILES: [2H]C([2H])([2H])c1cc2cnc(NC3CCN(S(C)(=O)=O)CC3)nc2n([C@H]2CC[C@H](O)[C@@H]2C)c1=O] | (+)-8-((1S,2R,3S)-3- hydroxy-2- methylcyclopentyl)-6- (methyl-d3)-2-((1- (methylsulfonyl)piperidin- 4-yl)amino)pyrido[2,3- d]pyrimidin-7(8H)-one | 439.2 |
| Comp. 201 | 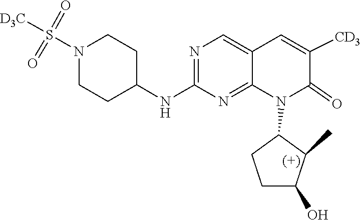 [SMILES: [2H]C([2H])([2H])c1cc2cnc(NC3CCN(S(=O)(=O)C([2H])([2H])[2H])CC3)nc2n([C@H]2CC[C@H](O)[C@@H]2C)c1=O] | (+)-8-((1S,2R,3S)-3- hydroxy-2- methylcyclopentyl)-6- (methyl-d3)-2-((1- ((methyl- d3)sulfonyl)piperidin-4- yl)amino)pyrido[2,3- d]pyrimidin-7(8H)-one | 442.2 |
| Comp. 202 | 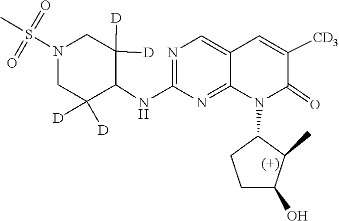 [SMILES: [2H]C([2H])([2H])c1cc2cnc(NC3C([2H])([2H])CN(S(C)(=O)=O)CC3([2H])[2H])nc2n([C@H]2CC[C@H](O)[C@@H]2C)c1=O] | (+)-8-((1S,2R,3S)-3- hydroxy-2- methylcyclopentyl)-6- (methyl-d3)-2-((1- (methylsulfonyl)piperidin- 4-yl-3,3,5,5)- amino)pyrido[2,3- d]pyrimidin-7(8H)-one | 443.2 |
| Comp. 203 | 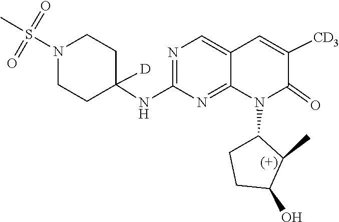 [SMILES: [2H]C1(Nc2ncc3cc(C([2H])([2H])[2H])c(=O)n([C@H]4CC[C@H](O)[C@@H]4C)c3n2)CCN(S(C)(=O)=O)CC1] | (+)-8-((1S,2R,3S)-3- hydroxy-2- methylcyclopentyl)-6- (methyl-d3)-2-((1- (methylsulfonyl)piperidin- 4-yl-4-d)- amino)pyrido[2,3- d]pyrimidin-7(8H)-one | 440.2 |
| Comp. 204 | 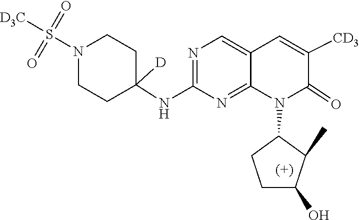 [SMILES: [2H]C1(Nc2ncc3cc(C([2H])([2H])[2H])c(=O)n([C@H]4CC[C@H](O)[C@@H]4C)c3n2)CCN(S(=O)(=O)C([2H])([2H])[2H])CC1] | (+)-8-((1S,2R,3S)-3- hydroxy-2- methylcyclopentyl)-6- (methyl-d3)-2-((1- ((methyl- d3)sulfonyl)piperidin-4- yl-4-d)-amino)pyrido[2,3- d]pyrimidin-7(8H)-one | |
| Comp. 205 | 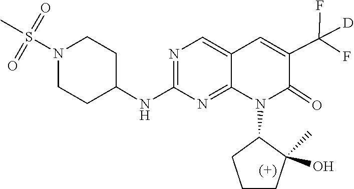 [SMILES: [2H]C(F)(F)c1cc2cnc(NC3CCN(S(C)(=O)=O)CC3)nc2n([C@H]2CCC[C@]2(C)O)c1=O] | (+)-6-(difluoromethyl-d)- 8-((1S,2S)-2-hydroxy-2- methylcyclopentyl)-2-((1- (methylsulfonyl)piperidin- 4-yl)amino)pyrido[2,3- d]pyrimidin-7(8H)-one | 473.2 |
| Comp. 206 | 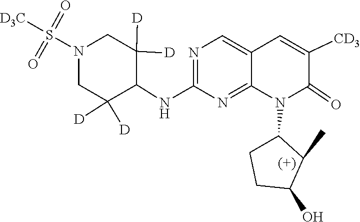 [SMILES: [2H]C([2H])([2H])c1cc2cnc(NC3C([2H])([2H])CN(S(=O)(=O)C([2H])([2H])[2H])CC3([2H])[2H])nc2n([C@H]2CC[C@H](O)[C@@H]2C)c1=O] | (+)-8-((1S,2R,3S)-3- hydroxy-2- methylcyclopentyl)-6- (methyl-d3)-2-((1- ((methyl- d3)sulfonyl)piperidin-4- yl-3,3,5,5-d4)- amino)pyrido[2,3- d]pyrimidin-7(8H)-one | 446.3 |
| Comp. 207 | 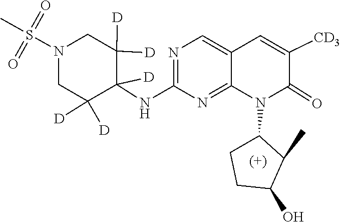 [SMILES: [2H]C([2H])([2H])c1cc2cnc(NC3([2H])C([2H])([2H])CN(S(C)(=O)=O)CC3([2H])[2H])nc2n([C@H]2CC[C@H](O)[C@@H]2C)c1=O] | (+)-8-((1S,2R,3S)-3- hydroxy-2- methylcyclopentyl)-6- (methyl-d3)-2-((1- (methylsulfonyl)piperidin- 4-yl-3,3,4,5,5-d5)- amino)pyrido[2,3- d]pyrimidin-7(8H)-one | 444.2 |
| Comp. 208 | 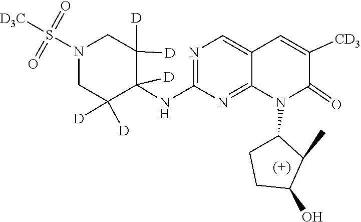 [SMILES: [2H]C([2H])([2H])c1cc2cnc(NC3([2H])C([2H])([2H])CN(S(=O)(=O)C([2H])([2H])[2H])CC3([2H])[2H])nc2n([C@H]2CC[C@H](O)[C@@H]2C)c1=O] | (+)-8-((1S,2R,3S)-3- hydroxy-2- methylcyclopentyl)-6- (methyl-d3)-2-((1- ((methyl- d3)sulfonyl)piperidin-4- yl-3,3,4,5,5-d5)- amino)pyrido[2,3- d]pyrimidin-7(8H)-one | 447.3 |
| Comp. 209 | 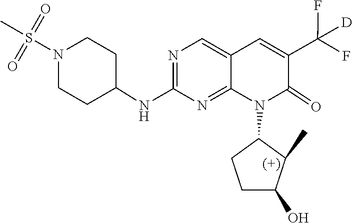 [SMILES: [2H]C(F)(F)c1cc2cnc(NC3CCN(S(C)(=O)=O)CC3)nc2n([C@H]2CC[C@H](O)[C@@H]2C)c1=O] | (+)-8-((1S,2R,3S)-3- hydroxy-2- methylcyclopentyl)-6- (difluoromethyl-d)-2-((1- (methylsulfonyl)piperidin- 4-yl)amino)pyrido[2,3- d]pyrimidin-7(8H)-one | 473.2 |
| Comp. 210 | 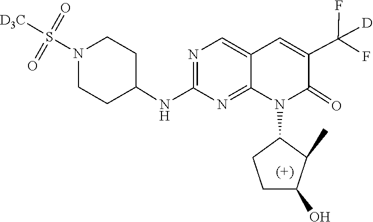 [SMILES: [2H]C(F)(F)c1cc2cnc(NC3CCN(S(=O)(=O)C([2H])([2H])[2H])CC3)nc2n([C@H]2CC[C@H](O)[C@@H]2C)c1=O] | (+)-8-((1S,2R,3S)-3- hydroxy-2- methylcyclopentyl)-6- (difluoromethyl-d)-2-((1- ((methyl- d3)sulfonyl)piperidin-4- yl)amino)pyrido[2,3- d]pyrimidin-7(8H)-one | 476.2 |
| Comp. 211 | 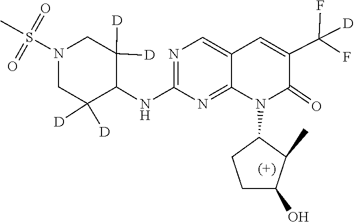 [SMILES: [2H]C(F)(F)c1cc2cnc(NC3C([2H])([2H])CN(S(C)(=O)=O)CC3([2H])[2H])nc2n([C@H]2CC[C@H](O)[C@@H]2C)c1=O] | (+)-8-((1S,2R,3S)-3- hydroxy-2- methylcyclopentyl)-6- (difluoromethyl-d)-2-((1- (methylsulfonyl)piperidin- 4-yl-3,3,5,5-d4)- amino)pyrido[2,3- d]pyrimidin-7(8H)-one | 477.2 |
| Comp. 212 | 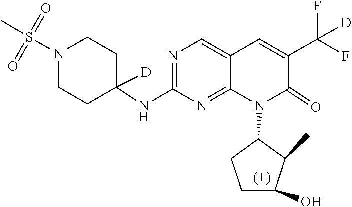 [SMILES: [2H]C1(Nc2ncc3cc(C([2H])(F)F)c(=O)n([C@H]4CC[C@H](O)[C@@H]4C)c3n2)CCN(S(C)(=O)=O)CC1] | (+)-8-((1S,2R,3S)-3- hydroxy-2- methylcyclopentyl)-6- (difluoromethyl-d)-2-((1- (methylsulfonyl)piperidin- 4-yl-4-d)- amino)pyrido[2,3- d]pyrimidin-7(8H)-one | 474.2 |
| Comp. 213 | 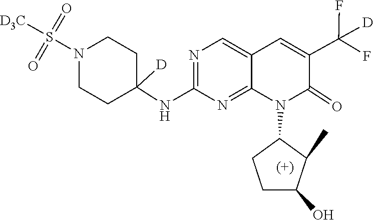 [SMILES: [2H]C1(Nc2ncc3cc(C([2H])(F)F)c(=O)n([C@H]4CC[C@H](O)[C@@H]4C)c3n2)CCN(S(=O)(=O)C([2H])([2H])[2H])CC1] | (+)-8-((1S,2R,3S)-3- hydroxy-2- methylcyclopentyl)-6- (difluoromethyl-d)-2-((1- ((methyl- d3)sulfonyl)piperidin-4- yl-4-d)-amino)pyrido[2,3- d]pyrimidin-7(8H)-one | 477.2 |
| Comp. 214 | 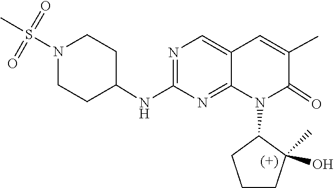 | (+)-8-((1S,2S)-2-hydroxy- 2-methylcyclopentyl)-6- methyl-2-((1- (methylsulfonyl)piperidin- 4-yl)amino)pyrido[2,3- d]pyrimidin-7(8H)-one | 436.2 |
| Comp. 215 | 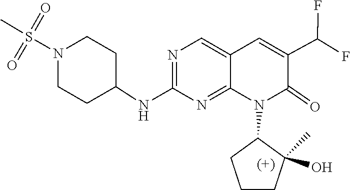 [SMILES: C[C@]1(O)CCC[C@@H]1n1c(=O)c(C(F)F)cc2cnc(NC3CCN(S(C)(=O)=O)CC3)nc21] | (+)-6-(difluoromethyl)-8- ((1S,2S)-2-hydroxy-2- methylcyclopentyl)-2-((1- (methylsulfonyl)piperidin- 4-yl)amino)pyrido[2,3- d]pyrimidin-7(8H)-one | 472.2 |

### Example 36. In Vitro Kinase Assays

Materials and Reagents

(1). Recombinant enzymes CDK1, CDK2, CDK4, CDK6, CDK9, cyclin B1, cyclin A2, cyclin D3, cyclin E1, cyclin T1 were cloned and purified at Wuxi Biortus Biochemistry Co. Ltd.

(2). Kinase buffer: 50 mM Tris HCl pH 8.0 (SCR, Cat. 30188360.) with 0.01% Tween 20 (Sigma, P2287), 50 μg/mL BSA (Aladdin, A104912), and 5 mM MgCl₂ (SCR, Cat. 10012818).

(3). U bottom 384-well microplate (Corning, cat. No. 4512).

(4). ATP, stored at 4° C. (VWR, Cat. 97061-226).

(5). MgCl₂: (SCR, Cat. 10012818).

(6). EDTA: (SCR, Cat. 10009717).

(7) Peptide substrate-CDK1, CDK2, CDK4, CDK6 (5-FAM-YSPTSPSYSPTSPSYSPTSPSKKKK-NH₂, Scilight-Peptide, Cat. C2546704).

(8) Peptide substrate-CDK9 (Ahx-GSRTPMY-NH₂, Scilight-Peptide, Cat. C0769102).

### 1). CDK1/Cyclin B1 Kinase Assay

CDK1/cyclin B1 kinase assays were performed in 384-well microplates (Corning, cat. No. 4512) and the final volume of the assay is 16 μL and reaction temperature is 27° C. Enzyme was diluted to a 2.4x dilution to provide a final concentration of 0.1 nM in kinase buffer containing 50 mM Tris HCl pH 8.0, 0.01% Tween 20, 50 μg/mL BSA, and 5 mM MgCl₂. The peptide substrate (5-FAM-YSPTSPSYSPTSPSYSPTSPSKKKK-NH₂) and ATP were diluted in kinase buffer at 2.4x dilution the final assay concentration to provide a mixture having final concentration of 2 μM of peptide substrate and 2 mM ATP. The test compounds were dissolved and diluted in DMSO. A stock DMSO solution of the test compound of the invention was prepared by dissolving the compound in DMSO to form a 10 mM solution. Ten points serial dilutions were made into DMSO using the 10 mM DMSO stock solutions of the test compounds to create a concentration gradient ranging from 25 nM to 500 μM (10 concentrations total). The test compound solutions of 10 gradient concentrations were further diluted at 8.3 fold in kinase buffer at 6x the final assay concentration. 2 μL of testing compound solutions and 5 μL of enzyme solution were added into the 384-well microplate followed by a 10 minute incubation. The reaction was then initiated by the addition of 5 μL of peptide substrate and ATP mixture solution and incubated at 27° C. for 50 minutes. The enzyme reaction in each well was terminated by adding 4 μL of 120 mM EDTA solution. Each assay was carried out in duplicates.

DMSO was used as negative control (0%) for enzyme activity. 20 μM Staurosporine was used as positive control (100%) for enzyme activity.

Plates were read for fluorescence on a Caliper EZ ReaderII® by separating the fluorescent labeled substrate from the enzyme phosphorylation product by electrophoresis. Data were collected and analyzed using GraphPad Prism® version 6.0 software.

Dose-response curves were plotted from inhibition data generated, each in duplicate, from 10 point serial dilutions of inhibitory compounds. Concentration of compound was plotted against % kinase activity. To generate IC₅₀ values, the dose-response curves were then fitted to a standard sigmoidal curve and IC₅₀ values were derived by nonlinear regression analysis.

### 2). CDK2/Cyclin A2 Kinase Assay

CDK2/cyclin A2 kinase assays were performed in 384-well microplates (Corning, cat. No. 4512) and the final volume of the assay is 16 μL and reaction temperature is 27° C. Enzyme was diluted to a 2.4x dilution to provide a final concentration of 0.05 nM in kinase buffer containing 20 mM MES pH=6.75, 0.01% Tween 20, 50 μg/mL BSA, and 5 mM MgCl₂. The peptide substrate (5-FAM-YSPTSPSYSPTSPSYSPTSPSKKKK-NH₂) and ATP were diluted in kinase buffer at 2.4x dilution the final assay concentration to provide a mixture having final concentration of 2 μM of peptide substrate and 2 mM ATP. The test compounds were dissolved and diluted in DMSO. A stock DMSO solution of the test compound of the invention was prepared by dissolving the compound in DMSO to form a 10 mM solution. Ten points serial dilutions were made into DMSO using the 10 mM DMSO stock solutions of the test compounds to create a concentration gradient ranging from 25 nM to 500 μM (10 concentrations total). The test compound solutions of 10 gradient concentrations were further diluted at 8.3 fold in kinase buffer at 6x the final assay concentration.

2 μL of testing compound solutions and 5 μL of enzyme solution were added into the 384-well microplate followed by a 10 minute incubation. The reaction was then initiated by the addition of 5 μL of peptide substrate and ATP mixture solution and incubated at 27° C. for 20 minutes. The enzyme reaction in each well was terminated by adding 4 μL of 120 mM EDTA solution. Each assay was carried out in duplicates.

DMSO was used as negative control (0%) for enzyme activity. 20 μM Staurosporine was used as positive control (100%) for enzyme activity.

Plates were read for fluorescence on a Caliper EZ ReaderII® by separating the fluorescent labeled substrate from the enzyme phosphorylation product by electrophoresis. Data were collected and analyzed using GraphPad Prism® version 6.0 software.

Dose-response curves were plotted from inhibition data generated, each in duplicate, from 10 point serial dilutions of inhibitory compounds. Concentration of compound was plotted against % kinase activity. To generate IC₅₀ values, the dose-response curves were then fitted to a standard sigmoidal curve and IC₅₀ values were derived by nonlinear regression analysis.

### 3) CDK2/Cyclin E1 Kinase Assay

CDK2/cyclin E1 kinase assays were performed in 384-well microplates (Corning, cat. No. 4512) and the final volume of the assay is 16 μL and reaction temperature is 27° C. Enzyme was diluted to a 2.4x dilution to provide a final concentration of 0.5 nM in kinase buffer containing 20 mM MES pH=6.75, 0.01% Tween 20, 50 μg/mL BSA, and 5 mM MgCl₂. The peptide substrate (5-FAM-YSPTSPSYSPTSPSYSPTSPSKKKK-NH2) and ATP were diluted in kinase buffer at 2.4x dilution the final assay concentration to provide a mixture having final concentration of 2 μM of peptide substrate and 2 mM ATP. The test compounds were dissolved and diluted in DMSO. A stock DMSO solution of the test compound of the invention was prepared by dissolving the compound in DMSO to form a 10 mM solution. Ten points serial dilutions were made into DMSO using the 10 mM DMSO stock solutions of the test compounds to create a concentration gradient ranging from 25 nM to 500 μM (10 concentrations total). The test compound solutions of 10 gradient concentrations were further diluted at 8.3 fold in kinase buffer at 6x the final assay concentration.

2 μL of testing compound solutions and 5 μL of enzyme solution were added into the 384-well microplate followed by a 10 minute incubation. The reaction was then initiated by the addition of 5 μL of peptide substrate and ATP mixture solution and incubated at 27° C. for 20 minutes. The enzyme reaction in each well was terminated by adding 4 μL of 120 mM EDTA solution. Each assay was carried out in duplicates.

DMSO was used as negative control (0%) for enzyme activity. 20 μM Staurosporine was used as positive control (100%) for enzyme activity.

Plates were read for fluorescence on a Caliper EZ Reader II @ by separating the fluorescent labeled substrate from the enzyme phosphorylation product by electrophoresis. Data were collected and analyzed using GraphPad Prism® version 6.0 software.

Dose-response curves were plotted from inhibition data generated, each in duplicate, from 10 point serial dilutions of inhibitory compounds. Concentration of compound was plotted against % kinase activity. To generate IC₅₀ values, the dose-response curves were then fitted to a standard sigmoidal curve and IC₅₀ values were derived by nonlinear regression analysis.

### 4) CDK4/Cyclin D3 Kinase Assay

CDK4/cyclin D3 kinase assays were performed in 384-well microplates (Corning, cat. No. 4512) and the final volume of the assay is 16 μL and reaction temperature is 27° C. Enzyme was diluted to a 2.4x dilution to provide a final concentration of 20 nM in kinase buffer containing 50 mM Tris HCl pH=8.0, 0.01% Tween 20, 50 μg/mL BSA, and 5 mM MgCl₂. The peptide substrate (5-FAM-YSPTSPSYSPTSPSYSPTSPSKKKK-NH₂) and ATP were diluted in kinase buffer at 2.4x dilution the final assay concentration to provide a mixture having final concentration of 2 μM of peptide substrate and 2 mM ATP. The test compounds were dissolved and diluted in DMSO. A stock DMSO solution of the test compound of the invention was prepared by dissolving the compound in DMSO to form a 10 mM solution. Ten points serial dilutions were made into DMSO using the 10 mM DMSO stock solutions of the test compounds to create a concentration gradient ranging from 25 nM to 500 μM (10 concentrations total). The test compound solutions of 10 gradient concentrations were further diluted at 8.3 fold in kinase buffer at 6x the final assay concentration.

2 μL of testing compound solutions and 5 μL of enzyme solution were added into the 384-well microplate followed by a 10 minute incubation. The reaction was then initiated by the addition of 5 μL of peptide substrate and ATP mixture solution and incubated at 27° C. for 180 minutes. The enzyme reaction in each well was terminated by adding 4 μL of 120 mM EDTA solution. Each assay was carried out in duplicates.

DMSO was used as negative control (0%) for enzyme activity. 20 μM Staurosporine was used as positive control (100%) for enzyme activity.

Plates were read for fluorescence on a Caliper EZ Reader II @ by separating the fluorescent labeled substrate from the enzyme phosphorylation product by electrophoresis. Data were collected and analyzed using GraphPad Prism® version 6.0 software.

Dose-response curves were plotted from inhibition data generated, each in duplicate, from 10 point serial dilutions of inhibitory compounds. Concentration of compound was plotted against % kinase activity. To generate IC₅₀ values, the dose-response curves were then fitted to a standard sigmoidal curve and IC₅₀ values were derived by nonlinear regression analysis.

### 5) CDK6/Cyclin D3 Kinase Assay

CDK6/cyclin D3 kinase assays were performed in 384-well microplates (Corning, cat. No. 4512) and the final volume of the assay is 16 μL and reaction temperature is 27° C. Enzyme was diluted to a 2.4× dilution to provide a final concentration of 20 nM in kinase buffer containing 20 mM MES pH=6.75, 0.01% Tween 20, 50 μg/mL BSA, and 5 mM MgCl₂. The peptide substrate (5-FAM-YSPTSPSYSPTSPSYSPTSPSKKKK-NH₂) and ATP were diluted in kinase buffer at 2.4× dilution the final assay concentration to provide a mixture having final concentration of 2 μM of peptide substrate and 2 mM ATP. The test compounds were dissolved and diluted in DMSO. A stock DMSO solution of the test compound of the invention was prepared by dissolving the compound in DMSO to form a 10 mM solution. Ten points serial dilutions were made into DMSO using the 10 mM DMSO stock solutions of the test compounds to create a concentration gradient ranging from 25 nM to 500 μM (10 concentrations total). The test compound solutions of 10 gradient concentrations were further diluted at 8.3 fold in kinase buffer at 6× the final assay concentration.

2 μL of testing compound solutions and 5 μL of enzyme solution were added into the 384-well microplate followed by a 10 minute incubation. The reaction was then initiated by the addition of 5 μL of peptide substrate and ATP mixture solution and incubated at 27° C. for 120 minutes. The enzyme reaction in each well was terminated by adding 4 μL of 120 mM EDTA solution. Each assay was carried out in duplicates.

DMSO was used as negative control (0%) for enzyme activity. 20 μM Staurosporine was used as positive control (100%) for enzyme activity.

Plates were read for fluorescence on a Caliper EZ ReaderII® by separating the fluorescent labeled substrate from the enzyme phosphorylation product by electrophoresis. Data were collected and analyzed using GraphPad Prism® version 6.0 software.

Dose-response curves were plotted from inhibition data generated, each in duplicate, from 10 point serial dilutions of inhibitory compounds. Concentration of compound was plotted against % kinase activity. To generate IC₅₀ values, the dose-response curves were then fitted to a standard sigmoidal curve and IC₅₀ values were derived by nonlinear regression analysis.

### 6) CDK9/Cyclin T1 Kinase Assay

CDK9/cyclin T1 kinase assays were performed in 384-well microplates (Corning, cat. No. 4512) and the final volume of the assay is 16 μL and reaction temperature is 27° C. Enzyme was diluted to a 2.4× dilution to provide a final concentration of 8 nM in kinase buffer containing 20 mM MES pH=6.75, 0.01% Tween 20, 50 μg/mL BSA, and 5 mM MgCl₂. The peptide substrate (Ahx-GSRTPMY-NH₂) and ATP were diluted in kinase buffer at 2.4× dilution the final assay concentration to provide a mixture having final concentration of 2 μM of peptide substrate and 2 mM ATP. The test compounds were dissolved and diluted in DMSO. A stock DMSO solution of the test compound of the invention was prepared by dissolving the compound in DMSO to form a 10 mM solution. Ten points serial dilutions were made into DMSO using the 10 mM DMSO stock solutions of the test compounds to create a concentration gradient ranging from 25 nM to 500 μM (10 concentrations total). The test compound solutions of 10 gradient concentrations were further diluted at 8.3 fold in kinase buffer at 6× the final assay concentration.

2 μL of testing compound solutions and 5 μL of enzyme solution were added into the 384-well microplate followed by a 10 minute incubation. The reaction was then initiated by the addition of 5 μL of peptide substrate and ATP mixture solution and incubated at 27° C. for 30 minutes. The enzyme reaction in each well was terminated by adding 4 μL of 120 mM EDTA solution. Each assay was carried out in duplicates.

DMSO was used as negative control (0%) for enzyme activity. 20 μM Staurosporine was used as positive control (100%) for enzyme activity.

Plates were read for fluorescence on a Caliper EZ Reader II by separating the fluorescent labeled substrate from the enzyme phosphorylation product by electrophoresis. Data were collected and analyzed using GraphPad Prism version 6.0 software.

Dose-response curves were plotted from inhibition data generated, each in duplicate, from 10 point serial dilutions of inhibitory compounds. Concentration of compound was plotted against % kinase activity. To generate IC₅₀ values, the dose-response curves were then fitted to a standard sigmoidal curve and IC₅₀ values were derived by nonlinear regression analysis.

The testing results for all enzyme assays are summarized in the Table 2 below.

TABLE 2
IC₅₀ values for the Deuterated Pyridopyrimidone Compounds
Tested in CDK Enzyme Selectivity Profiling Assays

| | IC₅₀ (nM) | | | | | |
|---|---|---|---|---|---|---|
| | CDK1/ | CDK2/ | CDK2/ | CDK4/ | CDK6/ | CDK9/ |
| Compound | CyclinB1 | CyclinA2 | CyclinE1 | CyclinD3 | CyclinD3 | CyclinT1 |
| Comp. 3 | 55.5 | 2.7 | 3.9 | 60.9 | 70.1 | 9272 |
| Comp. 4 | 22.3 | | 0.66 | 17 | 17.1 | 3211 |
| Comp. 5 | 25.5 | 1.5 | 1.9 | 34.3 | 32.8 | 3445 |
| Comp. 6 | 34.7 | 1.6 | 2.5 | 55.7 | 82.5 | 6449 |
| Comp. 11 | 52.8 | 2.4 | 3.1 | 51.2 | 64.8 | 8814 |
| Comp. 20 | 52.4 | 3.5 | 4.8 | 61.3 | 66.1 | 5998 |
| Comp. 28 | 22 | 1.2 | 1.5 | 32.4 | 30.8 | 3644 |
| Comp. 37 | 30 | | 0.8 | 28 | 40.8 | 4915 |
| Comp. 47 | 34.1 | | 1.8 | 47.6 | 76.5 | |
| Comp. 55 | 62.8 | | 3.3 | 75.9 | 104.2 | |
| Comp. 61 | 51.4 | | 2.14 | 73.9 | 152.8 | |
| Comp. 67 | 39 | | 0.86 | 34.4 | 62.5 | |
| Comp. 74 | 33.1 | | 0.77 | 35.6 | 37.5 | |
| Comp. 80 | 40.7 | | 1.01 | 37.2 | 47.81 | |
| Comp. 85 | 26.8 | | 1.07 | 32.6 | 45.7 | |
| Comp. 92 | 29.7 | | 1.16 | 38.5 | 37 | |
| Comp. 99 | 28.1 | | 1.02 | 36 | 30.1 | |
| Comp. 108 | 29.1 | | 1.08 | 34.2 | 58.4 | |
| Comp. 121 | 14.4 | | 0.8 | 17.4 | 24.4 | |
| Comp. 127 | 15.4 | | 1.2 | 28.8 | 36.4 | |
| Comp. 143 | 27.8 | | 1.2 | 33.3 | 30.1 | |
| Comp. 146 | 13.8 | | 0.7 | 21.3 | 24.0 | |
| Comp. 149 | 14.2 | | 1.0 | 24.4 | 33.0 | |
| Comp. 152 | 21.8 | | 1.2 | 29.7 | 30.0 | |
| Comp. 156 | 17.2 | | 0.7 | 19.7 | 27.1 | |
| Comp. 159 | 28.5 | | 1.1 | 25.2 | 30.0 | |
| Comp. 163 | 14.3 | | 0.7 | 22.8 | 28.3 | |
| Comp. 164 | 25.9 | | 1.4 | 42.7 | 40.7 | |
| Comp. 169 | 16.6 | | 0.8 | 23.4 | 33.6 | |
| LY2835219 | 3458 | 324.2 | 1302 | 12.1 | 22.3 | 1194 |
| (CDK4/6) | | | | | | |

CDK2 inhibitors having diverse structure features such as Comp. 3, Comp. 5, Comp. 6, Comp. 11, Comp. 20, and Comp. 28 gave nanomolar inhibition potency against CDK2 enzyme in in vitro enzyme assays. The IC₅₀ values for the CDK2 inhibition caused by the deuterated pyridopyrimidinone compounds as described herein is 10 fold or less than IC₅₀ values for the potency of the other CDKs such as CDK1, CDK4, CDK6, and CDK9 imparted by the same deuterated pyridopyrimidinone compounds.

### Example 37. Cell Proliferation Assay

The human ovarian carcinoma OVCAR3 cell line (ATCC HTB-161™) or human breast squamous HCC1806 cell line (ATCC CRL 2335) were obtained from American Type Culture Collection (ATCC), and maintained in culture media according to the supplier's specification.

The CyQUANT® Direct Cell Proliferation Assay Kit (Invitrogen™, catalog No. G35012) was utilized to test the inhibitory activities of the CDK2 inhibitors described in this disclosure against OVCAR3 or HCC1806 tumor cells.

CyQUANT® Direct Cell Proliferation Assay: The spent medium from the purchased tumor cell culture plate was removed and the tumor cells were washed with PBS buffer twice. Trypsin (VWR catalog No. 0458-25G) was added to digest the tumor cells until tumor cells appeared round shaped and to dissociate adherent cells from the plate wall in which they are being cultured. Then RPMI-1640 media (HCC1806, Gibco™ catalog No, A10491-01) with 10% fetal bovine serum (FBS) (Hyclone™ catalog No. SV30160.03), or RPMI-1640 media (OVCAR-3, Gibco™ catalog No. 10370-021) with 20% FBS and 0.01 mg/mL insulin was added to stop the trypsin digestion reaction. The tumor cells were lightly agitated with a liquid transfer pipette to cause tumor cells suspended in the culture media. The cells were counted. The complete medium was used to prepare tumor cell suspension. The concentration of HCC1806 cells in the suspension is 3.75×10³ cells/mL and the concentration of OVCAR-3 cells in the suspension is 3.75×10⁴ cells/mL.

OVCAR3 or HCC1806 cells were seeded at 40 μL/well in 384-well plates in growth media containing FBS and cultured 24 hours in the incubator at 37° C., 5% CO₂. The following day, compounds were dissolved in DMSO (BioRoYee. catalog No. AF0231) and serially diluted in growth media for final concentrations including 10, 3.33, 1.11, 0.37, 0.123, 0.041, 0.0137, 0.0046, 0.0015 μM/L. The concentration of DMSO is less than 0.1%. Compound solutions were added by Agilent Bravo liquid handler. Each group has three parallel wells and 10 μL of compound solution was added to each well. The control wells contained the growth medium having 0.1% DMSO. Cells were incubated at 37° C., 5% CO₂ for 7 days.

CYQUANT Direct Cell Proliferation Assay (Molecular Probes, Eugene, Oreg.) was then performed following manufacturer's instructions to prepare the detection reagents. 50 μL of detection reagent was aliquot and added to each well and the cells were incubated at 37° C. for 1 hr.

The cell culture plates were placed on TECAN M1000 microplate reader to detect the fluorescence intensity at 508 nM excitation and 527 nM emission wavelengths. Graphpad Prism 6.0 software was used for data processing, preparing cell growth curve, calculating percentage inhibition on cell growth. IC₅₀ values were calculated by concentration-response curve fitting utilizing a four-parameter analytical method using GraphPad Prism software.

The IC₅₀ results for the CDK2 inhibitors in the HCC1806 and OVCAR-3 cell proliferation assays are summarized in the Table 3 below.

TABLE 3
HCC1806 and OVCAR-3 cell
Proliferation Assay Results

| | IC₅₀ (nM) | |
|---|---|---|
| Entry | HCC1806 | OVCAR-3 |
| Comp. 1 | 86.49 | 54.31 |
| Comp. 2 | 58.99 | 115.72 |
| Comp. 3 | 53.38 | 38.53 |
| Comp. 4 | 41.5 | |
| Comp. 5 | 52.1 | 40.34 |
| Comp. 6 | 56.1 | 39.25 |
| Comp. 11 | 28.57 | 21.29 |
| Comp. 20 | 124.64 | 104.23 |
| Comp. 28 | 75 | 32.5 |
| Comp. 37 | 35.0 | |
| Comp. 61 | 69.2 | |
| Comp. 67 | 65.9 | |
| Comp. 74 | 83.7 | |
| Comp. 80 | 51.2 | |
| Comp. 85 | 84.5 | |
| Comp. 92 | 67.7 | |
| Comp. 99 | 65.5 | |
| LY2835219 | 160.7 | 4370.02 |

The structure (deuterium incorporation) and the activity (in vitro cell proliferation inhibitory activities) relationship for the compounds in Table 3 are surprising in that the impact of the deuterium substitution at a given substituent of the molecule on the potency of the cell proliferation inhibition is unpredictable. For example, deuterated Comp. 28 (R²=—CF₂D in Formula (3)) is about 1.4 fold less potent than that of non-deuterated Comp. 5 (R²=—CF₂H) in the cell proliferation inhibitory potency in HCC1806 (see entries for Comp. 5 and Comp. 28 in Table 3 above); whereas deuterated Comp. 11 (R²=—CD₃ in Formula (1)) is about two fold more potent than that of non-deuterated Comp. 3 (R²=—CH₃) in the cell proliferation inhibitory potency in HCC1806 and OVCAR-3 cell proliferation assays (see entries for Comp. 3 and Comp. 11 in Table 3 above).

### Example 38. In Vitro Liver Microsome Stability Studies

The metabolic stability of various deuterated pyridopyrimidinone compounds of Formulae (1)-(5) was examined using human and mouse liver microsomes.

### Material

Human liver microsomes (cat. No. 452161), mouse liver microsomes (cat. No. 452701), were purchased from Corning.

### Analytical LC/MS Operating Parameters

Samples were analyzed using LC-MS/MS using a SCIEX API 4000 mass spectrometer coupled with Agilent HPLC analytical system. After separation on a ZORBAX XDB-C18 column (5 μm, 50×2.1 mm, column No. 50-282) using an acetonitrile-water gradient system, peaks were analyzed by mass spectroscopy (MS) using ESI ionization in Q1 and Q3 scan mode.

The LC and MS are all controlled by Analyst software. LC operation conditions are as the following: mobile Phase A: water with 0.1% formic acid; mobile Phase B: acetonitrile with 0.1% formic acid; t=0.5 min, 25% Phase B and 75% Phase A; t=1.10 min, 98% Phase B and 2% Phase A; t=1.90 min, 98% Phase B and 2% Phase A; t=1.91 min, 25% Phase B and 75% Phase A; t=3.0 min, stop run.

### Liver Microsome Stability Profiling Tests

The liver microsomes were incubated with 1.0 μM of the testing compound at 37° C. The incubation mixture contained 100 mM PBS buffer (pH=7.4), 3 mM MgCl₂ and 1 mM NADPH. The concentration of liver microsome in the incubation mixture was 0.5 mg/mL for testing compound, and 0.2 mg/mL for MDZ and the total volume is 0.2 mL. Aliquots were withdrawn at time points of 0 min, 5 min, 15 min, 30 min, and 60 min for testing compounds. Aliquots were withdrawn at time points of 0 min, 5 min and 20 min for MDZ. The reactions were terminated by transferring the aliquots to a methanol solution containing the internal standard Midazolam (MDZ) for analytical quantification. The quenched samples were analyzed by LC-MS/MS. The incubations were carried out in duplicates. The metabolic stability of the testing compounds by the liver microsomes of human and mice is expressed as elimination half-life (t_{1/2}). The results are shown as the means of duplicate determinations±SD.

The testing results for the in vitro liver microsome stability profiling for the testing compounds are summarized in Table 4 below.

TABLE 4
t_{1/2} results in human and mouse
liver microsome stability tests

| | t_{1/2} (min) | |
|---|---|---|
| Compound | human | mouse |
| Comp. 4 | 462 | 16.5 |
| Comp. 5 | 2000 | 22.0 |
| Comp. 6 | 693 | 24.2 |
| Comp. 28 | 866.3 | 182 |
| Comp. 37 | 1732.5 | 72 |
| Comp. 47 | 990 | 24.5 |
| Comp. 55 | 577.5 | 23.5 |
| Comp. 61 | 346 | 21.4 |
| Midazolam | 16.3 | 4.59 |

According to the testing results in Table 4, it is found that about 4 fold of increase in half-life (t_{1/2} min) in human and mouse liver microsome incubations for the deuterated Comp. 37 (R²=—CD₃ in Formula (3), t_{1/2} human=1732 min, t_{1/2} mouse=72 min) as compared with the undeuterated Comp. 4 (R=—CH₃, t_{1/2} human=462 min, t_{1/2} mouse=16.5 min).

The liver microsome assay data in Table 4 also surprisingly showed that deuteration at different positions on the compounds of Formulae (1)-(5) do not always resulted in improved metabolic stability, for example, Comp. 47 having mono-deuteration at R¹⁵ position in Formula (3) (R¹⁵=D) resulted increased metabolic stability as evidenced by increased half-life t_{1/2} human=990 min, t_{1/2} mouse=24.5 min, whereas Comp. 61 having tetra-deuterium substitution at R⁶, R⁷, R¹³, R¹⁴ positions in Formula (3) (t_{1/2} human=577.5 min, t_{1/2} mouse=23.5 min) and Comp. 55 having tri-deuterium substitution at R¹⁰ position in Formula (3) (t₂ human=346 min, t_{1/2} mouse=21.4 min) resulted decreased metabolic stability as compared with that of the non-deuterated Comp. 6 (t_{1/2} human=693 min, t_{1/2} mouse=24.2 min); whereas as compared with the undeuterated compound Comp. 6 (t_{1/2} human=693 min, t_{1/2} mouse=24.2 min) (See Table 4). Thus, the liver microsome incubation profiling results disclosed herein indicated that the impacts of the deuterium substitution at a given location on the compound of Formula (1)-(5) upon the metabolic stability is unpredictable.

### Example 39. Pharmacokinetic Profiling in Mouse Model

### Materials

(1). Kolliphor® HS 15 (formerly regarded as Solutol® HS 15) a non-ionic surfactant, was used as a solubilizer in preparing the parenteral formulations; (2). DMA (N,N-dimethylacetamide); (3). saline; and (4). Tween 80; (5) acetyl nitrile. CDK2 inhibitors Comp. 4, Comp. 5, and Comp. 37 were prepared according to the procedures set forth in the Examples 4, 5 and 10 above.

### Test Sample Formulation Preparation

1). Test Sample Formulations for Intravenous Injections

Testing compound formulations of Comp. 4, Comp. 5, and Comp. 37 for intravenous injection (iv) were prepared at 1 mg/mL concentration in a liquid medium containing 10:10:80 (v/v/v) of DMA:30% solution HS 15:saline.

To a glass flask was added 6.0 μmol of the test compound, and 0.265 mL of DMA. The glass flask was vortexed to allow the complete dissolution of the solid. Then 0.265 mL of 30% Solution HS 15 was added to the test compound solution in DMA and was vortexed to allow homogeneous mixing. Lastly, 2.12 mL of saline was added to the mixture in the glass flask. The flask was vortexed to allow homogeneous mixing. The resulting liquid was filtered with a 0.45 μm nylon membrane (PALL™). A clear liquid test sample iv formulation was obtained. An aliquot of 100 μL of the test sample iv formulation was placed into a 1.5 mL EP tube and was kept at 2-8° C. for future sample concentration quantification by HPLC.

For sample concentration measurement, the stock aliquot in 1.5 mL EP tube was diluted to a concentration of 100 μg/mL in a medium made of acetonitrile/water (1:1 v/v). The injection volume for the concentration measurement is 200 μL.

2) Test Sample Formulation for Intragastric (IG) Administration

Testing compound formulations of Comp. 4, Comp. 5, and Comp. 37 for intragastric (IG) route of administration were prepared at 3 mg/mL concentration in an aqueous solution containing 0.5% methyl cellulose and 0.1% Tween 80.

To a glass flask was added 27 μmol of the test compound and 4.047 mL of 0.5% MC with 01. % Tween 80. The flask and the mixture was vortexed followed by sonication for about 15 minutes to allow even distribution of the solids. A white suspension of the text sample was obtained.

An aliquot of 100 μL of the test sample IG formulation was placed into a 1.5 mL EP tube and was kept at 2-8° C. for future sample concentration quantification by HPLC.

### Animal Preparation

A total of 54 male ICR mice having average 19.7 g to 23.5 g body weight were purchased from Vital River Laboratory Animal Technologies Co. Ltd. Before receiving the test compound formulations, the animals were on fasting overnight. On the day of dosing, the animals were fed at 4h after the dosing. During the tests, the animals were free to drink water.

Each test compound was tested in duplicate with different routes of administration: in one set of tests, mice received test compounds by iv injection route, and in the other set of tests, mice received the test compounds by IG administration. The schedules of the test for each mouse are illustrated in Table 5 below.

TABLE 5
Study Design

| | | | | | Dose | Cone. | Vol. | Sampling |
|---|---|---|---|---|---|---|---|---|
| | Animal | | | | (mg · | (mg · | (mL · | time |
| Group | ID | Comp. | N | Route | kg⁻¹) | mL⁻¹) | kg⁻¹) | point |
| A | A1 | Comp. | 3 | i.v. | 5 | 1 | 5 | before dosing, |
| | A2 | 4 | | | | | | 30 min and 4 h |
| | A3 | | | | | | | after dosing |
| | A4 | Comp. | 3 | i.v. | 5 | 1 | 5 | 5 min, 1 h, and |
| | A5 | 4 | | | | | | 8 h after dosing |
| | A6 | | | | | | | |
| | A7 | Comp. | 3 | i.v. | 5 | 1 | 5 | 15 min, 2h, |
| | A8 | 4 | | | | | | and 24 h after |
| | A9 | | | | | | | dosing |
| B | B1 | Comp. | 3 | i.g. | 30 | 3 | 10 | before dosing, |
| | B2 | 4 | | | | | | 1 h and 8 h |
| | B3 | | | | | | | after dosing |
| | B4 | Comp. | 3 | i.g. | 30 | 3 | 10 | 15 min, 2 h and |
| | B5 | 4 | | | | | | 24 h after |
| | B6 | | | | | | | dosing |
| | B7 | Comp. | 3 | i.g. | 30 | 3 | 10 | 30 min and 4 h |
| | B8 | 4 | | | | | | after dosing |
| | B9 | | | | | | | |
| C | C1 | Comp. | 3 | i.v. | 5 | 1 | 5 | before dosing, |
| | C2 | 37 | | | | | | 30 min and 4 h |
| | C3 | | | | | | | after dosing |
| | C4 | Comp. | 3 | i.v. | 5 | 1 | 5 | 5 min, 1 h and |
| | C5 | 37 | | | | | | 8 h |
| | C6 | | | | | | | |
| | C7 | Comp. | 3 | i.v. | 5 | 1 | 5 | 15 min, 2h and |
| | C8 | 37 | | | | | | 24 h after |
| | C9 | | | | | | | dosing |
| D | D1 | Comp. | 3 | i.g. | 30 | 3 | 10 | before dosing, |
| | D2 | 37 | | | | | | 1 h and 8 h |
| | D3 | | | | | | | after dosing |
| | D4 | Comp. | 3 | i.g. | 30 | 3 | 10 | 15 min, 2 h and |
| | D5 | 37 | | | | | | 24 h after |
| | D6 | | | | | | | dosing |
| | D7 | Comp. | 3 | i.g. | 30 | 3 | 10 | 30 min and 4 h |
| | D8 | 37 | | | | | | after dosing |
| | D9 | | | | | | | |
| E | E1 | Comp. | 3 | i.v. | 5 | 1 | 5 | before dosing, |
| | E2 | 5 | | | | | | 30 min and 4 h |
| | E3 | | | | | | | after dosing |
| | E4 | Comp. | 3 | i.v. | 5 | 1 | 5 | 5 min, 1 h, and |
| | E5 | 5 | | | | | | 8 h after dosing |
| | E6 | | | | | | | |
| | E7 | Comp. | 3 | i.v. | 5 | 1 | 5 | 15 min, 2 h, |
| | E8 | 5 | | | | | | 24 h after |
| | E9 | | | | | | | dosing |
| F | F1 | Comp. | 3 | i.g. | 30 | 3 | 10 | before dosing, |
| | F2 | 5 | | | | | | 1 h and 8 h after |
| | F3 | | | | | | | dosing |
| | F4 | Comp. | 3 | i.g. | 30 | 3 | 10 | 15 min, 2 h, |
| | F5 | 5 | | | | | | and 24 h after |
| | F6 | | | | | | | dosing |
| | F7 | Comp. | 3 | i.g. | 30 | 3 | 10 | 30 min, and 4 |
| | F8 | 5 | | | | | | h after dosing |
| | F9 | | | | | | | |

### Plasma Sample Preparation

100 μL blood sample was taken from the veins in animal eyelid. The whole blood sample was placed in anticoagulation tubes treated with EDTA-K2. The whole blood sample was centrifuged at 1500 g for 10 min. The plasma at the upper level was collected into test tubes. An aliquot of 10 μL of sample was added with 100 μL ACN which contains of Verapamil, 5 ng/mL, Glibenclamide, 50 ng/mL, Tolbutamide, 200 ng/mL and Diclofenac, 200 ng mL-1 for protein precipitation. The mixture was vortexed for 1 min, then centrifuged at 13000 rpm for 8 min. Then 70 μL of supernatant was added with 70 μL water, then vortexed for 10 min. An aliquot of 5 μL of the mixture was injected into the LC-MS/MS system.

### Analytical LC Operating Parameters

LC-MS/MS (API 4000: LC-MS-MS-001), column: Waters® Xbridge-C18 (5 μm, 50×2.1 mm) (column No. 50-223), mobile Phase A: water with 0.1% formic acid; mobile Phase B: acetonitrile with 0.1% formic acid. LC operation parameters: t=0.5 min, 25% Phase B and 75% Phase A; t=1.00 min, 98% Phase B and 2% Phase A; t=1.90 min, 98% Phase B and 2% Phase A; t=1.91 min, 25% Phase B and 75% Phase A; t=3.0 min, stop run. Sample injection volume is 5 μL. Flow rate is 0.8 mL/min. The detection wavelength is set at 254 nm.

The results for the pharmacokinetic studies in mice are summarized in Table 6 below.

TABLE 6
Mouse PK Test Results

| PK Parameters | Unit | Comp. 4 | Comp. 37 | Comp. 5 |
|---|---|---|---|---|
| | | i.v. 5 mg/kg | | |
| C₀ | ng · mL⁻¹ | 8300 | 7420 | 5020 |
| T_{1/2} | h | 0.195 | 0.330 | 0.367 |
| AUC₀₋ₜ | ng · h · mL⁻¹ | 967 | 1290 | 1360 |
| CL | mL · kg⁻¹ · min⁻¹ | 86.1 | 64.7 | 61.2 |
| Vdₛₛ | L · kg⁻¹ | 0.611 | 0.703 | 1.00 |
| | | i.g. 30 mg/kg | | |
| T_{1/2} | h | 2.24 | 1.72 | 2.76 |
| Tₘₐₓ | h | 0.250 | 0.250 | 0.250 |
| Cₘₐₓ | ng · mL⁻¹ | 211 | 522 | 1040 |
| AUC₀₋ₜ | ng · h · mL⁻¹ | 245 | 605 | 1190 |
| *F | % | 4.2 | 7.8 | 14.6 |

Note:
*F (bioavailability) was calculated by using the AUC₀₋ₜ.

### Example 40. Efficacy of Deuterated Pyridopyrimidinone Compounds in HCC1806 s.c. Breast Cancer Xenografts

The in vivo potency of anti-tumor activities of Comp. 4, Comp. 37, Comp. 5, Comp. 28, Comp. 163, and Comp. 164 have been evaluated on the growth of HCC1806 s.c. xenografts in BALB/c nude mice.

### Materials

The testing compounds Comp. 4, Comp. 37, Comp. 5, Comp. 28, Comp. 163, and Comp. 164 were prepared according the synthetic routes set forth in the above Examples. The testing formulations for compounds Comp. 4, Comp. 37, Comp. 5, Comp. 28, Comp. 163, and Comp. 164 were prepared as liquid formulation in a liquid vehicle containing 0.1% Tween-80 (purchased form Sinopharm Chemical Reagent Co., Ltd, Lot No. 20190322), 0.5% methyl cellulose (purchased from SIGMA, Lot No. SLBR8963V) in water.

RPMI-1640 medium used for cell culturing HCC1806 was purchased from Corning cellgro, Cat No.: 10-040-CV; FBS was purchased from GIBCO™, Cat No.: 10270-106; PBS buffer was purchased from Corning cellgro, Cat No.: 21-040-CVR; Trypsin-EDTA was purchased from GIBCO™, REF: 25200-072; Penicillin-Streptomycin was purchased from GIBCO™, REF: 15140-122.

### Equipment

Centrifuge: Thermo Fisher, model: Thermo Fisher legend MACH 1.6;

Inverted Microscope: Nikon, model: TS100;

Biological Safety Cabinet: Thermo Fisher, model: Thermo MSC-Advantage;

CO₂ Incubator: Thermo Fisher, model: Thermo HERA cell 150

### Animal Preparation

A total of 42+8 female BALB/c nude mice were ordered from Shanghai LC Laboratory Animal Co. Ltd. The animals were specific pathogen free and approximately 4-5 weeks old upon arrival at PharmaLegacy Laboratories.

The procedures that were applied on animals in this protocol have been approved by PharmaLegacy Laboratories IACUC in advance before their execution. The specifications of the animals are summarized in Table 7 below.

Upon receipt the animals were unpacked and placed in cages. A health inspection was performed on each animal to include evaluation of the coat, extremities and orifices. Each animal was also examined for any abnormal signs in posture or movement.

The animals were housed in the PharmaLegacy Laboratories vivarium in clear polycarbonate plastic cages (260 mm×160 mm×120 mm); 2-6 animals per cage. The bedding material was corn-cob bedding (irradiated, Shandong Goodway Biotechnology Co., Ltd., China) that was changed once a week. The room number in which the animals were housed throughout the study period was detailed in the study records. The room was supplied with HEPA filtered air at the rate of 15-25 air changes per hour. The temperature was maintained at 20° C. to 26° C. (68° F. to 79° F.) with a relative humidity of 40% to 70%. Temperature and humidity were continuously monitored and recorded. Illumination was fluorescent light for 12-hour light (08:00-20:00) and 12-hour dark.

Animals had ad libitum access to rodent food (irradiated, Nanjing Xietong Organism Technology Co. Ltd., China). The manufacturer had supplied a certificate of analysis for each batch of diet received by PharmaLegacy Laboratories. The Certificates of analysis were retained in the PharmaLegacy Laboratories archives.

Water, from the municipal water supply, was filtered by reverse osmosis or high-pressure sterilizer adjusted to pH 2-3 with HCl. Water analyses were performed twice per year and included analyses of heavy metals, nitrates, dissolved minerals, total plate count and coliforms. Certificates of analysis were retained in the PharmaLegacy Laboratories archives.

A unique number was assigned to each animal. Prior to the allocation of animals to inoculation groups, cages were labeled with cards identifying study number, species/strain, sex, cage number and animal number. After allocation to inoculation groups the cages were labeled with cards which were color coded, and identify inoculation groups as well as the information outlined above. Group allocation was documented in the randomization records. Cages were stratified within the racks to reduce the effect of any environmental influences on the study.

TABLE 7
Animal Specifications

| Animal species | |
|---|---|
| and strain: | BALB/c nude mice |
| History of treatment: | Naive |
| Sex, age and weight: | Female, 5-6 weeks |
| Breeder/supplier: | Shanghai LC Laboratory Animal Co. Ltd. |
| Test Facility: | PharmaLegacy Laboratories Vivarium |
| Adaptation: | At least 7 days |
| Room: | SPF Room |
| Room temperature: | 20-26° C. |
| Room relative | 40-70% |
| humidity: | |
| Light cycle: | Fluorescent light for 12-hour light (08:00-20:00) |
| | and 12-hour dark |
| Animal hosting: | 2-6 mice/cage each group |
| Food: | Free access to food (irradiated, Nanjing Xietong |
| | Organism Technology Co. Ltd., China) |
| Water: | Free access to water (municipal tap water filtered |
| | by reverse osmosis or high-pressure sterilizer |
| | adjusted to pH 2-3) |

### Experimental Procedures

HCC1806 cell cultures were maintained at 37° C. under 5% CO₂ in RPMI-1640 medium supplemented with 10% FBS and were subsequently cultured within 10 passages before inoculated into the mice.

Approximately 1×107 HCC1806 cells were suspended in PBS was injected via s.c. to each animal under anesthesia by 3% to 4% isoflurane. When the average tumor volume reached 80-120 mm³, 42 tumor-bearing mice were randomized into 7 groups (6 mice per group) according to tumor volume. The grouping day was defined as Day 0. Treatment was applied immediately after grouping animals on the same day. The groups were indicated in the Table 8 below.

TABLE 8
Grouping and Dosing Regimen

| | Test | | Dose | | Dosing |
|---|---|---|---|---|---|
| Group | Compound | N | (mg/kg) | Route | Regimen |
| 1 | Vehicle | 6 | N/A | P.O. | BID, 21 days |
| 2 | Comp. 4 | 6 | 30 | P.O. | BID, 21 days |
| 3 | Comp. 37 | 6 | 30 | P.O. | BID, 21 days |
| 4 | Comp. 5 | 6 | 30 | P.O. | BID, 21 days |
| 5 | Comp. 28 | 6 | 30 | P.O. | BID, 21 days |
| 6 | Comp. 163 | 6 | 30 | P.O. | BID, 21 days |
| 7 | Comp. 164 | 6 | 30 | P.O. | BID, 21 days |

N: animal number per group;
P.O.: oral administration;
BID: twice a day

Animals were observed every day for health status and general reaction from the day of inoculation. All exceptions to normal healthy appearance and behavior were recorded and detailed in standard PharmaLegacy Laboratories clinical observations forms.

The body weight of the testing animals were measured twice a week after grouping. Tumor volumes by the length and width of the tumor were measured by electric calipers and the tumor volume (V) was calculated as follows: V=(length×width²)/2. The individual relative tumor volume (RTV) was calculated as follows: RTV=Vₜ/V₀, where Vt was the volume on each day, and V0 was the volume at the beginning of the treatment.

Tumor growth inhibition (TGI)=(1−(Tᵢ−T₀)/(Cᵢ−C₀))×100%; Tᵢ and Cᵢ is the mean tumor volumes of the treatment and control groups on the measurement day; T₀ and C₀ is the mean tumor volumes of the treatment and control groups on day 0.

Survival rate of mice treated with control vehicle and the inventive compounds described herein were sacrificed before the end point if >20% weight loss occur or the tumor reach the maximum permitted size (2,000 mm³).

Plasma was collected at 10 min and 6 hr after the first administration of Day 0 and Day 20. Animals were anesthetized with 2.0% to 3.5% isoflurane. Approximately 0.1 mL blood was collected via orbital vein per time point. The blood was collected into anticoagulant tubes (EDTA-K2) and spun down immediately to generate plasma according to standard procedures. Plasma was frozen immediately in liquid nitrogen and placed in −70° C. to −80° C. for storage. The plasma samples were sent to Shuangliang for analysis.

Tumors were collected at the end of the study (Day 21), the mice were sacrificed. Tumor was collected, weighed, photographed.

To evaluate necropsy in the testing animals, animals were euthanized by CO₂ asphyxiation first, and then cervical dislocation at the end of the in vivo study, or when it met with any one of the following conditions (unless an exemption upon the sponsor's request was approved by the PharmaLegacy attending veterinarian or the IACUC in writing): (1) Tumor volume: the average tumor volume in one certain group exceeded 2000 mm³. (2) Tumor ulceration and necrosis: Tumor ulceration became approximately 25% or greater of the surface of the tumor, or the animal chewed on the lesion or pays undue attention to the ulcer. (3) Animal functions impairment: the tumor interfered with normal animal functions (e.g. eating, drinking, or ambulating). (4) Other disease symptoms.

### Testing Results

Results were expressed as mean±S.E.M. Comparisons between two groups were made by corresponding test, p<0.05 were considered to be significant.

i. Body Weight

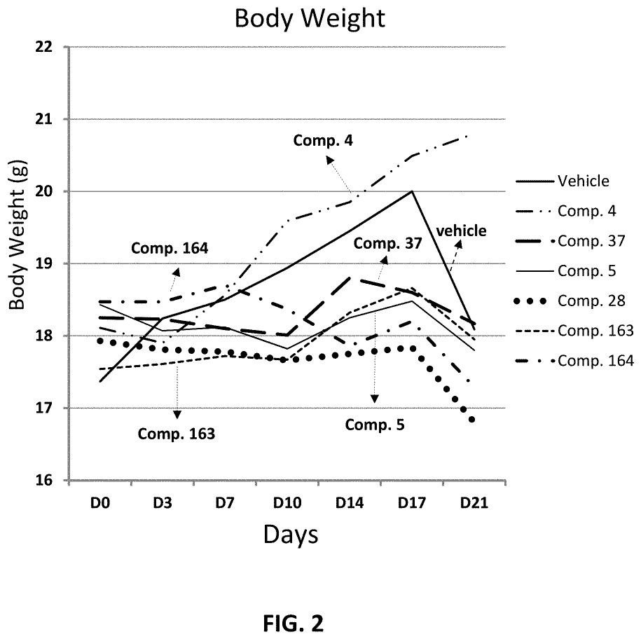

Body weight of each animal was recorded twice a week. As shown in Tables 9-10 and FIG. 2, when comparing with G1 vehicle, body weight of animals in G5 Comp. 28 was significant lower on day 17, and body weight of animals in G2 Comp. 4 was significant higher on day 21.

TABLE 9
Body weight
Body weight (g)

| Group | | D0 | D3 | D7 | D10 | D14 | D17 | D21 |
|---|---|---|---|---|---|---|---|---|
| G1 | Mean | 17.37 | 18.24 | 18.50 | 18.94 | 19.45 | 20.00 | 18.09 |
| Vehicle | SEM | 0.34 | 0.35 | 0.45 | 0.49 | 0.50 | 0.50 | 0.44 |
| G2 | Mean | 18.11 | 17.90 | 18.56 | 19.59 | 19.85 | 20.49 | 20.80 |
| Comp. 4 | SEM | 0.39 | 0.74 | 0.57 | 0.43 | 0.46 | 0.50 | 0.57 |
| G3 | Mean | 18.25 | 18.23 | 18.10 | 18.01 | 18.80 | 18.60 | 18.17 |
| Comp. | SEM | 0.43 | 0.44 | 0.49 | 0.58 | 0.50 | 0.65 | 0.77 |
| 37 | | | | | | | | |
| G4 | Mean | 18.43 | 18.07 | 18.12 | 17.82 | 18.25 | 18.48 | 17.80 |
| Comp. 5 | SEM | 0.56 | 0.51 | 0.56 | 0.49 | 0.57 | 0.58 | 0.72 |
| G5 | Mean | 17.93 | 17.81 | 17.78 | 17.66 | 17.75 | 17.84 | 16.80 |
| Comp. | SEM | 0.45 | 0.38 | 0.33 | 0.48 | 0.49 | 0.53 | 0.55 |
| 28 | | | | | | | | |
| G6 | Mean | 17.54 | 17.61 | 17.72 | 17.67 | 18.32 | 18.66 | 17.95 |
| Comp. | SEM | 0.45 | 0.51 | 0.54 | 0.53 | 0.46 | 0.45 | 0.72 |
| 163 | | | | | | | | |
| G7 | Mean | 18.47 | 18.47 | 18.70 | 18.37 | 17.86 | 18.20 | 17.29 |
| Comp. | SEM | 0.27 | 0.28 | 0.15 | 0.45 | 0.50 | 0.63 | 0.73 |
| 164 | | | | | | | | |

ii. Tumor Volume

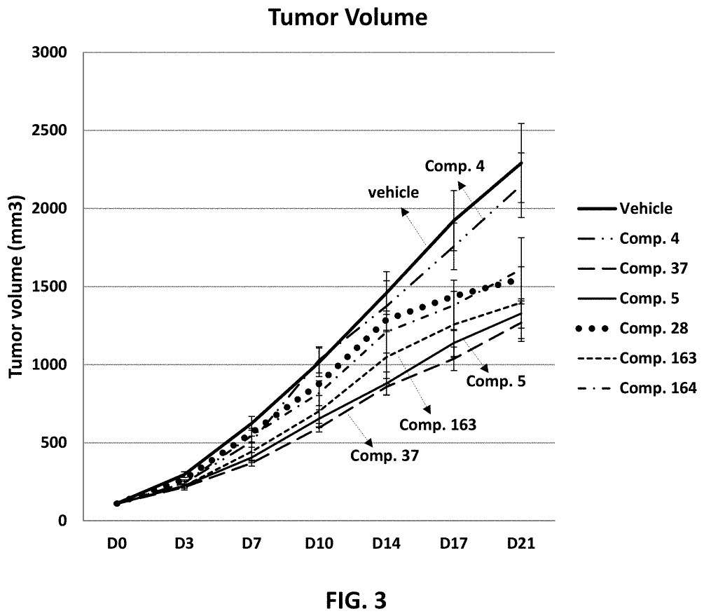

Tumor volume was measured and calculated twice a week and presented in Table 10 and FIG. 3. When comparing with G1 vehicle on each day, animals treated with CDK-1700225 (G3) showed a significant smaller tumor volume from day 3 to day 21, animals treated with CDK-1700226 (G4) showed a significant smaller tumor volume from day 7 to day 21, and animals treated with CDK-1700313 (G6) showed a significant smaller tumor volume on day 7, 17 and 21.

TABLE 10
Tumor volume
Tumor volume (mm³)

| Group | | D0 | D3 | D7 | D10 | D14 | D17 | D21 |
|---|---|---|---|---|---|---|---|---|
| G1 | Mean | 112.28 | 296.91 | 624.59 | 1015.12 | 1458.99 | 1922.30 | 2291.29 |
| Vehicle | SEM | 3.94 | 18.46 | 44.23 | 91.74 | 136.74 | 192.98 | 253.30 |
| G2 | Mean | 112.95 | 241.73 | 506.86 | 1030.80 | 1374.19 | 1757.63 | 2149.25 |
| Comp. 4 | SEM | 3.16 | 11.53 | 35.33 | 83.34 | 163.26 | 149.21 | 207.53 |
| G3 | Mean | 109.89 | 216.01 | 370.15 | 596.52 | 858.29 | 1037.86 | 1269.08 |
| Comp. | SEM | 3.83 | 18.09 | 19.26 | 27.04 | 53.71 | 75.08 | 120.16 |
| 37 | | | | | | | | |
| G4 | Mean | 109.77 | 225.24 | 405.37 | 654.51 | 880.11 | 1139.14 | 1327.79 |
| Comp. 5 | SEM | 2.85 | 13.36 | 32.93 | 57.39 | 73.14 | 85.91 | 93.57 |
| G5 | Mean | 111.13 | 267.25 | 564.40 | 876.82 | 1288.11 | 1438.12 | 1549.07 |
| Comp. | SEM | 4.73 | 32.17 | 54.91 | 110.16 | 195.24 | 192.14 | 202.85 |
| 28 | | | | | | | | |
| G6 | Mean | 110.66 | 223.62 | 443.83 | 702.37 | 1048.55 | 1257.00 | 1397.61 |
| Comp. | SEM | 4.21 | 15.12 | 60.82 | 100.21 | 171.52 | 211.92 | 229.90 |
| 163 | | | | | | | | |
| G7 | Mean | 112.74 | 243.14 | 545.66 | 817.53 | 1209.55 | 1380.25 | 1610.30 |
| Comp. | SEM | 3.80 | 20.15 | 47.73 | 79.26 | 134.48 | 161.19 | 202.50 |
| 164 | | | | | | | | |

iii. Relative Tumor Volume

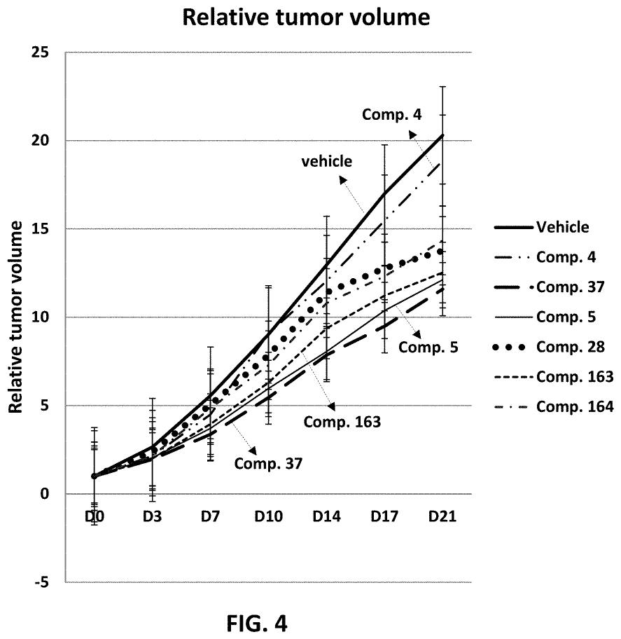

As shown in Table 11 and FIG. 4, relative tumor volume of each group was gradually increased throughout the study. When comparing with vehicle, Comp. 37 (G3) and Comp. 5 (G4) were able to decrease the relative tumor weight from day 3 to day 21 significantly, Comp. 163 (G6) could decrease the relative tumor weight on day 3, 7, 10, 17 and 21 and Comp. 28 (G5) and Comp. 164 (G7) were able to decrease the relative tumor weight on day 21.

TABLE 11
Relative tumor volume

| Group | | D0 | D3 | D7 | D10 | D14 | D17 | D21 |
|---|---|---|---|---|---|---|---|---|
| G1 | Mean | 1.00 | 2.65 | 5.56 | 9.03 | 12.96 | 17.00 | 20.30 |
| Vehicle | SEM | 0.00 | 0.14 | 0.31 | 0.72 | 1.06 | 1.37 | 1.98 |
| G2 | Mean | 1.00 | 2.15 | 4.49 | 9.08 | 12.05 | 15.47 | 18.87 |
| Comp. 4 | SEM | 0.00 | 0.10 | 0.27 | 0.56 | 1.21 | 1.01 | 1.44 |
| G3 | Mean | 1.00 | 1.96 | 3.37 | 5.45 | 7.86 | 9.49 | 11.59 |
| 37 | | | | | | | | |
| Comp. | SEM | 0.00 | 0.13 | 0.11 | 0.22 | 0.61 | 0.78 | 1.19 |
| G4 | Mean | 1.00 | 2.05 | 3.71 | 5.97 | 8.06 | 10.38 | 12.12 |
| Comp. 5 | SEM | 0.00 | 0.10 | 0.34 | 0.53 | 0.74 | 0.75 | 0.86 |
| G5 | Mean | 1.00 | 2.38 | 5.05 | 7.85 | 11.40 | 12.77 | 13.76 |
| 28 | | | | | | | | |
| Comp. | SEM | 0.00 | 0.23 | 0.36 | 0.83 | 1.45 | 1.38 | 1.46 |
| G6 | Mean | 1.00 | 2.01 | 3.95 | 6.30 | 9.37 | 11.22 | 12.53 |
| 163 | | | | | | | | |
| Comp. | SEM | 0.00 | 0.07 | 0.45 | 0.79 | 1.37 | 1.69 | 1.86 |
| G7 | Mean | 1.00 | 2.15 | 4.85 | 7.29 | 10.80 | 12.31 | 14.35 |
| 164 | | | | | | | | |
| Comp. | SEM | 0.00 | 0.14 | 0.39 | 0.72 | 1.26 | 1.50 | 1.79 |

iv. Tumor Weight

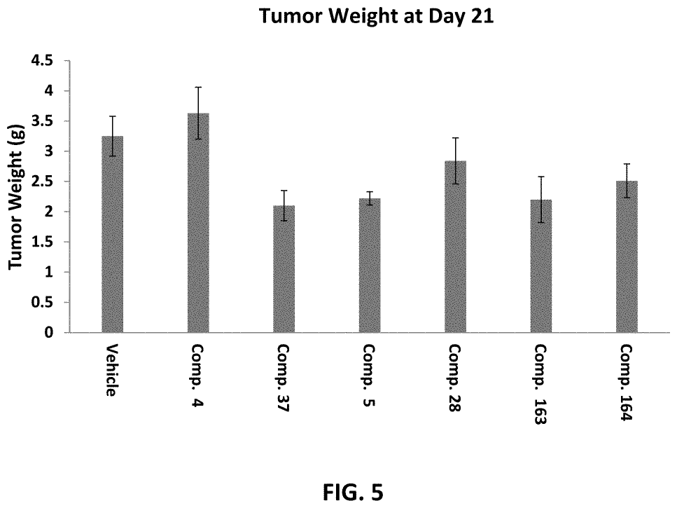

Tumor of each animal was collected and weighed at the end of the study. Ad shown in Table 12 and FIG. 5, animals treated with Comp. 37 (G3), Comp. 5 (G4), Comp. 163 (G6) and Comp. 164 (G7) had a lighter tumor but no statistic significance was seen in this study.

TABLE 12
Tumor Weight
Tumor weight (g)

| Group | Mean | SEM |
|---|---|---|
| G1 | 3.25 | 0.33 |
| Vehicle | | |
| G2 | 3.63 | 0.43 |
| Comp. 4 | | |
| G3 | 2.10 | 0.25 |
| Comp. 37 | | |
| G4 | 2.22 | 0.11 |
| Comp. 5 | | |
| G5 | 2.84 | 0.38 |
| Comp. 28 | | |
| G6 | 2.20 | 0.38 |
| Comp. 163 | | |
| G7 | 2.51 | 0.28 |
| Comp. 164 | | |

v. Tumor Growth Inhibition

Tumor growth inhibition (TGI %) for testing compounds as compared with the control vehicle are summarized in Table 13.

TABLE 13
Tumor Growth Inhibition (%)

| Group | D3 | D7 | D10 | D14 | D17 | D21 |
|---|---|---|---|---|---|---|
| G2 | 30.25 | 23.11 | −1.66 | 6.35 | 9.13 | 6.55 |
| Comp. 4 | | | | | | |
| G3 | 42.52 | 49.20 | 46.10 | 44.43 | 48.73 | 46.80 |
| Comp. 37 | | | | | | |
| G4 | 37.46 | 42.30 | 39.66 | 42.80 | 43.13 | 44.10 |
| Comp. 5 | | | | | | |
| G5 | 15.44 | 11.52 | 15.19 | 12.60 | 26.69 | 34.01 |
| Comp. 28 | | | | | | |
| G6 | 38.81 | 34.97 | 34.46 | 30.36 | 36.67 | 40.94 |
| Comp. 163 | | | | | | |
| G7 | 29.37 | 15.49 | 21.94 | 18.56 | 29.97 | 31.27 |
| Comp. 164 | | | | | | |

In this study, HCC1806 s.c. xenograft model was established in BALB/c nude mice to evaluate the efficacy of the testing compounds Comp. 4, Comp. 37, Comp. 5, Comp. 28, Comp. 163 and Comp. 164.

The HCC1806 xenograft grew well with vehicle treatment (Group 1) demonstrating that the HCC1806 xenograft model was successfully established in nude mice in this study. The mean tumor size of mice reached 2291.29 mm³ in Group 1, 2149.25 mm³ in Group 2, 1269.08 mm³ in Group 3, 1327.79 mm³ in Group 4, 1549.07 mm³ in Group 5, 1397.61 mm³ in Group 6 and 1610.30 mm³ in Group 7 on day 21 post treatment.

The testing compound Comp. 37, Comp. 5, Comp. 28, Comp. 163 and Comp. 164 showed anti-tumor response as compared with vehicle treatment with TGI % of 46.80%, 44.10%, 34.01%, 40.94% and 31.27% respectively in HCC1806 s.c. xenograft model. The testing compound Comp. 4 didn't showed significant anti-tumor response as compared with vehicle treatment with TGI of 6.55%.

The excellent in vivo antitumor potency exhibited by deuterated Comp. 37 (R²=—CD₃ in Formula (3), 46.8% reduction in tumor burden at day 21) is surprising as compared to that of Comp. 28 (R²=—CF₂D in Formula (3), 34.01% reduction in tumor burden at day 21) (See Table 13 above) in light of the fact that Comp. 37 have lower metabolic stability (shorter half-life t_{1/2}) in mouse liver microsome incubations than that of Comp. 28 (t_{1/2} mouse=182.0 min for Comp. 28 vs. t_{1/2} mouse=72 min for Comp. 37) (See Table 5 above in Example 38).

It is also surprisingly to find that deuterated Comp. 28 (R²=—CF₂D in Formula (3)) gave decreased in vivo antitumor potency (34.01% reduction in tumor burden at day 21) as compared to that of non-deuterated Comp. 5 (R²=—CF₂H), 44.1% reduction in tumor burden at day 21) (See Table 13 above) in light of the fact that Comp. 28 have higher metabolic stability (longer half-life t_{1/2}) in mouse liver microsome incubation than that of Comp. 5 (t_{1/2} mouse=182.0 min for Comp. 28 vs. t_{1/2} mouse=22.0 min for Comp. 5) (See Table 5 above in Example 38). The testing results for in vivo xenograft mouse tumor model as disclosed herein indicated that the impacts of the deuteration on the selective locations on the molecules of Formula (1)-(5) upon the in vivo antitumor efficacy is unpredictable. Further, the improvement in metabolic stability resulted from deuteration on the selective locations on the molecules of Formula (1)-(5) did not always cause enhancement to in vivo antitumor efficacy.

1. A compound of formula (1): 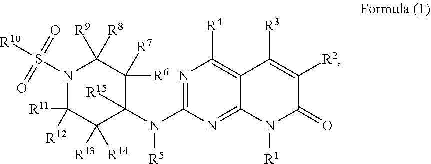
wherein,
R¹ is a cycloalkyl group having 5 to 6 carbon atoms optionally substituted with one or more substituents selected from the group of D, F, —OH, and C1-C4 alkyl group optionally substituted with one or more D, —OH, —CN, —C(═O)—NH₂, CH₃—(CH₂)ₙ—O—, and C1-C4 fluoroalkoxyl group,
R² is a substituent selected from the group of H, D, F, Cl, Br, C1-C4 alkyl group optionally substituted with one or more of D, —OH, —CN, Cl, —C(═O)—NH₂, CH₃—(CH₂)ₙ—O—, and C1-C4 fluoroalkoxyl group, or C1-C4 fluoroalkyl group optionally substituted with one or more D, —OH, —CN, —C(═O)—NH₂, CH₃—(CH₂)ₙ—O—, and C1-C4 fluoroalkoxyl group,
R³, R⁴, R⁵, R⁶, R⁷, R⁸, R⁹, R¹¹, R¹², R¹³, R¹⁴ and R¹⁵ are each independently hydrogen or D,
R¹⁰ is a substituent selected from the group of —NHR¹⁶; C1-C2 alkyl group optionally substituted with one or more of D, F, CH₃—(CH₂)ₙ—O—, C3-C5 cycloalkyl group, and C1-C2 fluoroalkoxyl group; C1-C2 fluoroalkyl group; cyclopropyl group; 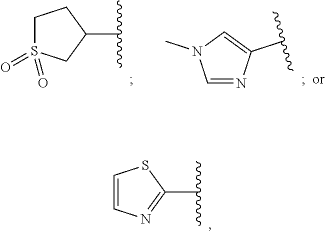
R¹⁶ is a substituent selected from the group of H, Me, C1-C3 fluoroalkoxyl group, 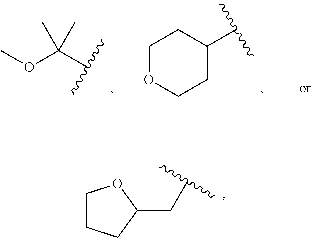
n is 0, 1, 2, or 3,
wherein the compound of Formula (1) is substituted with at least one deuterium atom, and
pharmaceutically acceptable salt, stereoisomer, cocrystal, prodrug, solvate, hydrate, or polymorph thereof.

2. The compound of claim 1, wherein R¹ in the Formula (1) is selected from the group of

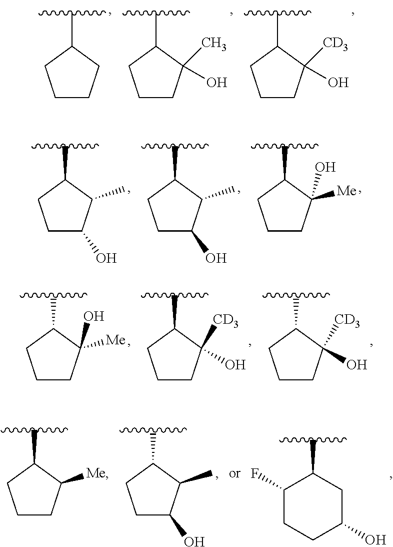

3. The compound of claim 1, wherein R¹ is

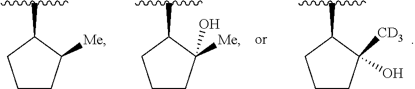 [SMILES: *[C@@H]1CCC[C@@H]1C ; *[C@@H]1CCC[C@@]1(C)O ; *[C@@H]1CCC[C@]1(O)C([2H])([2H])[2H]]

4. The compound of claim 1, wherein R¹ is

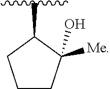 [SMILES: *[C@@H]1CCC[C@@]1(C)O]

5. The compound of claim 1, wherein R² in Formula (1) is selected from the group of H, D, F, Cl, CH₃—, CH₃—CH₂—, —CH₂—OH, —CH₂—CN, —CH₂—C(═O)NH₂, —CH₂—CH₂—OH, —CH₂—CH₂—OMe, —CF₂H, —CFH₂, —CF₃, —CH₂—CF₂H, —CFD₂, —CF₂D, —CH₂—CF₂D, —CD₂-CF₂H, —CD₂-CF₂D, or —CD₃.

6. The compound of claim 1, wherein R² in Formula (1) is selected from the group of CH₃—, CF₂H—, CF₂D-, or CD₃-.

7. The compound of claim 1, wherein R¹⁰ in Formula (1) is selected from the group of —NH₂, —NHMe, —CH₃, —CH₂F, —CD₃, ethyl, cyclopropyl, or —CH₂—CH₂—OMe.

8. The compound of claim 1, wherein R¹⁰ in Formula (1) is selected from the group of —CH₃ or —CD₃.

9.-11. (canceled)

12. The compound of claim 1, wherein R¹ is selected from the group of 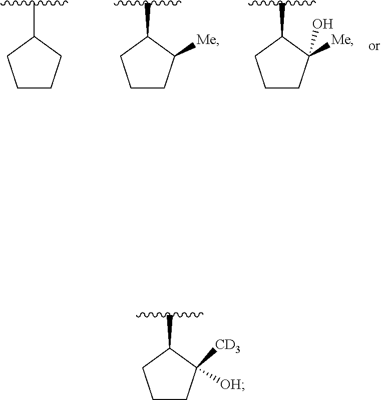 [SMILES: *C1CCCC1 ; *[C@@H]1CCC[C@@H]1C ; *[C@@H]1CCC[C@@]1(C)O ; *[C@@H]1CCC[C@]1(O)C([2H])([2H])[2H]]
R² is selected from the group of CH₃—, CF₂H—, CF₂D-, or CD₃-; and R¹⁰ is selected from the group of —NH₂, —NHMe, —CH₃, —CH₂F, —CD₃, ethyl, cyclopropyl, or —CH₂—CH₂—OMe.

13. The compound of claim 1, wherein R¹ is 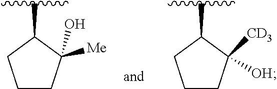 [SMILES: *[C@@H]1CCC[C@@]1(C)O ; *[C@@H]1CCC[C@]1(O)C([2H])([2H])[2H]]
R² is a substituent selected from the group of CH₃—, CF₂H—, CF₂D-, or CD₃-; and R¹⁰ is a substituent selected from CH₃—, or CD₃-.

14. The compound of claim 1, wherein R¹ is 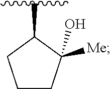 [SMILES: *[C@@H]1CCC[C@@]1(C)O]
R² is a substituent selected from the group of CH₃—, or CD₃-; and R¹⁰ is CH₃—.

15. The compound of claim 1, wherein the compound of Formula (1) is selected from any one of the deuterated pyridopyrimidinone Comps. 7-213 disclosed in Table 1.

16. A compound of Formula (2): 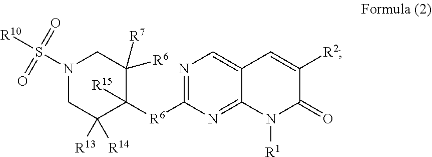
wherein,
R¹ is selected from the group of 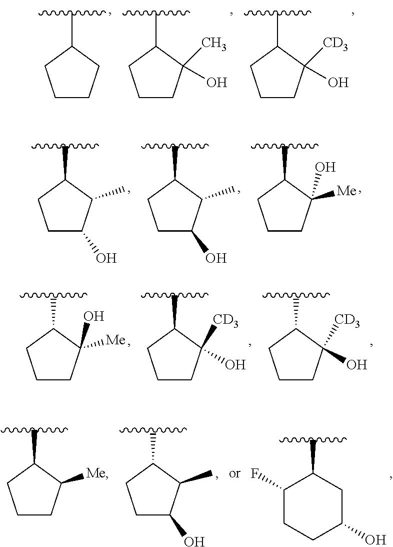
R² is a substituent selected from the group of H, D, F, Cl, Br, C1-C4 alkyl group optionally substituted with one or more D, —OH, —CN, Cl, —C(═O)—NH₂, CH₃—(CH₂)ₙ—O—, and C1-C4 fluoroalkoxyl group, or C1-C4 fluoroalkyl group optionally substituted with one or more D, —OH, —CN, —C(═O)—NH₂, CH₃—(CH₂)ₙ—O—, and C1-C4 fluoroalkoxyl group,
R⁶, R⁷, R¹³, R¹⁴ and R¹⁵ are each independently hydrogen or deuterium, and R⁶=R⁷=R¹³=R¹⁴;
R¹⁰ is a substituent selected from the group of —NHR¹⁶; C1-C2 alkyl group optionally substituted with one or more of D, F, CH₃—(CH₂)ₙ—O—, C3-C5 cycloalkyl group, and C1-C2 fluoroalkoxyl group; C1-C2 fluoroalkyl group; cyclopropyl group; 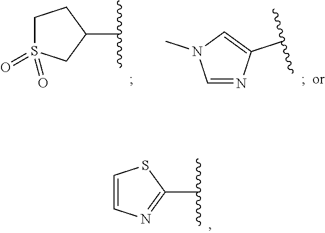
R¹⁶ is a substituent selected from the group of H, Me, C1-C3 fluoroalkoxyl group, 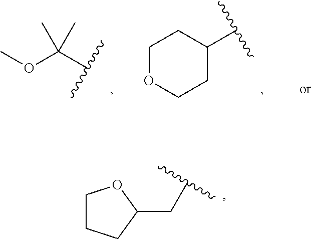
n is 0, 1, 2, or 3,
wherein the compound of Formula (2) is substituted with at least one deuterium atom, and
pharmaceutically acceptable salt, stereoisomer, cocrystal, prodrug, solvate, hydrate, or polymorph thereof.

17. The compound according to claim 16, wherein R¹ is

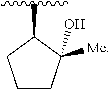 [SMILES: *[C@@H]1CCC[C@@]1(C)O]

18. The compound according to claim 16, wherein R² is selected from the group of CH₃—, CF₂H—, CF₂D-, or CD₃-.

19. The compound according to claim 16, wherein R¹⁰ is a substituent selected from the group of —CH₃ or —CD₃.

20.-47. (canceled)

48. The compound of claim 1, wherein the compound of any one of Formulae (1)-(3) is selected from the group of:
(±)-8-(2-hydroxy-2-methylcyclopentyl)-6-(methyl-d3)-2-((1-(methylsulfonyl)piperidin-4-yl)amino)pyrido[2,3-d]pyrimidin-7(8H)-one,
(±)-6-(difluoromethyl-d)-8-(2-hydroxy-2-methylcyclopentyl)-2-((1-(methylsulfonyl)piperidin-4-yl)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-yl)amino)pyrido[2,3-d]pyrimidin-7(8H)-one,
(−)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one,
(−)-8-((1R,2R)-2-hydroxy-2-methylcyclopentyl)-6-methyl-2-((1-(methylsulfonyl)piperidin-4-yl-3,3,5,5-d4)amino)pyrido[2,3-d]pyrimidin-7(8H)-one,
(−)-8-((1R,2R)-2-hydroxy-2-methylcyclopentyl)-6-methyl-2-((1-((methyl-d3)sulfonyl)piperidin-4-yl)amino)pyrido[2,3-d]pyrimidin-7(8H)-one,
(−)-8-((1R,2R)-2-hydroxy-2-methylcyclopentyl)-6-methyl-2-((1-(methylsulfonyl)piperidin-4-yl-4-d)amino)pyrido[2,3-d]pyrimidin-7(8H)-one,
(−)-8-((1R,2R)-2-hydroxy-2-methylcyclopentyl)-6-(methyl-d3)-2-((1-((methyl-d3)sulfonyl)piperidin-4-yl-3,3,4,5,5-d5)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-yl-3,3,4,5,5-d5)amino)pyrido[2,3-d]pyrimidin-7(8H)-one,
(−)-8-((1R,2R)-2-hydroxy-2-methylcyclopentyl)-6-(methyl-d3)-2-((1-(methylsulfonyl)piperidin-4-d-4-yl)amino)pyrido[2,3-d]pyrimidin-7(8H)-one,
(−)-6-(difluoromethyl-d)-8-((1R,2R)-2-hydroxy-2-methylcyclopentyl)-2-((1-(methylsulfonyl)piperidin-4-yl)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl)amino)pyrido[2,3-d]pyrimidin-7(8H)-one,
(−)-6-(difluoromethyl)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-6-(difluoromethyl-d)-8-((1R,2R)-2-hydroxy-2-methylcyclopentyl)-2-((1-(methylsulfonyl)piperidin-4-yl-4-d)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl-4-d)amino)pyrido[2,3-d]pyrimidin-7(8H)-one,
(−)-6-(difluoromethyl)-8-((1R,2R)-2-hydroxy-2-methylcyclopentyl)-2-((1-methylsulfonyl)piperidin-4-yl-3,3,5,5-d4)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl-3,3,5,5-d4)amino)pyrido[2,3-d]pyrimidin-7(8H)-one,
(−)-6-(difluoromethyl-d)-8-((1R,2R)-2-hydroxy-2-methylcyclopentyl)-2-((1-(methylsulfonyl)piperidin-4-yl-3,3,5,5-d4)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl-3,3,5,5-d4)amino)pyrido[2,3-d]pyrimidin-7(8H)-one,
(−)-6-(difluoromethyl)-8-((1R,2R)-2-hydroxy-2-methylcyclopentyl)-2-((1-((methyl-d3)sulfonyl)piperidin-4-yl-3,3,4,5,5-d5)amino)pyrido[2,3-d]pyrimidin-7(8H)-one,
(−)-6-(difluoromethyl-d)-8-((1R,2R)-2-hydroxy-2-methylcyclopentyl)-2-((1-((methyl-d3)sulfonyl)piperidin-4-yl-3,3,4,5,5-d5)amino)pyrido[2,3-d]pyrimidin-7(8H)-one,
(+)-8-((1S,2S)-2-hydroxy-2-methylcyclopentyl)-6-(methyl-d3)-2-((1-(methylsulfonyl)piperidin-4-yl)amino)pyrido[2,3-d]pyrimidin-7(8H)-one,
(+)-6-(difluoromethyl-d)-8-((1S,2S)-2-hydroxy-2-methylcyclopentyl)-2-((1-(methylsulfonyl)piperidin-4-yl)amino)pyrido[2,3-d]pyrimidin-7(8H)-one, and
pharmaceutically acceptable salt, stereoisomer, crystal, prodrug, solvate, and polymorph thereof.

49. A pharmaceutical composition comprising the compound of claim 1 and at least one pharmaceutically acceptable carrier or diluent.

50.-55. (canceled)

56. A method for treating a cancer in a subject in need thereof comprising administering to the subject a therapeutically effective amount of the compound of claim 1.

57. The method of claim 56, wherein the cancer is selected from breast cancer, triple negative breast cancer, ovarian cancer, neuroblastoma, glioblastoma, B-cell lymphoma, prostate cancer, liver cancer, acute myeloid leukemia, or melanoma.

58.-70. (canceled)
